(19) United States
(12) Patent Application Publication
(10) Pub. No.: US 2025/0084402 A1
(43) Pub. Date: Mar. 13, 2025

(54) METHODS OF PREPARING LIBRARIES FOR SEQUENCING AND METHODS OF ANALYSIS

(71) Applicant: Illumina, Inc., San Diego, CA (US)

(72) Inventors: Eli CARRAMI, Cambridge (GB); Jonathan BOUTELL, Cambridge (GB); Oliver MILLER, Cambridge (GB); Aathavan KARUNAKARAN, San Diego, CA (US); Stephen BRUINSMA, San Diego, CA (US); Niall GORMLEY, Cambridge (GB); Gery VESSERE, San Diego, CA (US); Roberto ANDRES, Cambridge (GB); Michael BUREK, San Diego, CA (US); Shagesh SRIDHARAN, San Diego, CA (US); Nileshi SARAF, San Diego, CA (US); Come RACZY, San Diego, CA (US); Jeff GAU, San Diego, CA (US)

(21) Appl. No.: 18/885,319

(22) Filed: Sep. 13, 2024

Related U.S. Application Data
(63) Continuation-in-part of application No. PCT/EP2023/056641, filed on Mar. 15, 2023.
(63) Continuation-in-part of application No. PCT/EP2023/056634, filed on Mar. 15, 2023.
(63) Continuation-in-part of application No. PCT/EP2023/056656, filed on Mar. 15, 2023.
(63) Continuation-in-part of application No. PCT/EP2023/056669, filed on Mar. 15, 2023.
(63) Continuation-in-part of application No. PCT/EP2023/056626, filed on Mar. 15, 2023.
(63) Continuation-in-part of application No. PCT/EP2023/056653, filed on Mar. 15, 2023.
(63) Continuation-in-part of application No. PCT/EP2023/056672, filed on Mar. 15, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.
(60) Provisional application No. 63/439,519, filed on Jan. 17, 2023.
(60) Provisional application No. 63/269,383, filed on Mar. 15, 2022.
(60) Provisional application No. 63/439,417, filed on Jan. 17, 2023.
(60) Provisional application No. 63/439,438, filed on Jan. 17, 2023.
(60) Provisional application No. 63/439,415, filed on Jan. 17, 2023.
(60) Provisional application No. 63/439,466, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/439,501, filed on Jan. 17, 2023.
(60) Provisional application No. 63/439,443, filed on Jan. 17, 2023.

(51) Int. Cl. C12N 15/10; C12Q 1/6874; G16B 30/10

(52) U.S. Cl. CPC C12N 15/1068; C12Q 1/6874; G16B 30/10

(57) ABSTRACT

The invention relates to methods and kits for use in nucleic acid sequencing, in particular methods for use in concurrent sequencing, including concurrent sequencing of tandem insert libraries. Further, the invention relates to methods of detecting mismatched base pairs in nucleic acid sequences. In another embodiment, the disclosed technology relates to using next generation sequencing to determine the nucleotide sequences of two or more polynucleotide sequence portions in a single sequencing run.

### CROSS REFERENCE TO RELATED APPLICATIONS

This application is a continuation-in-part application of PCT Application No. PCT/EP2023/056641 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

This application is also a continuation-in-part application of PCT Application No/PCT/EP23/56634 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

This application is also a continuation-in-part application of PCT/EP23/56656 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

This application is also a continuation-in-part application of PCT/EP23/56669 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

This application is also a continuation-in-part application of PCT/EP23/56626 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

This application is also a continuation-in-part application of PCT/EP23/56653 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

This application is also a continuation-in-part application of PCT/EP23/56672 filed on Mar. 15, 2023, which claims priority to U.S. Provisional Application No. 63/439,519 filed on Jan. 17, 2023, U.S. Provisional Application No. 63/269,383 filed on Mar. 15, 2022, U.S. Provisional Application No. 63/439,415 filed on Jan. 17, 2023, U.S. Provisional Application No. 63/439,417 filed on Jan. 17, 2023, U.S. Provisional Application No. 63/439,438 filed on Jan. 17, 2023, U.S. Provisional Application No. 63/439,443 filed on Jan. 17, 2023, U.S. Provisional Application No. 63/439,466 filed on Jan. 17, 2023, U.S. Provisional Application No. 63/439,491 filed on Jan. 17, 2023, U.S. Provisional Application No. 63/439,501 filed on Jan. 17, 2023 and U.S. Provisional Application No. 63/439,522 filed on Jan. 17, 2023.

The contents of each of the aforementioned applications are incorporated by reference in their entireties.

### SEQUENCE LISTING

The present application is being filed along with a Sequence Listing in electronic format. The Sequence Listing is provided as a file entitled PC932615US.xml, created and last saved on Sep. 13, 2024, which is 82,879 bytes in size. The information in the electronic format of the Sequence Listing is hereby incorporated by reference in its entirety.

### FIELD OF THE INVENTION

The invention relates to methods and kits for use in nucleic acid sequencing, in particular methods for use in concurrent sequencing, including concurrent sequencing of tandem insert libraries. Further, the invention relates to methods of detecting mismatched base pairs in nucleic acid sequences. In another embodiment, the disclosed technology relates to using next generation sequencing to determine the nucleotide sequences of two or more polynucleotide sequence portions in a single sequencing run.

### BACKGROUND

The common expectation is that the complementary sequences of a double-stranded DNA molecule should carry identical information, and as such, sequencing one strand of the molecule should be sufficient to determine the sequence. In practice, however, this notion is not accurate.

The most common occasion where the symmetry of information between complementary strands may break is due to DNA damage. Different bases of DNA have different susceptibilities to different forms of damage. For instance, G is very sensitive to oxidative damage leading to the formation of oxo-G, the formation of which is one of the main reasons of library prep dependent sequencing errors, as DNA polymerases often unfaithfully pair oxo-G with A, leading to high quality C>A sequencing errors. This results in the creation of mismatched base pairs. Another situation in which the symmetry of information between the strands may break is during methyl-C(mC) sequencing. Standard protocols modify C or mC to alternative bases such as U, thereby changing the sequence information only in one strand.

Various strategies have been proposed to enable the sequencing of both strands of a double-stranded DNA molecule, commonly known as duplex sequencing.

Original methods of duplex sequencing used bioinformatics methods or high-depth sequencing data to identify clusters corresponding to each of the strands in original template DNA molecules and used this information to correct potential sequencing errors. Other methods used physical separation or UMI index sequences to discriminately label strands of DNA that originate from the same double-stranded template. Naturally, such methods are either very complex or are inefficient at identifying the correct duplex molecules.

Recently, a more efficient strategy for generating duplex sequencing information for the purpose of sequencing error correction was proposed. This method generates tandem insert libraries containing the sequence information from each strand of a double-stranded template in a direct repeat fashion. The direct repeat format of this library is essential for its functionality as it avoids the rehybridization of the sequencing template during sequencing by synthesis (SBS). This method, while compatible with SBS, suffers from very low conversion efficiency during library preparation.

There therefore exists a need to develop improved methods that can sequence both strands of a double-stranded DNA molecule (duplex sequencing), and in particular a need for methods that are compatible with SBS; and there remains a need to develop more accurate nucleic acid sequencing methods. Identifying such mismatched base pairs would allow such sequencing errors to be identified.

### SUMMARY OF THE INVENTION

According to an aspect of the present invention, there is provided a method of preparing at least one polynucleotide library strand template, wherein the method comprises:
attaching a first adaptor to a first end of a double-stranded polynucleotide sequence, wherein the first end comprises 3′ end of the forward strand and 5′ end of the reverse strand of the double-stranded polynucleotide sequence; and
attaching a second adaptor to a second end of a double-stranded polynucleotide sequence, wherein the second end comprises 5′ end of the forward strand and 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease and/or the second adaptor further comprises at least one cleavable site and/or a complement of a cleavable site.

In one embodiment, the first adaptor comprises a base-paired stem and a loop, wherein the first restriction site is in the base-paired stem. Alternatively or additionally, the first restriction site is in the loop.

In one embodiment, the first restriction site is a restriction site for a nicking endonuclease or a restriction endonuclease.

In one embodiment, the second adaptor further comprises at least one cleavable site and/or a complement of a cleavable site. In one example, the second adaptor comprises a base-paired stem and a fork, wherein the fork comprises a primer-binding complement sequence and a primer-binding sequence. In one embodiment, the cleavable site and/or a complement of a cleavable site is in the base-paired stem. In an alternative embodiment, the second adaptor comprises a base-paired stem and a loop, wherein the loop comprises a second cleavable site.

In one embodiment, the at least one cleavable site and/or a complement of a cleavable site is a restriction site for a nicking endonuclease, wherein the restriction site may be a second restriction site.

In one embodiment, the first adaptor further comprises an affinity tag.

In another aspect of the invention there is provided a polynucleotide library strand for sequencing comprising a first adaptor, a double-stranded polynucleotide sequence to be identified and a second adaptor;
wherein the first adaptor is attached to a first end of the double-stranded polynucleotide sequence, wherein the first end comprises 3′ end of the forward strand and 5′ end of the reverse strand of the double-stranded polynucleotide sequence; and wherein the second adaptor is attached to a second end of the double-stranded polynucleotide sequence, wherein the second end comprises 5′ end of the forward strand and 3′ end of the reverse strand of the double-stranded polynucleotide sequence;
wherein the first adaptor comprises a base-paired stem and a loop; and
wherein the second adaptor comprises a base-paired stem, a primer-binding complement sequence and a primer-binding sequence; and
wherein the first adaptor comprises at least one restriction site for an endonuclease.

In one embodiment, the second adaptor comprises at least one cleavable site and/or a complement of a cleavable site, wherein the cleavable site and/or a complement of a cleavable site may be a restriction site for a nicking endonuclease.

In another aspect of the invention, there is provided a method of identifying at least a first region of a polynucleotide sequence, wherein the method comprises:
a. preparing at least one polynucleotide library strand as described above;
b. amplifying the polynucleotide library strand to generate a first and second library strand, wherein each library strand comprises a first and second region;
c. hybridising the first or second library strands to first and second immobilised primers respectively on a solid support and carrying out a first extension reaction to generate a first or second immobilised template strand;
d. hybridising the first or second immobilised template strands to a second or first immobilised primer respectively and carrying out a second extension reaction to generate a second and first immobilised template strand;
e. hybridising the first and second immobilised template strands;
f. applying a first endonuclease; and
g. sequencing the first and second immobilised template strands, wherein sequencing the first and second immobilised template strands identifies the first region.

In one embodiment, identifying comprises determining the sequences of a first region and/or identifying any epigenetic modification, wherein the epigenetic modification may be a modified cytosine.

In one embodiment, each first and second library strands comprise a primer-binding complement sequence, a first portion, a first adaptor sequence, a second portion and a primer-binding sequence, and wherein the first adaptor comprises a first restriction site for an endonuclease.

In one embodiment, the first restriction site is a restriction site for a nicking endonuclease or a restriction endonuclease.

In one embodiment, the primer-binding sequence and primer-binding complement sequence comprise at least one cleavable and/or a complement of a cleavable site. In one embodiment, the cleavable site and/or a complement of a cleavable site is a second restriction site.

In one embodiment, following cleavage of the first restriction site, non-immobilised library strands are de-hybridised and the immobilised template strands are sequenced by single-stranded SBS (sequencing by synthesis). Alternatively, following cleavage of the first restriction site, the immobilised template strands are sequenced by double-stranded SBS (sequencing by synthesis).

In one embodiment, the at least one nicking endonuclease cleaves the second restriction site and the immobilised strands are sequenced by double-stranded SBS (sequencing by synthesis).

In one embodiment, the method further comprises blocking all or substantially all 3′ ends of the sequenced immobilised strands.

In one embodiment, the method further comprises applying a second nicking endonuclease and sequencing the first and second immobilised template strands identifies the second region, wherein the second nicking endonuclease cleaves a different restriction site from the first nicking endonuclease.

In one embodiment, the method further comprises carrying out an extension reaction to regenerate the first and second immobilised strands.

In one embodiment, the method further comprises applying a second nicking endonuclease and sequencing the first and second immobilised template strands identifies the second region, wherein the second nicking endonuclease cleaves a different restriction site from the first nicking endonuclease.

In another aspect of the invention there is provided an inverted-repeat tandem-insert polynucleotide library strand for sequencing, wherein the library strand comprises a primer-binding complement sequence, a first portion to be identified, a first adaptor sequence, a second portion to be identified and a primer-binding sequence, wherein the sequence of the second portion is inverted with respect to the first portion, and wherein the loop sequence comprises at least one restriction site.

In another aspect of the invention there is provided a library preparation kit comprising of a plurality of first adaptors and a plurality of second adaptors, wherein the first adaptors comprise a base-paired stem and a loop, and wherein the first adaptors comprise at least one restriction site, and wherein the second adaptors comprise a base-paired stem, a primer-binding sequence and a primer-binding complement sequence, wherein optionally the second adaptors comprise at least one restriction site.

According to an aspect of the present invention, there is provided a method of preparing polynucleotide sequences for detection of mismatched base pairs, comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template.

In one embodiment, the forward strand of the template is not identical to the reverse complement strand of the template.

In one aspect, the method further comprises a step of preparing the first portion and the second portion for concurrent sequencing.

In one embodiment, the method comprises simultaneously contacting first sequencing primer binding sites located after a 3′-end of the first portions with first primers and second sequencing primer binding sites located after a 3′-end of the second portions with second primers.

In one example, the method comprises nicking the at least one first polynucleotide sequence and nicking the at least one second polynucleotide sequence.

In one embodiment, a proportion of first portions is capable of generating a first signal and a proportion of second portions is capable of generating a second signal, wherein an intensity of the first signal is substantially the same as an intensity of the second signal.

In another embodiment, the method further comprises a step of selectively processing the at least one first polynucleotide sequence comprising a first portion and the at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

In one embodiment, a concentration of the first portions capable of generating the first signal is greater than a concentration of the second portions capable of generating the second signal.

In one embodiment, a ratio between the concentration of the first portions capable of generating the first signal and the concentration of the second portions capable of generating the second signal is between 1.25:1 to 5:1, or between 1.5:1 to 3:1, or about 2:1.

In one embodiment, selective processing comprises preparing for selective sequencing or conducting selective sequencing.

In another embodiment, selectively processing comprises conducting selective amplification.

In one embodiment, selectively processing comprises contacting first sequencing primer binding sites located after a 3′-end of the first portions with first primers and contacting second sequencing primer binding sites located after a 3′-end of the second portions with second primers, wherein the second primers comprises a mixture of blocked second primers and unblocked second primers.

In one embodiment, the blocked second primer comprises a blocking group at a 3′ end of the blocked second primer.

In one example, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, the selective processing comprises selectively removing some or substantially all of second immobilised primers that are not yet extended, and conducting a further amplification cycle in order to selectively amplify the first polynucleotide sequence(s) relative to the second polynucleotide sequence(s).

In another embodiment, selectively processing comprises selectively blocking some or substantially all of second immobilised primers that are not yet extended using a primer blocking agent, wherein the primer blocking agent is configured to limit or prevent synthesis of a strand extending from the second immobilised primer, and conducting a further amplification cycle in order to selectively amplify the first polynucleotide sequence(s) relative to the second polynucleotide sequence(s).

In one aspect, the primer blocking agent is added whilst first polynucleotide sequence(s) are hybridised to the second immobilised primers.

In one embodiment, the method comprises contacting some or substantially all of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to 5′ additional nucleotide.

In one embodiment, the primer blocking agent is a blocked nucleotide. In one example, the blocked nucleotide comprises a blocking group at a 3′ end of the blocked nucleotide.

In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, the blocked nucleotide is A or G. In one aspect, the first signal and the second signal are spatially unresolved.

In one embodiment, the at least one first polynucleotide sequence comprising the first portion and the at least one second polynucleotide sequence comprising the second portion are attached to a solid support, wherein the solid support may be a flow cell.

In another embodiment, the at least one first polynucleotide sequence comprising the first portion and the at least one second polynucleotide sequence comprising the second portion forms a cluster on the solid support.

In one embodiment, the cluster is formed by bridge amplification.

In one embodiment, the at least one first polynucleotide sequence comprising the first portion and the at least one second polynucleotide sequence comprising the second portion form a duoclonal cluster.

In one aspect, the solid support comprises at least one first immobilised primer and at least one second immobilised primer.

In one embodiment, the first immobilised primer comprises a sequence as defined in SEQ ID NO. 1 or 5, or a variant or fragment thereof; and the second immobilised primer comprises a sequence as defined in SEQ ID NO. 2, or a variant or fragment thereof.

In one embodiment, each first polynucleotide sequence is attached to a first immobilised primer, and wherein each second polynucleotide sequence is attached a second immobilised primer.

In one embodiment, each first polynucleotide sequence comprises a second adaptor sequence and wherein each second polynucleotide sequence comprises a first adaptor sequence, wherein the second adaptor sequence is substantially complementary to the second immobilised primer and wherein the first adaptor sequence is substantially complementary to the first immobilised primer.

In one embodiment, the step of synthesising at least one first polynucleotide sequence comprising a first portion and at least one second polynucleotide sequence comprising a second portion comprises:
synthesising a loop-ligated precursor polynucleotide by connecting a 3′-end of the forward strand of the target polynucleotide and a 5′-end of the reverse strand of the target polynucleotide with a loop, or connecting a 5′-end of the forward strand of the target polynucleotide and a 3′-end of the reverse strand of the target polynucleotide with a loop,
synthesising the at least one first polynucleotide sequence comprising the first portion by forming a complement of the loop-ligated precursor polynucleotide, and
synthesising the at least one second polynucleotide sequence comprising the at least one second polynucleotide sequence by forming a complement of the at least one first polynucleotide sequence.

In one embodiment, the method further comprises concurrently sequencing nucleobases in the first portion and the second portion.

In one embodiment, the first portion is at least 25 base pairs and the second portion is at least 25 base pairs.

According to another aspect of the present invention, there is provided a method of sequencing polynucleotide sequences to detect mismatched base pairs, comprising:
preparing polynucleotide sequences for detection of mismatched base pairs using a method as described herein;
concurrently sequencing nucleobases in the first portion and the second portion; and
identifying mismatched base pairs by detecting differences when comparing a sequence output from the first portion with a sequence output from the second portion.

In one embodiment, the step of concurrently sequencing nucleobases comprises performing sequencing-by-synthesis or sequencing-by-ligation.

In one example, the step of preparing the polynucleotide sequences comprises using a method as described herein; and wherein the step of concurrent sequencing nucleobases in the first portion and the second portion is based on the intensity of the first signal and the intensity of the second signal.

In one embodiment, the mismatched base pair comprises an oxo-G to A base pair.

In one embodiment, the method further comprises a step of conducting paired-end reads.

In one embodiment, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

In one embodiment, selecting the classification based on the first and second intensity data comprises selecting the classification based on the combined intensity of the first and second signal components and the combined intensity of the third and fourth signal components.

In one embodiment, the plurality of classifications comprises sixteen classifications, each classification representing one of sixteen unique combinations of first and second nucleobases.

In one embodiment, the first signal component, second signal component, third signal component and fourth signal component are generated based on light emissions associated with the respective nucleobase.

In one embodiment the light emissions are detected by a sensor, wherein the sensor is configured to provide a single output based upon the first and second signals.

In one example, the sensor comprises a single sensing element.

In one embodiment, the method further comprises repeating steps (a) to (d) for each of a plurality of base calling cycles.

In one embodiment, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the first portion and the second portion.

In one embodiment, selecting the classification based on the first and second intensity data comprises selecting the classification based on the combined intensity of the first and second signal components and the combined intensity of the third and fourth signal components.

In one embodiment, when based on a nucleobase of the same identity, an intensity of the first signal component is substantially the same as an intensity of the second signal component and an intensity of the third signal component is substantially the same as an intensity of the fourth signal component.

In one embodiment, the plurality of classifications consists of a predetermined number of classifications.

In one aspect, the plurality of classifications comprises:
one or more classifications representing matching first and second nucleobases; and
one or more classifications representing mismatching first and second nucleobases, and
wherein determining sequence information of the first portion and second portion comprises:
in response to selecting a classification representing matching first and second nucleobases, determining a match between the first and second nucleobases; or
in response to selecting a classification representing mismatching first and second nucleobases, determining a mismatch between the first and second nucleobases.

In one embodiment, determining sequence information of the first portion and the second portion comprises, in response to selecting a classification representing a match between the first and second nucleobases, base calling the first and second nucleobases.

In another embodiment, determining sequence information of the first portion and the second portion comprises, based on the selected classification, determining that the second portion is modified relative to the first portion at a location associated with the first and second nucleobases.

In one aspect, the first signal component, second signal component, third signal component and fourth signal component are generated based on light emissions associated with the respective nucleobase.

In one embodiment, the light emissions are detected by a sensor, wherein the sensor is configured to provide a single output based upon the first and second signals.

In one example, the sensor comprises a single sensing element.

In one embodiment, the method further comprises repeating steps (a) to (d) for each of a plurality of base calling cycles.

According to another aspect of the present invention, there is provided a kit comprising instructions for preparing polynucleotide sequences for detection of mismatched base pairs as described herein, and/or for sequencing polynucleotide sequences to detect mismatched base pairs as described herein.

According to another aspect of the present invention, there is provided a data processing device comprising means for carrying out a method as described herein.

In one embodiment, the data processing device is a polynucleotide sequencer.

According to another aspect of the present invention, there is provided a computer program product comprising instructions which, when the program is executed by a processor, cause the processor to carry out a method as described herein.

According to another aspect of the present invention, there is provided a computer-readable storage medium comprising instructions which, when executed by a processor, cause the processor to carry out a method as described herein.

According to another aspect of the present invention, there is provided a computer-readable data carrier having stored thereon a computer program product as described herein.

According to another aspect of the present invention, there is provided a data carrier signal carrying a computer program product as described herein.

According to an aspect of the present invention, there is provided a method of preparing at least one polynucleotide sequence for detection of mismatched base pairs, comprising:
synthesising at least one polynucleotide sequence comprising a first portion and a second portion,
wherein the at least one polynucleotide sequence comprises portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template.

In one embodiment, the forward strand of the template is not identical to the reverse complement strand of the template.

In one embodiment, the method further comprises a step of preparing the first portion and the second portion for concurrent sequencing.

In one embodiment, the method comprises simultaneously contacting first sequencing primer binding sites located after a 3′-end of the first portions with first primers and second sequencing primer binding sites located after a 3′-end of the second portions with second primers.

In one embodiment, a proportion of first portions is capable of generating a first signal and a proportion of second portions is capable of generating a second signal, wherein an intensity of the first signal is substantially the same as an intensity of the second signal.

In one embodiment, the method further comprises a step of selectively processing the at least one polynucleotide sequence comprising the first portion and the second portion, such that a proportion of first portions are capable of generating a first signal and a proportion of second portions are capable of generating a second signal, wherein the selective processing causes an intensity of the first signal to be greater than an intensity of the second signal.

In one embodiment, a concentration of the first portions capable of generating the first signal is greater than a concentration of the second portions capable of generating the second signal.

In one embodiment, a ratio between the concentration of the first portions capable of generating the first signal and the concentration of the second portions capable of generating the second signal is between 1.25:1 to 5:1, or between 1.5:1 to 3:1, or about 2:1.

In one embodiment, selective processing comprises preparing for selective sequencing or conducting selective sequencing.

In one embodiment, selectively processing comprises contacting first sequencing primer binding sites located after a 3′-end of the first portions with first primers and contacting second sequencing primer binding sites located after a 3′-end of the second portions with second primers, wherein the second primers comprises a mixture of blocked second primers and unblocked second primers.

In one embodiment, the blocked second primer comprises a blocking group at a 3′ end of the blocked second primer.

In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, the blocked second primer comprises a sequence as defined in SEQ ID NO. 31-36 or a variant or fragment thereof and/or the unblocked second primer comprises a sequence as defined in SEQ ID NO. 31-34 or a variant or fragment thereof.

In one embodiment, the first signal and the second signal are spatially unresolved.

In one embodiment, the at least one polynucleotide sequence comprising the first portion and the second portion is/are attached to a solid support, wherein the solid support may be a flow cell.

In one embodiment, the at least one polynucleotide sequence comprising the first portion and the second portion forms a cluster on the solid support.

In one embodiment, the cluster is formed by bridge amplification.

In one embodiment, the at least one polynucleotide sequence comprising the first portion and the second portion forms a monoclonal cluster.

In one embodiment, the solid support comprises at least one first immobilised primer and at least one second immobilised primer.

In one embodiment, the first immobilised primer comprises a sequence as defined in SEQ ID NO. 1 or 5, or a variant or fragment thereof; and the second immobilised primer comprises a sequence as defined in SEQ ID NO. 2, or a variant or fragment thereof.

In one embodiment, each polynucleotide sequence comprising the first portion and the second portion is attached to a first immobilised primer.

In one embodiment, each polynucleotide sequence comprising the first portion and the second portion further comprises a second adaptor sequence, wherein the second adaptor sequence is substantially complementary to the second immobilised primer.

In one embodiment, the step of synthesising the at least one polynucleotide sequence comprising a first portion and a second portion comprises:
synthesising a first precursor polynucleotide fragment comprising a complement of the first portion and a hybridisation complement sequence,
synthesising a second precursor polynucleotide fragment comprising a second portion and a hybridisation sequence,
annealing the hybridisation complement sequence of the first precursor polynucleotide fragment with the hybridisation sequence on the second precursor polynucleotide fragment to form a hybridised adduct,
synthesising a first precursor polynucleotide sequence by extending the first precursor polynucleotide fragment to form a complement of the second portion, and
synthesising the at least one polynucleotide sequence by forming a complement of the first precursor polynucleotide sequence.

In one embodiment, the first precursor polynucleotide fragment comprises a first sequencing primer binding site complement.

In one embodiment, the first sequencing primer binding site complement is located before a 5′-end of the complement of the first portion, such as immediately before 5′end of the complement of the first portion.

In one embodiment, the first precursor polynucleotide fragment comprises a second adaptor complement sequence.

In one embodiment, the second adaptor complement sequence is located before a 5′-end of the complement of the first portion.

In one embodiment, the first precursor polynucleotide fragment comprises a first sequencing primer binding site complement and a second adaptor complement sequence.

In one embodiment, the first sequencing primer binding site complement is located before a 5′-end of the complement of the first portion, and wherein the second adaptor complement sequence is located before a 5′-end of the first sequencing primer binding site complement.

In one embodiment, the first precursor polynucleotide fragment comprises a second sequencing primer binding site complement.

In one embodiment, the hybridisation sequence complement comprises the second sequencing primer binding site complement.

In one embodiment, the second precursor polynucleotide fragment comprises a first adaptor complement sequence.

In one embodiment, the method further comprises concurrently sequencing nucleobases in the first portion and the second portion.

In one embodiment, the first portion is at least 25 base pairs and the second portion is at least 25 base pairs.

According to another aspect of the present invention, there is provided a method of sequencing at least one polynucleotide sequence to detect mismatched base pairs, comprising:
preparing at least one polynucleotide sequence for detection of mismatched base pairs using a method as described herein;
concurrently sequencing nucleobases in the first portion and the second portion; and
identifying mismatched base pairs by detecting differences when comparing a sequence output from the first portion with a sequence output from the second portion.

In one embodiment, the step of concurrently sequencing nucleobases comprises performing sequencing-by-synthesis or sequencing-by-ligation.

In one embodiment, the step of preparing the at least one polynucleotide sequence comprises using a method as described herein; and wherein the step of concurrent sequencing nucleobases in the first portion and the second portion is based on the intensity of the first signal and the intensity of the second signal.

In one embodiment, the mismatched base pair comprises an oxo-G to A base pair.

In one embodiment, the method further comprises a step of conducting paired-end reads.

In one embodiment, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

In one embodiment, selecting the classification based on the first and second intensity data comprises selecting the classification based on the combined intensity of the first and second signal components and the combined intensity of the third and fourth signal components.

In one embodiment, the plurality of classifications comprises sixteen classifications, each classification representing one of sixteen unique combinations of first and second nucleobases.

In one embodiment, the first signal component, second signal component, third signal component and fourth signal component are generated based on light emissions associated with the respective nucleobase.

In one embodiment, the light emissions are detected by a sensor, wherein the sensor is configured to provide a single output based upon the first and second signals.

In one embodiment, the sensor comprises a single sensing element.

In one embodiment, the method further comprises repeating steps (a) to (d) for each of a plurality of base calling cycles.

In one embodiment, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the first portion and the second portion.

In one embodiment, selecting the classification based on the first and second intensity data comprises selecting the classification based on the combined intensity of the first and second signal components and the combined intensity of the third and fourth signal components.

In one embodiment, when based on a nucleobase of the same identity, an intensity of the first signal component is substantially the same as an intensity of the second signal component and an intensity of the third signal component is substantially the same as an intensity of the fourth signal component.

In one embodiment, the plurality of classifications consists of a predetermined number of classifications.

In one embodiment, the plurality of classifications comprises:
one or more classifications representing matching first and second nucleobases; and
one or more classifications representing mismatching first and second nucleobases, and
wherein determining sequence information of the first portion and second portion comprises:
   in response to selecting a classification representing matching first and second nucleobases, determining a match between the first and second nucleobases; or
   in response to selecting a classification representing mismatching first and second nucleobases, determining a mismatch between the first and second nucleobases.

In one embodiment, determining sequence information of the first portion and the second portion comprises, in response to selecting a classification representing a match between the first and second nucleobases, base calling the first and second nucleobases.

In one embodiment, determining sequence information of the first portion and the second portion comprises, based on the selected classification, determining that the second portion is modified relative to the first portion at a location associated with the first and second nucleobases.

In one embodiment, the first signal component, second signal component, third signal component and fourth signal component are generated based on light emissions associated with the respective nucleobase.

In one embodiment, the light emissions are detected by a sensor, wherein the sensor is configured to provide a single output based upon the first and second signals.

In one embodiment, the sensor comprises a single sensing element.

In one embodiment, the method further comprises repeating steps (a) to (d) for each of a plurality of base calling cycles.

According to another aspect of the present invention, there is provided a kit comprising instructions for preparing at least one polynucleotide sequence for detection of mismatched base pairs as described herein, and/or for sequencing at least one polynucleotide sequence to detect mismatched base pairs as described herein.

According to another aspect of the present invention, there is provided a data processing device comprising means for carrying out a method as described herein.

In one embodiment, the data processing device is a polynucleotide sequencer.

According to another aspect of the present invention, there is provided a computer program product comprising instructions which, when the program is executed by a processor, cause the processor to carry out a method as described herein.

According to another aspect of the present invention, there is provided a computer-readable storage medium comprising instructions which, when executed by a processor, cause the processor to carry out a method as described herein.

According to another aspect of the present invention, there is provided a computer-readable data carrier having stored thereon a computer program product as described herein.

According to another aspect of the present invention, there is provided a data carrier signal carrying a computer program product as described herein.

According to an aspect of the present invention, there is provided a method of preparing at least one polynucleotide sequence for identification, comprising:
selectively processing at least one polynucleotide sequence comprising a first portion and a second portion, or at least one first polynucleotide sequence comprising a first portion and at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

In one embodiment, the concentration of the first portions capable of generating the first signal is greater than a concentration of the second portions capable of generating the second signal.

In one embodiment, the ratio between the concentration of the first portions capable of generating the first signal and the concentration of the second portions capable of generating the second signal is between 1.25:1 to 5:1, preferably between 1.5:1 to 3:1, more preferably about 2:1.

In one embodiment, the method comprises selectively processing at least one polynucleotide sequence comprising a first portion and a second portion.

In one embodiment, the method comprises selectively processing at least one first polynucleotide sequence comprising a first portion and at least one second polynucleotide sequence comprising a second portion.

In one embodiment, the first signal and the second signal are spatially unresolved.

In one embodiment, the at least one polynucleotide sequence comprises portions of a double-stranded nucleic acid template, and each of the first portions comprise a forward strand of the template, and each of the second portions comprise a reverse strand of the template or a forward complement strand of the template; or wherein each of the first portions comprise a reverse strand of the template, and each of the second portions comprise a forward strand of the template or a reverse complement strand of the template.

In one embodiment, the at least one polynucleotide sequence comprises portions of a double-stranded nucleic acid template, and each of the first portions comprises a forward strand of a template, and each of the second portions comprises a reverse complement strand of the template; or wherein each of the first portions comprises a reverse strand of a template, and each of the second portions comprises a forward complement strand of the template.

In one embodiment, the at least one polynucleotide sequence comprising the first portion and the second portion, or the at least one first polynucleotide sequence comprising the first portion and the at least one second polynucleotide sequence comprising the second portion, is/are attached to a solid support, preferably wherein the solid support is a flow cell.

In one embodiment the at least one polynucleotide sequence comprising the first portion and the second portion, or the at least one first polynucleotide sequence comprising the first portion and the at least one second polynucleotide sequence comprising the second portion, form a cluster on the solid support.

In one embodiment, the cluster is formed by bridge amplification.

In one embodiment, the at least one polynucleotide sequence comprising the first portion and the second portion forms a monoclonal cluster.

In one embodiment, the at least one first polynucleotide sequence comprising the first portion and the at least one second polynucleotide sequence comprising the second portion form a duoclonal cluster.

In one embodiment, a first region occupied by the at least one first polynucleotide sequence comprising the first portion within the duoclonal cluster is the same as, or substantially overlapping with, a second region occupied by the at least one second polynucleotide sequence comprising the second portion within the duoclonal cluster.

In one embodiment, the solid support comprises at least one first immobilised primer and at least one second immobilised primer.

In one embodiment, the first immobilised primer comprises a sequence as defined in SEQ ID NO: 1 or 5, or a variant or fragment thereof; and the second immobilised primer comprises a sequence as defined in SEQ ID NO: 2, or a variant or fragment thereof.

In one embodiment, the method comprises selectively processing at least one polynucleotide sequence comprising a first portion and a second portion, and wherein each polynucleotide sequence is attached to a first immobilised primer.

In one embodiment, each polynucleotide sequence comprising the first portion and the second portion further comprises a second adaptor sequence, wherein the second adaptor sequence is substantially complementary to the second immobilised primer.

In one embodiment, the method comprises selectively processing at least one first polynucleotide sequence comprising a first portion and at least one second polynucleotide sequence comprising a second portion, wherein each first polynucleotide sequence is attached to a first immobilised primer, and wherein each second polynucleotide sequence is attached a second immobilised primer.

In one embodiment each first polynucleotide sequence comprises a second adaptor sequence and each second polynucleotide sequence comprises a first adaptor sequence, wherein the second adaptor sequence is substantially complementary to the second immobilised primer and wherein the first adaptor sequence is substantially complementary to the first immobilised primer.

In one embodiment, selectively processing comprises conducting selective sequencing. Preferably, selectively processing comprises conducting selective amplification.

In one embodiment, selectively processing comprises contacting first sequencing primer binding sites located after a 3′-end of the first portions with first primers and contacting second sequencing primer binding sites located after a 3′-end of the second portions with second primers, wherein the second primers comprises a mixture of blocked second primers and unblocked second primers.

In one embodiment, the blocked second primer comprises a blocking group at a 3′ end of the blocked second primer. Preferably, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, the blocked second primer comprises a sequence as defined in SEQ ID NO: 31-36 or a variant or fragment thereof and/or the unblocked second primer comprises a sequence as defined in SEQ ID NO: 31-34 or a variant or fragment thereof.

In one embodiment, the blocked second primer comprises a sequence as defined in SEQ ID NO: 33 or 34 or a variant or fragment thereof and/or the unblocked second primer comprises a sequence as defined in SEQ ID NO: 35 or 36 or a variant or fragment thereof.

In one embodiment, selective processing comprises selectively removing some or substantially all of second immobilised primers that are not yet extended, and conducting a further amplification cycle in order to selectively amplify the first polynucleotide sequence(s) relative to the second polynucleotide sequence(s).

In one embodiment, selectively processing comprises selectively blocking some or substantially all of second immobilised primers that are not yet extended using a primer blocking agent, wherein the primer blocking agent is configured to limit or prevent synthesis of a strand extending from the second immobilised primer, and conducting a further amplification cycle in order to selectively amplify the first polynucleotide sequence(s) relative to the second polynucleotide sequence(s).

In one embodiment, the primer blocking agent is added whilst first polynucleotide sequence(s) are hybridised to the second immobilised primers.

In one embodiment, the method comprises contacting some or substantially all of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to 5′ additional nucleotide.

In one embodiment, the primer blocking agent is a blocked nucleotide. Preferably, the blocked nucleotide comprises a blocking group at a 3′ end of the blocked nucleotide.

In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase. In one embodiment, the blocked nucleotide is A or G.

In another aspect of the invention, there is provided a method of sequencing at least one polynucleotide sequence, comprising:
preparing at least one polynucleotide sequence for identification using a method as described herein; and
concurrently sequencing nucleobases in the first portion and the second portion based on the intensity of the first signal and the intensity of the second signal.

In one embodiment, concurrently sequencing nucleobases comprises performing sequencing-by-synthesis or sequencing-by-ligation.

In one embodiment, the method further comprises a step of conducting paired-end reads. Preferably, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

Preferably, selecting the classification based on the first and second intensity data comprises selecting the classification based on the combined intensity of the first and second signal components and the combined intensity of the third and fourth signal components.

Preferably, the plurality of classifications comprises sixteen classifications, each classification representing one of sixteen unique combinations of first and second nucleobases.

Preferably, the first signal component, second signal component, third signal component and fourth signal component are generated based on light emissions associated with the respective nucleobase.

Preferably, the light emissions are detected by a sensor, wherein the sensor is configured to provide a single output based upon the first and second signals.

Preferably, the sensor comprises a single sensing element.

Preferably, the method further comprises repeating steps (a) to (d) for each of a plurality of base calling cycles.

In another aspect of the invention, there is provided a primer, wherein the primer comprises a sequence as defined in SEQ ID NO: 31-36, or a variant or fragment thereof.

In one embodiment 3′ end of the primer comprises a 3′-OH group. In one embodiment, 3′ end of the primer comprises a blocking group at a 3′ end of the primer. Preferably, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In another aspect of the invention, there is provided use of a primer as described herein in preparing at least one polynucleotide sequence for identification as described herein.

In another aspect of the invention, there is provided a kit comprising instructions for preparing at least one polynucleotide sequence for identification as described herein; and/or sequencing at least one polynucleotide sequence as described herein.

In one embodiment the kit further comprises a primer as described herein.

In one embodiment the kit further comprises an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

In one embodiment the kit further comprises a primer-blocking agent(s), wherein the primer-blocking agent is preferably a blocked nucleotide, more preferably a blocked A or G.

In one embodiment the kit further comprises at least one extended primer sequence(s), wherein the extended primer sequence is selected from SEQ ID NO: 13 to 24, and wherein the extended primer sequence further comprise a 5′ additional nucleotide, wherein 5′ additional nucleotide is complementary to the primer-blocking agent.

In another aspect of the invention, there is provided an amplification composition comprising a recombinase, a DNA polymerase, a single-stranded DNA binding protein (SSB) and primer-blocking agent, wherein the primer-blocking agent is preferably a blocked nucleotide, more preferably a blocked A or G.

In one embodiment, the amplification composition further comprises at least one extended primer sequence(s), wherein the extended primer sequence is selected from SEQ ID NO: 13 to 24, and wherein the extended primer sequence further comprises a 5′ additional nucleotide, wherein 5′ additional nucleotide is complementary to the primer-blocking agent.

In another aspect of the invention, there is provided a data processing device comprising means for carrying out a method as described herein. In one embodiment, the data processing device is a polynucleotide sequencer.

In another aspect of the invention, there is provided a computer program product comprising instructions, which when the program is executed by a processor, cause the processor to carry out a method as described herein.

In another aspect of the invention, there is provided a computer-readable storage medium comprising instructions which, when executed by a processor, cause the processor to carry out a method as described herein.

In another aspect of the invention, there is provided a computer-readable data carrier having stored thereon a computer program product as described herein.

In another aspect of the invention, there is provided a data carrier signal carrying a computer program product as described herein.

According to an aspect of the present invention, there is provided a method of base calling nucleobases of two or more polynucleotide sequence portions, the method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion;
(c) selecting one of a plurality of classifications based on the first and the second intensity data, wherein each classification represents a possible combination of respective first and second nucleobases; and
(d) based on the selected classification, base calling the respective first and second nucleobases, wherein said polynucleotide sequence portions have been selectively processed such that an intensity of the signals obtained based upon the respective first nucleobase is greater than an intensity of the signals obtained based upon the respective second nucleobase.

In embodiments, the first and second signals and/or the third and fourth signals may be obtained substantially simultaneously.

In embodiments, selecting the classification based on the first and second intensity data may comprise selecting the classification based on the combined intensity of the first and second signals and the combined intensity of the third and fourth signals.

In embodiments, the plurality of classifications may comprise sixteen classifications, each classification representing one of sixteen unique combinations of first and second nucleobases.

In embodiments, the polynucleotide sequence portions may have been selectively processed such that, during sequencing, a greater number of the first polynucleotide sequence portions are capable of generating a signal than a number of the second polynucleotide sequence portions that are capable of generating a signal.

In embodiments, a ratio between the number of the first polynucleotide sequence portions capable of generating a signal and the number of the second polynucleotide sequence portions capable of generating a signal may be between 1.25:1 to 5:1, between 1.5:1 to 3:1, or about 2:1.

In embodiments, the first signal, second signal, third signal and fourth signal may be generated based on light emissions associated with the respective nucleobase.

In embodiments, the obtained signals are generated by:
contacting a plurality of polynucleotide molecules comprising the first and second polynucleotide sequence portions with first primers for sequencing the first polynucleotide sequence portions and second primers for sequencing the second polynucleotide sequence portions;
extending the first primers and the second primers by contacting the polynucleotide molecules with labeled nucleobases to form first labeled primers and second labeled primers;
stimulating the light emissions from the first and second labeled primers; and
detecting the light emissions at a sensor.

In embodiments:
the first and second signals may be based on light emissions detected in a first range of optical frequencies;
the third and fourth signals may be based on light emissions detected in a second range of optical frequencies; and
the first range of optical frequencies and the second range of optical frequencies may be not identical.

For example, the first range of optical frequencies may correspond to the color red, e.g., 400-484 THz (or equivalently, 620-750 nm in terms of wavelength), and the second range of optical frequencies may correspond to the color green, e.g., 526-606 THz (or equivalently, 495-570 nm in terms of wavelength).

In embodiments, the plurality of polynucleotide molecules may be attached to a substrate, and the light emissions from the first labeled primers and the light emissions from the second labeled primers may be emitted from the same region or substantially overlapping regions of the substrate.

In embodiments, the light emissions detected at the sensor may be spatially unresolved.

In embodiments, the sensor may be configured to provide a single output based upon the first and second signals.

In embodiments, the sensor may comprise a single sensing element.

In embodiments, each polynucleotide molecule may comprise one or more copies of the first polynucleotide sequence portion and one or more copies of the second polynucleotide sequence portion.

In embodiments, the first and second polynucleotide sequence portions may be respective portions of different polynucleotide molecules.

In embodiments, the polynucleotide sequence portions may have been selectively processed by contacting the plurality of polynucleotide molecules with unblocked first primers and a predetermined fraction of second primers which have a blocked 3′ end.

In embodiments, selectively processing may comprise preparing for selective sequencing or conducting selective sequencing. For example, selective sequencing may be achieved using a mixture of unblocked and blocked sequencing primers.

In embodiments, the polynucleotide sequence portions may have been selectively processed to provide a greater total number of the first polynucleotide sequence portions than a total number of the second polynucleotide sequence portions.

In embodiments, selectively processing may comprise conducting selective amplification.

In embodiments, the at least one first polynucleotide sequence portion and the at least one second polynucleotide sequence portion may be present in a cluster.

In embodiments, the one of the plurality of classifications may be selected based on the first and the second intensity data using a Gaussian mixture model.

In embodiments, the method may further comprise, based on the selected classification, determining that the second polynucleotide sequence portion is modified relative to the first polynucleotide sequence portion, at a location associated with the first and second nucleobases.

Said modification may have been made to any of the first polynucleotide sequence portion, the second polynucleotide sequence portion, or any sequence from which either of the first and second portions are derived, provided that it results in the modification of the sequences of the first and second portions relative to one another.

In embodiments, the second polynucleotide sequence portion may be modified relative to the first polynucleotide sequence portion resulting from a library preparation and/or sequencing error. The method may, therefore, further comprise: based on the selected classification, determining that a library preparation and/or sequencing errors has occurred.

In embodiments, the second polynucleotide sequence portion may be modified relative to the first polynucleotide sequence portion resulting from conversion of a modified cytosine to thymine or a nucleobase which is read as thymine/uracil, and/or of an unmodified cytosine to uracil or a nucleobase which is read as thymine/uracil. The method may, therefore, further comprise: based on the selected classification, determining the identity and the methylation status of a nucleobase at a corresponding position of a polynucleotide molecule from which the first and second polynucleotide sequence portions are derived.

In embodiments, the method may further comprise repeating steps (a) to (d) for each of a plurality of base calling cycles.

According to a second aspect of the present invention, there is provided a method of base calling nucleobases of n polynucleotide sequence portions, the method comprising:
(a) obtaining first intensity data comprising a combined intensity of respective first signal components generated by each of the nth portions obtained based upon respective nth nucleobases in each of the n portions, wherein the respective first signal components are obtained simultaneously;
(b) obtaining second intensity data comprising a combined intensity of respective second signal components generated by each of the nth portions obtained based upon respective nth nucleobases in each of the n portions, wherein the respective second signal components are obtained simultaneously;
(c) selecting one of a plurality of classifications based on the first and the second intensity data, wherein each classification represents a possible combination of respective nth nucleobases; and
(d) based on the selected classification, base calling the respective nth nucleobases for all n portions, wherein said polynucleotide sequence portions have been selectively processed such that an intensity of the signals obtained based upon the respective first nucleobase is greater than an intensity of the signals obtained based upon the respective second nucleobase.

In embodiments, selecting the classification based on the first and second intensity data may comprise selecting the classification based on the combined intensity of respective first signal components and second signal components.

In embodiments, the plurality of classifications may comprise 4n classifications, each classification representing one of 4″ unique combinations of nth nucleobases.

In embodiments, the method may further comprise repeating steps (a) to (d) for each of a plurality of base calling cycles.

According to a third aspect of the present invention, there is provided a data processing device comprising means for carrying out a method as described above.

In embodiments, the data processing device may be a polynucleotide sequencer.

According to a fourth aspect of the present invention, there is provided a computer program product comprising instructions which, when the program is executed by a processor, cause the processor to carry out a method as described above.

According to a fifth aspect of the present invention, there is provided a computer-readable storage medium comprising instructions which, when executed by a processor, cause the processor to carry out a method as described above.

According to a sixth aspect of the present invention, there is provided a computer-readable data carrier having stored thereon a computer program product as described above.

According to a seventh aspect of the present invention, there is provided a data carrier signal carrying a computer program product as described above.

In one embodiment, the disclosed technology provides systems and methods for determining the nucleobase sequences of two or more polynucleotide sequence portions in parallel (i.e., substantially simultaneously, using the same sequencing run), while the two sequence portions are co-localized within the same nucleic acid cluster. Thus, in some embodiments, the sequencing yield per flow cell area may be doubled as compared to prior systems which performed the sequencing serially.

In one embodiment, the disclosed method can include providing a substrate comprising a plurality of single or double stranded polynucleotide molecules in a cluster. The disclosed method can further include contacting the plurality of polynucleotide molecules with first primers for sequencing a first polynucleotide sequence portion and second primers for sequencing a second polynucleotide sequence portion. The first and second polynucleotide sequence portions may be present as respective portions of the same polynucleotide molecules. Alternatively, the first and second polynucleotide sequence portions may be present in different polynucleotide molecules of the plurality of polynucleotide molecules. The disclosed method can further include extending the first primers and the second primers by contacting the cluster with labeled nucleobases to form first labeled primers and second labeled primers. The disclosed method can further include stimulating light emissions from the first and second labelled primers, wherein an amplitude of the signal generated by the first labeled primers is greater than an amplitude of the signal generated by the second labeled primers (or vice versa). The disclosed method can further include identifying the labeled nucleobases added to the first primers and the second primers based on the amplitude of the signal generated by the labelled nucleobases. In some embodiments, the first primers are index primers that hybridize to a site adjacent to a barcode index portion associated with the first sequence portion. In some embodiments, the second primers are index primers that hybridize to a site adjacent to a barcode index portion associated with the second sequence portion. In some embodiments, the first primers are index primers that hybridize to a site adjacent to a barcode index portion associated with the first sequence portion, and the second primers are index primers that hybridize to a site adjacent to a barcode index portion associated with the second sequence portion.

In some embodiments, identifying the labeled nucleobases added to the first primers and identifying the labeled nucleobases added to the second primers are performed substantially simultaneously. In some embodiments, the signal generated by the first labelled primers and the signal generated by the second labeled primers are emitted from the same region or substantially overlapping regions of the substrate. In some embodiments, each polynucleotide molecule is attached to the substrate. In some embodiments, the plurality of polynucleotide molecules in the cluster are generated by a bridge amplification process, an exclusion amplification process, a rolling circle amplification process, or any other suitable amplification process. In some embodiments, the substrate comprises a plurality of clusters of nucleic acids, the clusters being randomly distributed on the substrate. In alternative embodiments, the clusters are arranged in a patterned array.

In some embodiments, the amplitude of the signal generated by the first labeled primers corresponds with a first quantity of the first labeled primers in the cluster, and the amplitude of the signal generated by the second labeled primers corresponds with a second quantity of the second labeled primers in the cluster. In some embodiments, contacting the plurality of polynucleotide molecules with first primers for sequencing the first sequence portion and second primers for sequencing the second sequence portion comprises contacting the molecules with unblocked first primers and a predetermined fraction of second primers which have a blocked 3′-end. The blocked 3′-end may be formed by any way of blocking the ability of a primer to extend a nucleic acid strand, for example by modifications in the sugar or nucleobase. In some embodiments, the blocked 3′-end comprises a hairpin loop, a deoxynucleotide, a phosphate group, a propyl spacer, a modification blocking the 3′-hydroxyl group, or an inverted nucleobase. In some embodiments, the first primers are formed of a locked nucleic acid or a peptide nucleic acid. In some embodiments, the second primers are formed of a locked nucleic acid or a peptide nucleic acid.

In some embodiments, the disclosed method further includes: detecting the signal generated by the first labeled primers in a first range of optical frequencies and a second range of optical frequencies; and detecting the signal generated by the second labeled primers in the first range of optical frequencies and the second range of optical frequencies, wherein the first range of optical frequencies and the second range of optical frequencies are not identical. For example, the first range of optical frequencies may correspond to the color red, e.g., 400-484 THz (or equivalently, 620-750 nm in terms of wavelength), and the second range of optical frequencies may correspond to the color green, e.g., 526-606 THz (or equivalently, 495-570 nm in terms of wavelength).

In some embodiments, the disclosed method further includes: acquiring a first fluorescent image of the cluster in a first range of optical frequencies; acquiring a second fluorescent image of the cluster in a second range of optical frequencies, wherein the first range of optical frequencies and the second range of optical frequencies are not identical; and obtaining the signals generated by the first and second labeled primers by extracting fluorescence intensities from the first and second fluorescent images of the cluster. In some examples, the first range of optical frequencies and the second range of optical frequencies may partially overlap. For example, the first range of optical frequencies may be 500-580 THz, and the second range of optical frequencies may be 540-620 THz.

In some embodiments, the disclosed method further includes extracting fluorescence intensities from the first and second fluorescent images of the same region or substantially overlapping regions of the substrate. In some embodiments, identifying the labeled nucleobases added to the first primers and the second primers is based on a combination of the extracted fluorescence intensities from the first and second fluorescent images. In some embodiments, a combination of identities of the labeled nucleobases added to the first primers and the second primers is classified as one of sixteen combinations of types of nucleobases, based on the combination of the extracted fluorescence intensities and predetermined fluorescence intensity distributions for the sixteen combinations of types of nucleobases. In some embodiments, the disclosed method further includes: normalizing the extracted fluorescence intensities; and classifying a combination of identities of the labeled nucleobases added to the first primers and the second primers as one of sixteen combinations of types of nucleobases, based on a combination of the normalized extracted fluorescence intensities and predetermined normalized fluorescence intensity distributions for the sixteen combinations of types of nucleobases.

In some embodiments, the disclosed method further includes stimulating fluorescent emissions from the first labeled primers and second labeled primers in the cluster with light at a predetermined optical frequency. In some embodiments, the disclosed method further includes stimulating fluorescent emissions from the first labeled primers and second labeled primers in the cluster with light at two predetermined optical frequencies. In some embodiments, the disclosed method further includes identifying whether the labeled nucleobases are associated with the first sequence portion or the second sequence portion based on the amplitude of the signal generated by the labeled nucleobases.

The systems, devices, kits, and methods disclosed herein each have several aspects, no single one of which is solely responsible for their desirable attributes. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

It is to be understood that any features of the systems disclosed herein may be combined together in any desirable manner and/or configuration. Further, it is to be understood that any features of the methods disclosed herein may be combined together in any desirable manner. Moreover, it is to be understood that any combination of features of the methods and/or the systems may be used together, and/or may be combined with any of the examples disclosed herein. It should be appreciated that all combinations of the foregoing concepts and additional concepts discussed in greater detail below are contemplated as being part of the inventive subject matter disclosed herein and may be used to achieve the benefits and advantages described herein.

According to an aspect of the present invention, there is provided a method of preparing at least one polynucleotide sequence for identification, comprising:
selectively processing at least one polynucleotide sequence comprising n portions, such that a proportion of each of the n portions are each capable of generating a respective n^{th} signal,
wherein n is 2 or more, and
wherein the selective processing causes an intensity of an i^{th} signal to be different compared to an intensity of a j^{th} signal, for all i between 1 to n, and for all j between 1 to n, and where i is not equal to j.

In one embodiment, a concentration of each of the i^{th} portions capable of generating the i^{th} signal is different compared to a concentration of each of the j^{th} portions capable of generating the j^{th} signal.

In one embodiment, a ratio between a concentration of one of the n portions capable of generating the (m−1)^{th} most intense signal and a concentration of another of the n portions capable of generating the m^{th} most intense signal is between 1.25:1 to 5:1, between 1.5:1 to 3:1, or about 2:1, wherein m is between 2 to n.

In another embodiment, a ratio between each concentration of one of the n portions capable of generating the (m−1)^{th} most intense signal and each concentration of another of the n portions capable of generating the m^{th} most intense signal is between 1.25:1 to 5:1, between 1.5:1 to 3:1, or about 2:1, for all m between 2 to n.

In one example, each of the n^{th} signals are spatially unresolved.

In one embodiment, selectively processing comprises preparing for selective sequencing or conducting selective sequencing.

In one embodiment, selectively processing comprises contacting n^{th} sequencing primer binding sites located after a 3′-end of each of the respective n portions with respective n^{th} primers, wherein at least one of the n^{th} primers comprises a mixture of blocked n^{th} primers and unblocked n^{th} primers, and
of the n^{th} primers that do comprise a mixture of blocked n^{th} primers and unblocked n^{th} primers, a ratio of blocked n^{th} primers to unblocked n^{th} primers is different compared to a ratio of blocked primers and unblocked primers of all other primers comprising a mixture of respective blocked and unblocked primers.

In one embodiment, all but one of the n^{th} primers comprises a mixture of blocked n^{th} primers and unblocked n^{th} primers.

In one embodiment, the blocked n^{th} primer comprises a blocking group at a 3′ end of the blocked n^{th} primer.

In one example, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, one of the blocked n^{th} primers comprises a sequence as defined in SEQ ID NO. 31 to 36 or a variant or fragment thereof and/or the corresponding unblocked n^{th} primer comprises a sequence as defined in SEQ ID NO. 31 to 36 or a variant or fragment thereof.

In one embodiment, n is between 2 to 6, or between 2 to 4.

In another embodiment, n is 3 or more, or between 3 to 6, or 3 or 4.

In one aspect, one of the n portions has a different polynucleotide sequence compared to another of the n portions, wherein the respective sequences may be genetically unrelated and/or obtained from different sources.

In one embodiment, each of the n portions has a different polynucleotide sequence compared to each of the other n portions, wherein the respective sequences may be genetically unrelated and/or obtained from different sources.

In one embodiment, the at least one polynucleotide sequence comprising the n portions is/are attached to a solid support, wherein the solid support may be a flow cell.

In one embodiment, the at least one polynucleotide sequence comprising the n portions forms a cluster on the solid support.

In one embodiment, the cluster is formed by bridge amplification.

In one embodiment, the at least one polynucleotide sequence comprising the n portions forms a monoclonal cluster.

In one embodiment, the solid support comprises at least one first immobilised primer and at least one second immobilised primer.

In one embodiment, the first immobilised primer comprises a sequence as defined in SEQ ID NO. 1 or 5, or a variant or fragment thereof; and the second immobilised primer comprises a sequence as defined in SEQ ID NO. 2, or a variant or fragment thereof.

In one embodiment, each polynucleotide sequence comprising the n portions is attached to a first immobilised primer.

In another embodiment, each polynucleotide sequence comprising the n portions further comprises a second adaptor sequence, wherein the second adaptor sequence is substantially complementary to the second immobilised primer.

In one embodiment, the method further comprises:
providing a solid support comprising a plurality of first immobilised primers and a plurality of second immobilised primers, wherein an initial proportion of the first immobilised primers have each been extended to form the polynucleotide sequence comprising n portions and substantially all of the second immobilised primers have not been extended, wherein each polynucleotide sequence comprising n portions comprises a second adaptor sequence which is substantially complementary to the second immobilised primer,
selectively blocking a proportion of second immobilised primers that have not been extended using a primer blocking agent, wherein the primer blocking agent is configured to limit or prevent synthesis of a strand extending from the second immobilised primer, and
conducting at least two amplification cycles in order provide a new proportion of first immobilised primers that have been extended to form the polynucleotide sequence comprising n portions and a proportion of second immobilised primers that have been extended to form polynucleotide complement sequences comprising n complement portions, wherein the new proportion of first immobilised primers is greater than the initial proportion of first immobilised primers.

In one embodiment, the method further comprises a step of cleaving substantially all of the polynucleotide complement sequences comprising n complement portions.

In one embodiment, between 60% to 95% of second immobilised primers that have not been extended are blocked using the primer blocking agent; between 75% to 90%, between 80% to 90%, or between 85% to 90%.

In one embodiment, the method comprises contacting some of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to 5′ additional nucleotide.

In one embodiment, the primer blocking agent is a blocked nucleotide.

In one embodiment, the blocked nucleotide comprises a blocking group at a 3′ end of the blocked nucleotide.

In one example, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one aspect, the blocked nucleotide is A or G.

In one embodiment, the extended primer sequence comprises a first extended primer sequence which is substantially complementary to the second immobilised primer and comprises a first 5′ additional nucleotide, and a second extended primer sequence which is substantially complementary to the second immobilised primer and comprises a second 5′ additional nucleotide, wherein the first 5′ additional nucleotide and the second 5′ additional nucleotide are configured to base pair with different nucleotides, and the primer blocking agent is complementary to the first 5′ additional nucleotide.

In one embodiment, the first extended primer sequence forms between 60% to 95% of the total population of extended primer sequences; between 75% to 90%, 80% to 90%, or between 85% to 90%.

In one embodiment, the primer blocking agent is provided as a mixture of blocked nucleotides and unblocked nucleotides, wherein the blocked nucleotide and the unblocked nucleotide comprise the same base.

In one embodiment, the blocked nucleotide forms between 60% to 95% of the total population of the mixture; between 75% to 90%, between 80% to 90%, or between 85% to 90%.

In one embodiment, each of the n portions comprises a sequence derived from a nucleic acid sample (e.g. an insert).

In one embodiment, each of the n portions is at least 25 base pairs.

According to another aspect of the present invention, there is provided a method of sequencing at least one polynucleotide sequence, comprising:
preparing at least one polynucleotide sequence for identification using a method as described herein; and
concurrently sequencing nucleobases in each of the n portions based on the intensity of each of the n^{th} signals.

In one embodiment, the step of concurrently sequencing nucleobases comprises performing sequencing-by-synthesis or sequencing-by-ligation.

In one embodiment, the method further comprises a step of conducting paired-end reads.

In one embodiment, the step of concurrently sequencing nucleobases comprises:
(a) obtaining first intensity data comprising a combined intensity of respective first signal components generated by each of the n portions obtained based upon respective n^{th} nucleobases in each of the n portions, wherein each of the respective first signal components are obtained simultaneously;
(b) obtaining second intensity data comprising a combined intensity of respective second signal components generated by each of the n portions obtained based upon respective n^{th} nucleobases in each of the n portions, wherein each of the respective second signal components are obtained simultaneously;
(c) selecting one of a plurality of classifications based on the first and the second intensity data, wherein each classification represents a possible combination of respective n^{th} nucleobases; and
(d) based on the selected classification, base calling the respective n^{th} nucleobases for all n portions.

In one example, selecting the classification based on the first and second intensity data comprises selecting the classification based on the combined intensity of respective first signal components and second signal components.

In one embodiment, the plurality of classifications comprises 4″ classifications, each classification representing one of 4″ unique combinations of n^{th} nucleobases.

In one embodiment, the first signal components and the second signal components are generated based on light emissions associated with the respective nucleobase.

In one aspect, the light emissions are detected by a sensor, wherein the sensor is configured to provide a single output based upon the n signals.

In one example, the sensor comprises a single sensing element.

In one embodiment, the method further comprises repeating steps (a) to (d) for each of a plurality of base calling cycles.

According to another aspect of the present invention, there is provided a method of synthesising template polynucleotides, comprising:
providing a solid support comprising a plurality of first immobilised primers and a plurality of second immobilised primers, wherein an initial proportion of the first immobilised primers have each been extended to form a template polynucleotide and substantially all of the second immobilised primers have not been extended, wherein each template polynucleotide comprises a second adaptor sequence which is substantially complementary to the second immobilised primer,
selectively blocking a proportion of second immobilised primers that have not been extended using a primer blocking agent, wherein the primer blocking agent is configured to limit or prevent synthesis of a strand extending from the second immobilised primer, and
conducting at least two amplification cycles in order provide a new proportion of first immobilised primers that have been extended to form template polynucleotides and a proportion of second immobilised primers that have been extended to form template complement polynucleotides, wherein the new proportion of first immobilised primers is greater than the initial proportion of first immobilised primers.

In one embodiment, the method further comprises a step of cleaving substantially all of the polynucleotide complement sequences comprising n complement portions.

In one embodiment, between 60% to 95% of second immobilised primers that have not been extended are blocked using the primer blocking agent; or between 75% to 90%, or between 80% to 90%, or between 85% to 90%.

In one embodiment, the method comprises contacting some of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to 5′ additional nucleotide.

In one embodiment, the primer blocking agent is a blocked nucleotide.

In one embodiment, the blocked nucleotide comprises a blocking group at a 3′ end of the blocked nucleotide.

In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, the blocked nucleotide is A or G.

In one embodiment, the extended primer sequence comprises a first extended primer sequence which is substantially complementary to the second immobilised primer and comprises a first 5′ additional nucleotide, and a second extended primer sequence which is substantially complementary to the second immobilised primer and comprises a second 5′ additional nucleotide, wherein the first 5′ additional nucleotide and the second 5′ additional nucleotide are configured to base pair with different nucleotides, and the primer blocking agent is complementary to the first 5′ additional nucleotide.

In one embodiment, the first extended primer sequence forms between 60% to 95% of the total population of extended primer sequences; between 75% to 90%, between 80% to 90%, or between 85% to 90%.

In one embodiment, the primer blocking agent is provided as a mixture of blocked nucleotides and unblocked nucleotides, wherein the blocked nucleotide and the unblocked nucleotide comprise the same base.

In one embodiment, the blocked nucleotide forms between 60% to 95% of the total population of the mixture; between 75% to 90%, between 80% to 90%, or between 85% to 90%.

According to another aspect of the present invention, there is provided a kit comprising instructions for preparing at least one polynucleotide sequence for identification as described herein; and/or sequencing at least one polynucleotide sequence as described herein.

According to another aspect of the present invention, there is provided a data processing device comprising means for carrying out a method as described herein.

In one aspect, the data processing device is a polynucleotide sequencer.

According to another aspect of the present invention, there is provided a computer program product comprising instructions which, when the program is executed by a processor, cause the processor to carry out a method as described herein.

According to another aspect of the present invention, there is provided a computer-readable storage medium comprising instructions which, when executed by a processor, cause the processor to carry out a method as described herein.

According to another aspect of the present invention, there is provided a computer-readable data carrier having stored thereon a computer program product as described herein.

According to another aspect of the present invention, there is provided a data carrier signal carrying a computer program product as described herein.

### DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows a typical solid support.

FIG. 2 shows the stages of bridge amplification and the generation of an amplified cluster comprising (A) a library strand hybridising to an immobilised primer; (B) generation of a template strand from the library strand; (C) dehybridisation and washing away the library strand; (D) hybridisation of the template strand to another immobilised primer; (E) generation of a template complement strand from the template strand via bridge amplification; (F) dehybridisation of the sequence bridge; (G) hybridisation of the template strand and template complement strand to immobilised primers; and (H) subsequent bridge amplification to provide a plurality of template and template complement strands.

FIG. 3 shows the detection of nucleobases using 4-channel, 2-channel and 1-chanl chemistry.

FIG. 4 shows starting from a double-stranded polynucleotide sequence comprising a forward strand of the sequence and a reverse strand of the sequence adaptors may be ligated to generate a loop fork ligated polynucleotide sequence and subsequent amplification using PCR to generate a self-tandem insert library.

FIG. 5 shows three adaptor configurations are produced after ligation of the adapters, one which represents the desired loop/fork configuration. PCR and/or clustering steps eliminate the loop/loop configuration, due to it lacking any primer binding sites. A single affinity-based system eliminates unwanted fork/fork molecules.

FIG. 6 shows the binding of primers to a primer-binding sequence on a template duplex, thus preparing a tandem library fragment for sequencing.

FIG. 7 shows that a 9QAM encoding scheme can be used to accurately differentiate between two simultaneously received base calls; plotting relative intensities of light signals obtained from Read 1.1 and Read 1.2 generates a constellation of 9 clouds. The four corner clouds represent high quality and accurate base calls, while off-corner clouds represent potential library prep/sequencing errors, which could be eliminated.

FIG. 8 shows that a 9QAM encoding scheme can be used to simultaneously sequence genomic and epigenetic data; epigenetic conversion of the polynucleotide library strand by, for example, Bisulfite/EM-Seq or TAPS and subsequent sequencing enables mC and the canonical bases to be identified simultaneously.

FIG. 9 shows an exemplar nicking arrangement to facilitate sequencing of the entire inverted-repeat tandem-insert duplex. Following nicking of the lawn primers and sequencing of the first strand (read 1), the free ends of the sequenced strands are blocked. Nicking enzymes, specific for an alternative recognition site, are added to nick a recognition site within the loop sequence to generate two start sites for simultaneous sequencing of the other strand of the original polynucleotide duplex.

FIG. 10 shows an exemplar nicking arrangement to facilitate sequencing of the entire inverted-repeat tandem-insert duplex. The first nicking event can occur within the loop sequence, and the polynucleotides sequences dehybridized for the first read. The sequenced strands are extended to regenerate 3′ primer-binding sequences. Nicking enzymes may be applied to nick the lawn primers, to produce two sequencing start sites that allow simultaneous sequencing from opposite end of both inserts

FIG. 11 shows a nick arrangement at the loop sequence that generates two immobilised extended strands, effectively halving the tandem insert. Following dehybridisation, a first and second sequencing primers can be applied and bind to their respective primer-binding sequences to facilitate Read 1.1 and Read 1.2.

FIG. 12 shows an example of a method of sequencing an inverted-repeat tandem-insert library strand. Following library preparation, cluster generation occurs and a loop-hybridised sequence bridge forms. Nicking enzymes may be applied to nick the sequence bridges at a pair of recognition sequences in the loop stem simultaneously, providing sequencing start sites for different strands of the original duplex template. The strands can be simultaneously sequenced by standard SBS or double-stranded SBS (e.g. strand displacement SBS). In standard SBS sequencing, the non-immobilised sequences—i.e., the sequences 3′ of the nicked site—are washed off before the sequences steps for R1.1 and R1.2. In double-stranded SBS (e.g. strand displacement SBS), the non-immobilised sequences 3′ of the nicked site are not washed off.

FIG. 13(A) is a plot showing graphical representations of sixteen distributions of signals generated by polynucleotide sequences according to one embodiment. FIG. 13(B) shows a method of selective sequencing.

FIG. 14 is a flow diagram showing a method for base calling according to one embodiment.

FIG. 15 is a plot showing graphical representations of nine distributions of signals generated by polynucleotide sequences according to one embodiment.

FIG. 16 shows the effect of unmodified cytosine to uracil conversion treatment of a double-stranded polynucleotide, and a scatter plot showing the resulting distributions of signals generated by polynucleotide sequences.

FIG. 17 shows the effect of modified cytosine to thymine conversion treatment of a double-stranded polynucleotide, and a scatter plot showing the resulting distributions of signals generated by polynucleotide sequences.

FIG. 18 shows alternative signal distributions using a different dye-encoding scheme.

FIG. 19 shows alternative signal distributions using a different dye-encoding scheme.

FIG. 20 shows alternative signal distributions using a different dye-encoding scheme.

FIG. 21 is a flow diagram showing a method for determining sequence information according to one embodiment.

FIG. 22 shows a 9 QaM analysis conducted on the signals obtained from the custom second hyb run of Example 1. The x-axis shows signal intensity from a “red” wavelength channel, whilst the y-axis shows signal intensity from a “green” wavelength channel. G is not associated with any dyes and as such appears contributes no intensity for both “red” and “green” channels. C is associated with a “red” dye and as such contributes intensity to the “red” channel, but not the “green” channel. T is associated with a “green” dye and as such contributes intensity to the “green” channel, but not the “red channel. A is associated with both a “red” dye and a “green” dye, and as such contributes intensity to both the “red” channel and “green” channel. Since the template comprises forward and reverse complement strands that are sequenced simultaneously, most of the readout will generate (G,G) read (bottom left corner), (C,C) read (bottom right corner), (T,T) read (top left corner), and (A,A) read (top right corner) clouds. However, a central cloud corresponding to (C,T) or (T,C) reads corresponds with the presence of modified cytosines.

FIGS. 23A to 23F show 9 QaM analysis conducted on the signals obtained from Example 2 (library fragments 1 to 6). The x-axis shows signal intensity from a “red” wavelength channel, whilst the y-axis shows signal intensity from a “green” wavelength channel. A CA dye swap has been performed in this MiniSeq run compared to a standard MiniSeq run. G is not associated with any dyes and as such appears contributes no intensity for both “red” and “green” channels. A is associated with a “red” dye and as such contributes intensity to the “red” channel, but not the “green” channel. T is associated with a “green” dye and as such contributes intensity to the “green” channel, but not the “red channel. C is associated with both a “red” dye and a “green” dye, and as such contributes intensity to both the “red” channel and “green” channel. Since the template comprises forward and reverse complement strands that are sequenced simultaneously, the readout will generate (T,T) reads (top left corner), (T,C) reads (top middle), (C,C) reads (top right corner), (G,G) reads (bottom left corner), (G,A) reads (bottom middle), and (A,A) reads (bottom right corner). The top right corner corresponds to a (5-mC)-G base pair, whilst the bottom left corner corresponds to a G-(5-mC) base pair, thus corresponding with the presence of modified cytosines. Groupings are as follows: T in forward strand of library in top left (marked as “T”); C in forward strand of library in top middle (marked as “C”); 5-mC in forward strand of library in top right (marked as “c”); G in forward strand of library and associated with 5-mC in reverse strand of library in bottom left (marked as “g”); G in forward strand of library and associated with C in reverse strand of library in bottom middle (marked as “G”); and A in forward strand of library in bottom right (marked as “A”). In FIGS. 23A to 23C, two scatter-plots are shown: the plot marked “read-color coded” corresponds to assignments for each base to particular groups during the read process; the plot marked “ref-color coded” shows the true assignments for each base to particular groups and is indicative of where errors have occurred in the read process. FIGS. 23D to 23F show combined “read-color coded” and “ref-color coded” plots—where the read and the reference differ, a border is shown for the read assignment, whilst the central portion of the circle shows the actual assignment. In addition, FIGS. 23A to 23F show sequence alignment of the read sequence to the true methylated pUC19 sample-“m” above or below a C represents 5-mC, whilst “m” above or below a G represents G that is base-paired with 5-mC; red boxes indicate errors in read (of sequence or methylation status).

FIG. 24 shows a forward strand, reverse strand, forward complement strand, and reverse complement strand of a polynucleotide molecule.

FIG. 25 shows the steps involved in a loop fork method.

FIG. 26 shows an example of a polynucleotide sequence prepared using a loop fork method.

FIG. 27 shows an example of a polynucleotide sequence prepared using a loop fork method.

FIG. 28 shows the stages of bridge amplification for polynucleotide templates prepared using a loop fork method and the generation of an amplified cluster, comprising (A) a concatenated library strand hybridising to a immobilised primer; (B) generation of a template strand from the library strand; (C) dehybridisation and washing away the library strand; (D) generation of a template complement strand from the template strand via bridge amplification and dehybridisation of the sequence bridge; and (E) further amplification to provide a plurality of template and template complement strands.

FIG. 29 shows a method of selective sequencing in panels (A) and (B).

FIG. 30 shows a method of selective amplification comprising (A) starting from a plurality of template and template complement strands; (B) selective cleavage of one type of immobilised primer from the support; (C) only template (or template complement) strands complementary to the free immobilised primer anneal and undergo bridge amplification, (D) producing different proportions of template and template complement strands; (E) subsequent standard (non-selective) sequencing occurs in different proportions enabling signal differentiation.

FIG. 31 shows a method of selective amplification comprising (A) template and template complement strands annealing to immobilised primers; (B) addition of a primer-blocking agent that binds only to one type of immobilised primer, preventing the extension from that one type of immobilised primer, preventing the extension from one type of immobilised primer; (C) producing different proportions of template and template complement strands; (D) subsequent standard (non-selective) sequencing occurs in different proportions enabling signal differentiation.

FIG. 32 shows a method of selective amplification comprising (A) flowing a (or a plurality of) extended primer sequence(s) containing at least one additional 5′ nucleotide across the surface of the solid support; (B) addition of a primer-blocking agent that binds only to one type of immobilised primer and is complementary to the additional 5′ nucleotide of the extended primer sequence, preventing the extension from one type of immobilised primer.

FIG. 33 is a plot showing graphical representations of nine distributions of signals generated by polynucleotide sequences according to one embodiment, highlighting distributions that may be associated with library preparation errors.

FIG. 34 shows a nicking strategy and subsequent sequencing using double stranded SBS (strand displacement SBS).

FIG. 35 shows the preparation of a concatenated polynucleotide sequence comprising a first portion and a second portion using a tandem insert method, comprising (A) preparation of a desired first (forked) adaptor and second (forked) adaptor from three oligos; (B) different types of first (forked) adaptors and second (forked) adaptors that do not anneal to each other due to the presence of a third oligo on at least one of the first (forked) adaptor and/or the second (forked) adaptor; (C) ligation of the template polynucleotide strand and adaptors generates three products, with the desired product containing both types of adaptor being produced at a proportion of 50%; (D) synthesis of concatenated strands from the desired product; and (E) completion of the synthesis of the concatenated strands from the desired product.

FIG. 36 shows an example of a concatenated polynucleotide sequence comprising a first portion and a second portion, as well as terminal and internal adaptor sequences.

FIG. 37A shows 9 QaM analysis conducted on the signals obtained from the custom second hyb run of Example 3. The x-axis shows signal intensity from a “red” wavelength channel, whilst the y-axis shows signal intensity from a “green” wavelength channel. G is not associated with any dyes and as such appears contributes no intensity for both “red” and “green” channels. C is associated with a “red” dye and as such contributes intensity to the “red” channel, but not the “green” channel. T is associated with a “green” dye and as such contributes intensity to the “green” channel, but not the “red channel. A is associated with both a “red” dye and a “green” dye, and as such contributes intensity to both the “red” channel and “green” channel. Since the template comprises forward and reverse complement strands that are sequenced simultaneously, most of the readout will generate (G,G) read (bottom left corner), (C,C) read (bottom right corner), (T,T) read (top left corner), and (A,A) read (top right corner) clouds. However, any mismatched base pairs will appear in regions other than the four corner clouds. A central cloud corresponding to (C,T) or (T,C) reads corresponds with the presence of modified cytosines; in addition, side clouds located at the top middle, bottom middle, centre left and centre right sections corresponds with the presence of other mismatched base pairs.

FIG. 37B shows sequence data generated from two different primers used (HYB2′-ME and HP10) in the custom second hyb run of Example 3. Mismatches between the two sequences allow identification of modified cytosines. For example, 5-mC present in the original forward strand of the target polynucleotide is read as T in the HP10 read, whereas C present in the original reverse complement strand of the target polynucleotide (corresponding to the same position as 5-mC in the original forward strand of the target polynucleotide) is read as C in the HYB2′-ME read.

FIG. 38A shows the sequencing primer binding modes used in Example 4-Read 1 (control) is conducted using only a single sequencing primer type (HP21 mix), Read 2 (control) is conducted using a single sequencing primer type (HYB2′-ME), and Read 3 is conducted using two sequencing primer types (HP10 mix and HYB2′-ME) to enable concurrent sequencing to generate a 9 QaM signal.

FIG. 38B shows the results from the Read 1, Read 2 and Read 3 runs in Example 4. The plot is arranged so that G is disposed on the bottom left corner, C is disposed on the top left corner, T is disposed on the bottom right corner, and A is disposed on the top right corner. The Read 1 plot has a T base call for one of the reads (highlighted as a circled point). The Read 2 plot has a C base call for the read corresponding to the same position (highlighted as a circled point). The Read 3 plot contains (G,G) reads at the bottom left corner, (C,C) reads at the top left corner, (T,T) reads at the bottom right corner, and (A,A) reads at the top right corner. An mismatched base pair error was detected due to the presence of a (C,T) read in the central middle portion of the plot.

FIG. 39 shows a typical polynucleotide with 5′ and 3′ adaptor sequences.

FIG. 40 shows an example of PCR stitching. Here, two sequences-a strand of a human library and a strand of a phix library are joined together to create a single polynucleotide strand comprising both a first portion (comprising the strand of the human sequence) and a second portion (comprising the strand of the phix sequence), as well as terminal and internal adaptor sequences.

FIG. 41 shows (A) that by plotting relative intensities of light signals obtained from a first channel (ch1) and a second channel (ch2), a constellation of 16 clouds is obtained; (B) alignment of R1 and R2 (minor and major reads respectively) with the known human and Phix sequence.

FIG. 42 shows that by plotting relative intensities of light signals obtained from a first channel (ch1) and a second channel (ch2), a constellation of 16 clouds is obtained.

FIG. 43 shows (A) that by plotting relative intensities of light signals obtained from a first channel (ch1) and a second channel (ch2), a constellation of 16 clouds is obtained for R1 and R2 concurrently and R3 and R4 concurrently; (B) alignment of R1, R2, R3 and R4 with the known sequence; (C) annotation of where R1, R2, R3 and R4 appear on the known sequence.

FIG. 44 shows a block diagram which schematically illustrates an example sequencing system that may be used to perform the disclosed methods.

FIG. 45 shows a block diagram which schematically illustrates an example imaging system that may be used in conjunction with the example sequencing system of FIG. 44.

FIG. 46 shows a functional block diagram of an example computer system that may be used in the example sequencing system of FIG. 44.

FIG. 47A and FIG. 47B schematically illustrate nucleic acid clusters comprising two or more polynucleotide sequence portions for sequencing by the present methods.

FIG. 48 are charts which shows example dye labeling schemes that may be used in conjunction with the present methods.

FIG. 49 schematically illustrates how library preparation errors can obscure true variants in NGS methods.

FIG. 50 schematically illustrates the use of unique molecular indices (UMIs) for eliminating library preparation errors.

FIG. 51 shows schematically how library preparation errors and true variants can be associated with different distributions of combined signal intensities.

FIG. 52 shows the effect of methylcytosine (mC) to thymine conversion treatment of a top strand of a tandem polynucleotide molecule, and a scatter plot showing the resulting distributions of signals from a nucleic acid cluster generated from the treated molecule.

FIG. 53 shows the effect of cytosine to uracil conversion treatment of top and bottom strands of a tandem polynucleotide molecule, and a scatter plot showing the resulting distributions of signals from a nucleic acid cluster generated from the treated molecule.

FIG. 54 is a diagram showing a method for classifying combined signal intensities into one of a plurality of distributions.

FIG. 55 is a flow diagram showing a method for generating signals for use in the method of base calling shown in FIG. 14.

FIG. 56A shows the effect of pre-treatment of library strands using C to U conversion on bases in template strands.

FIG. 56B shows the effect of pre-treatment of library strands using mC to T conversion on bases in template strands.

### DETAILED DESCRIPTION

All patents, patent applications, and other publications referred to herein, including all sequences disclosed within these references, are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

Embodiments of the present invention can be used in sequencing, in particular duplex sequencing. Methodologies applicable to embodiments of the present invention have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in US20060024681, US20060292611, WO 06/110855, WO 06/135342, WO 03/074734, WO07/010252, WO 07/091077, WO 00/179553, WO 98/44152 and WO 2022/087150, the contents of which are herein incorporated by reference.

Embodiments of the present invention also can be used in sequencing, in particular concurrent sequencing. Methodologies applicable to embodiments have been described in WO 08/041002, WO 07/052006, WO 98/44151, WO 00/18957, WO 02/06456, WO 07/107710, WO05/068656, U.S. Ser. No. 13/661,524 and US 2012/0316086, the contents of which are herein incorporated by reference. Further information can be found in US20060024681, US 20060292611, WO 06/110855, WO 06/135342, WO 03/074734, WO07/010252, WO 07/091077, WO 00/179553, WO 98/44152 and WO 2022/087150, the contents of which are herein incorporated by reference.

As used herein, the term “variant” refers to a variant polypeptide sequence or part of the polypeptide sequence that retains desired function of the full non-variant sequence. For example, a desired function of the immobilised primer retains the ability to bind (i.e. hybridise) to a target sequence.

As used in any aspect described herein, a “variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid sequence. The sequence identity of a variant can be determined using any number of sequence alignment programs known in the art. As an example, Emboss Stretcher from the EMBL-EBI may be used: https://www.ebi.ac.uk/Tools/psa/emboss_stretcher/(using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “fragment” refers to a functionally active series of consecutive nucleic acids from a longer nucleic acid sequence. The fragment may be at least 99%, at least 95%, at least 90%, at least 80%, at least 70%, at least 60%, at least 50%, at least 40% or at least 30% the length of the longer nucleic acid sequence. A fragment as used herein may also retain the ability to bind (i.e. hybridise) to a target sequence.

Sequencing typically comprises four fundamental steps: 1) library preparation to form a plurality of target polynucleotides for identification; 2) cluster generation to form an array of amplified template polynucleotides; 3) sequencing the cluster array of amplified template polynucleotides; and 4) data analysis to identify characteristics of the target polynucleotides from the amplified template polynucleotide sequences. These steps are described in greater detail below.

### Library Strands and Template Terminology

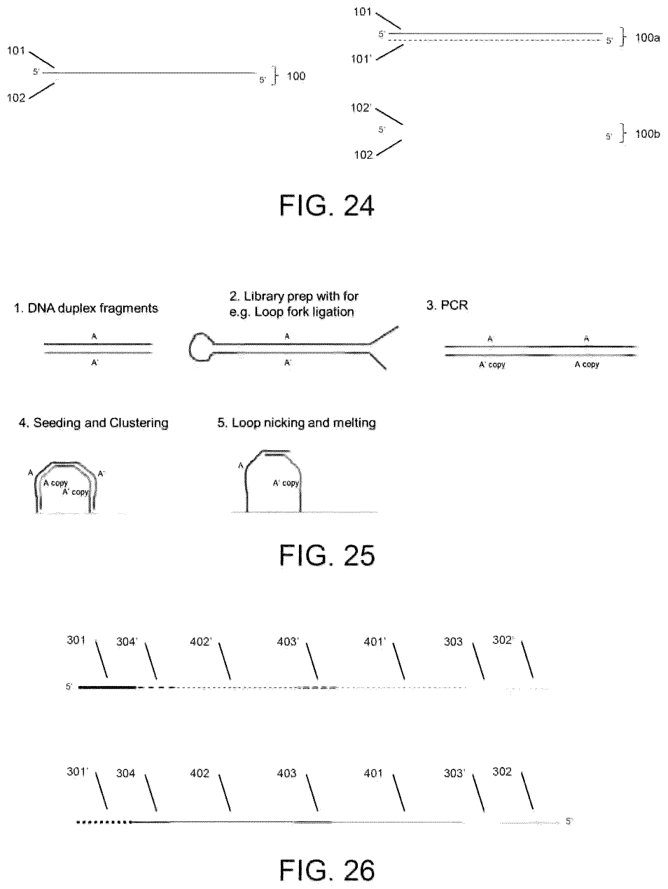

For a given double-stranded polynucleotide sequence 100 (also referred to herein as a polynucleotide library) to be identified, the polynucleotide sequence 100 comprises a forward strand of the sequence 101 and a reverse strand of the sequence 102. See, e.g., FIG. 24.

Typically, when the polynucleotide sequence is replicated (e.g. using a DNA/RNA polymerase), complementary versions of the forward strand 101 of the sequence 100 and the reverse strand 102 of the sequence 100 are generated. These may be referred to as the forward complement strand of the sequence and the reverse complement strand of the sequence respectively. Thus, replication of the polynucleotide sequence 100 provides a double-stranded polynucleotide sequence 100a that comprises a forward strand of the sequence 101 and a forward complement strand of the sequence 101′, and a double-stranded polynucleotide sequence 100b that comprises a reverse strand of the sequence 102 and a reverse complement strand of the sequence 102′.

By using the forward complement strand of the sequence as a template for complementary base pairing, a sequencing process (e.g. a sequencing-by-synthesis or a sequencing-by-ligation process) reproduces information that was present in the original forward strand of the sequence. The forward complement strand of the sequence may be referred to as the forward strand of the template.

Similarly, by using the reverse complement strand of the sequence as a template for complementary base pairing, a sequencing process (e.g. a sequencing-by-synthesis or a sequencing-by-ligation process) reproduces information that was present in the original reverse strand of the sequence. The reverse complement strand of the sequence may be referred to as the reverse strand of the template. The term “template” may be used to describe a complementary version of the double-stranded polynucleotide sequence 100. As such, the “template” comprises a forward complement strand of the sequence 101′ and a reverse complement strand of the sequence 102′. Thus, by using the forward complement strand of the sequence 101′ as a template for complementary base pairing, a sequencing process (e.g. a sequencing-by-synthesis or a sequencing-by-ligation process) reproduces information that was present in the original forward strand of the sequence 101. Similarly, by using the reverse complement strand of the sequence 102′ as a template for complementary base pairing, a sequencing process (e.g. a sequencing-by-synthesis or a sequencing-by-ligation process) reproduces information that was present in the original reverse strand of the sequence 102.

The two strands in the template may also be referred to as a forward strand of the template 101′ and a reverse strand of the template 102′. The complement of the forward strand of the template 101′ is termed the forward complement strand of the template 101, whilst the complement of the reverse strand of the template 102′ is termed the reverse complement strand of the template 102.

Generally, where forward strand, reverse strand, forward complement strand, and reverse complement strand are used herein without qualifying whether they are with respect to the original polynucleotide sequence 100 or with respect to the “template”, these terms may be interpreted as referring to the “template”.

| Language for original | Corresponding language for the |
|---|---|
| polynucleotide sequence 100 | “template” |
| Forward strand of the | Forward complement strand of the |
| sequence 101 | template 101 (sometimes referred to |
| | herein as forward complement strand 101) |
| Reverse strand of the | Reverse complement strand of the |
| sequence 102 | template 102 (sometimes referred to |
| | herein as reverse complement strand 102) |
| Forward complement strand | Forward strand of the template 101′ |
| of the sequence 101′ | (sometimes referred to herein as forward |
| | strand 101′) |
| Reverse complement strand | Reverse strand of the template 102′ |
| of the sequence 102′ | (sometimes referred to herein as reverse |
| | strand 102′) |

### Library Preparation

Library preparation is the first step in any high-throughput sequencing platform. These libraries allow templates to be generated via complementary base pairing that can subsequently be clustered and amplified. During library preparation, nucleic acid sequences, for example genomic DNA sample, or cDNA or RNA sample, are converted into polynucleotide templates or a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adaptors (adaptor sequences). The original sample DNA fragments are referred to as “inserts”. The target polynucleotides may advantageously also be size-fractionated prior to modification with the adaptor sequences. As described herein, typically the templates to be generated from the libraries may include separate polynucleotide sequences, in particular a first polynucleotide sequence comprising a first portion and a second polynucleotide sequence comprising a second portion, for example, duplexes comprising a first portion, that is the forward strand (of the template) and a second portion, that is the reverse strand (of the template). For example, as described herein, typically the templates to be generated from the libraries may include a concatenated polynucleotide sequence comprising a first portion and a second portion. Alternatively, the templates to be generated typically include separate polynucleotide sequences, in particular a first polynucleotide sequence comprising a first portion and a second polynucleotide sequence comprising a second portion. Generating these templates from particular libraries may be performed according to methods known to persons of skill in the art. However, some example approaches of preparing libraries suitable for generation of such templates are described below.

In some embodiments, the library is prepared by ligating adaptor sequences to the duplex, as described in more detail in e.g. WO 07/052006, which is incorporated herein by reference. In some cases, “tagmentation” can be used to attach the sample DNA to the adaptors, as described in more detail in e.g. WO 10/048605, US 2012/0301925, US 2013/0143774 and WO 2016/189331, each of which are incorporated herein by reference. In tagmentation, double-stranded DNA is simultaneously fragmented and tagged with adaptor sequences and PCR primer binding sites. The combined reaction eliminates the need for a separate mechanical shearing step during library preparation. In tagmentation, double-stranded DNA is simultaneously fragmented and tagged with adaptor sequences and PCR primer binding sites. The combined reaction eliminates the need for a separate mechanical shearing step during library preparation. These procedures may be used, for example, for preparing templates including a first polynucleotide sequence comprising a first portion and a second polynucleotide sequence comprising a second portion, wherein the first portion is a forward strand of the template, and the second portion is a forward complement strand of the template—i.e. a copy of the forward strand (or alternatively, wherein the first portion is a reverse strand of the template, and the second portion is a reverse complement strand of the template). Where features are described herein in relation to the “forward” strand, it should be considered that these features could equally be applied to the “reverse strand”. In one embodiment, as described in further detail below, the library may be prepared using a loop fork method. This procedure may be used, for example, for preparing templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template). A representative process for conducting a loop fork method is shown in FIG. 25. This procedure may also be used, for example, for preparing templates comprising concatenated polynucleotide sequences comprising a first portion and a second portion, wherein the first portion is a forward strand of the template, and the second portion is a reverse strand of the template. Such libraries may also be referred to as self-tandem inserts. A representative process for conducting a loop fork method is shown in FIG. 25.

This procedure may also be used, for example, for preparing templates comprising concatenated polynucleotide sequences, wherein a single sequence comprises both the forward and reverse strands of the template—or a copy of the forward strand of the template (i.e. a forward complement strand of the template) and a copy of the reverse strand of the template (i.e. a reverse complement strand of the template). In one aspect, the present invention describes methods of preparing an inverted-repeat tandem-insert polynucleotide, where the orientation of the forward strand with respect to the reverse strand (or the copy of the forward strand with respect to the reverse strand) is inverted.

Starting from a double-stranded polynucleotide sequence comprising a forward strand of the sequence and a reverse strand of the sequence, adaptors may be ligated to a first end of the sequence (e.g. using processes as described in more detail in e.g. WO 07/052006, or “tagmentation” methods as described above). A second end of the sequence (different from the first end) may be ligated to a loop, which connects the forward strand of the sequence and the reverse strand of the sequence, thus generating a loop fork ligated polynucleotide sequence. Conducting PCR on the loop fork ligated polynucleotide sequence produces a new double-stranded polynucleotide sequence, one strand comprising the forward strand of the sequence and the reverse strand of the sequence, and the other strand comprising a forward complement strand of the sequence and a reverse complement strand of the sequence. The library is now ready for seeding, clustering and amplification.

As will be described later, during clustering and amplification, further processes may be used to generate templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template).

The processes described above in relation to loop fork methods generate libraries that have self-tandem insert polynucleotides.

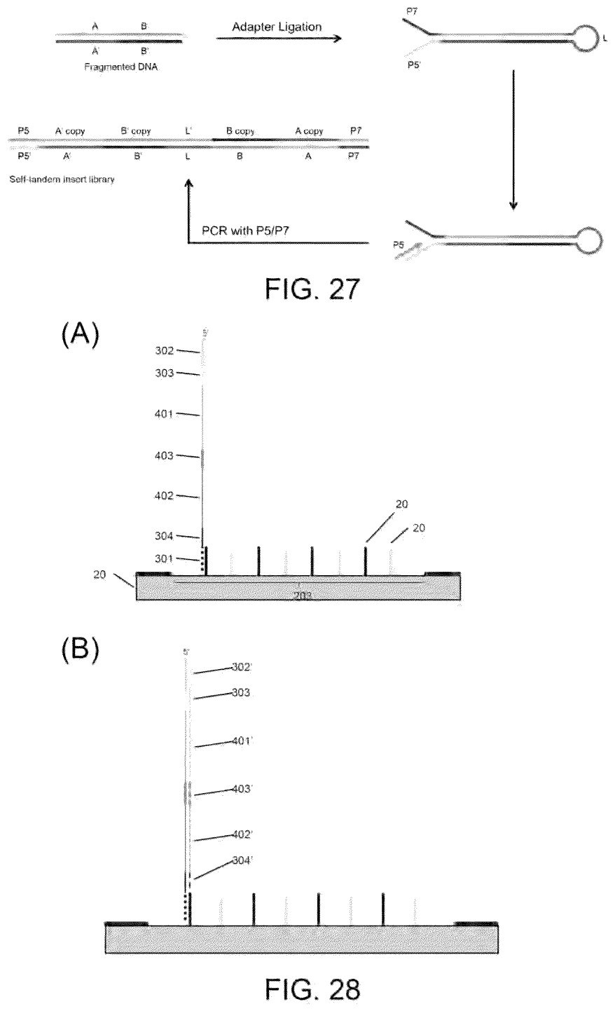

Thus, one strand of a polynucleotide within a polynucleotide library may comprise, in a 5′ to 3′ direction, a second primer-binding complement sequence 302 (e.g. P7), an optional first terminal sequencing primer binding site complement 303′, a first insert sequence 401 (A and B), a loop sequence 403 (L), a second insert sequence 402 (B′ and A′), an optional second terminal sequencing primer binding site 304, and a first primer-binding sequence 301′ (e.g. P5′) (FIGS. 26 and 27—bottom strand).

Alternatively, or in addition, one or more sequencing primer binding sites (or complements) may be provided within the loop sequence 403 (L).

Although not shown in FIGS. 26 and 27, the strand may further comprise one or more index sequences. As such, a first index sequence (e.g. i7) may be provided between the second primer-binding complement sequence 302 (e.g. P7) and the optional first terminal sequencing primer binding site complement 303′. Separately, or in addition, a second index complement sequence (e.g. i5′) may be provided between the optional second terminal sequencing primer binding site 304 and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), an optional first terminal sequencing primer binding site complement 303′, a first insert sequence 401 (A and B), a loop sequence 403 (L), a second insert sequence 402 (B′ and A′), an optional second terminal sequencing primer binding site 304, a second index complement sequence (e.g. i5′), and a first primer-binding sequence 301′ (e.g. P5′).

Alternatively, or in addition, one or more index sequences (or complements) may be provided within the loop sequence 403 (L).

Another strand of a polynucleotide within a polynucleotide library may comprise, in a 5′ to 3′ direction, a first primer-binding complement sequence 301 (e.g. P5), an optional second terminal sequencing primer binding site complement 304′, a second insert complement sequence 402′ (A′ copy and B′ copy), a loop complement sequence 403′ (L′), a first insert complement sequence 401′ (B copy and A copy), an optional first terminal sequencing primer binding site 303, and a second primer-binding sequence 302′ (e.g. P7′) (FIGS. 26 and 27—top strand).

Alternatively, or in addition, one or more sequencing primer binding sites (or complements) may be provided within the loop complement sequence 403′ (L′).

Although not shown in FIG. 26 or 27, the another strand may further comprise one or more index sequences. As such, a second index sequence (e.g. i5) may be provided between the first primer-binding complement sequence 301 (e.g. P5) and the optional second terminal sequencing primer binding site complement 304′. Separately, or in addition, a first index complement sequence (e.g. i7′) may be provided between the optional first terminal sequencing primer binding site 303 and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), an optional second terminal sequencing primer binding site complement 304′, a second insert complement sequence 402′ (A′ copy and B′ copy), a loop complement sequence 403′ (L′), a first insert complement sequence 401′ (B copy and A copy), an optional first terminal sequencing primer binding site 303, a first index complement sequence (e.g. i7′), and a second primer-binding sequence 302′ (e.g. P7′).

Alternatively, or in addition, one or more index sequences (or complements) may be provided within the loop complement sequence 403′ (L′).

In one embodiment, the first insert sequence 401 may comprise a forward strand of the sequence 101, and the second insert complement sequence 402′ may comprise a reverse complement strand of the sequence 102′ (or the first insert sequence 401 may comprise a reverse strand of the sequence 102, and the second insert complement sequence 402′ may comprise a forward complement strand of the sequence 101′), for example where the library is prepared using a loop fork method.

Although FIG. 26 shows the presence of a first terminal sequencing primer binding site complement 303′, a second terminal sequencing primer binding site 304, a second terminal sequencing primer binding site complement 304′, and a first terminal sequencing primer binding site 303, these are optional as mentioned above. Accordingly, these sections may be omitted from the library.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (a primer-binding sequence and a sequencing primer binding site, or a combination of a primer-binding sequence, an index sequence and a sequencing primer binding site) may be referred to herein as an adaptor sequence, and an insert (or inserts in concatenated strands) is flanked by a 5′ adaptor sequence and a 3′ adaptor sequence. The primer-binding sequence may also comprise a sequencing primer for the index read.

As also described herein, typically the templates to be generated from the libraries may include a concatenated polynucleotide sequence comprising a first portion and a second portion. Alternatively, the templates to be generated typically include separate polynucleotide sequences, in particular a first polynucleotide sequence comprising a first portion and a second polynucleotide sequence comprising a second portion. Generating these templates from particular libraries may be performed according to methods known to persons of skill in the art. However, some example approaches of preparing libraries suitable for generation of such templates are described below.

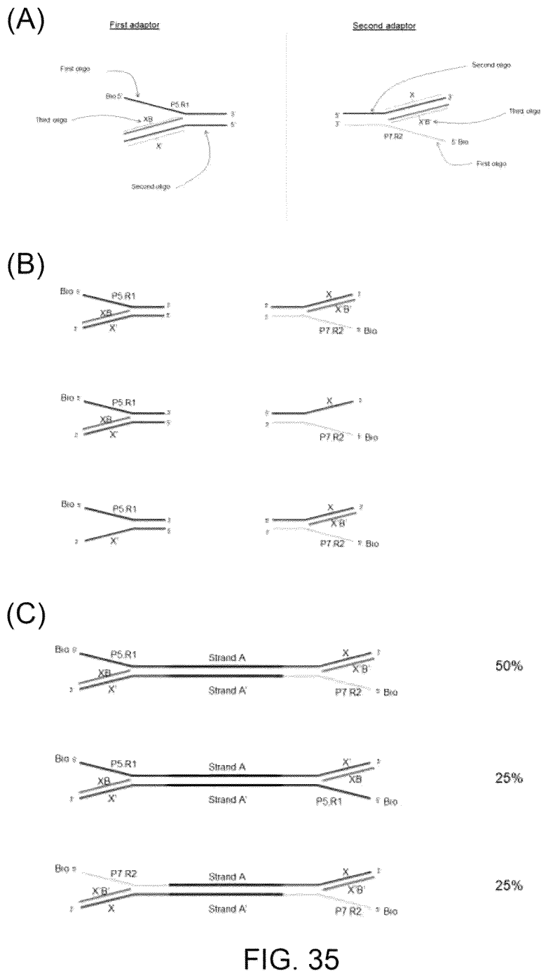

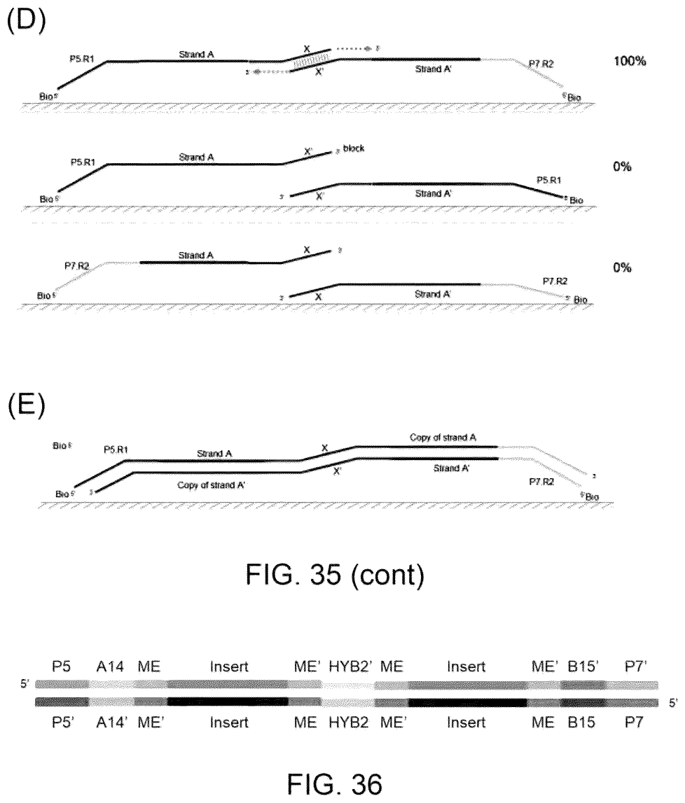

In some embodiments, the library may be prepared by using a tandem insert method described in more detail in e.g. WO 2022/087150, which is incorporated herein by reference. This procedure may be used, for example, for preparing templates comprising concatenated polynucleotide sequences comprising a first portion and a second portion, wherein the first portion is a forward strand of the template, and the second portion is a reverse complement strand of the template (or alternatively, wherein the first portion is a reverse strand of the template, and the second portion is a forward complement strand of the template). Such libraries may also be referred to as cross-tandem inserts. A representative process for conducting a tandem insert method is shown in FIG. 35A to 35E.

The processes described above in relation to tandem insert methods generate libraries that have concatenated polynucleotides.

Thus, one strand of a concatenated polynucleotide within a polynucleotide library may comprise, in a 5′ to 3′ direction, a second primer-binding complement sequence 302 (e.g. P7), a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), and a first primer-binding sequence 301′ (e.g. P5′) (e.g., FIGS. 26 and 36—bottom strand).

Although not shown in FIGS. 26 and 36, the strand may further comprise one or more index sequences. As such, a first index sequence (e.g. i7) may be provided between the second primer-binding complement sequence 302 (e.g. P7) and the first terminal sequencing primer binding site complement 303′ (e.g. B15-ME; or if ME is not present, then B15). Separately, or in addition, a second index complement sequence (e.g. i5′) may be provided between the second terminal sequencing primer binding site 304 (e.g. ME′-A14′) and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), a second index complement sequence (e.g. i5′), and a first primer-binding sequence 301′ (e.g. P5′)

Another strand of a concatenated polynucleotide within a polynucleotide library may comprise, in a 5′ to 3′ direction, a first primer-binding complement sequence 301 (e.g. P5), a second terminal sequencing primer binding site complement 304′ (e.g. A14-ME; or if ME is not present, then A14), a second insert complement sequence 402′, a hybridisation sequence 403′ (e.g. ME′-HYB2′-ME; or if ME′ and ME are not present, then HYB2′), a first insert complement sequence 401′, a first terminal sequencing primer binding site 303 (e.g. ME′-B15′; or if ME′ is not present, then B15′), and a second primer-binding sequence 302′ (e.g. P7′) (FIGS. 26 and 36—top strand).

Although not shown in FIGS. 26 and 36, the another strand may further comprise one or more index sequences. As such, a second index sequence (e.g. i5) may be provided between the first primer-binding complement sequence 301 (e.g. P5) and the second terminal sequencing primer binding site complement 304′ (e.g. A14-ME; or if ME is not present, then A14). Separately, or in addition, a first index complement sequence (e.g. i7′) may be provided between the first terminal sequencing primer binding site 303 (e.g. ME′-B15′; or if ME′ is not present, then B15′) and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), a second terminal sequencing primer binding site complement 304′ (e.g. A14-ME; or if ME is not present, then A14), a second insert complement sequence 402′, a hybridisation sequence 403′ (e.g. ME′-HYB2′-ME; or if ME′ and ME are not present, then HYB2′), a first insert complement sequence 401′, a first terminal sequencing primer binding site 303 (e.g. ME′-B15′; or if ME′ is not present, then B15′), a first index complement sequence (e.g. i7′), and a second primer-binding sequence 302′ (e.g. P7′).

As described herein, the first insert sequence 401 and the second insert sequence 402 may comprise different types of library sequences.

In one embodiment, the first insert sequence 401 may comprise a forward strand of the sequence 101, and the second insert sequence may comprise a reverse complement strand of the sequence 102′ (or the first insert sequence 401 may comprise a reverse strand of the sequence 102, and the second insert sequence 402 may comprise a forward complement strand of the sequence 101′), for example where the library is prepared using a tandem insert method.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (a primer-binding sequence and a sequencing primer binding site, or a combination of a primer-binding sequence, an index sequence and a sequencing primer binding site) may be referred to herein as an adaptor sequence, and an insert (or inserts in concatenated strands) is flanked by a 5′ adaptor sequence and a 3′ adaptor sequence. The primer-binding sequence may also comprise a sequencing primer for the index read.

In concatenated strands, the hybridisation sequence (or the hybridisation sequence complement) may comprise an internal sequencing primer binding site. In other words, an internal sequencing primer binding site may form part of the hybridisation sequence. For example, ME′-HYB2 (or ME′-HYB2′) may act as an internal sequencing primer binding site to which a sequencing primer can bind. Alternatively, the hybridisation sequence may be an internal sequencing primer binding site. For example, HYB2 (or HYB2′) may act as an internal sequencing primer binding site to which a sequencing primer can bind. Accordingly, we may refer to the hybridisation site herein as comprising a second sequencing primer binding site, or as a second sequencing primer binding site.

As used herein, an “adaptor” refers to a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the P5′ and P7′ primer-binding sequences are complementary to short primer sequences (or lawn primers) present on the surface of a flow cell. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

In concatenated strands, the hybridisation sequence (or the hybridisation sequence complement) may comprise an internal sequencing primer binding site. In other words, an internal sequencing primer binding site may form part of the hybridisation sequence. For example, ME′-HYB2 (or ME′-HYB2′) may act as an internal sequencing primer binding site to which a sequencing primer can bind. Alternatively, the hybridisation sequence may be an internal sequencing primer binding site. For example, HYB2 (or HYB2′) may act as an internal sequencing primer binding site to which a sequencing primer can bind. Accordingly, we may refer to the hybridisation site herein as comprising a second sequencing primer binding site, or as a second sequencing primer binding site.

The loop complement (or the loop) may comprise an internal sequencing primer binding site. In other words, an internal sequencing primer binding site may form part of the loop complement. Alternatively, the loop complement may be an internal sequencing primer binding site. Accordingly, we may refer to the loop complement herein as comprising a second sequencing primer binding site, or as a second sequencing primer binding site.

In some embodiments, the library may be prepared by ligating adaptor sequences to double-stranded polynucleotide sequences, each comprising a forward strand of the sequence and a reverse strand of the sequence, as described in more detail in e.g. WO 07/052006, which is incorporated herein by reference. In some cases, “tagmentation” can be used to attach the sample DNA to the adaptors, as described in more detail in e.g. WO 10/048605, US 2012/0301925, US 2013/0143774 and WO 2016/189331, each of which are incorporated herein by reference. In tagmentation, double-stranded DNA is simultaneously fragmented and tagged with adaptor sequences and PCR primer binding sites. The combined reaction eliminates the need for a separate mechanical shearing step during library preparation. These procedures may be used, for example, for preparing templates including a first polynucleotide sequence comprising a first portion and a second polynucleotide sequence comprising a second portion, wherein the first portion is a forward strand of the template, and the second portion is a forward complement strand of the template—i.e. a copy of the forward strand (or alternatively, wherein the first portion is a reverse strand of the template, and the second portion is a reverse complement strand of the template). Where features herein are described in relation to the “forward” strand, it should be considered that these features could equally be applied to the “reverse strand”.

Where libraries are prepared by ligating adaptor sequences to double-stranded polynucleotide sequences as described above, library preparation may comprise ligating a first primer-binding sequence 301′ (e.g. P5′, such as SEQ ID NO: 3) and a second terminal sequencing primer binding site 304 (e.g. SBS3′, for example, SEQ ID NO: 44) to a 3′-end of a forward strand of a sequence 101. See FIG. 26. The library preparation may be arranged such that the second terminal sequencing primer binding site 304 is attached (e.g. directly attached) to 3′-end of the forward strand of the sequence 101, and such that the first primer-binding sequence 301′ is attached (e.g. directly attached) to 3′-end of the second terminal sequencing primer binding site 304.

The library preparation may further comprise ligating a complement of first terminal sequencing primer binding site 303′ (e.g. SBS12, such as SEQ ID NO: 45) (also referred to herein as a first terminal sequencing primer binding site complement 303′) and a complement of a second primer-binding sequence 302 (also referred to herein as a second primer-binding complement sequence 302) (e.g. P7, such as SEQ ID NO: 2) to a 5′-end of the forward strand of the sequence 101. The library preparation may be arranged such that first terminal sequencing primer binding site complement 303′ is attached (e.g. directly attached) to 5′-end of the forward strand of the sequence 101, and such that second primer-binding complement sequence 302 is attached (e.g. directly attached) to 5′-end of first terminal sequencing primer binding site complement 303′.

Thus, one strand of a polynucleotide within a polynucleotide library may comprise, in a 5′ to 3′ direction, a second primer-binding complement sequence 302 (e.g. P7), a first terminal sequencing primer binding site complement 303′ (e.g. SBS12), a forward strand of the sequence 101, a second terminal sequencing primer binding site 304 (e.g. SBS3′), and a first primer-binding sequence 301′ (e.g. P5′) (FIG. 26-bottom strand).

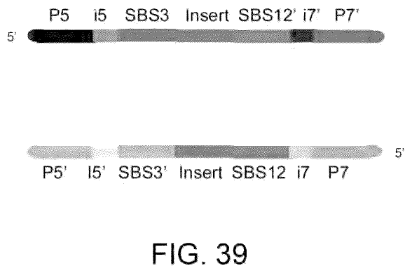

Although not shown in FIG. 26, the strand may further comprise one or more index sequences. As such, a first index sequence (e.g. i7) may be provided between the second primer-binding complement sequence 302 (e.g. P7) and the first terminal sequencing primer binding site complement 303′ (e.g. SBS12). Separately, or in addition, a second index complement sequence (e.g. i5′) may be provided between the second terminal sequencing primer binding site 304 (e.g. SBS3′) and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), a first terminal sequencing primer binding site complement 303′ (e.g. SBS12), a forward strand of the sequence 101, a second terminal sequencing primer binding site 304 (e.g. SBS3′), a second index complement sequence (e.g. i5′), and a first primer-binding sequence 301′ (e.g. P5′). A typical polynucleotide is shown in FIG. 39 (bottom strand).

When a double-stranded sequence 100 is used, the library preparation may also comprise ligating a second primer-binding sequence 302′ (e.g. P7′) and a first terminal sequencing primer binding site 303 (e.g. SBS12′) to a 3′-end of a reverse strand of a sequence 102. The library preparation may be arranged such that first terminal sequencing primer binding site 303 is attached (e.g. directly attached) to 3′-end of the reverse strand of the sequence 102, and such that the second primer-binding sequence 302′ is attached (e.g. directly attached) to 3′-end of first terminal sequencing primer binding site 303.

The library preparation may further comprise ligating a complement of a second terminal sequencing primer binding site 304′ (e.g. SBS3) (also referred to herein as a second terminal sequencing primer binding site complement 304′) and a complement of a first primer-binding sequence 301 (also referred to herein as a first primer-binding complement sequence 301) (e.g. P5) to a 5′-end of the reverse strand of the sequence 102. The library preparation may be arranged such that the second terminal sequencing primer binding site complement 304′ is attached (e.g. directly attached) to 5′-end of the reverse strand of the sequence 102, and such that the first primer-binding complement sequence 301 is attached (e.g. directly attached) to 5′-end of the second terminal sequencing primer binding site complement 304′.

Thus, another strand of a polynucleotide within a polynucleotide library may comprise, in a 5′ to 3′ direction, a first primer-binding complement sequence 301 (e.g. P5), a second terminal sequencing primer binding site complement 304′ (e.g. SBS3), a reverse strand of the sequence 102, a first terminal sequencing primer binding site 303 (e.g. SBS12′), and a second primer-binding sequence 302′ (e.g. P7′) (FIG. 26-top strand).

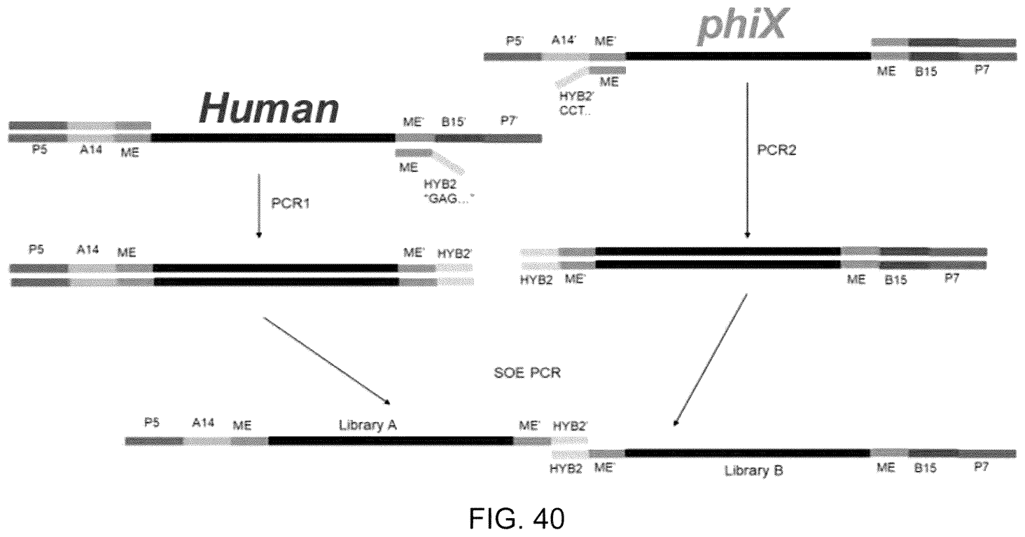

Although not shown in FIG. 26, the another strand may further comprise one or more index sequences. As such, a second index sequence (e.g. i5) may be provided between the first primer-binding complement sequence 301 (e.g. P5) and the second terminal sequencing primer binding site complement 304′ (e.g. SBS3). Separately, or in addition, a first index complement sequence (e.g. i7′) may be provided between the first terminal sequencing primer binding site 303 (e.g. SBS12′) and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), a second terminal sequencing primer binding site complement 304′ (e.g. SBS3), a reverse strand of the sequence 102, a first terminal sequencing primer binding site 303 (e.g. SBS12′), a first index complement sequence (e.g. i7′), and a second primer-binding sequence 302′ (e.g. P7′). A typical polynucleotide is shown in FIG. 39 (top strand). In some embodiments, the library may be prepared using PCR stitching methods, such as (splicing by) overlap extension PCR (also known as OE-PCR or SOE-PCR), as described in more detail in e.g. Higuchi et al. (Nucleic Acids Res., 1988, vol. 16, pp. 7351-7367), which is incorporated herein by reference. This procedure may be used, for example, for preparing templates including concatenated polynucleotide sequences comprising a first portion and a second portion, wherein the first portion and the second portion are different polynucleotide sequences (e.g. genetically unrelated, and/or obtained from different sources). A representative process for conducting PCR stitching for a human and PhiX library is shown in FIG. 40.

As used herein, the term “genetically unrelated” refers to portions which are not related in the sense of being any two of the group consisting of: forward strands, reverse strands, forward complement strands, and reverse complement strands. However, the “genetically unrelated” sequences could be different fragment sequences which are derived from the same source, but are different fragments from that source (e.g. from the same fragmented library preparation process). This includes sequences that can be overlapping in sequence (but not identical in sequence).

The processes described above in relation to PCR stitching methods generate libraries that have concatenated polynucleotides.

Starting from a double-stranded polynucleotide sequence comprising a forward strand of the sequence and a reverse strand of the sequence, adaptors may be ligated to a first end of the sequence (e.g. using processes as described in more detail in e.g. WO 07/052006, or “tagmentation” methods as described above). A second end of the sequence (different from the first end) may be ligated to a loop, which connects the forward strand of the sequence and the reverse strand of the sequence, thus generating a loop fork ligated polynucleotide sequence. Conducting PCR on the loop fork ligated polynucleotide sequence produces a new double-stranded polynucleotide sequence, one strand comprising the forward strand of the sequence and the reverse strand of the sequence, and the other strand comprising a forward complement strand of the sequence and a reverse complement strand of the sequence. The library is now ready for seeding, clustering and amplification.

As will be described later, during clustering and amplification, further processes may be used to generate templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template).

The processes described above in relation to PCR stitching, tandem insert methods, and loop fork methods generate libraries that have concatenated polynucleotides.

The processes described above in relation to PCR stitching methods generate libraries that have concatenated polynucleotides.

Thus, in an illustrative example where n is 2, one strand of a concatenated polynucleotide within a polynucleotide library may comprise, in a 5′ to 3′ direction, a second primer-binding complement sequence 302 (e.g. P7), a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), and a first primer-binding sequence 301′ (e.g. P5′) (FIGS. 26 and 36-bottom strand).

Although not shown in FIGS. 26 and 36, the strand may further comprise one or more index sequences. As such, a first index sequence (e.g. i7) may be provided between the second primer-binding complement sequence 302 (e.g. P7) and the first terminal sequencing primer binding site complement 303′ (e.g. B15-ME; or if ME is not present, then B15). Separately, or in addition, a second index complement sequence (e.g. i5′) may be provided between the second terminal sequencing primer binding site 304 (e.g. ME′-A14′) and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), a second index complement sequence (e.g. i5′), and a first primer-binding sequence 301′ (e.g. P5′)

Another strand of a concatenated polynucleotide within a polynucleotide library may comprise, in a 5′ to 3′ direction, a first primer-binding complement sequence 301 (e.g. P5), a second terminal sequencing primer binding site complement 304′ (e.g. A14-ME; or if ME is not present, then A14), a second insert complement sequence 402′, a hybridisation sequence 403′ (e.g. ME′-HYB2′-ME; or if ME′ and ME are not present, then HYB2′), a first insert complement sequence 401′, a first terminal sequencing primer binding site 303 (e.g. ME′-B15′; or if ME′ is not present, then B15′), and a second primer-binding sequence 302′ (e.g. P7′) (FIGS. 26 and 36—top strand).

Although not shown in FIGS. 26 and 36, the another strand may further comprise one or more index sequences. As such, a second index sequence (e.g. i5) may be provided between the first primer-binding complement sequence 301 (e.g. P5) and the second terminal sequencing primer binding site complement 304′ (e.g. A14-ME; or if ME is not present, then A14). Separately, or in addition, a first index complement sequence (e.g. i7′) may be provided between the first terminal sequencing primer binding site 303 (e.g. ME′-B15′; or if ME′ is not present, then B15′) and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), a second terminal sequencing primer binding site complement 304′ (e.g. A14-ME; or if ME is not present, then A14), a second insert complement sequence 402′, a hybridisation sequence 403′ (e.g. ME′-HYB2′-ME; or if ME′ and ME are not present, then HYB2′), a first insert complement sequence 401′, a first terminal sequencing primer binding site 303 (e.g. ME′-B15′; or if ME′ is not present, then B15′), a first index complement sequence (e.g. i7′), and a second primer-binding sequence 302′ (e.g. P7′).

As described herein, the first insert sequence 401 and the second insert sequence 402 may comprise different types of library sequences.

In one embodiment, the first insert sequence 401 may be different to the second insert sequence 402 (e.g. genetically unrelated, and/or obtained from different sources), for example where the library is prepared using PCR stitching.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (a primer-binding sequence and a sequencing primer binding site, or a combination of a primer-binding sequence, an index sequence and a sequencing primer binding site) may be referred to herein as an adaptor sequence, and an insert (or inserts in concatenated strands) is flanked by a 5′ adaptor sequence and a 3′ adaptor sequence. The primer-binding sequence may also comprise a sequencing primer for the index read.

As used herein, an “adaptor” refers to a sequence that comprises a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the P5′ and P7′ primer-binding sequences are complementary to short primer sequences (or lawn primers) present on the surface of a flow cell. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

In concatenated strands, the hybridisation sequence (or the hybridisation sequence complement) may comprise an internal sequencing primer binding site. In other words, an internal sequencing primer binding site may form part of the hybridisation sequence. For example, ME′-HYB2 (or ME′-HYB2′) may act as an internal sequencing primer binding site to which a sequencing primer can bind. Alternatively, the hybridisation sequence may be an internal sequencing primer binding site. For example, HYB2 (or HYB2′) may act as an internal sequencing primer binding site to which a sequencing primer can bind. Accordingly, we may refer to the hybridisation site herein as comprising a sequencing primer binding site (e.g. a second sequencing primer binding site), or as a sequencing primer binding site (e.g. a second sequencing primer binding site).

### Cluster Generation and Amplification

Once a double stranded nucleic acid template is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 4th Ed, Cold Spring Harbor Laboratory Press, Cold Spring Harbor Laboratory Press, NY; Current Protocols, eds Ausubel et al). In one embodiment, chemical denaturation may be used.

Following denaturation, a single-stranded library may be contacted in free solution onto a solid support comprising surface capture moieties (for example P5 and P7 lawn primers).

Thus, embodiments of the present invention may be performed on a solid support 200, such as a flowcell. However, in alternative embodiments, seeding and clustering can be conducted off-flowcell using other types of solid support, for example, beads or wells.

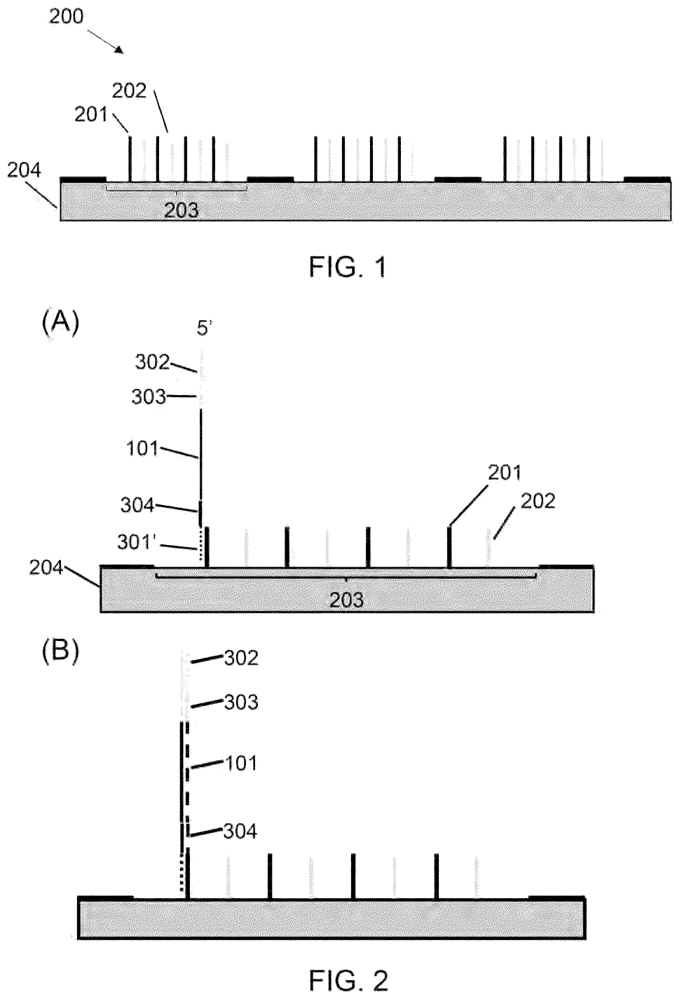

The solid support 200 may comprise a substrate 204. See FIG. 1. The substrate 204 comprises at least one well 203 (e.g. a nanowell), and typically comprises a plurality of wells 203 (e.g. a plurality of nanowells).

In one embodiment, the solid support comprises at least one first immobilised primer and at least one second immobilised primer. These immobilised primers may also be known as lawn primers.

Thus, each well 203 may comprise at least one first immobilised primer 201, and typically may comprise a plurality of first immobilised primers 201. In addition, each well 203 may comprise at least one second immobilised primer 202, and typically may comprise a plurality of second immobilised primers 202. Thus, each well 203 may comprise at least one first immobilised primer 201 and at least one second immobilised primer 202, and typically may comprise a plurality of first immobilised primers 201 and a plurality of second immobilised primers 202.

The first immobilised primer 201 may be attached via a 5′-end of its polynucleotide chain to the solid support 200. When extension occurs from the first immobilised primer 201, the extension may be in a direction away from the solid support 200.

The second immobilised primer 202 may be attached via a 5′-end of its polynucleotide chain to the solid support 200. When extension occurs from second immobilised primer 202, the extension may be in a direction away from the solid support 200.

The first immobilised primer 201 may be different to the second immobilised primer 202 and/or a complement of the second immobilised primer 202. The second immobilised primer 202 may be different to the first immobilised primer 201 and/or a complement of the first immobilised primer 201.

The (or each of the) first immobilised primer(s) 201 may comprise a sequence as defined in SEQ ID NO. 1 or 5, or a variant or fragment thereof. The second immobilised primer(s) 202 may comprise a sequence as defined in SEQ ID NO. 2, or a variant or fragment thereof.

By way of brief example, following attachment of the P5 and P7 primers to the solid support, the solid support may be contacted with the template to be amplified under conditions which permit hybridisation (or annealing-such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. The resulting structure is referred to herein as a sequence bridge. Further rounds of amplification (analogous to a standard PCR reaction) leads to the formation of clusters or colonies of template molecules bound to the solid support. This is called clustering.

Thus, solid-phase amplification by either a method analogous to that of WO 98/44151 or that of WO 00/18957 (the contents of which are incorporated herein in their entirety by reference) will result in production of a clustered array comprised of colonies of “bridged” amplification products (or sequence bridges). This process is known as bridge amplification. Both strands of the amplification products will be immobilised on the solid support at or near the 5′ end, this attachment being derived from the original attachment of the amplification primers. Typically, the amplification products within each colony will be derived from amplification of a single template molecule. Other amplification procedures may be used, and will be known to the skilled person. For example, amplification may be isothermal amplification using a strand displacement polymerase; or may be exclusion amplification as described in WO 2013/188582. Further information on amplification can be found in WO 02/06456 and WO 07/107710, the contents of which are incorporated herein in their entirety by reference.

Through such approaches, a cluster of template molecules is formed, comprising copies of a template strand and copies of the complement of the template strand.

In some cases, to facilitate sequencing, one set of strands (either the original template strands or the complement strands thereof) may be removed from the solid support leaving either the original template strands or the complement strands. Suitable methods for removing such strands are described in more detail in application number WO 07/010251, the contents of which are incorporated herein by reference in their entirety.

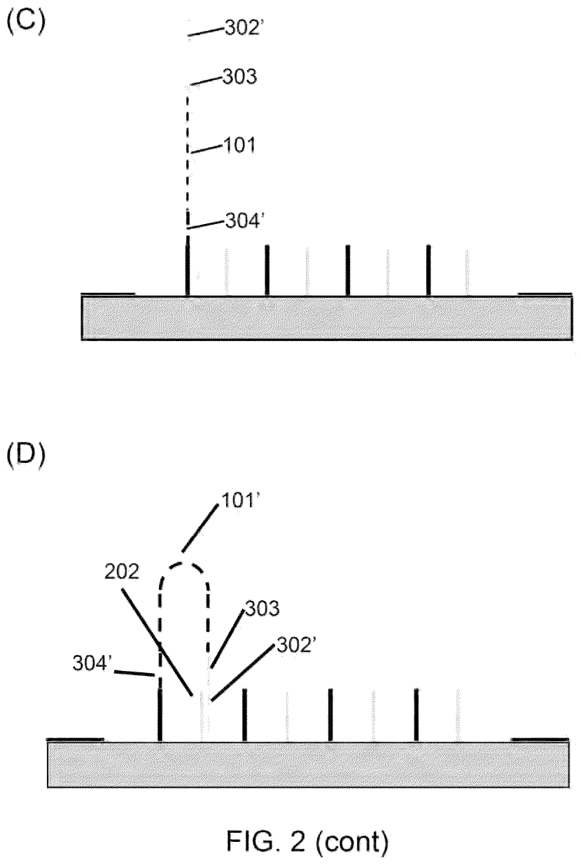

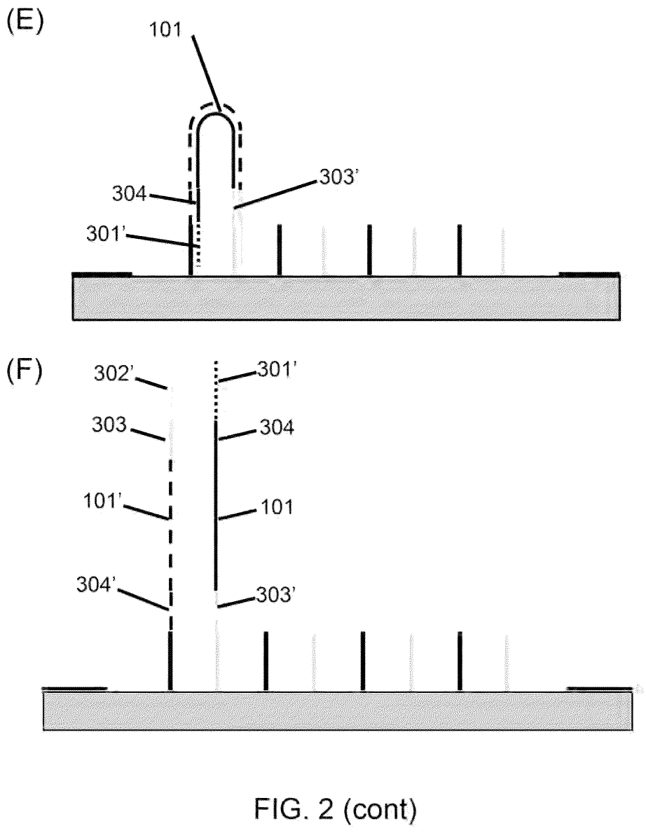

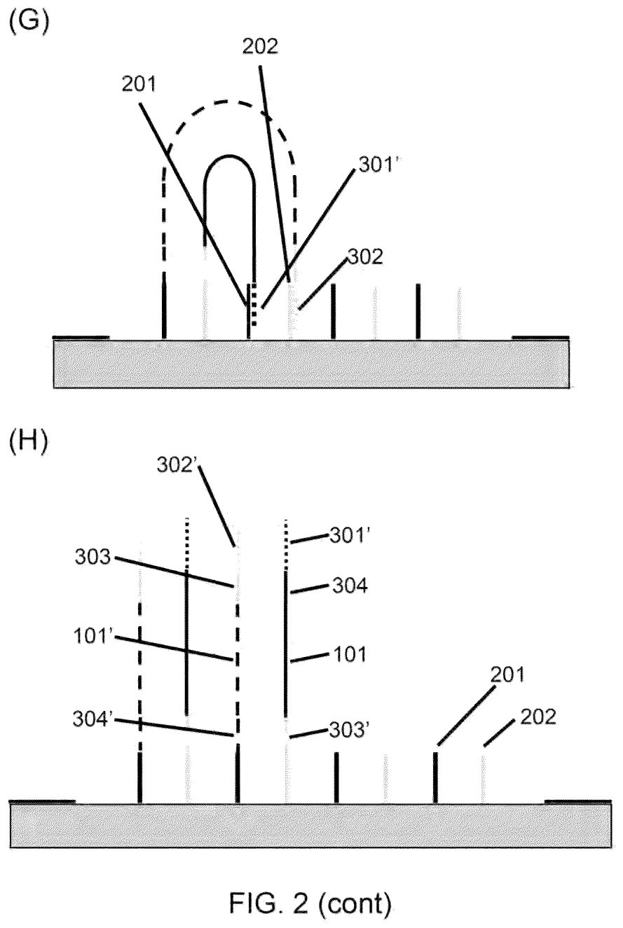

The steps of cluster generation and amplification for templates comprising a first portion and a second portion are illustrated below and in FIG. 2.

The steps of cluster generation and amplification for templates including a concatenated polynucleotide sequence comprising a first portion and a second portion, as well as templates including a first polynucleotide sequence comprising a first portion and a second polynucleotide sequence comprising a second portion, are illustrated below and in FIG. 26.

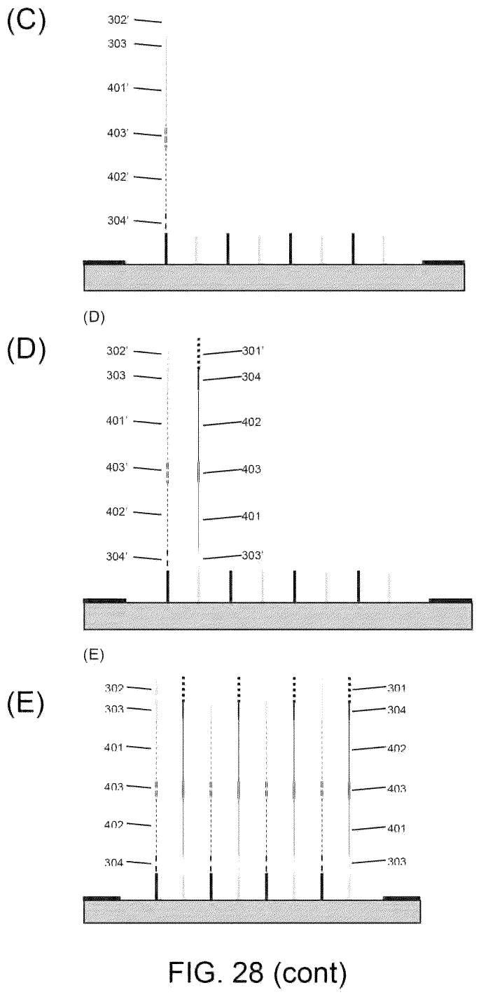

The steps of cluster generation and amplification for templates including a first polynucleotide sequence comprising a first portion and a second polynucleotide sequence comprising a second portion, are illustrated below and in FIG. 28.

In cases where (separate) polynucleotide strands are used, each first polynucleotide sequence may be attached (via 5′-end of the first polynucleotide sequence) to a first immobilised primer, and wherein each second polynucleotide sequence is attached (via the 5′-end of the second polynucleotide sequence) to a second immobilised primer. Each first polynucleotide sequence may comprise a second adaptor sequence, wherein the second adaptor sequence comprises a portion which is substantially complementary to the second immobilised primer (or is substantially complementary to the second immobilised primer). The second adaptor sequence may be at a 3′-end of the first polynucleotide sequence. Each second polynucleotide sequence may comprise a first adaptor sequence, wherein the first adaptor sequence comprises a portion which is substantially complementary to the first immobilised primer (or is substantially complementary to the first immobilised primer). The first adaptor sequence may be at a 3′-end of the second polynucleotide sequence.

In an embodiment, a solution comprising a polynucleotide library prepared by a loop fork method as described above may be flowed across a flowcell.

A particular polynucleotide strand from the polynucleotide library to be sequenced comprising, in a 5′ to 3′ direction, a second primer-binding complement sequence 302 (e.g. P7), an optional first terminal sequencing primer binding site complement 303′, a first insert sequence 401 (A and B), a loop sequence 403 (L′), a second insert sequence 402 (B′ and A′), an optional second terminal sequencing primer binding site 304, and a first primer-binding sequence 301′ (e.g. P5′), may anneal (via the first primer-binding sequence 301′) to the first immobilised primer 201 (e.g. P5 lawn primer) located within a particular well 203 (FIG. 28A).

The polynucleotide library may comprise other polynucleotide strands with different first insert sequences 401 and second insert sequences 402. Such other polynucleotide strands may anneal to corresponding first immobilised primers 201 (e.g. P5 lawn primers) in different wells 203, thus enabling parallel processing of the various different strands within the polynucleotide library.

A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, an optional second terminal sequencing primer binding site complement 304′, a second insert complement sequence 402′ (A′ copy and B′ copy), a loop complement sequence 403′ (L′), a first insert complement sequence 401′ (B copy and A copy), an optional first terminal sequencing primer binding site 303, and a second primer-binding sequence 302′ (e.g. P7′) (FIG. 28B). Such a process may utilise a polymerase, such as a DNA or RNA polymerase.

If the polynucleotides in the library comprise index sequences, then corresponding index sequences are also produced in the template.

The polynucleotide strand from the polynucleotide library may then be dehybridised and washed away, leaving a template strand attached to the first immobilised primer 201 (e.g. P5 lawn primer) (FIG. 28C).

The second primer-binding sequence 302′ (e.g. P7′) on the template strand may then anneal to a second immobilised primer 202 (e.g. P7 lawn primer) located within the well 203. This forms a “bridge”.

A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, an optional first terminal sequencing primer binding site complement 303′, a first insert sequence 401 (A and B), a loop sequence 403 (L), a second insert sequence 402 (B′ and A′), an optional second terminal sequencing primer binding site 304, and a first primer-binding sequence 301′ (e.g. P5′). Again, such a process may utilise a polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 28D).

A subsequent bridge amplification cycle can then lead to amplification of the strand attached to the first immobilised primer 201 (e.g. P5 lawn primer) and the strand attached to the second immobilised primer 202 (e.g. P7 lawn primer). The second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203.

Completion of bridge amplification and dehybridisation may then provide an amplified cluster, thus providing a plurality of polynucleotide sequences comprising a first insert complement sequence 401′ and a second insert complement sequence 402′, as well as a plurality of polynucleotide sequences comprising a first insert sequence 401 and a second insert sequence 402 (FIG. 28E).

If desired, further bridge amplification cycles may be conducted to increase the number of polynucleotide sequences within the well 203.

Once again, although FIG. 28 shows the presence of a first terminal sequencing primer binding site complement 303′, a second terminal sequencing primer binding site 304, a second terminal sequencing primer binding site complement 304′, and a first terminal sequencing primer binding site 303, these are optional as mentioned above. Accordingly, these sections may be omitted from the template and template complement strands.

The methods for clustering and amplification described above generally relate to conducting non-selective amplification. However, methods of the present invention relating to selective processing may comprise conducting selective amplification, which is described in further detail below under selective processing.

In cases where single (concatenated) polynucleotide strands are used, each polynucleotide sequence may be attached (via 5′-end of the (concatenated) polynucleotide sequence) to a first immobilised primer. Each polynucleotide sequence may comprise a second adaptor sequence, wherein the second adaptor comprises a portion which is substantially complementary to the second immobilised primer (or is substantially complementary to the second immobilised primer). The second adaptor sequence may be at a 3′-end of the (concatenated) polynucleotide sequence. In an embodiment, a solution comprising a polynucleotide library prepared by a tandem insert method as described above may be flowed across a flowcell.

A particular concatenated polynucleotide strand from the polynucleotide library to be sequenced comprising, in a 5′ to 3′ direction, a second primer-binding complement sequence 302 (e.g. P7), a first terminal sequencing primer binding site complement 303′ (e.g. B15-ME), a first insert sequence 401, a hybridisation complement sequence 403 (e.g. ME′-HYB2-ME), a second insert sequence 402, a second terminal sequencing primer binding site 304 (e.g. ME′-A14′), and a first primer-binding sequence 301′ (e.g. P5′), may anneal (via the first primer-binding sequence 301′) to the first immobilised primer 201 (e.g. P5 lawn primer) located within a particular well 203 (FIG. 28A).

The polynucleotide library may comprise other concatenated polynucleotide strands with different first insert sequences 401 and second insert sequences 402. Such other polynucleotide strands may anneal to corresponding first immobilised primers 201 (e.g. P5 lawn primers) in different wells 203, thus enabling parallel processing of the various different concatenated strands within the polynucleotide library.

A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, a second terminal sequencing primer binding site complement 304′ (e.g. A14-ME; or if ME is not present, then A14), a second insert complement sequence 402′ (which represents a type of “second portion”), a hybridisation sequence 403′ (which comprises a type of “second sequencing primer binding site”) (e.g. ME′-HYB2′-ME; or if ME′ and ME are not present, then HYB2′), a first insert complement sequence 401′ (which represents a type of “first portion”), a first terminal sequencing primer binding site 303 (which represents a type of “first sequencing primer binding site”) (e.g. ME′-B15′; or if ME′ is not present, then B15′), and a second primer-binding sequence 302′ (e.g. P7′) (FIG. 28B). Such a process may utilise a polymerase, such as a DNA or RNA polymerase. If the polynucleotides in the library comprise index sequences, then corresponding index sequences are also produced in the template. The concatenated polynucleotide strand from the polynucleotide library may then be dehybridised and washed away, leaving a template strand attached to the first immobilised primer 201 (e.g. P5 lawn primer) (FIG. 28C).

The second primer-binding sequence 302′ (e.g. P7′) on the template strand may then anneal to a second immobilised primer 202 (e.g. P7 lawn primer) located within the well 203. This forms a “bridge”. A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), and a first primer-binding sequence 301′ (e.g. P5′). Again, such a process may utilise a polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 28D).

A subsequent bridge amplification cycle can then lead to amplification of the strand attached to the first immobilised primer 201 (e.g. P5 lawn primer) and the strand attached to the second immobilised primer 202 (e.g. P7 lawn primer). The second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203.

Completion of bridge amplification and dehybridisation may then provide an amplified cluster, thus providing a plurality of concatenated polynucleotide sequences comprising a first insert complement sequence 401′ (i.e. “first portions”) and a second insert complement sequence 402′ (i.e. second portions”), as well as a plurality of concatenated polynucleotide sequences comprising a first insert sequence 401 and a second insert sequence 402 (FIG. 28E).

If desired, further bridge amplification cycles may be conducted to increase the number of polynucleotide sequences within the well 203.

In one aspect, before sequencing, one group of strands (either the group of template polynucleotides, or the group of template complement polynucleotides thereof) is removed from the solid support to form a (monoclonal) cluster, leaving either the templates or the template complements (FIG. 28F).

In some example, the “first portion” corresponds with the forward strand of the template 101′, and the “second portion” corresponds with the forward complement strand of the template 101.

However, other set-ups may be obtained by changing the library used. For example, by using a loop fork method to prepare a library, a portion at or close to the loop (or the loop complement) may be cleaved (e.g. by nicking). In these cases, the loop may comprise a cleavage site (e.g. a restriction recognition site, a cleavable linker, a modified nucleotide, or the like). By conducting cleavage at the loop, it is possible to produce a well 203, where the “first portion” corresponds with a forward strand of the template, and the “second portion” corresponds with a reverse complement strand of the template. As such, different types of strands for the “first portions” and “second portions” may be prepared for templates including a first polynucleotide sequence comprising a first portion and a second polynucleotide sequence comprising a second portion.

In an embodiment, a solution comprising a polynucleotide library prepared by ligating adaptor sequences to double-stranded polynucleotide sequences as described above may be flown across a flowcell.

A particular polynucleotide strand from the polynucleotide library to be sequenced comprising, in a 5′ to 3′ direction, a second primer-binding complement sequence 302 (e.g. P7), a first terminal binding site complement 303′ (e.g. SBS12), a forward strand of the sequence 101, a second terminal sequencing primer binding site 304 (e.g. SBS3′) and a first primer-binding sequence 301′ (e.g. P5′), may anneal (via the first primer-binding sequence 301′) to the first immobilised primer 201 (e.g. P5 lawn primer) located within a particular well 203 (FIG. 2A).

The polynucleotide library may comprise other polynucleotide strands with different forward strands of the sequence 101. Such other polynucleotide strands may anneal to corresponding first immobilised primers 201 (e.g. P5 lawn primers) in different wells 203, thus enabling parallel processing of the various different strands within the polynucleotide library. A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, a second terminal sequencing primer binding site complement 304′ (e.g. SBS3), a forward strand of the template 101′ (which represents a type of “first portion”), a first terminal sequencing primer binding site 303 (which represents a type of “first sequencing primer binding site”) (e.g. SBS12′), and a second primer-binding sequence 302′ (e.g. P7′) (FIG. 2B). Such a process may utilise an appropriate polymerase, such as a DNA or RNA polymerase.

If the polynucleotides in the library comprise index sequences, then corresponding index sequences are also produced in the template.

The polynucleotide strand from the polynucleotide library may then be dehybridised and washed away, leaving a template strand attached to the first immobilised primer 201 (e.g. P5 lawn primer) (FIG. 2C).

The second primer-binding sequence 302′ (e.g. P7′) on the template strand may then anneal to a second immobilised primer 202 (e.g. P7 lawn primer) located within the well 203. This forms a “bridge” (FIG. 2D).

A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, a first terminal sequencing primer binding site complement 303′ (e.g. SBS12), a forward complement strand of the template 101 (which represents a type of “second portion”), a second terminal sequencing primer binding site 304 (which represents a type of “second sequencing primer binding site”) (e.g. SBS3′), and a first primer-binding sequence 301′ (e.g. P5′) (FIG. 2E). Again, such a process may utilise a suitable polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 2F).

A subsequent bridge amplification cycle can then lead to amplification of the strand attached to the first immobilised primer 201 (e.g. P5 lawn primer) and the strand attached to the second immobilised primer 202 (e.g. P7 lawn primer). Similar to FIG. 2D, the second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203 (FIG. 2G).

Completion of bridge amplification and dehybridisation may then provide an amplified (duoclonal) cluster, thus providing a plurality of first polynucleotide sequences comprising the forward strand of the template 101′ (i.e. “first portions”), and a plurality of second polynucleotide sequences comprising the forward complement strand of the template 101 (i.e. “second portions”) (FIG. 2H).

If desired, further bridge amplification cycles may be conducted to increase the number of first polynucleotide sequences and second polynucleotide sequences within the well 203.

Preferably before sequencing, one group of strands (either the group of template polynucleotides, or the group of template complement polynucleotides thereof) is removed from the solid support to form a (monoclonal) cluster, leaving either the templates or the template complements (FIG. 28F).

The methods for clustering and amplification described above generally relate to conducting non-selective amplification. However, methods of the present invention relating to selective processing may comprise conducting selective amplification, which is described in further detail below under selective processing.

The steps of cluster generation and amplification for templates including a concatenated polynucleotide sequence comprising n portions (e.g. a concatenated polynucleotide sequence comprising a first portion and a second portion) are illustrated below and in FIG. 28.

In cases where single (concatenated) polynucleotide strands are used, each polynucleotide sequence may be attached (via 5′-end of the (concatenated) polynucleotide sequence) to a first immobilised primer. Each polynucleotide sequence may comprise a second adaptor sequence, wherein the second adaptor comprises a portion which is substantially complementary to the second immobilised primer (or is substantially complementary to the second immobilised primer). The second adaptor sequence may be at a 3′-end of the (concatenated) polynucleotide sequence.

In an embodiment, a solution comprising a polynucleotide library prepared by a PCR stitching method as described above may be flowed across a flowcell.

In an illustrative case where n is 2, a particular concatenated polynucleotide strand from the polynucleotide library to be sequenced comprising, in a 5′ to 3′ direction, a second primer-binding complement sequence 302 (e.g. P7), a first terminal sequencing primer binding site complement 303′ (e.g. B15-ME), a first insert sequence 401, a hybridisation complement sequence 403 (e.g. ME′-HYB2-ME), a second insert sequence 402, a second terminal sequencing primer binding site 304 (e.g. ME′-A14′), and a first primer-binding sequence 301′ (e.g. P5′), may anneal (via the first primer-binding sequence 301′) to the first immobilised primer 201 (e.g. P5 lawn primer) located within a particular well 203 (FIG. 28A).

The polynucleotide library may comprise other concatenated polynucleotide strands with different first insert sequences 401 and second insert sequences 402. Such other polynucleotide strands may anneal to corresponding first immobilised primers 201 (e.g. P5 lawn primers) in different wells 203, thus enabling parallel processing of the various different concatenated strands within the polynucleotide library.

A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, a second terminal sequencing primer binding site complement 304′ (e.g. A14-ME; or if ME is not present, then A14), a second insert complement sequence 402′ (which represents a type of “second portion”), a hybridisation sequence 403′ (which comprises a type of “second sequencing primer binding site”) (e.g. ME′-HYB2′-ME; or if ME′ and ME are not present, then HYB2′), a first insert complement sequence 401′ (which represents a type of “first portion”), a first terminal sequencing primer binding site 303 (which represents a type of “first sequencing primer binding site”) (e.g. ME′-B15′; or if ME′ is not present, then B15′), and a second primer-binding sequence 302′ (e.g. P7′) (FIG. 28B). Such a process may utilise a polymerase, such as a DNA or RNA polymerase.

If the polynucleotides in the library comprise index sequences, then corresponding index sequences are also produced in the template.

The concatenated polynucleotide strand from the polynucleotide library may then be dehybridised and washed away, leaving a template strand attached to the first immobilised primer 201 (e.g. P5 lawn primer) (FIG. 28C).

The second primer-binding sequence 302′ (e.g. P7′) on the template strand may then anneal to a second immobilised primer 202 (e.g. P7 lawn primer) located within the well 203. This forms a “bridge”.

A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), and a first primer-binding sequence 301′ (e.g. P5′). Again, such a process may utilise a polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 28D).

A subsequent bridge amplification cycle can then lead to amplification of the strand attached to the first immobilised primer 201 (e.g. P5 lawn primer) and the strand attached to the second immobilised primer 202 (e.g. P7 lawn primer). The second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203.

Completion of bridge amplification and dehybridisation may then provide an amplified cluster, thus providing a plurality of concatenated polynucleotide sequences comprising a first insert complement sequence 401′ (i.e. “first portions”) and a second insert complement sequence 402′ (i.e. second portions”), as well as a plurality of concatenated polynucleotide sequences comprising a first insert sequence 401 and a second insert sequence 402 (FIG. 28E).

If desired, further bridge amplification cycles may be conducted to increase the number of polynucleotide sequences within the well 203.

In one example, before sequencing, one group of strands (either the group of template polynucleotides, or the group of template complement polynucleotides thereof) is removed from the solid support to form a (monoclonal) cluster, leaving either the templates or the template complements (FIG. 28F).

### Sequencing

As described herein, the template provides information (e.g. identification of the genetic sequence, identification of epigenetic modifications) on the original target polynucleotide sequence. For example, a sequencing process (e.g. a sequencing-by-synthesis (referred to herein as SBS) or sequencing-by-ligation process) may reproduce information that was present in the original target polynucleotide sequence, by using complementary base pairing.

In one embodiment, sequencing may be carried out using any suitable “sequencing-by-synthesis” technique, wherein nucleotides are added successively in cycles to the free 3′ hydroxyl group, resulting in synthesis of a polynucleotide chain in 5′ to 3′ direction. The nature of the nucleotide added may be determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the contents of which are incorporated herein by reference in their entirety. Alternatively, a separate reaction may be carried out containing each of the modified nucleotides added individually.

The modified nucleotides may carry a label to facilitate their detection. Such a label may be configured to emit a signal, such as an electromagnetic signal, or a (visible) light signal.

In a particular embodiment, the label is a fluorescent label (e.g. a dye). Thus, such a label may be configured to emit an electromagnetic signal, or a (visible) light signal. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

However, the detectable label need not be a fluorescent label. Any label can be used which allows the detection of the incorporation of the nucleotide into the DNA sequence.

Each cycle may involve simultaneous delivery of four different nucleotide types to the array of template molecules. Alternatively, different nucleotide types can be added sequentially and an image of the array of template molecules can be obtained between each addition step.

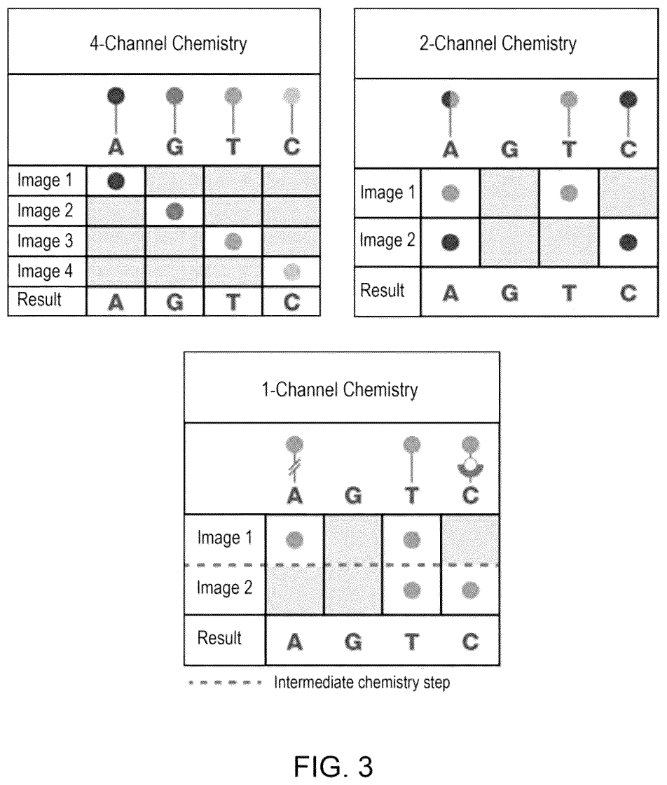

In some embodiments, each nucleotide type may have a (spectrally) distinct label. In other words, four channels may be used to detect four nucleobases (also known as 4-channel chemistry) (FIG. 3—left). For example, a first nucleotide type (e.g. A) may include a first label (e.g. configured to emit a first wavelength, such as red light), a second nucleotide type (e.g. G) may include a second label (e.g. configured to emit a second wavelength, such as blue light), a third nucleotide type (e.g. T) may include a third label (e.g. configured to emit a third wavelength, such as green light), and a fourth nucleotide type (e.g. C) may include a fourth label (e.g. configured to emit a fourth wavelength, such as yellow light). Four images can then be obtained, each using a detection channel that is selective for one of the four different labels. For example, the first nucleotide type (e.g. A) may be detected in a first channel (e.g. configured to detect the first wavelength, such as red light), the second nucleotide type (e.g. G) may be detected in a second channel (e.g. configured to detect the second wavelength, such as blue light), the third nucleotide type (e.g. T) may be detected in a third channel (e.g. configured to detect the third wavelength, such as green light), and the fourth nucleotide type (e.g. C) may be detected in a fourth channel (e.g. configured to detect the fourth wavelength, such as yellow light). Although specific pairings of bases to signal types (e.g. wavelengths) are described above, different signal types (e.g. wavelengths) and/or permutations may also be used.

In some embodiments, detection of each nucleotide type may be conducted using fewer than four different labels. For example, sequencing-by-synthesis may be performed using methods and systems described in US 2013/0079232, which is incorporated herein by reference.

Thus, in some embodiments, two channels may be used to detect four nucleobases (also known as 2-channel chemistry) (FIG. 3—middle). For example, a first nucleotide type (e.g. A) may include a first label (e.g. configured to emit a first wavelength, such as green light) and a second label (e.g. configured to emit a second wavelength, such as red light), a second nucleotide type (e.g. G) may not include the first label and may not include the second label, a third nucleotide type (e.g. T) may include the first label (e.g. configured to emit the first wavelength, such as green light) and may not include the second label, and a fourth nucleotide type (e.g. C) may not include the first label and may include the second label (e.g. configured to emit the second wavelength, such as red light). Two images can then be obtained, using detection channels for the first label and the second label. For example, the first nucleotide type (e.g. A) may be detected in both a first channel (e.g. configured to detect the first wavelength, such as red light) and a second channel (e.g. configured to detect the second wavelength, such as green light), the second nucleotide type (e.g. G) may not be detected in the first channel and may not be detected in the second channel, the third nucleotide type (e.g. T) may be detected in the first channel (e.g. configured to detect the first wavelength, such as red light) and may not be detected in the second channel, and the fourth nucleotide type (e.g. C) may not be detected in the first channel and may be detected in the second channel (e.g. configured to detect the second wavelength, such as green light). Although specific pairings of bases to signal types (e.g. wavelengths) and/or combinations of channels are described above, different signal types (e.g. wavelengths) and/or permutations may also be used.

In some embodiments, one channel may be used to detect four nucleobases (also known as 1-channel chemistry) (FIG. 3—right). For example, a first nucleotide type (e.g. A) may include a cleavable label (e.g. configured to emit a wavelength, such as green light), a second nucleotide type (e.g. G) may not include a label, a third nucleotide type (e.g. T) may include a non-cleavable label (e.g. configured to emit the wavelength, such as green light), and a fourth nucleotide type (e.g. C) may include a label-accepting site which does not include the label. A first image can then be obtained, and a subsequent treatment carried out to cleave the label attached to the first nucleotide type, and to attach the label to the label-accepting site on the fourth nucleotide type. A second image may then be obtained. For example, the first nucleotide type (e.g. A) may be detected in a channel (e.g. configured to detect the wavelength, such as green light) in the first image and not detected in the channel in the second image, the second nucleotide type (e.g. G) may not be detected in the channel in the first image and may not be detected in the channel in the second image, the third nucleotide type (e.g. T) may be detected in the channel (e.g. configured to detect the wavelength, such as green light) in the first image and may be detected in the channel (e.g. configured to detect the wavelength, such as green light) in the second image, and the fourth nucleotide type (e.g. C) may not be detected in the channel in the first image and may be detected in the channel in the second image (e.g. configured to detect the wavelength, such as green light). Although specific pairings of bases to signal types (e.g. wavelengths) and/or combinations of images are described above, different signal types (e.g. wavelengths), images and/or permutations may also be used.

In some embodiments, the fluorescent labels are selected from the group consisting of polymethine derivatives, coumarin derivatives, benzopyran derivatives, chromenoquinoline derivatives, compounds containing bis-boron heterocycles such as BOPPY and BOPYPY. In some embodiments, the fluorescent label is attached to the nucleotide through a cleavable linker. In some further embodiments, the labeled nucleotide may have the fluorescent label attached to the C5 position of a pyrimidine base or the C7 position of a 7-deaza purine base, optionally through a cleavable linker moiety. For example, the nucleobase may be 7-deaza adenine and the dye is attached to the 7-deaza adenine at the C7 position, optionally through a cleavable linker. The nucleobase may be 7-deaza guanine and the dye is attached to the 7-deaza guanine at the C7 position, optionally through a cleavable linker. The nucleobase may be cytosine and the dye is attached to the cytosine at the C5 position, optionally through a cleavable linker. As another example, the nucleobase may be thymine or uracil and the dye is attached to the thymine or uracil at the C5 position, optionally through a cleavable linker. In some further embodiments, the cleavable linker may comprise similar or the same chemical moiety as the reversible terminator 3′ hydroxy blocking group such that 3′ hydroxy blocking group and the cleavable linker may be removed under the same reaction condition or in a single chemical reaction. Non-limiting example of the cleavable linker include the LN3 linker, the sPA linker, and the AOL linker, each of which is exemplified below.

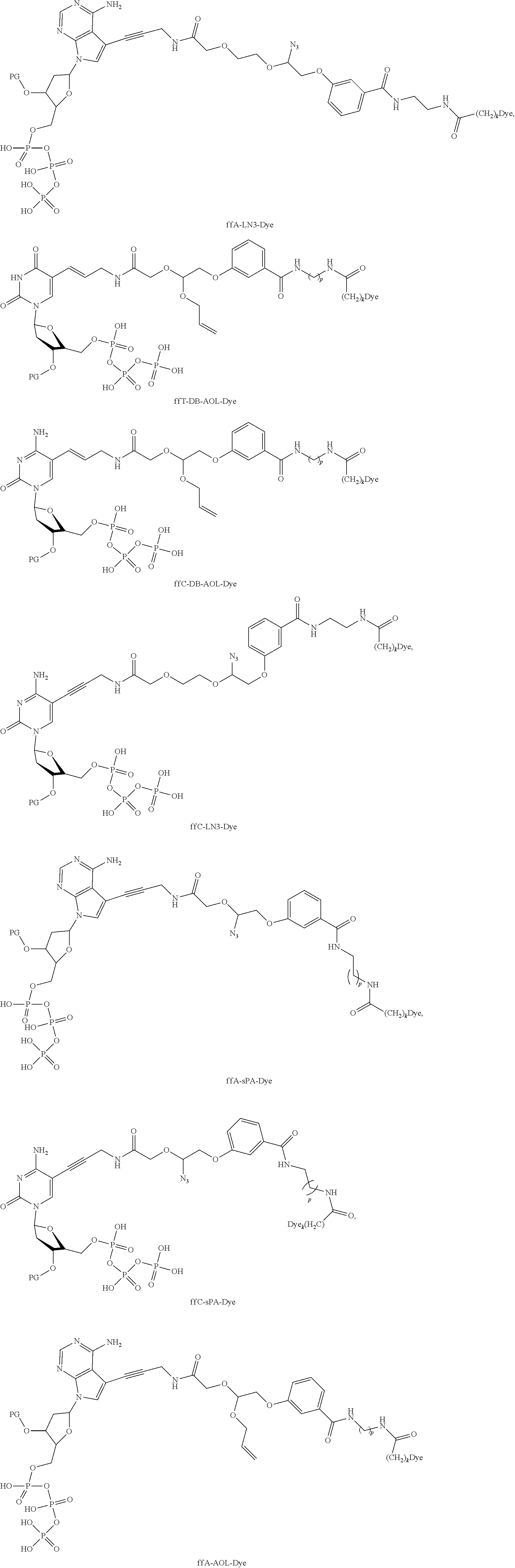

In some embodiments, the nucleotides are selected from the group consisting of an analog of dGTP, an analog of dTTP, an analog of dUTP, an analog of dCTP, and an analog of dATP. In some embodiments, the first nucleotide is a first reversibly blocked nucleotide triphosphate (rbNTP), the second nucleotide is a second rbNTP, the third nucleotide is a third rbNTP, and the fourth nucleotide is a fourth rbNTP, wherein each of the first nucleotide, second nucleotide, third nucleotide and fourth nucleotide is a different type of nucleotide from the other. In some embodiments, the four rbNTPs are selected from the group consisting of rbATP, rbTTP, rbUTP, rbCTP, and rbGTP. In some embodiments, each of the four rbNTPs includes a modified base and a reversible terminator 3′ blocking group. Non-limiting example of 3′ blocking group include azidomethyl (*—CH2N3), substituted azidomethyl (e.g., *—CH(CHF2)N3 or *—CH(CH2F)N3) and *—CH2—O—CH2—CH═CH2, where the asterisk * indicates the point attachment to 3′ oxygen of the ribose or deoxyribose ring of the nucleotide.

Further details about the dyes and the fully functionalized nucleotides can be found in U.S. Patent Application Publication Numbers 2018/0094140 and 2020/0277670, International Patent Application Publication Number 2017/051201, and U.S. Provisional Patent Application Nos. 63/057,758 and 63/127,061, the disclosures of which are incorporated herein by reference in their entireties.

In one embodiment, the sequencing process comprises a first sequencing read (referred to herein as R1) and second sequencing read (referred to herein as R2). As described below, in each read at least two different polynucleotide strands may be sequenced simultaneously, generating a R1.1 and R1.2 read and a R2.1 and R2.2 read. The first sequencing read and the second sequencing read may also be conducted concurrently. In other words, the first sequencing read and the second sequencing read may be conducted at the same time.

The first sequencing read may comprise the binding of a first sequencing primer (also known as a read 1 sequencing primer) to the first sequencing primer binding site. The second sequencing read may comprise the binding of a second sequencing primer (also known as a read 2 sequencing primer) to the second sequencing primer binding site.

The first sequencing read may comprise the binding of a first sequencing primer (also known as a read 1.1 sequencing primer) to the first sequencing primer binding site (e.g. within loop complement sequence 403′). The second sequencing read may comprise the binding of a second sequencing primer (also known as a read 1.2 sequencing primer) to the second sequencing primer binding site (e.g. within loop sequence 403).

This leads to sequencing of the first portion (e.g. second insert complement sequence 402′) and the second portion (e.g. first insert sequence 401).

Other embodiments may involve strand displacement sequencing-by-synthesis (strand displacement SBS). In such a case, a strand displacement polymerase may initiate SBS from a nick. Further examples of strand displacement SBS are described in greater detail below.

The first sequencing read may comprise the binding of a first sequencing primer (also known as a read 1 sequencing primer) to the first sequencing primer binding site (e.g. first terminal sequencing primer binding site 303 in templates including a concatenated polynucleotide sequence comprising a first portion and a second portion). The second sequencing read may comprise the binding of a second sequencing primer (also known as a read 2 sequencing primer) to the second sequencing primer binding site (e.g. a portion of hybridisation sequence 403′ in templates including a concatenated polynucleotide sequence comprising a first portion and a second portion).

The second sequencing read may comprise the binding of a second sequencing primer (also known as a read 2 sequencing primer) to the second sequencing primer binding site (e.g. second terminal sequencing primer binding site 1304 in templates including a first polynucleotide sequence comprising a first portion and a second polynucleotide sequence comprising a second portion, or a portion of hybridisation sequence 1403′ in templates including a concatenated polynucleotide sequence comprising a first portion and a second portion).

This leads to sequencing of the first portion (e.g. first insert complement sequence 401′ in templates including a concatenated polynucleotide sequence comprising a first portion and a second portion) and the second portion (e.g. second insert complement sequence 402′ in templates including a concatenated polynucleotide sequence comprising a first portion and a second portion)

This leads to sequencing of the first portion (e.g. forward strand of the template 101′ in templates including a first polynucleotide sequence comprising a first portion and a second polynucleotide sequence comprising a second portion, or first insert complement sequence 401′ in templates including a concatenated polynucleotide sequence comprising a first portion and a second portion) and the second portion (e.g. forward complement strand of the template 101 in templates including a first polynucleotide sequence comprising a first portion and a second polynucleotide sequence comprising a second portion, or second insert complement sequence 402′ in templates including a concatenated polynucleotide sequence comprising a first portion and a second portion).

Alternative methods of sequencing include sequencing by ligation, for example as described in U.S. Pat. No. 6,306,597 or WO 06/084132, the contents of which are incorporated herein by reference.

Also described herein is a method of sequencing polynucleotide sequences to detect mismatched base pairs, comprising:
preparing polynucleotide sequences for detection of mismatched base pairs using a method as described herein;
concurrently sequencing nucleobases in the first portion and the second portion; and
identifying mismatched base pairs by detecting differences when comparing a sequence output from the first portion with a sequence output from the second portion.

In one embodiment, sequencing is performed by sequencing-by-synthesis or sequencing-by-ligation.

In one aspect, the step of preparing the polynucleotide sequences comprises using a selective processing method as described herein; and wherein the step of concurrent sequencing nucleobases in the first portion and the second portion is based on the intensity of the first signal and the intensity of the second signal.

In one example, the mismatched base pair comprises an oxo-G to A base pair.

In one embodiment, the method may further comprise a step of conducting paired-end reads.

In some embodiments, where the method comprises a step of selectively processing the at least one polynucleotide sequence comprising the first portion and the second portion, such that a proportion of first portions are capable of generating a first signal and a proportion of second portions are capable of generating a second signal, wherein the selective processing causes an intensity of the first signal to be greater than an intensity of the second signal, the data may be analysed using 16 QAM as mentioned herein. Accordingly, the step of concurrently sequencing nucleobases may comprise:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

In one embodiment, selecting the classification based on the first and second intensity data may comprise selecting the classification based on the combined intensity of the first and second signal components and the combined intensity of the third and fourth signal components.

In one embodiment, the plurality of classifications may comprise sixteen classifications, each classification representing one of sixteen unique combinations of first and second nucleobases.

In one embodiment, the first signal component, second signal component, third signal component and fourth signal component may be generated based on light emissions associated with the respective nucleobase.

In one example, the light emissions may be detected by a sensor, wherein the sensor is configured to provide a single output based upon the first and second signals.

In one embodiment, the sensor may comprise a single sensing element.

In one embodiment, the method may further comprise repeating steps (a) to (d) for each of a plurality of base calling cycles.

In some embodiments, where a proportion of first portions is capable of generating a first signal and a proportion of second portions is capable of generating a second signal, wherein an intensity of the first signal is substantially the same as an intensity of the second signal, the data may be analysed using 9 QAM as mentioned herein.

Accordingly, the step of concurrently sequencing nucleobases may comprise:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification of the plurality of classifications represents one or more possible combinations of respective first and second nucleobases, and wherein at least one classification of the plurality of classifications represents more than one possible combination of respective first and second nucleobases; and
(d) based on the selected classification, determining sequence information from the first portion and the second portion.

In one embodiment, selecting the classification based on the first and second intensity data may comprise selecting the classification based on the combined intensity of the first and second signal components and the combined intensity of the third and fourth signal components.

In one aspect, when based on a nucleobase of the same identity, an intensity of the first signal component may be substantially the same as an intensity of the second signal component and an intensity of the third signal component is substantially the same as an intensity of the fourth signal component.

In one embodiment, the plurality of classifications may consist of a predetermined number of classifications.

In one embodiment, the plurality of classifications may comprise:
one or more classifications representing matching first and second nucleobases; and
one or more classifications representing mismatching first and second nucleobases, and
wherein determining sequence information of the first portion and second portion comprises:
   in response to selecting a classification representing matching first and second nucleobases, determining a match between the first and second nucleobases; or
   in response to selecting a classification representing mismatching first and second nucleobases, determining a mismatch between the first and second nucleobases.

In one embodiment, determining sequence information of the first portion and the second portion may comprise, in response to selecting a classification representing a match between the first and second nucleobases, base calling the first and second nucleobases.

In another embodiment, determining sequence information of the first portion and the second portion may comprise, based on the selected classification, determining that the second portion is modified relative to the first portion at a location associated with the first and second nucleobases.

In one example, the first signal component, second signal component, third signal component and fourth signal component may be generated based on light emissions associated with the respective nucleobase.

In one aspect, the light emissions may be detected by a sensor, wherein the sensor is configured to provide a single output based upon the first and second signals.

In one embodiment, the sensor may comprise a single sensing element.

In one embodiment, the method may further comprise repeating steps (a) to (d) for each of a plurality of base calling cycles.

Also described herein is a method of sequencing at least one polynucleotide sequence, comprising preparing at least one polynucleotide sequence for identification using a method as described herein; and concurrently sequencing nucleobases in the first portion and the second portion based on the intensity of the first signal and the intensity of the second signal. Preferably sequencing is performed by sequencing-by-synthesis or sequencing-by-ligation.

Preferably, the method may further comprise a step of conducting paired-end reads.

The data may be analysed using 16 QAM as mentioned herein.

Accordingly, the step of concurrently sequencing nucleobases may comprise:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

Preferably, selecting the classification based on the first and second intensity data may comprise selecting the classification based on the combined intensity of the first and second signal components and the combined intensity of the third and fourth signal components.

Preferably, the plurality of classifications may comprise sixteen classifications, each classification representing one of sixteen unique combinations of first and second nucleobases.

Preferably, the first signal component, second signal component, third signal component and fourth signal component may be generated based on light emissions associated with the respective nucleobase.

Preferably, the light emissions may be detected by a sensor, wherein the sensor is configured to provide a single output based upon the first and second signals.

Preferably, the sensor may comprise a single sensing element.

Preferably, the method may further comprise repeating steps (a) to (d) for each of a plurality of base calling cycles.

The methods for sequencing described above generally relate to conducting non-selective sequencing. However, methods of the present invention relating to selective processing may comprise conducting selective sequencing, which is described in further detail below under selective processing.

In one embodiment, for example in an illustrative case where n is 2, the sequencing process comprises a first sequencing read and second sequencing read. The first sequencing read and the second sequencing read may be conducted concurrently. In other words, the first sequencing read and the second sequencing read may be conducted at the same time. Similar considerations apply when n is more than 2, where n sequencing reads are conducted. The first sequencing read may comprise the binding of a first sequencing primer (also known as a read 1 sequencing primer) to the first sequencing primer binding site (e.g. first terminal sequencing primer binding site 303 in templates including a concatenated polynucleotide sequence comprising a first portion and a second portion). The second sequencing read may comprise the binding of a second sequencing primer (also known as a read 2 sequencing primer) to the second sequencing primer binding site (e.g. a portion of hybridisation sequence 403′ in templates including a concatenated polynucleotide sequence comprising a first portion and a second portion). Similar considerations apply when n is more than 2, where n sequencing primers are used.

This leads to sequencing of the first portion (e.g. first insert complement sequence 401′ in templates including a concatenated polynucleotide sequence comprising a first portion and a second portion) and the second portion (e.g. second insert complement sequence 402′ in templates including a concatenated polynucleotide sequence comprising a first portion and a second portion). Similar considerations apply when n is more than 2, where sequencing of the n portions is conducted.

Alternative methods of sequencing include sequencing by ligation, for example as described in U.S. Pat. No. 6,306,597 or WO 06/084132, the contents of which are incorporated herein by reference.

The methods for sequencing described above generally relate to conducting non-selective sequencing. However, methods of the present invention relating to selective processing may comprise conducting selective sequencing, which is described in further detail below under selective processing.

### Selective Processing Methods

In some embodiments, selective processing methods may be used to generate signals of different intensities. Accordingly, in some embodiments, the method may comprise selectively processing the at least one first polynucleotide sequence comprising a first portion and the at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

The method may comprise selectively processing a plurality of first polynucleotide sequences each comprising a first portion and a plurality of second polynucleotide sequences each comprising a second portion, such that a proportion of first portions are capable of generating a first signal and a proportion of second portions are capable of generating a second signal, wherein the selective processing causes an intensity of the first signal to be greater than an intensity of the second signal.

By “selective processing” is meant here performing an action that changes relative properties of the first portion and the second portion in the at least one first polynucleotide sequence comprising a first portion and at least one second polynucleotide sequence comprising a second portion (or the plurality of first polynucleotide sequences each comprising a first portion and the plurality of second polynucleotide sequences each comprising a second portion), so that the intensity of the first signal is greater than the intensity of the second signal. The property may be, for example, a concentration of first portions capable of generating the first signal relative to a concentration of second portions capable of generating the second signal. The action may include, for example, conducting selective amplification, conducting selective sequencing, or preparing for selective sequencing.

In one embodiment, the selective processing results in the concentration of the first portions capable of generating the first signal being greater than the concentration of the second portions capable of generating the second signal. In other words, the method of the invention results in an altered ratio of R1: R2 molecules, such as within a single cluster or a single well.

In one embodiment, the ratio may be between 1.25:1 to 5:1, or between 1.5:1 to 3:1, or about 2:1.

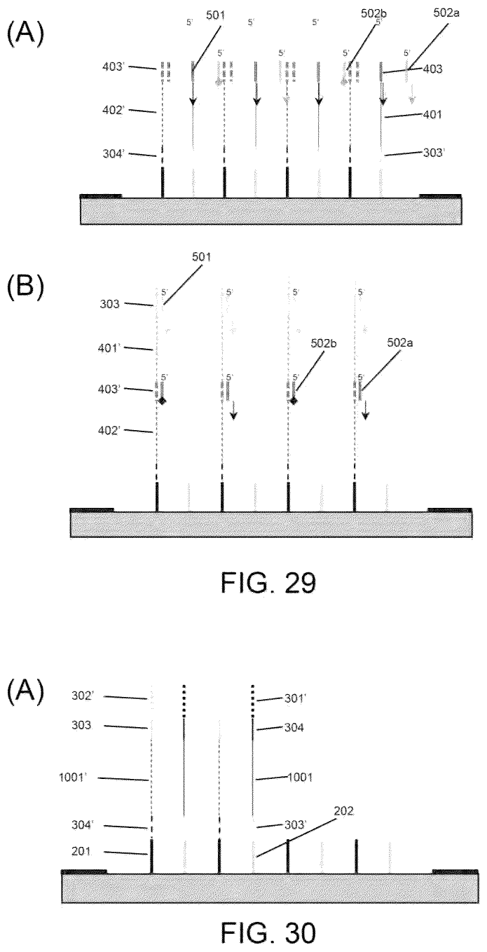

Selective processing may refer to conducting selective sequencing. Alternatively, selective processing may refer to preparing for selective sequencing. As shown in FIG. 29, in one example, selective sequencing may be achieved using a mixture of unblocked and blocked sequencing primers.

Where the method of the invention involves (separate) polynucleotide strands, with a first polynucleotide strand with a first portion, and a second polynucleotide strand with a second portion, the first polynucleotide strand may comprise a first sequencing primer binding site, and the second polynucleotide strand may comprise a second sequencing primer binding site, where the first sequencing primer binding site and second sequencing primer binding site are of a different sequence to each other and bind different sequencing primers.

In one embodiment, binding of first sequencing primers to the first sequencing primer site generates a first signal and binding of second sequencing primers to the second sequencing primer site generates a second signal, where the intensity of the first signal is greater than the intensity of the second signal. This may be applied to embodiments where the first polynucleotide strand comprises a first sequencing primer binding site, and the second polynucleotide strand comprises a second sequencing primer binding site. This is achieved using a mixed population of blocked and unblocked second sequencing primers that bind the second sequencing primer site. Any ratio of blocked: unblocked second primers can be used that generates a second signal that is of a lower intensity than the first signal, for example, the ratio of blocked: unblocked primers may be: 20:80 to 80:20, or 1:2 to 2:1.

In one aspect, a ratio of 50:50 of blocked: unblocked second primers is used, which in turn generates a second signal that is around 50% of the intensity of the first signal.

The first and second sequencing primers may be added to the flow cell at the same time, or separately but sequentially.

In some embodiments, selective processing methods may be used to generate signals of different intensities. Accordingly, in some embodiments, the method may comprise selectively processing at least one polynucleotide sequence comprising a first portion and a second portion, such that a proportion of first portions are capable of generating a first signal and a proportion of second portions are capable of generating a second signal, wherein the selective processing causes an intensity of the first signal to be greater than an intensity of the second signal.

The method may comprise selectively processing a plurality of polynucleotide sequences each comprising a first portion and a second portion, such that a proportion of first portions are capable of generating a first signal and a proportion of second portions are capable of generating a second signal, wherein the selective processing causes an intensity of the first signal to be greater than an intensity of the second signal.

By “selective processing” is meant here performing an action that changes relative properties of the first portion and the second portion in the at least one polynucleotide sequence comprising a first portion and a second portion (or the plurality of polynucleotide sequences each comprising a first portion and a second portion), so that the intensity of the first signal is greater than the intensity of the second signal. The property may be, for example, a concentration of first portions capable of generating the first signal relative to a concentration of second portions capable of generating the second signal. The action may include, for example, conducting selective sequencing, or preparing for selective sequencing.

In one embodiment, the selective processing results in the concentration of the first portions capable of generating the first signal being greater than the concentration of the second portions capable of generating the second signal. In other words, the method of the invention results in an altered ratio of R1: R2 molecules, such as within a single cluster or a single well. In one embodiment, the ratio may be between 1.25:1 to 5:1, or between 1.5:1 to 3:1, or about 2:1.

Selective processing may refer to conducting selective sequencing. Alternatively, selective processing may refer to preparing for selective sequencing. As shown in FIG. 29, in one example, selective sequencing may be achieved using a mixture of unblocked and blocked sequencing primers.

Where the method of the invention involves a single (concatenated) polynucleotide strand with a first and second portion, the single (concatenated) polynucleotide strand may comprise a first sequencing primer binding site and a second sequencing primer binding site, where the first sequencing primer binding site and second sequencing primer binding site are of a different sequence to each other and bind different sequencing primers.

In one embodiment, binding of first sequencing primers to the first sequencing primer site generates a first signal and binding of second sequencing primers to the second sequencing primer site generates a second signal, where the intensity of the first signal is greater than the intensity of the second signal. This may be applied to embodiments where the single (concatenated) polynucleotide strand comprises a first sequencing primer binding site and a second sequencing primer binding site. This is achieved using a mixed population of blocked and unblocked second sequencing primers that bind the second sequencing primer site. Any ratio of blocked: unblocked second primers can be used that generates a second signal that is of a lower intensity than the first signal, for example, the ratio of blocked: unblocked primers may be: 20:80 to 80:20, or 1:2 to 2:1.

In one embodiment, a ratio of 50:50 of blocked: unblocked second primers is used, which in turn generates a second signal that is around 50% of the intensity of the first signal.

The first and second sequencing primers may be added to the flow cell at the same time, or separately but sequentially.

In one embodiment, the first sequencing primer binding site may be selected from ME′-A14′ (as defined in SEQ ID NO. 37 or a variant or fragment thereof), A14′ (as defined in SEQ ID NO. 38 or a variant or fragment thereof), ME′-B15′ (as defined in SEQ ID NO. 39 or a variant or fragment thereof) and B15′ (as defined in SEQ ID NO. 40 or a variant or fragment thereof); and the second sequencing primer binding site may be selected from ME′-HYB2 (as defined in SEQ ID NO. 41 or a variant or fragment thereof), HYB2 (as defined in SEQ ID NO. 11 or a variant or fragment thereof), ME′-HYB2′ (as defined in SEQ ID NO. 42 or a variant or fragment thereof) and HYB2′ (as defined in SEQ ID NO. 33 or a variant or fragment thereof).

In another embodiment, the first sequencing primer binding site is ME′-B15′ (as defined in SEQ ID NO. 39 or a variant or fragment thereof), and the second sequencing primer binding site is ME′-HYB2′ (as defined in SEQ ID NO. 42 or a variant or fragment thereof). Alternatively, the first sequencing primer binding site is B15′ (as defined in SEQ ID NO. 40 or a variant or fragment thereof), and the second sequencing primer binding site is HYB2′ (as defined in SEQ ID NO. 33 or a variant or fragment thereof). The first and second sequencing primer sites may be located after (e.g. immediately after) a 3′-end of the first and second portions to be identified.

In another embodiment, the first sequencing primer binding site is ME′-A14′ (as defined in SEQ ID NO. 37 or a variant or fragment thereof), and the second sequencing primer binding site is ME′-HYB2 (as defined in SEQ ID NO. 41 or a variant or fragment thereof). Alternatively, the first sequencing primer binding site may be A14′ (as defined in SEQ ID NO. 38 or a variant or fragment thereof) and the second sequencing primer binding site may be HYB2 (as defined in SEQ ID NO. 31 or a variant or fragment thereof). The first and second sequencing primer sites may be located after (e.g. immediately after) a 3′-end of the first and second portions to be identified.

In one example, the sequencing primer (which may be referred to herein as the second sequencing primer) comprises or consists of a sequence as defined in SEQ ID NO. 31-36, or a variant or fragment thereof. The sequencing primer may further comprise a 3′ blocking group as described above to create a blocked sequencing primer. Alternatively, the primer comprises a 3′-OH group. Such a primer is unblocked and can be elongated with a polymerase.

By “blocked” is meant that the sequencing primer comprises a blocking group at a 3′ end of the sequencing primer. Suitable blocking groups include a hairpin loop (e.g. a polynucleotide attached to 3′ end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase.

The sequence of the sequencing primers and the sequence primer binding sites are not material to the methods of the invention, as long as the sequencing primers are able to bind to the sequence primer binding site to enable amplification and sequencing of the regions to be identified.

In one embodiment, the unblocked and blocked second sequencing primers are present in the sequencing composition in equal concentrations. That is, the ratio of blocked: unblocked second sequencing primers is around 50:50. The sequencing composition may further comprise at least one additional (first) sequencing primer. In one example, the sequencing composition comprises blocked second sequencing primers, unblocked second sequencing primers and at least one first sequencing primer.

In some embodiment, this additional sequencing primer may be selected from A14-ME (as defined in SEQ ID NO. 29 or a variant or fragment thereof), A14 (as defined in SEQ ID NO. 23 or a variant or fragment thereof), B15-ME (as defined in SEQ ID NO. 30 or a variant or fragment thereof) and B15 (as defined in SEQ ID NO. 38 or a variant or fragment thereof). In one embodiment, the sequencing composition comprises blocked second sequencing primers, unblocked second sequencing primers and at least one first sequencing primer, wherein the first sequencing primer is A14, or B15, or is both A14 and B15.

As shown in FIG. 29 selective sequencing may be conducted on the amplified (duoclonal) cluster shown in FIG. 28E or, after restriction sites in the loop complement sequence 403′ and the loop sequence 403 are cleaved by an endonuclease, as described in further detail below. A plurality of first sequencing primers 501 are added. These sequencing primers 501 anneal to a sequencing primer binding site present in the loop complement sequence 403′. A plurality of second unblocked sequencing primers 502a and a plurality of second blocked sequencing primers 502b are added, either at the same time as the first sequencing primers 501, or sequentially (e.g. prior to or after addition of first sequencing primers 501). These second unblocked sequencing primers 502a and second blocked sequencing primers 502b anneal to a sequencing primer binding site present in the loop sequence 403. This then allows the second insert complement sequences 402′ (i.e. “first portions”) to be sequenced and the first insert sequences 401 (i.e. “second portions”) to be sequenced, wherein a greater proportion of second insert complement sequences 402′ are sequenced (black arrow) compared to a proportion of first insert sequences 401 (grey arrow).

In other embodiments, the positioning of first sequencing primers and second sequencing primers may be swapped. In other words, the first sequencing binding primers may anneal instead to the loop sequence 403, and the second sequencing binding primers may anneal instead to the loop complement sequence 403′.

Alternatively, or in addition, selective processing may refer to selective amplification. That is, selectively amplifying one portion (e.g. the first or second portion) on a first or second polynucleotide strand.

In one example, selective processing comprises selectively removing some or substantially all of second immobilised primers that have not yet been extended (extended to form a second polynucleotide strand), and conducting at least one further amplification cycle in order to selectively amplify the first polynucleotide sequence(s) relative to the second polynucleotide sequence(s). Immobilised primers that have not yet been extended may be referred to herein as free or un-extended second immobilised primers.

Accordingly, in this example, selective removal of some or substantially all free second immobilised primers is carried out before at least one further round of bridge amplification and before any sequencing of the target regions. As a consequence, the ratio of first polynucleotide capable of generating a first signal to the second polynucleotide that is capable of generating a second signal is altered, which in turn leads to two signals of different intensities, permitting concurrent sequencing of both sequences (or the target regions within those sequences).

By “some or substantially all” is meant that at least 75%, at least 80%, at least 90% or between 95% and 100% of free second immobilised primers are removed.

The selective removal of all or substantially all free second immobilised primers may be carried out using a reagent capable of cleaving the immobilised primer from the solid support. This reagent may be added following at least 5, at least 10, at least 15 or following 20 to 24 rounds of bridge amplification. The reagent may be added separately or together with the amplification reagents for performing the at least one further round of amplification.

As described above, and described in further detail in WO 2008/041002, the first and second immobilised primers may be attached to the surface of a solid support though a linker. The linker may be different for the first and second immobilised primers. The linker may be any cleavable linker; that is the linker may comprise one or more moieties, such as modified nucleotides, that enable selective cleavage of the immobilised primer from the surface of the solid support. By way of non-limiting example, the linker may comprise uracil bases, phosphorothioate groups, ribonucleotides, diol linkages, disulphide linkages, peptides etc. which may be included, not only to allow covalent attachment to a solid support, but also to allow selective cleavage of the linker.

In one example, the first immobilised primer is attached to a solid support though a first linker, where the linker comprises 8-oxoguanine. In this example, free first immobilised primers (that is, primers that are not extended) can be removed using a FPG glycosylase.

In one example, the sequence of the first immobilised primer comprises the following sequence or a variant of fragment thereof:

(SEQ ID NO. 11)
5′-PS-TTTTTTTTTTAATGATACGGCGACCACCGAUCTACAC-3′
where U = 2-deoxyuridine.

In another example, the second immobilised primer is attached to a solid support through a second linker, where the linker comprises uracil or 2-deoxyuridine. In this example, free second immobilised primers (that is, primers that are not extended) can be removed using uracil glycosylase. In one embodiment, free second immobilised primers can be removed using a USER enzyme mix (which is a cocktail of uracil glycosylase and endonuclease VIII).

In one example, the sequence of the second immobilised primer comprises the following sequence or a variant of fragment thereof:

(SEQ ID NO. 12)
5′-PS-TTTTTTTTTTCAAGCAGAAGACGGCATACGA[G^{oxo}]AT-3′,
where [G^{oxo}] = 8-oxoguanine.

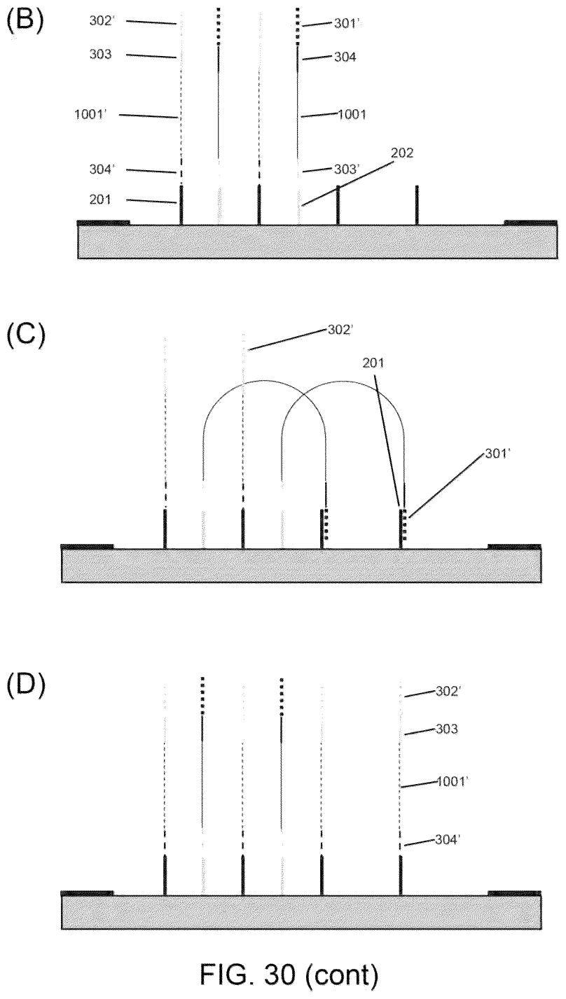

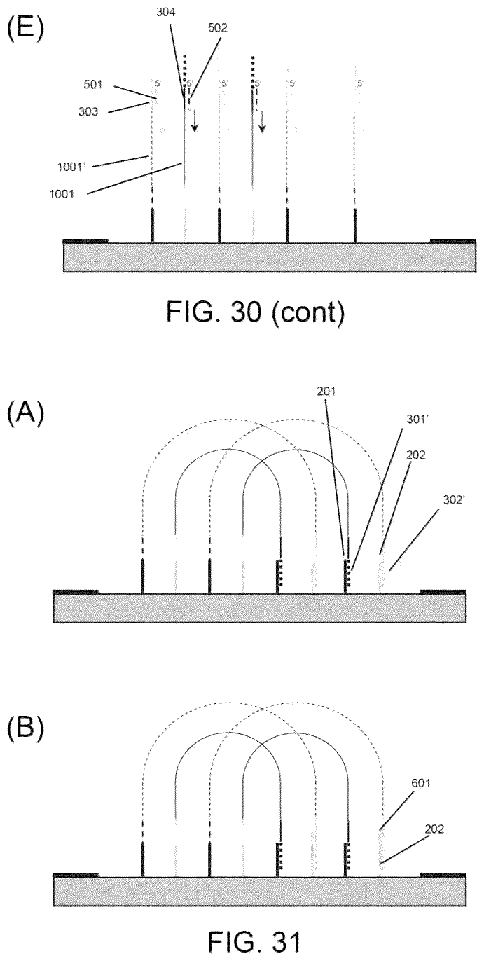

One example of this method is shown in FIG. 30. Selective amplification may be conducted on the amplified (duoclonal) cluster as shown in FIG. 28E. The solid support 200 comprises free first immobilised primers 201 and free second immobilised primers 202 (FIG. 30A). For simplicity, strand 1001′ represents second insert complement sequence 402′, loop complement sequence 403′ and first insert complement sequence 401′, whilst strand 1001 represents first insert sequence 401, loop sequence 403 and second insert sequence 402. Free second immobilised primers 202 are cleaved from the solid support 200, thus leaving behind free first immobilised primers 201 (FIG. 30B).

The first primer-binding sequence 301′ (e.g. P5′) on one set of template strands may then anneal to the free first immobilised primers 201 (e.g. P5 lawn primer) located within the well 203. By contrast, since free second immobilised primers 202 (e.g. P7 lawn primer) have been removed, second primer-binding sequences 302′ (e.g. P7′) are not able to anneal (FIG. 30C).

After conducting a cycle of bridge amplification, this leads to selective amplification of the strand 1001′, relative to the strand 1001 (FIG. 30D).

Conducting standard (non-selective) sequencing then allows strands 1001′ and strands 1001 to be sequenced, wherein a greater proportion of strands 1001′ are sequenced (grey arrow) compared to a proportion of strands 1001 (black arrow) (FIG. 30E).

In another example, selectively processing comprises selectively blocking the extension of some or substantially all of the second immobilised primers that have not yet been extended (extended to form a second polynucleotide strand). Again, these primers may be referred to herein as free or un-extended second immobilised primers. The method may involve using a primer-blocking agent, wherein the primer-blocking agent is configured to limit or prevent synthesis of a strand (i.e. a polynucleotide strand) extending from the second immobilised primer. The method may further involve conducting at least one further amplification cycle. As the free second immobilised primers are blocked from being extended by the primer-blocking agent, only the first immobilised primers can be extended. This leads to amplification of only the first polynucleotide strand (i.e. not the second polynucleotide strand), and as a consequence, an increase in the amount of first polynucleotide sequences relative to the second polynucleotide sequences.

By “some or substantially all” is meant that at least 75%, at least 80%, at least 90% or between 95% and 100% of free second immobilised primers are blocked.

The primer-blocking agent may be flowed across the solid support following bridge amplification. In one embodiment, the primer-blocking agent is flowed across the solid support following at least 1, 2, 3, 4, 5, 6, 7, 8, 9 or 10 cycles, following at least 15, following at least 20 or following at least 25 rounds of bridge amplification.

In one example, the primer-blocking agent is added whilst first polynucleotide sequence(s) are hybridised to the second immobilised primers. That is, the primer-blocking agent is added during amplification and following extension of at least the first polynucleotide strand. At this stage the extended first polynucleotide strand bends (bridges) and hybridises at its 5′ end to the second immobilised primer. Addition of the primer-blocking agent at this stage prevents extension of the second immobilised primer, which would normally occur using the first polynucleotide strand as its template.

In one embodiment, the primer-blocking agent is a blocked nucleotide. For example, the blocked nucleotide may be A, C, T or G, but may be selected from A or G.

Again, by “blocked” is meant that the sequencing primer comprises a blocking group at a 3′ end of the sequencing primer. Suitable blocking groups include a hairpin loop (e.g. a polynucleotide attached to 3′ end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group)), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase. The block may be reversible or irreversible.

The blocked nucleotide may be added as part of a mixture comprising both blocked and unblocked nucleotides. Alternatively, the blocked nucleotide may be added to the flow cell separately and either before or after unblocked nucleotides are added. Following addition of the blocked nucleotide, at least one more round of bridge amplification is performed.

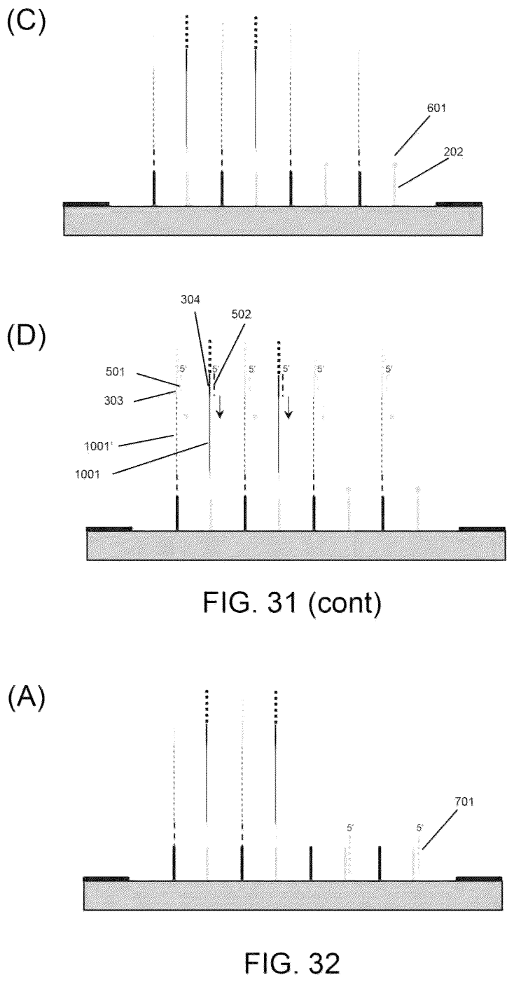

One example of this method is shown in FIG. 31 Selective amplification may be conducted on the amplified (duoclonal) cluster as shown in FIG. 30A. The first primer-binding sequence 301′ (e.g. P5′) on one set of template strands may anneal to first immobilised primers 201 (e.g. P5 lawn primer), and the second primer-binding sequence 302′ (e.g. P7′) on another set of template strands may anneal to second immobilised primers 202 (e.g. P7 lawn primer) (FIG. 31A).

Whilst the second primer-binding sequence 302′ (e.g. P7′) is annealed to the second immobilised primer 202, a primer-blocking agent 601 is selectively installed onto a 3′-end of the second immobilised primer 202, whilst no installation occurs to 3′-end of the first immobilised primer 201 (FIG. 31B).

After conducting a cycle of bridge amplification, this leads to selective amplification of the strands 1001′, relative to the strands 1001. The primer-blocking agent 601 prevents extension from the second immobilised primer 202 (FIG. 31C).

Conducting standard (non-selective) sequencing then allows strands 1001′ and strands 1001 to be sequenced, wherein a greater proportion of strands 1001′ are sequenced (grey arrow) compared to a proportion of strands 1001 (black arrow) (FIG. 31D).

In an alternative example, the method comprises flowing at least one, or a plurality of, extended primer sequence(s) across the surface of the solid support (e.g. a flow cell), wherein such sequences can bind (e.g. hybridise) free immobilised primers (e.g. P5 or P7) and wherein the extended primer sequences further comprise at least one 5′ additional nucleotide; and (b) adding the primer blocking agent, where the primer blocking agent is complementary to 5′ additional nucleotide.

In one embodiment, the extended primer sequences are substantially complementary to the first or second immobilised primers (e.g. P5 or P7), or substantially complementary to a portion of the first or second immobilised primer.

The 5′ additional nucleotide may be selected from A, T, C or G, but may be T (or U) or C. In some aspects, 5′ additional nucleotide is not a complement of 3′ nucleotide of the second immobilised primer (where the extended primer sequence binds the first immobilised primer) or is not a complement of 3′ nucleotide of the first immobilised primer (where the extended primer sequence binds the second immobilised primer). For example, where the first immobilised primer is P5 (for example as defined in SEQ ID NO. 1) and the second immobilised primer is P7 for example as defined in SEQ ID NO. 2), and where the extended primer sequence binds the first immobilised primer, 5′ additional nucleotide is not A. Similarly, where the extended primer sequence binds the second immobilised primer, the 5′ additional nucleotide is not G.

In one embodiment, the primer-blocking agent is a blocked nucleotide, for example, as described above. For example, the blocked nucleotide may be A, C, T or G, but may be is selected from A or G. Accordingly, where 5′ additional nucleotide is T or U, the primer-blocking agent is A, and where 5′ additional nucleotide is C, the primer-blocking agent is G.

Again, the extended primer sequence(s) and primer-blocking agent may be flowed across the solid support following bridge amplification. In one embodiment, the primer-blocking agent is flowed across the solid support following at least 1, at least 2, at least 3, at least 4, at least 5, at least 6, at least 7, at least 8, at least 9, at least 10, at least 15, at least 20 or following at least 25 rounds of bridge amplification.

In one embodiment, the extended primer sequence is selected from SEQ ID NO. 13 to 24 or a variant or fragment thereof.

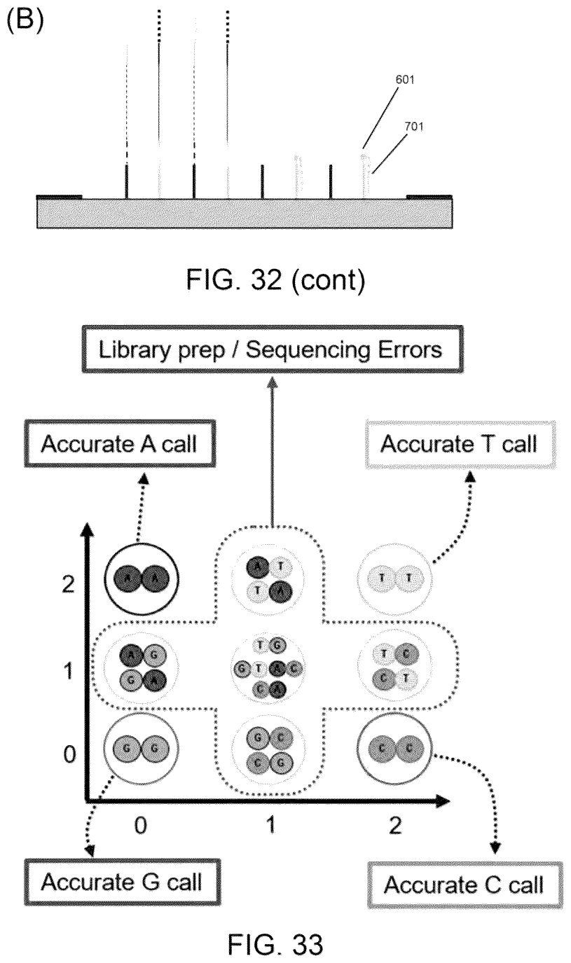

One example of this method is shown in FIG. 32. Selective amplification may be conducted on the amplified (duoclonal) cluster as shown in FIG. 30A; as such following a number of rounds of amplification, a cluster is formed comprising both extended first (e.g. P5) and second (e.g. P7) immobilised polynucleotide strands. Before the next round of amplification, a (or a plurality of) extended primer sequence(s) is flowed across the surface of the solid support 200. The extended primer sequence 701 is substantially complementary to at least a portion, if not all of the immobilised primer (e.g. either P5 or P7) and binds to the immobilised primer (e.g. P5 or P7) as shown in FIG. 32A. As also shown in FIG. 32A, the extended primer sequence 701 comprises at least one additional 5′ nucleotide.

Following addition of the extended primer sequence 701, a primer blocking agent 601 is added and flowed across the surface of the solid support (e.g. flow cell). As the primer-blocking agent 601 is complementary to 5′ additional nucleotide of the extended primer sequence 701 the primer-blocking agent 601 binds to 3′-end of the immobilised strands that are hybridised to the extended primer sequence 701, as shown in FIG. 21B. As a consequence, addition of the primer-blocking agent 601 prevents not only extension of the immobilised strand (e.g. P5 or P7) but renders the immobilised primer (P5 or P7) unavailable for hybridisation and subsequent bridge amplification for other extended strands (e.g. 101′) (see FIG. 32B).

Performing at least one more cycle of bridge amplification, leads to selective amplification of strands 1001′ (in a 2:1 ratio of 1001′ to 1001). Again, similar to FIG. 31D, conducting standard (non-selective) sequencing then allows strands 1001′ and strands 1001 to be sequenced, wherein a greater proportion of strands 1001′ are sequenced (grey arrow) compared to a proportion of strands 1001 (black arrow) (FIG. 31D).

The extended primer sequences may be added as part of the amplification mixture described above. Alternatively, the blocked immobilised primer-binding sequence may be added to the flow cell separately and may be before the amplification mixture is added. Following addition of the blocked immobilised primer-binding sequence, at least one more round of bridge amplification is performed.

As shown in FIG. 29, selective sequencing may be conducted on the amplified (monoclonal) cluster shown in FIG. 28F. A plurality of first sequencing primers 501 are added. These first sequencing primers 501 (e.g. B15-ME; or if ME is not present, then B15) anneal to the first terminal sequencing primer binding site 303 (which represents a type of “first sequencing primer binding site”) (e.g. ME′-B15′; or if ME′ is not present, then B15′). A plurality of second unblocked sequencing primers 502a and a plurality of second blocked sequencing primers 502b are added, either at the same time as the first sequencing primers 501, or sequentially (e.g. prior to or after addition of first sequencing primers 501). These second unblocked sequencing primers 502a (e.g. HYB2-ME; or if ME is not present, then HYB2) and second blocked sequencing primers 502b (e.g. blocked HYB2-ME; or if ME is not present, then blocked HYB2) anneal to an internal sequencing primer binding site in the hybridisation sequence 403′ (which represents a type of “second sequencing primer binding site”) (e.g. ME′-HYB2′; or if ME′ is not present, then HYB2′). This then allows the first insert complement sequences 401′ (i.e. “first portions”) to be sequenced and the second insert complement sequences 402′ (i.e. “second portions”) to be sequenced, wherein a greater proportion of first insert complement sequences 401′ are sequenced (grey arrow) compared to a proportion of second insert complement sequences 402′ (black arrow).

Although FIG. 29 shows selective sequencing being conducted on a template strand attached to first immobilised primer 201, in some embodiments the (monoclonal) cluster may instead have template strands attached to second immobilised primer 202. In such a case, the first sequencing primers may instead correspond to A14-ME (or if ME is not present, then A14), and the second unblocked sequencing primers may instead correspond to HYB2′-ME (or if ME is not present, then HYB2′) and second blocked sequencing primers may instead correspond to blocked HYB2′-ME (or if ME is not present, then blocked HYB2′).

In yet other embodiments, the positioning of first sequencing primers and second sequencing primers may be swapped. In other words, the first sequencing binding primers may anneal instead to the internal sequencing primer binding site, and the second sequencing binding primers may anneal instead to the terminal sequencing primer binding site.

FIG. 29 shows concurrent sequencing of a concatenated strand according to the above method. As shown in FIG. 29, a polynucleotide strand with a first portion (insert) and second portion (insert) can be accurately and simultaneously sequenced by a selective sequencing method that uses a mixture of unblocked and blocked sequencing primers as described above. Embodiments of the present invention are directed to methods of preparing a polynucleotide strand or strands for identification such that where the strand comprises two portions (in other words, a concatenated polynucleotide sequence comprising a first portion and a second portion) to be identified, or where separate strands each comprise a portion to be identified (in other words, a first polynucleotide sequence comprising a first portion and a second polynucleotide sequence comprising a second portion), such portions can be identified concurrently. This may be achieved by altering the ratio of the different portions which are capable of emitting a signal, which in turn means that during sequencing the signal from the first portion will be greater than the signal from the second portion. It is this difference in the intensity of the first and second signals that allows for the two portions, either on the same or different polynucleotide strands, to be identified simultaneously. It is of course desirable to be able to maximise the throughput and decrease the run time of a sequencing reaction. Concurrent sequencing, achieved by the methods of the present invention, enables at least a doubling of the throughput of a sequencing reaction (i.e. increased sequencing efficiency) as well as a decrease in the time taken to sequence a target polynucleotide strand(s).

Accordingly, we describe a method of preparing at least one polynucleotide sequence (or strand, such terms may be used interchangeably herein) for identification, where the method comprises selectively processing at least one polynucleotide sequence comprising a first portion and a second portion, or at least one first polynucleotide sequence comprising a first portion and at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

The at least one polynucleotide sequence comprising the first portion and a second portion may be a plurality of polynucleotide sequences each comprising a first portion and a second portion.

The at least one first polynucleotide sequence comprising a first portion and the at least one second polynucleotide sequence comprising a second portion may be a plurality of first polynucleotide sequences each comprising a first portion, and a plurality of second polynucleotide sequences each comprising a second portion.

Accordingly, the method may comprise selectively processing a plurality of polynucleotide sequences each comprising a first portion and a second portion, or a plurality of first polynucleotide sequences each comprising a first portion and a plurality of second polynucleotide sequences each comprising a second portion, such that a proportion of first portions are capable of generating a first signal and a proportion of second portions are capable of generating a second signal, wherein the selective processing causes an intensity of the first signal to be greater than an intensity of the second signal.

Embodiments may be applied to a single (concatenated) polynucleotide strand that comprises, on the same strand, a first portion and a second portion to be identified. As explained above, such a strand can be produced using known techniques in the art, such as PCR stitching, tandem insert methods or loop fork methods.

The first portions and second portions may be different polynucleotide sequences. That is, the sequences may be genetically unrelated and/or derived from different sources.

Alternatively, the first portions and second portions may be genetically related.

For example, the first portion may comprise (or be) the forward strand of a polynucleotide sequence (e.g. forward strand of a template), and the second portion may comprise (or be) the reverse strand of the polynucleotide sequence (e.g. reverse strand of the template) or the forward complement strand of the polynucleotide sequence (e.g. forward complement strand of the template). As a further alternative, the first portion may comprise (or be) the reverse strand of a polynucleotide sequence (e.g. reverse strand of a template), and the second portion may comprise (or be) the forward strand of the polynucleotide sequence (e.g. forward strand of the template) or the reverse complement strand of the polynucleotide sequence (e.g. reverse complement strand of the template).

Alternatively, the first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). As a further alternative, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand). In some embodiments, the first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced. In these particular embodiments, concurrent sequencing of both the forward and reverse complement strands (or the reverse and forward complement strands) allows mismatched base pairs and/or epigenetic modification to be detected.

The first portion may be referred to herein as read 1 (R1). The second portion may be referred to herein as read 2 (R2).

In embodiments relating to a single (concatenated) polynucleotide strand, the single polynucleotide strand may be attached to a solid support. Preferably, this solid support is a flow cell. Preferably, the polynucleotide strand is attached to the solid support in a single well of the solid support.

Accordingly, the method may comprise selectively processing at least one polynucleotide sequence comprising a first portion and a second portion, wherein each polynucleotide sequence is attached to a first immobilised primer. Preferably, the method may comprise selectively processing a plurality of polynucleotide sequences each comprising a first portion and a second portion, wherein each polynucleotide sequence is attached to a first immobilised primer.

Alternatively, Embodiments can be applied to (separate) polynucleotide strands where a first strand comprises a first portion to be identified and a second strand comprises a second portion to be identified.

The first portions and second portions may be different polynucleotide sequences. That is, the sequences may be genetically unrelated and/or derived from different sources.

Alternatively, the first portions and second portions may be genetically related.

For example, the (separate) polynucleotide strands may comprise a first strand that comprises a first portion that may comprise (or be) the forward strand of a polynucleotide sequence (e.g. forward strand of a template), and a second strand that comprises a second portion that may comprise (or be) the reverse strand of the polynucleotide sequence (e.g. reverse strand of the template) or the forward complement strand of the polynucleotide sequence (e.g. forward complement strand of the template). As a further alternative, the (separate) polynucleotide strands may comprise a first strand that comprises a first portion that may comprise (or be) the reverse strand of a polynucleotide sequence (e.g. reverse strand of a template), and a second strand that comprises a second portion that may comprise (or be) the forward strand of the polynucleotide sequence (e.g. forward strand of the template) or the reverse complement strand of the polynucleotide sequence (e.g. reverse complement strand of the template).

Alternatively, the (separate) polynucleotide strands may comprise a first strand that comprises a first portion that may comprise (or be) the forward strand of a polynucleotide sequence (e.g. forward strand of a template), and a second strand that comprises a second portion that may comprise (or be) the reverse complement strand of the polynucleotide sequence (e.g. reverse complement strand of the template) (in effect, a reverse complement strand may be considered a “copy” of the forward strand). As a further alternative, the (separate) polynucleotide strands may comprise a first strand that comprises a first portion that may comprise (or be) the reverse strand of a polynucleotide sequence (e.g. reverse strand of a template), and a second strand that comprises a second portion that may comprise (or be) the forward complement strand of the polynucleotide sequence (e.g. forward complement strand of the template) (in effect, a forward complement strand may be considered a “copy” of the reverse strand). In some embodiments, the first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced. In these particular embodiments, concurrent sequencing of both the forward and reverse complement strands (or the reverse and forward complement strands) allows mismatched base pairs and/or epigenetic modification to be detected.

Again, the first portion may be referred to herein as read 1 (R1). The second portion may be referred to herein as read 2 (R2).

Preferably, in embodiments relating to (separate) polynucleotide strands, the first and second strand may be separately attached to a solid support. Preferably, this solid support is a flow cell. Preferably, each of the first and second strands are attached to the solid support (e.g. flow cell) in a single well of the solid support.

Accordingly, the method may comprise selectively processing at least one first polynucleotide sequence comprising a first portion and at least one second polynucleotide sequence comprising a second portion, wherein each first polynucleotide sequence is attached to a first immobilised primer, and each second polynucleotide sequence is attached to a second immobilised primer. Preferably, the method may comprise selectively processing a plurality of first polynucleotide sequences each comprising a first portion and a plurality of at least one second polynucleotide sequences each comprising a second portion, wherein each first polynucleotide sequence is attached to a first immobilised primer, and each second polynucleotide sequence is attached to a second immobilised primer.

Where the method of the invention involves a single polynucleotide strand with a first and second portion, before sequencing one group of strands (either the group of template polynucleotides, or the group of template complement polynucleotides thereof) may be removed from the solid support, leaving either the templates or the template complements, as explained above. Such a cluster may be considered to be a “monoclonal” cluster.

Where the method of the invention involves a first polynucleotide strand and a second polynucleotide strand, the cluster formed may be a duoclonal cluster.

Preferably, the selective processing results in the concentration of the first portions capable of generating the first signal being greater than the concentration of the second portions capable of generating the second signal. In other words, the method of the invention results in an altered ratio of R1: R2 molecules, preferably within a single cluster or a single well. It is this altered ratio that primes the first portions and second portions to be ready for concurrent sequencing.

Preferably, the ratio may be between 1.25:1 to 5:1, preferably between 1.5:1 to 3:1, more preferably about 2:1.

The first signal and the second signal may be spatially unresolved (e.g. generated from the same region or substantially overlapping regions). Preferably, a first region occupied by the at least one first polynucleotide sequence comprising the first portion within the duoclonal cluster is the same as, or substantially overlapping with, a second region occupied by the at least one second polynucleotide sequence comprising the second portion within the duoclonal cluster.

Selective processing may refer to conducting selective sequencing. Alternatively, selective processing may refer to preparing for selective sequencing. As shown in FIG. 29, in one example, selective sequencing may be achieved using a mixture of unblocked and blocked sequencing primers.

Where the method of the invention involves a single (concatenated) polynucleotide strand with a first and second portion, the single (concatenated) polynucleotide strand may comprise a first sequencing primer binding site and a second sequencing primer binding site, where the first sequencing primer binding site and second sequencing primer binding site are of a different sequence to each other and bind different sequencing primers.

Where the method of the invention involves (separate) polynucleotide strands, with a first polynucleotide strand with a first portion, and a second polynucleotide strand with a second portion, the first polynucleotide strand may comprise a first sequencing primer binding site, and the second polynucleotide strand may comprise a second sequencing primer binding site, where the first sequencing primer binding site and second sequencing primer binding site are of a different sequence to each other and bind different sequencing primers.

Preferably, binding of first sequencing primers to the first sequencing primer site generates a first signal and binding of second sequencing primers to the second sequencing primer site generates a second signal, where the intensity of the first signal is greater than the intensity of the second signal. This may be applied to embodiments where the single (concatenated) polynucleotide strand comprises a first sequencing primer binding site and a second sequencing primer binding site, or to embodiments where the first polynucleotide strand comprises a first sequencing primer binding site, and the second polynucleotide strand comprises a second sequencing primer binding site. In other embodiments, the binding of first sequencing primers and second sequencing primers may not be applied to cases where the first polynucleotide strand comprises a first sequencing primer binding site, and the second polynucleotide strand comprises a second sequencing primer binding site. This is achieved using a mixed population of blocked and unblocked second sequencing primers that bind the second sequencing primer site. Any ratio of blocked: unblocked second primers can be used that generates a second signal that is of a lower intensity than the first signal, for example, the ratio of blocked: unblocked primers may be: 20:80 to 80:20, preferably 1:2 to 2:1.

Most preferably, a ratio of 50:50 of blocked: unblocked second primers is used, which in turn generates a second signal that is around 50% of the intensity of the first signal.

The first and second sequencing primers may be added to the flow cell at the same time, or separately but sequentially.

The sequence of the sequencing primers and the sequence primer binding sites are not material to the methods of the invention, as long as the sequencing primers are able to bind to the sequence primer binding site to enable amplification and sequencing of the regions to be identified.

In one embodiment, the first sequencing primer binding site may be selected from ME′-A14′ (as defined in SEQ ID NO: 37 or a variant or fragment thereof), A14′ (as defined in SEQ ID NO: 38 or a variant or fragment thereof), ME′-B15′ (as defined in SEQ ID NO:39 or a variant or fragment thereof) and B15′ (as defined in SEQ ID NO: 40 or a variant or fragment thereof); and the second sequencing primer binding site may be selected from ME′-HYB2 (as defined in SEQ ID NO: 41 or a variant or fragment thereof), HYB2 (as defined in SEQ ID NO: 31 or a variant or fragment thereof), ME′-HYB2′ (as defined in SEQ ID NO: 42 or a variant or fragment thereof) and HYB2′ (as defined in SEQ ID NO: 13 or a variant or fragment thereof).

In another embodiment, the first sequencing primer binding site is ME′-B15′ (as defined in SEQ ID NO: 39 or a variant or fragment thereof), and the second sequencing primer binding site is ME′-HYB2′ (as defined in SEQ ID NO: 42 or a variant or fragment thereof). Alternatively, the first sequencing primer binding site is B15′ (as defined in SEQ ID NO: 40 or a variant or fragment thereof), and the second sequencing primer binding site is HYB2′ (as defined in SEQ ID NO: 33 or a variant or fragment thereof). The first and second sequencing primer sites are preferably located after (e.g. immediately after) a 3′-end of the first and second portions to be identified.

In another embodiment, the first sequencing primer binding site is ME′-A14′ (as defined in SEQ ID NO: 37 or a variant or fragment thereof), and the second sequencing primer binding site is ME′-HYB2 (as defined in SEQ ID NO: 41 or a variant or fragment thereof). Alternatively, the first sequencing primer binding site may be A14′ (as defined in SEQ ID NO: 38 or a variant or fragment thereof) and the second sequencing primer binding site may be HYB2 (as defined in SEQ ID NO: 31 or a variant or fragment thereof). The first and second sequencing primer sites are preferably located after (e.g. immediately after) a 3′-end of the first and second portions to be identified.

In one example, the sequencing primer (which may be referred to herein as the second sequencing primer) comprises or consists of a sequence as defined in SEQ ID NO: 31 to 36, or a variant or fragment thereof. The sequencing primer may further comprise a 3′ blocking group as described above to create a blocked sequencing primer. Alternatively, the primer comprises a 3′-OH group. Such a primer is unblocked and can be elongated with a polymerase.

Accordingly, in an aspect of the invention, there is provided a sequencing primer comprising or consisting of a sequence selected from SEQ ID NO: 31 to 36 or a variant or fragment thereof.

In another aspect of the invention there is provided a sequencing composition (also referred to herein as a sequencing mix), comprising a blocked second sequencing primer selected from SEQ ID NO: 35 and 36 or a variant or fragment thereof, and an unblocked second sequencing primer selected from SEQ ID NO: 33 and 34, or a variant or fragment thereof. In one embodiment, the sequencing composition comprises a blocked sequencing primer selected from SEQ ID NO: 35 or a variant or fragment thereof, and an unblocked sequencing primer selected from SEQ ID NO: 33 or a variant or fragment thereof. In another embodiment, the sequencing composition comprises a blocked sequencing primer selected from SEQ ID NO: 36 or a variant or fragment thereof, and an unblocked sequencing primer selected from SEQ ID NO: 34, or a variant or fragment thereof.

Preferably, the unblocked and blocked second sequencing primers are present in the sequencing composition in equal concentrations. That is, the ratio of blocked: unblocked second sequencing primers is around 50:50. The sequencing composition may further comprise at least one additional (first) sequencing primer. This additional sequencing primer may be selected from A14-ME (as defined in SEQ ID NO: 29 or a variant or fragment thereof), A14 (as defined in SEQ ID NO: 7 or a variant or fragment thereof), B15-ME (as defined in SEQ ID NO: 30 or a variant or fragment thereof) and B15 (as defined in SEQ ID NO: 28 or a variant or fragment thereof). Preferably, the sequencing composition comprises blocked second sequencing primers, unblocked second sequencing primers and at least one first sequencing primer, wherein the first sequencing primer is A14, or B15, or is both A14 and B15.

In another aspect of the invention, there is provided the use of a blocked sequencing primer, preferably a blocked sequencing primer comprising SEQ ID NO: 31 to 36 or a variant or fragment thereof in preparing at least one polynucleotide sequence, preferably a plurality of polynucleotide sequences, for identification.

As shown in FIG. 29B, selective sequencing may be conducted on the amplified (monoclonal) cluster shown in FIG. 28F. A plurality of first sequencing primers 501 are added. These first sequencing primers 501 (e.g. B15-ME; or if ME is not present, then B15) anneal to the first terminal sequencing primer binding site 303 (which represents a type of “first sequencing primer binding site”) (e.g. ME′-B15′; or if ME′ is not present, then B15′). A plurality of second unblocked sequencing primers 502a and a plurality of second blocked sequencing primers 502b are added, either at the same time as the first sequencing primers 501, or sequentially (e.g. prior to or after addition of first sequencing primers 501). These second unblocked sequencing primers 502a (e.g. HYB2-ME; or if ME is not present, then HYB2) and second blocked sequencing primers 502b (e.g. blocked HYB2-ME; or if ME is not present, then blocked HYB2) anneal to an internal sequencing primer binding site in the hybridisation sequence 403′ (which represents a type of “second sequencing primer binding site”) (e.g. ME′-HYB2′; or if ME′ is not present, then HYB2′). This then allows the first insert complement sequences 401′ (i.e. “first portions”) to be sequenced and the second insert complement sequences 402′ (i.e. “second portions”) to be sequenced, wherein a greater proportion of first insert complement sequences 401′ are sequenced (grey arrow) compared to a proportion of second insert complement sequences 402′ (black arrow).

Although FIG. 29B shows selective sequencing being conducted on a template strand attached to first immobilised primer 201, in some embodiments the (monoclonal) cluster may instead have template strands attached to second immobilised primer 202. In such a case, the first sequencing primers may instead correspond to A14-ME (or if ME is not present, then A14), and the second unblocked sequencing primers may instead correspond to HYB2′-ME (or if ME is not present, then HYB2′) and second blocked sequencing primers may instead correspond to blocked HYB2′-ME (or if ME is not present, then blocked HYB2′).

In yet other embodiments, the positioning of first sequencing primers and second sequencing primers may be swapped. In other words, the first sequencing binding primers may anneal instead to the internal sequencing primer binding site, and the second sequencing binding primers may anneal instead to the terminal sequencing primer binding site.

FIG. 29B shows concurrent sequencing of a concatenated strand according to the above method. As shown in FIG. 29B, a polynucleotide strand with a first portion (insert) and second portion (insert) can be accurately and simultaneously sequenced by a selective sequencing method that uses a mixture of unblocked and blocked sequencing primers as described above.

Alternatively, or in addition, selective processing may refer to selective amplification. That is, selectively amplifying one portion (e.g. the first or second portion) of a single (concatenated) polynucleotide strand or selectively amplifying one portion (e.g. the first or second portion) on a first or second polynucleotide strand.

In one example, selective processing comprises selectively removing some or substantially all of second immobilised primers that have not yet been extended (extended to form a second polynucleotide strand), and conducting at least one further amplification cycle in order to selectively amplify the first polynucleotide sequence(s) relative to the second polynucleotide sequence(s). Immobilised primers that have not yet been extended may be referred to herein as free or un-extended second immobilised primers.

Accordingly, in this example, selective removal of some or substantially all free second immobilised primers is carried out before at least one further round of bridge amplification and before any sequencing of the target regions. As a consequence, the ratio of first polynucleotide capable of generating a first signal to the second polynucleotide that is capable of generating a second signal is altered, which in turn leads to two signals of different intensities, permitting concurrent sequencing of both sequences (or the target regions within those sequences).

By “some or substantially all” is meant that at least 75%, preferably at least 80%, more preferably at least 90% and most preferably between 95% and 100% of free second immobilised primers are removed.

The selective removal of all or substantially all free second immobilised primers may be carried out using a reagent capable of cleaving the immobilised primer from the solid support. This reagent may be added following at least 5, more preferably at least 10, even more preferably at least 15 and most preferably 20 to 24 rounds of bridge amplification. The reagent may be added separately or together with the amplification reagents for performing the at least one further round of amplification.

As described above, and described in further detail in WO 2008/041002, the first and second immobilised primers may be attached to the surface of a solid support though a linker. The linker is preferably different for the first and second immobilised primers. The linker may be any cleavable linker; that is the linker may comprise one or more moieties, such as modified nucleotides, that enable selective cleavage of the immobilised primer from the surface of the solid support. By way of non-limiting example, the linker may comprise uracil bases, phosphorothioate groups, ribonucleotides, diol linkages, disulphide linkages, peptides etc. which may be included, not only to allow covalent attachment to a solid support, but also to allow selective cleavage of the linker.

In one example, the first immobilised primer is attached to a solid support though a first linker, where the linker comprises 8-oxoguanine. In this example, free first immobilised primers (that is, primers that are not extended) can be removed using a FPG glycosylase.

In one example, the sequence of the first immobilised primer comprises the following sequence or a variant of fragment thereof: 5′-PS-TTTTTTTTTTAATGATACGGCGACCACCGAUCTACAC-3′ where U=2-deoxyuridine (SEQ ID NO: 11).

In another example, the second immobilised primer is attached to a solid support through a second linker, where the linker comprises uracil, and more preferably 2-deoxyuridine. In this example, free second immobilised primers (that is, primers that are not extended) can be removed using uracil glycosylase. More preferably, free second immobilised primers can be removed using a USER enzyme mix (which is a cocktail of uracil glycosylase and endonuclease VIII). In one example, the sequence of the second immobilised primer comprises the following sequence or a variant of fragment thereof: 5′-PS-TTTTTTTTTTCAAGCAGAAGACGGCATACGA [G^{oxo}]AT-3′, where [G^{oxo}]=8-oxoguanine (SEQ ID NO: 12).

Accordingly, in a further aspect of the invention, there is provided an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

One example of this method is shown in FIG. 30. Selective amplification may be conducted on the amplified (duoclonal) cluster as shown in FIG. 2H. The solid support 200 comprises free first immobilised primers 201 and free second immobilised primers 202. Free second immobilised primers 202 are cleaved from the solid support 200, thus leaving behind free first immobilised primers 201 (FIG. 30A).

The first primer-binding sequence 301′ (e.g. P5′) on one set of template strands may then anneal to the free first immobilised primers 201 (e.g. P5 lawn primer) located within the well 203. By contrast, since free second immobilised primers 202 (e.g. P7 lawn primer) have been removed, second primer-binding sequences 302′ (e.g. P7′) are not able to anneal (FIG. 30B).

After conducting a cycle of bridge amplification, this leads to selective amplification of the template strands comprising the forward strand of the template 101′ and the first terminal sequencing primer binding site 303, relative to the template strands comprising the forward complement strand of the template 101 and the second terminal sequencing primer binding site 304 (FIG. 30C).

Conducting standard (non-selective) sequencing then allows the forward strands of the template 101′ (i.e. “first portions”) to be sequenced and the forward complement strands of the template 101 (i.e. “second portions”) to be sequenced, wherein a greater proportion of forward strands of the template 101′ are sequenced (grey arrow) compared to a proportion of forward complement strands of the template 101 (black arrow) (FIG. 30D).

In another example, selectively processing comprises selectively blocking the extension of some or substantially all of the second immobilised primers that have not yet been extended (extended to form a second polynucleotide strand). Again, these primers may be referred to herein as free or un-extended second immobilised primers. The method may involve using a primer-blocking agent, wherein the primer-blocking agent is configured to limit or prevent synthesis of a strand (i.e. a polynucleotide strand) extending from the second immobilised primer. The method may further involve conducting at least one further amplification cycle. As the free second immobilised primers are blocked from being extended by the primer-blocking agent, only the first immobilised primers can be extended. This leads to amplification of only the first polynucleotide strand (i.e. not the second polynucleotide strand), and as a consequence, an increase in the amount of first polynucleotide sequences relative to the second polynucleotide sequences.

By “some or substantially all” is meant that at least 75%, preferably at least 80%, more preferably at least 90% and most preferably between 95% and 100% of free second immobilised primers are blocked.

The primer-blocking agent is preferably flowed across the solid support following bridge amplification. More preferably, the primer-blocking agent is flowed across the solid support following at least 1, 2, 3, 4, 5, 6, 7, 8, 9 or 10 cycles, preferably at least 15, more preferably at least 20 and even more preferably at least 25 rounds of bridge amplification.

In one example, the primer-blocking agent is added whilst first polynucleotide sequence(s) are hybridised to the second immobilised primers. That is, the primer-blocking agent is added during amplification and following extension of at least the first polynucleotide strand. At this stage the extended first polynucleotide strand bends (bridges) and hybridises at its 5′ end to the second immobilised primer. Addition of the primer-blocking agent at this stage prevents extension of the second immobilised primer, which would normally occur using the first polynucleotide strand as its template.

Preferably, the primer-blocking agent is a blocked nucleotide. More preferably, the blocked nucleotide may be A, C, T or G, but most preferably is selected from A or G.

The blocked nucleotide may be added as part of a mixture comprising both blocked and unblocked nucleotides. Alternatively, the blocked nucleotide may be added to the flow cell separately and either before or after unblocked nucleotides are added. Following addition of the blocked nucleotide, at least one more round of bridge amplification is performed.

One example of this method is shown in FIG. 31. Selective amplification may be conducted on the amplified (duoclonal) cluster as shown in FIG. 2H. The first primer-binding sequence 301′ (e.g. P5′) on one set of template strands may anneal to first immobilised primers 201 (e.g. P5 lawn primer), and the second primer-binding sequence 302′ (e.g. P7′) on another set of template strands may anneal to second immobilised primers 202 (e.g. P7 lawn primer) (FIG. 31A).

Whilst the second primer-binding sequence 302′ (e.g. P7′) is annealed to the second immobilised primer 202, a primer-blocking agent 601 is selectively installed onto a 3′-end of the second immobilised primer 202, whilst no installation occurs to 3′-end of the first immobilised primer 201 (FIG. 31B).

Conducting cycle(s) of bridge amplification leads to selective amplification of the template strands comprising the forward strand of the template 101′ and the first terminal sequencing primer binding site 303, relative to the template strands comprising the forward complement strand of the template 101 and the second terminal sequencing primer binding site 304. The primer-blocking agent 601 prevents extension from the second immobilised primer 202. (FIG. 31C).

Conducting standard (non-selective) sequencing then allows the forward strands of the template 101′ (i.e. “first portions”) to be sequenced and the forward complement strands of the template 101 (i.e. “second portions”) to be sequenced, wherein a greater proportion of forward strands of the template 101′ are sequenced (grey arrow) compared to a proportion of forward complement strands of the template 101 (black arrow) (FIG. 31D).

In an alternative example, the method comprises flowing at least one, preferably a plurality of, extended primer sequence(s) across the surface of the solid support (e.g. a flow cell), wherein such sequences can bind (e.g. hybridise) free immobilised primers (e.g. P5 or P7) and wherein the extended primer sequences further comprise at least one 5′ additional nucleotide; and (b) adding the primer blocking agent, where the primer blocking agent is complementary to 5′ additional nucleotide.

Preferably, the extended primer sequences are substantially complementary to the first or second immobilised primers (e.g. P5 or P7), or substantially complementary to a portion of the first or second immobilised primer.

The 5′ additional nucleotide may be selected from A, T, C or G, but most preferably is T (or U) or C. Preferably 5′ additional nucleotide is not a complement of 3′ nucleotide of the second immobilised primer (where the extended primer sequence binds the first immobilised primer) or is not a complement of 3′ nucleotide of the first immobilised primer (where the extended primer sequence binds the second immobilised primer). For example, where the first immobilised primer is P5 (for example as defined in SEQ ID NO: 1 or 5) and the second immobilised primer is P7 for example as defined in SEQ ID NO: 2), and where the extended primer sequence binds the first immobilised primer, 5′ additional nucleotide is not A. Similarly, where the extended primer sequence binds the second immobilised primer, the 5′ additional nucleotide is not G.

Preferably, the primer-blocking agent is a blocked nucleotide, for example, as described above. More preferably, the blocked nucleotide may be A, C, T or G, but most preferably is selected from A or G. Accordingly, where 5′ additional nucleotide is T or U, the primer-blocking agent is A, and where 5′ additional nucleotide is C, the primer-blocking agent is G.

Again, the extended primer sequence(s) and primer-blocking agent is preferably flowed across the solid support following bridge amplification. More preferably, the primer-blocking agent is flowed across the solid support following at least 1, at least 2, at least 3, at least 4, at least 5, at least 6, at least 7, at least 8, at least 9, at least 10, preferably 15, more preferably 20 and even more preferably 25 rounds of bridge amplification.

In one embodiment, the extended primer sequence is selected from SEQ ID NO: 13 to 24 or a variant or fragment thereof.

One example of this method is shown in FIG. 32. Selective amplification may be conducted on the amplified (duoclonal) cluster as shown in FIG. 2H; as such following a number of rounds of amplification, a cluster is formed comprising both extended first (e.g. P5) and second (e.g. P7) immobilised polynucleotide strands. Before the next round of amplification, a (or a plurality of) extended primer sequence(s) is flowed across the surface of the solid support 200. The extended primer sequence 701 is substantially complementary to at least a portion, if not all of the immobilised primer (e.g. either P5 or P7) and binds to the immobilised primer (e.g. P5 or P7) as shown in FIG. 32A. As also shown in FIG. 32A, the extended primer sequence 701 comprises at least one additional 5′ nucleotide.

Following addition of the extended primer sequence 701, a primer blocking agent 601 is added and flowed across the surface of the solid support (e.g. flow cell). As the primer-blocking agent 601 is complementary to 5′ additional nucleotide of the extended primer sequence 701 the primer-blocking agent 601 binds to 3′-end of the immobilised strands that are hybridised to the extended primer sequence 701, as shown in FIG. 32B. As a consequence, addition of the primer-blocking agent 601 prevents not only extension of the immobilised strand (e.g. P5 or P7) but renders the immobilised primer (P5 or P7) unavailable for hybridisation and subsequent bridge amplification for other extended strands (e.g. 101′) (see FIG. 32B).

Performing at least one more cycle of bridge amplification, leads to selective amplification of the template strands comprising the forward strand of the template 101′ (in a 2:1 ratio of 101′ to 101). Again, similar to FIG. 2D, conducting standard (non-selective) sequencing then allows the forward strands of the template 101′ (i.e. “first portions”) to be sequenced and the forward complement strands of the template 101 (i.e. “second portions”) to be sequenced, wherein a greater proportion of forward strands of the template 101′ are sequenced (grey arrow) compared to a proportion of forward complement strands of the template 101 (black arrow) (FIG. 2D).

The extended primer sequences may be added as part of the amplification mixture described above. Alternatively, the blocked immobilised primer-binding sequence may be added to the flow cell separately and preferably before the amplification mixture is added. Following addition of the blocked immobilised primer-binding sequence, at least one more round of bridge amplification is performed.

Accordingly, in a further aspect of the invention, there is provided an extended primer sequence comprising a sequence selected from SEQ ID NO: 13 to 23 or a variant or fragment thereof.

In a further aspect of the invention, there is provided an amplification composition comprising a recombinase, a DNA polymerase, a single-stranded DNA binding protein (SSB) and at least one blocked immobilised-primer-binding sequence.

By “amplification composition” is meant a composition that is suitable for the amplification of a target nucleic acid template.

In another aspect of the invention, there is provided the use of a blocked immobilised-primer-binding sequence, preferably a blocked immobilised primer-binding sequence comprising a sequence selected from SEQ ID NO: 13 to 23, in preparing at least one polynucleotide sequence for identification.

In some embodiments, selective processing methods may be used to generate signals of different intensities. Accordingly, in some embodiments, the method may comprise selectively processing at least one polynucleotide sequence comprising n portions, such that a proportion of each of the n portions are each capable of generating a respective n^{th} signal, wherein n is 2 or more, and wherein the selective processing causes an intensity of an i^{th} signal to be different compared to an intensity of a j^{th} signal, for all i between 1 to n, and for all j between 1 to n, and where i is not equal to j (e.g. selectively processing at least one polynucleotide sequence comprising a first portion and a second portion, such that a proportion of first portions are capable of generating a first signal and a proportion of second portions are capable of generating a second signal, wherein the selective processing causes an intensity of the first signal to be greater than an intensity of the second signal).

The method may comprise selectively processing a plurality of polynucleotide sequences each comprising n portions, such that a proportion of each of the n portions are each capable of generating a respective n^{th} signal, wherein n is 2 or more, and wherein the selective processing causes an intensity of an i^{th} signal to be different compared to an intensity of a j^{th} signal, for all i between 1 to n, and for all j between 1 to n, and where i is not equal to j (e.g. selectively processing a plurality of polynucleotide sequences each comprising a first portion and a second portion, such that a proportion of first portions are capable of generating a first signal and a proportion of second portions are capable of generating a second signal, wherein the selective processing causes an intensity of the first signal to be greater than an intensity of the second signal).

In one example, by “selective processing” is meant here performing an action that changes relative properties of the n portions in the at least one polynucleotide sequence comprising n portions (or the plurality of polynucleotide sequences each comprising n portions), so that an intensity of an i^{th} signal is different compared to an intensity of a j^{th} signal, for all i between 1 to n, and for all j between 1 to n, and where i is not equal to j (e.g. performing an action that changes relative properties of a first portion and a second portion in a at least one polynucleotide sequence comprising a first portion and a second portion (or a plurality of polynucleotide sequences each comprising a first portion and a second portion), so that the intensity of the first signal is greater than the intensity of the second signal). The property may be, for example, a concentration of each of the i^{th} portions capable of generating the i^{th} signal may be different compared to a concentration of each of the j^{th} portions capable of generating the j^{th} signal (e.g. a concentration of first portions capable of generating the first signal relative to a concentration of second portions capable of generating the second signal). The action may include, for example, conducting selective sequencing, or preparing for selective sequencing.

Selective processing may refer to conducting selective sequencing. Alternatively, selective processing may refer to preparing for selective sequencing. As shown in FIG. 29, in one example, selective sequencing may be achieved using a mixture of unblocked and blocked sequencing primers.

For the purposes of illustration, the disclosure below describes a case where n is 2. However, as will be described in further detail herein, the methods of selective processing are generalizable to cases where n is 2 or more.

Where the method of the invention involves a single (concatenated) polynucleotide strand with a first and second portion, the single (concatenated) polynucleotide strand may comprise a first sequencing primer binding site and a second sequencing primer binding site, where the first sequencing primer binding site and second sequencing primer binding site are of a different sequence to each other and bind different sequencing primers.

In one embodiment, binding of first sequencing primers to the first sequencing primer site generates a first signal and binding of second sequencing primers to the second sequencing primer site generates a second signal, where the intensity of the first signal is greater than the intensity of the second signal. This may be applied to embodiments where the single (concatenated) polynucleotide strand comprises a first sequencing primer binding site and a second sequencing primer binding site. This is achieved using a mixed population of blocked and unblocked second sequencing primers that bind the second sequencing primer site. Any ratio of blocked: unblocked second primers can be used that generates a second signal that is of a lower intensity than the first signal, for example, the ratio of blocked: unblocked primers may be: 20:80 to 80:20, or 1:2 to 2:1.

In one embodiment, a ratio of 50:50 of blocked: unblocked second primers is used, which in turn generates a second signal that is around 50% of the intensity of the first signal.

The first and second sequencing primers may be added to the flow cell at the same time, or separately but sequentially.

The sequence of the sequencing primers and the sequence primer binding sites are not material to the methods of the invention, as long as the sequencing primers are able to bind to the sequence primer binding site to enable amplification and sequencing of the regions to be identified.

In one embodiment, the first sequencing primer binding site may be selected from ME′-A14′ (as defined in SEQ ID NO. 37 or a variant or fragment thereof), A14′ (as defined in SEQ ID NO. 38 or a variant or fragment thereof), ME′-B15′ (as defined in SEQ ID NO. 39 or a variant or fragment thereof) and B15′ (as defined in SEQ ID NO. 40 or a variant or fragment thereof); and the second sequencing primer binding site may be selected from ME′-HYB2 (as defined in SEQ ID NO. 41 or a variant or fragment thereof), HYB2 (as defined in SEQ ID NO. 31 or a variant or fragment thereof), ME′-HYB2′ (as defined in SEQ ID NO. 42 or a variant or fragment thereof) and HYB2′ (as defined in SEQ ID NO. 33 or a variant or fragment thereof).

In another embodiment, the first sequencing primer binding site is ME′-B15′ (as defined in SEQ ID NO. 39 or a variant or fragment thereof), and the second sequencing primer binding site is ME′-HYB2′ (as defined in SEQ ID NO. 42 or a variant or fragment thereof). Alternatively, the first sequencing primer binding site is B15′ (as defined in SEQ ID NO. 40 or a variant or fragment thereof), and the second sequencing primer binding site is HYB2′ (as defined in SEQ ID NO. 33 or a variant or fragment thereof). The first and second sequencing primer sites may be located after (e.g. immediately after) a 3′-end of the first and second portions to be identified.

In another embodiment, the first sequencing primer binding site is ME′-A14′ (as defined in SEQ ID NO. 37 or a variant or fragment thereof), and the second sequencing primer binding site is ME′-HYB2 (as defined in SEQ ID NO. 41 or a variant or fragment thereof). Alternatively, the first sequencing primer binding site may be A14′ (as defined in SEQ ID NO. 38 or a variant or fragment thereof) and the second sequencing primer binding site may be HYB2 (as defined in SEQ ID NO. 31 or a variant or fragment thereof). The first and second sequencing primer sites may be located after (e.g. immediately after) a 3′-end of the first and second portions to be identified.

In one example, the sequencing primer (which may be referred to herein as the second sequencing primer) comprises or consists of a sequence as defined in SEQ ID NO. 31 to 36, or a variant or fragment thereof. The sequencing primer may further comprise a 3′ blocking group as described above to create a blocked sequencing primer. Alternatively, the primer comprises a 3′-OH group. Such a primer is unblocked and can be elongated with a polymerase.

In one embodiment, the unblocked and blocked second sequencing primers are present in the sequencing composition in equal concentrations. That is, the ratio of blocked: unblocked second sequencing primers is around 50:50. The sequencing composition may further comprise at least one additional (first) sequencing primer. This additional sequencing primer may be selected from A14-ME (as defined in SEQ ID NO. 29 or a variant or fragment thereof), A14 (as defined in SEQ ID NO. 27 or a variant or fragment thereof), B15-ME (as defined in SEQ ID NO. 30 or a variant or fragment thereof) and B15 (as defined in SEQ ID NO. 28 or a variant or fragment thereof). In one embodiment, the sequencing composition comprises blocked second sequencing primers, unblocked second sequencing primers and at least one first sequencing primer, wherein the first sequencing primer is A14, or B15, or is both A14 and B15.

As shown in FIG. 28, selective sequencing may be conducted on the amplified (monoclonal) cluster shown in FIG. 28F. A plurality of first sequencing primers 501 are added. These first sequencing primers 501 (e.g. B15-ME; or if ME is not present, then B15) anneal to the first terminal sequencing primer binding site 303 (which represents a type of “first sequencing primer binding site”) (e.g. ME′-B15′; or if ME′ is not present, then B15′). A plurality of second unblocked sequencing primers 502a and a plurality of second blocked sequencing primers 502b are added, either at the same time as the first sequencing primers 501, or sequentially (e.g. prior to or after addition of first sequencing primers 501). These second unblocked sequencing primers 502a (e.g. HYB2-ME; or if ME is not present, then HYB2) and second blocked sequencing primers 502b (e.g. blocked HYB2-ME; or if ME is not present, then blocked HYB2) anneal to an internal sequencing primer binding site in the hybridisation sequence 403′ (which represents a type of “second sequencing primer binding site”) (e.g. ME′-HYB2′; or if ME′ is not present, then HYB2′). This then allows the first insert complement sequences 401′ (i.e. “first portions”) to be sequenced and the second insert complement sequences 402′ (i.e. “second portions”) to be sequenced, wherein a greater proportion of first insert complement sequences 401′ are sequenced (grey arrow) compared to a proportion of second insert complement sequences 402′ (black arrow).

Although FIG. 29 shows selective sequencing being conducted on a template strand attached to first immobilised primer 201, in some embodiments the (monoclonal) cluster may instead have template strands attached to second immobilised primer 202. In such a case, the first sequencing primers may instead correspond to A14-ME (or if ME is not present, then A14), and the second unblocked sequencing primers may instead correspond to HYB2′-ME (or if ME is not present, then HYB2′) and second blocked sequencing primers may instead correspond to blocked HYB2′-ME (or if ME is not present, then blocked HYB2′).

In yet other embodiments, the positioning of first sequencing primers and second sequencing primers may be swapped. In other words, the first sequencing binding primers may anneal instead to the internal sequencing primer binding site, and the second sequencing binding primers may anneal instead to the terminal sequencing primer binding site.

FIG. 29 shows concurrent sequencing of a concatenated strand according to the above method. As shown in FIG. 29, a polynucleotide strand with a first portion (insert) and second portion (insert) can be accurately and simultaneously sequenced by a selective sequencing method that uses a mixture of unblocked and blocked sequencing primers as described above.

### Signal Processing

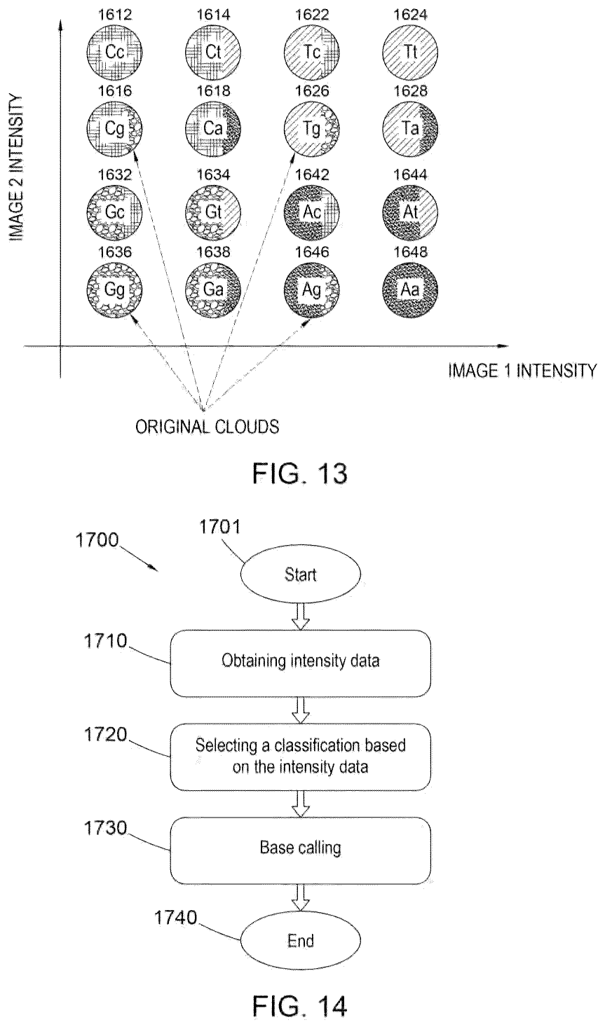

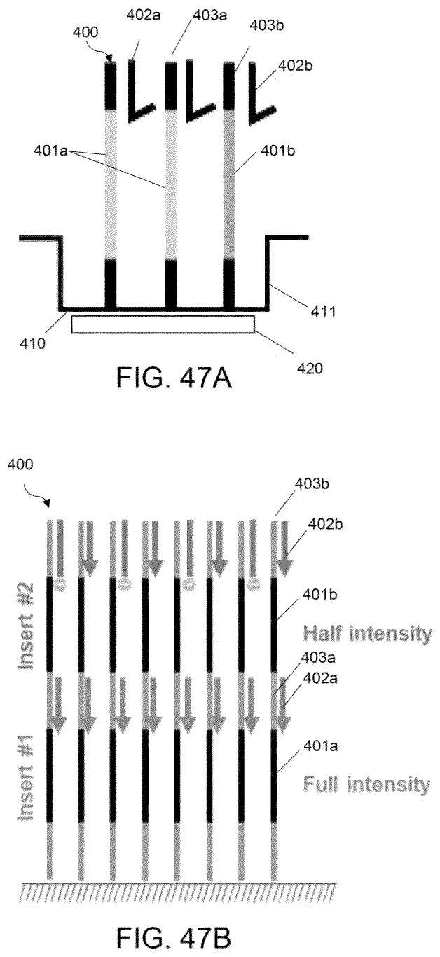

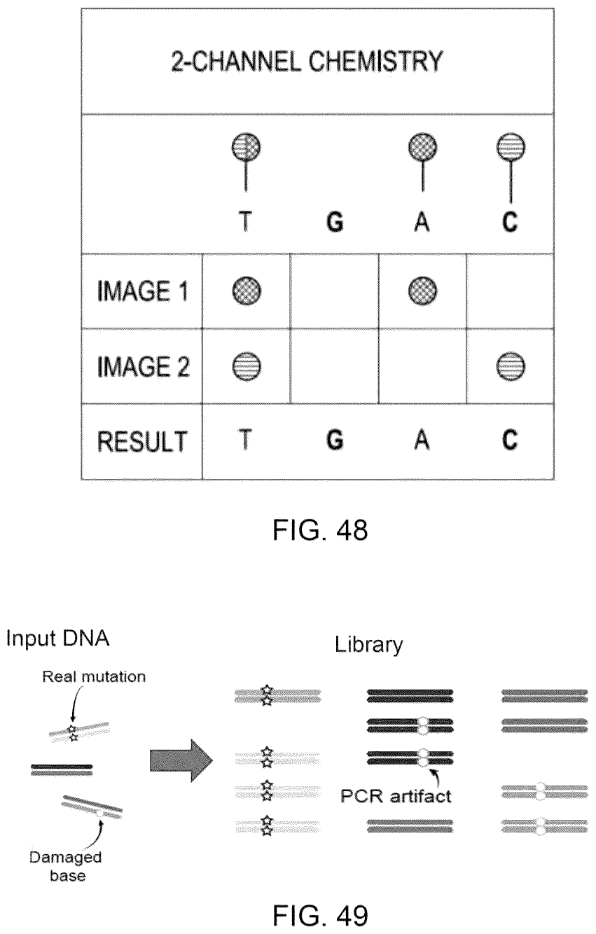

FIG. 13 is a scatter plot showing an example of sixteen distributions of signals from a nucleic acid cluster as illustrated in FIGS. 47A-47B, which may be implemented with the dye labeling scheme shown in FIG. 48 in one example. As explained in connection with FIGS. 47A-47B, in one embodiment, the fluorescent signal coming from the collection of extended first portion sequencing primers 402a will be brighter than the fluorescent signal coming from the collection of extended second portion sequencing primers 402b in the same cluster. The scatter plot of FIG. 13 shows sixteen distributions (or “bins”/“classifications”) of intensity values from the combination of a brighter signal and a dimmer signal; the two signals may be co-localized and may not be optically resolved as described above. The intensity values shown in FIG. 13 may be up to a scale or normalization factor; the units of the intensity values may be arbitrary or relative (i.e., representing the ratio of the actual intensity to a reference intensity). The sum of the brighter signal from the extended first portion primers 402a and the dimmer signal from the extended second portion primers 402b results in a combined signal. The combined signal may be captured by the first optical channel and the second optical channel (e.g., the “IMAGE 1” channel and the “IMAGE 2” channel in FIG. 48). Since the brighter signal may be A, T, C or G, and the dimmer signal may be A, T, C or G, there are sixteen possibilities for the combined signal, corresponding to sixteen distinguishable patterns when optically captured according to the embodiment shown in connection with FIG. 48. That is, each of the sixteen possibilities corresponds to a bin shown in FIG. 13. The computer system can map the combined signal from a cluster into one of the sixteen bins, and thus determine the added nucleobase at the extended first portion primers 402a and the added nucleobase at the extended second portion primers 402b, respectively.

For example, when the combined signal is mapped to bin 612 for a base calling cycle, the computer processor base calls both the added nucleobase at the extended first portion primers 402a and the added nucleobase at the extended second portion primers 402b as C. When the combined signal is mapped to bin 614 for the base calling cycle, the processor base calls the added nucleobase at the extended first portion primers 402a as C and the added nucleobase at the extended second portion primers 402b as T. When the combined signal is mapped to bin 616 for the base calling cycle, the processor base calls the added nucleobase at the extended first portion primers 402a as C and the added nucleobase at the extended second portion primers 402b as G. When the combined signal is mapped to bin 618 for the base calling cycle, the processor base calls the added nucleobase at the extended first portion primers 402a as C and the added nucleobase at the extended second portion primers 402b as A.

When the combined signal is mapped to bin 622 for the base calling cycle, the processor base calls the added nucleobase at the extended first portion primers 402a as T and the added nucleobase at the extended second portion primers 402b as C. When the combined signal is mapped to bin 624 for the base calling cycle, the processor base calls both the added nucleobase at the extended first portion primers 402a and the added nucleobase at the extended second portion primers 402b as T. When the combined signal is mapped to bin 626 for the base calling cycle, the processor base calls the added nucleobase at the extended first portion primers 402a as T and the added nucleobase at the extended second portion primers 402b as G. When the combined signal is mapped to bin 628 for the base calling cycle, the processor base calls the added nucleobase at the extended first portion primers 402a as T and the added nucleobase at the extended second portion primers 402b as A.

When the combined signal is mapped to bin 632 for the base calling cycle, the processor base calls the added nucleobase at the extended first portion primers 402a as G and the added nucleobase at the extended second portion primers 402b as C. When the combined signal is mapped to bin 634 for the base calling cycle, the processor base calls the added nucleobase at the extended first portion primers 402a as G and the added nucleobase at the extended second portion primers 402b as T. When the combined signal is mapped to bin 636 for the base calling cycle, the processor base calls both the added nucleobase at the extended first portion primers 402a and the added nucleobase at the extended second portion primers 402b as G. When the combined signal is mapped to bin 638 for the base calling cycle, the processor base calls the added nucleobase at the extended first portion primers 402a as G and the added nucleobase at the extended first portion primers 402b as A.

When the combined signal is mapped to bin 642 for the base calling cycle, the processor base calls the added nucleobase at the extended first portion primers 402a as A and the added nucleobase at the extended second portion primers 402b as C. When the combined signal is mapped to bin 644 for the base calling cycle, the processor base calls the added nucleobase at the extended first portion primers 402a as A and the added nucleobase at the extended second portion primers 402b as T. When the combined signal is mapped to bin 646 for the base calling cycle, the processor base calls the added nucleobase at the extended first portion primers 402a as A and the added nucleobase at the extended second portion primers 402b as G. When the combined signal is mapped to bin 648 for the base calling cycle, the processor base calls both the added nucleobase at the extended first portion primers 402a and the added nucleobase at the extended second portion primers 402b as A. Further details regarding performing base-calling based on a scatter plot having sixteen bins may be found in U.S. Patent Application Publication No. 2019/0212294, the disclosure of which is incorporated herein by reference.

It will be appreciated that the number of distributions may be different, depending upon the number of sequence portions of interest which are concurrently sequenced. For example, the plurality of classifications may comprise 4ⁿ classifications, each classification representing one of 4ⁿ unique combinations of n^{th} nucleobases in each of n sequence portions.

Accordingly, in one aspect of the present disclosure, there is provided a method of base calling nucleobases of n polynucleotide sequence portions, the method comprising:
(a) obtaining first intensity data comprising a combined intensity of respective first signal components generated by each of the n^{th} portions obtained based upon respective n^{th} nucleobases in each of the n portions, wherein the respective first signal components are obtained simultaneously;
(b) obtaining second intensity data comprising a combined intensity of respective second signal components generated by each of the n^{th} portions obtained based upon respective n^{th} nucleobases in each of the n portions, wherein the respective second signal components are obtained simultaneously;
(c) selecting one of a plurality of classifications based on the first and the second intensity data, wherein each classification represents a possible combination of respective n^{th} nucleobases; and
(d) based on the selected classification, base calling the respective n^{th} nucleobases for all n portions,
wherein said polynucleotide sequence portions have been selectively processed such that an intensity of the signals obtained based upon the respective first nucleobase is greater than an intensity of the signals obtained based upon the respective second nucleobase.

Selecting the classification based on the first and second intensity data may comprise selecting the classification based on the combined intensity of respective first signal components and second signal components. The plurality of classifications may comprise 4ⁿ classifications, each classification representing one of 4ⁿ unique combinations of n^{th} nucleobases.

In addition to base calling the first and second polynucleotide sequence portions, the mapping of the combined signal to each of the different bins (e.g. in combination with additional knowledge, such as the library preparation methods used) can provide additional information about the first and second polynucleotide sequence portions, or about sequences from which the first and second portions were derived. For example, given the nucleic acid material input and the processing methods used to generate the nucleic acid clusters, the first and second sequence portions may be expected to be identical at a given position. In this case, the mapping of the combined signal to a bin representing a mismatch may be indicative of an error introduced during library preparation. Alternatively, the first and second sequence portions may be expected to be different, for example due to deliberate sequence modifications introduced during library preparation.

### Detecting Library Preparation Errors

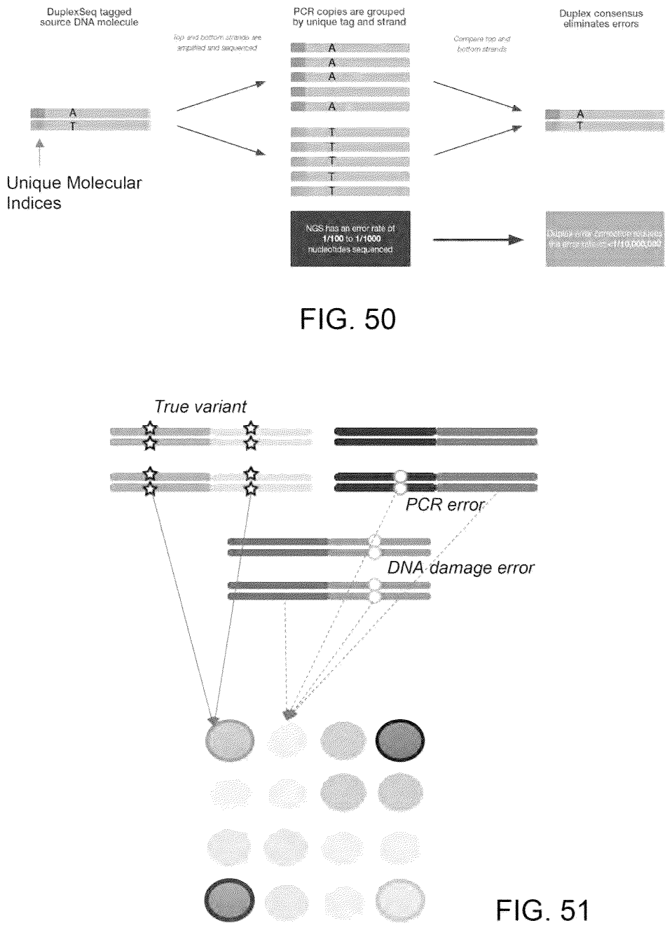

Errors arise during NGS library preparation, for example due to PCR artefacts or DNA damage. The error rate is determined by the library preparation method used, for example the number of cycles of PCR amplification carried out, and a typical error rate may be of the order of 0.1%. This limits the sensitivity of diagnostic assays based on the sequencing method, and may obscure true variants (as shown in FIG. 49). One solution to this problem is to use unique molecular identifiers or indices (UMIs) to distinguish true (e.g. rare) mutations from mutations arising due to library preparation errors (as shown in FIG. 50). The present methods, however, allow for the identification of library preparation errors from fewer sequencing reads.

In one example, a plurality of clusters are generated, each comprising at least one first polynucleotide sequence portion and at least one second polynucleotide sequence portion, wherein the first and second portions will be the same in the absence of any library preparation or sequencing errors. For example, the nucleic acid clusters may be processed such that each of the one or more first portions corresponds to the sequence of a forward strand (or a reverse strand) of a double-stranded template molecule, and the one or more second portions corresponds to the complement of the reverse strand (or the forward strand) of the template. Examples of suitable methods for generating such clusters are described below. In the absence of any library preparation/sequencing errors, the signals produced by subjecting the two sequence portions to sequencing-by-synthesis will match. The combined signal may therefore be mapped to one of the four “corner” clouds shown in FIG. 51. Should the identity of the nucleobase at that position suggest a rare, or even unknown, variant, it can be determined with a high level of confidence that the base call represents a true variant, as opposed to a library preparation error. If, on the other hand, the combined signal is mapped to any of the other clouds, this indicates that the sequences of the first and second sequence portions do not match, and that an error has occurred in library preparation. Therefore, in response to mapping the combined signal to a classification representing a mismatch between the two nucleobases, a library preparation error may be identified.

Depending upon the library preparation methods used, it is not necessarily the case, however, that a match between the first and second nucleobases added to the first and second polynucleotide sequence portions is indicative of the absence of a library preparation error. Similarly, it is not necessarily the case that a mismatch between the first and second nucleobases is indicative of the presence of a library preparation error. For example, one or more sequence modifications may be intentionally introduced during library preparation as described in the example below.

### Detecting Sequence Modifications

The present technology also allows for the detection of sequence modifications (e.g. deliberate sequence modifications) made during library preparation. In particular, a priori information of a modification performed to obtain a sequence may be used to obtain information associated with the modification.

Of the many possible DNA modifications, the methylation of cytosines is the most frequently observed in relation to gene regulation. In order to determine the methylation profile of a nucleic acid sequence, it is known to treat the nucleic acid sequence (e.g. chemically or enzymatically) to convert either methylated or unmethylated cytosine to a different base. For example, bisulfite treatment may be used to convert unmethylated cytosine to uracil. Alternatively, borane treatment, or enzymatic conversion (e.g. using an APOBEC or activation-induced cytidine deaminase (AID) enzyme) may be used to convert 5-methylcytosine to thymine. According to prior methods, inferring methylation status requires conversion, sequencing, and comparison either to a database reference, to an unconverted reference, or to an opposite strand in a consensus pileup. The present methods, however, allow for the methylation status of a sequence to be determined in real-time, from a single sequencing run, and without the need for alignment.

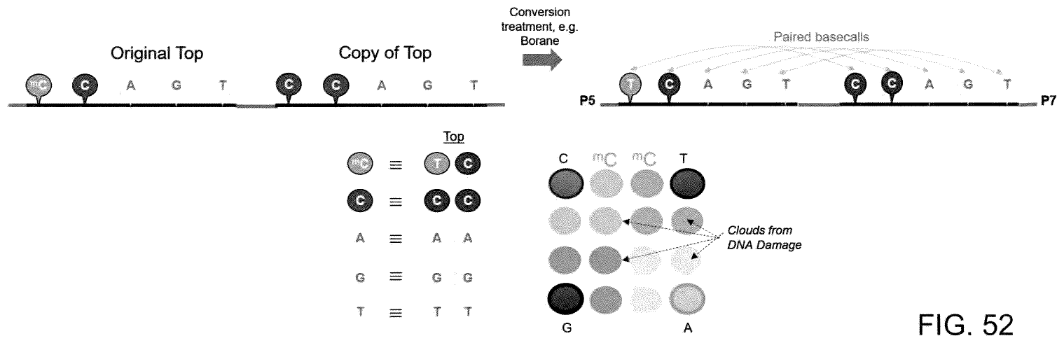

FIG. 52 shows an example of a method for the identification of methylated bases according to the present disclosure. Concatenated polynucleotide sequences comprising a first portion and a second portion are prepared using a tandem insert method as described below. A first portion will have the methylation fingerprint of the original molecule, while the second portion will have lost the methylation fingerprint but will retain the original base information. Following conversion treatment of methylcytosines to thymine (e.g. using Borane or APOBEC treatment), cytosines will be converted to the modified base, while the second portion retains the original base information. The sequence of the original molecule, including methylation status, can then be determined based upon the distribution to which the combined signal intensities from the two portions are mapped.

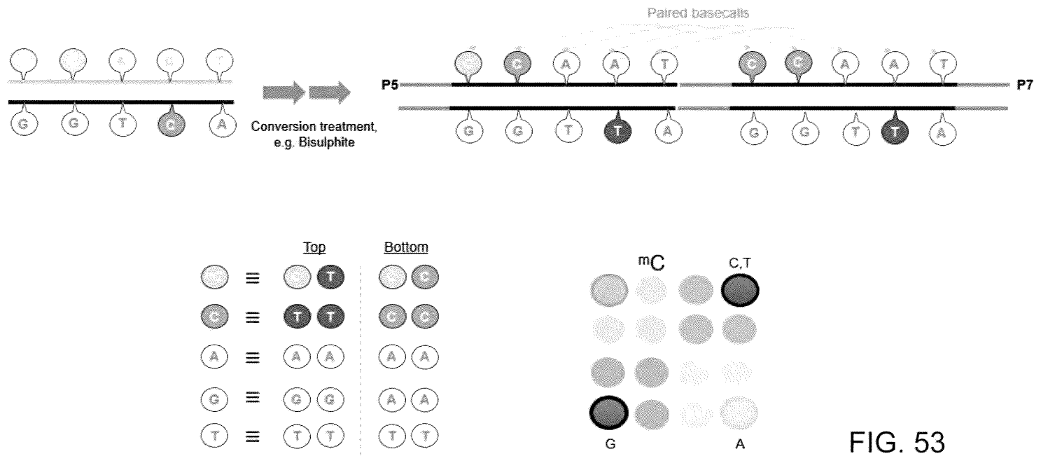

Alternatively, as shown in FIG. 53, unmodified cytosines may be converted to uracil, for example using bisulfite treatment. Here, the complementary strand may also be used in order to extract the full original sequence information.

Alternative library preparation and cluster generation methods may also be used for the purpose of determining methylation status. For example, nucleic acid clusters may be prepared, each comprising one or more first polynucleotide sequence portions corresponding to the sequence of a forward strand (or a reverse strand) of a treated molecule, and one or more second polynucleotide sequence portions corresponding to the complement of the reverse strand (or the forward strand) of the treated molecule. By virtue of the methylation conversion happening prior to the copying process, methylation information of both strands of the original molecule can be determined in a single sequencing run.

### Signal Mapping

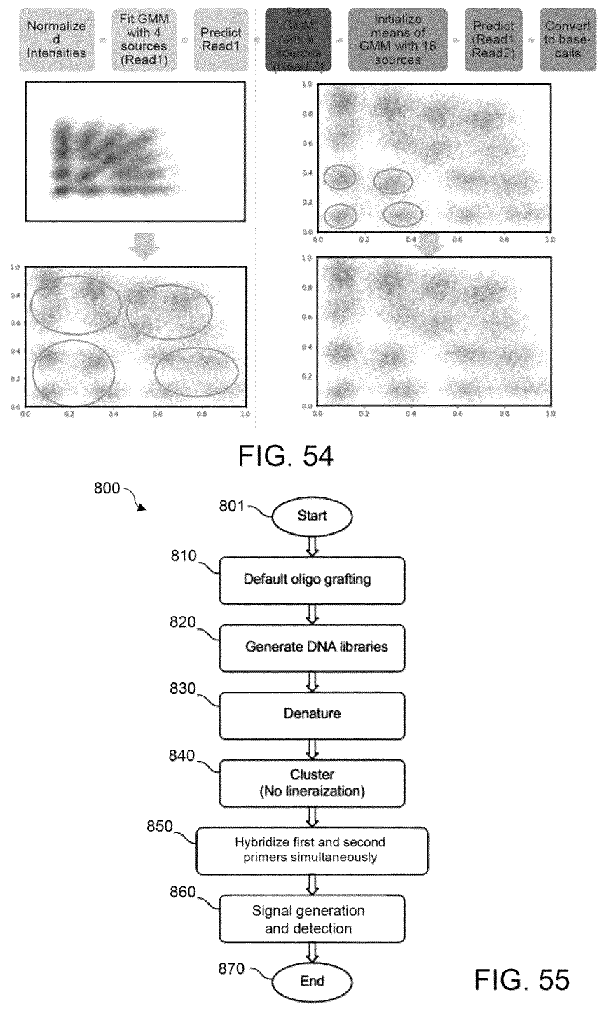

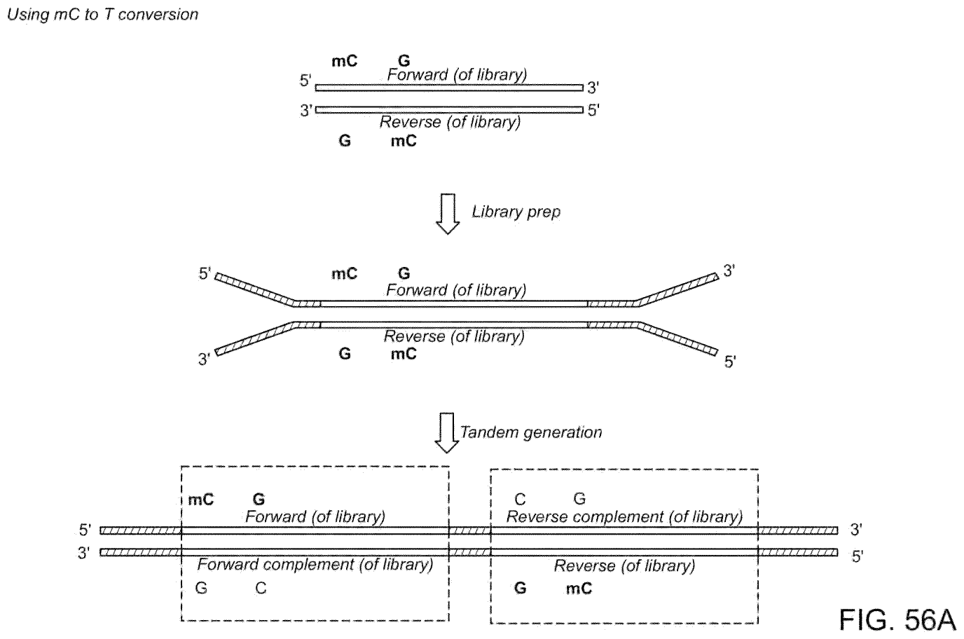

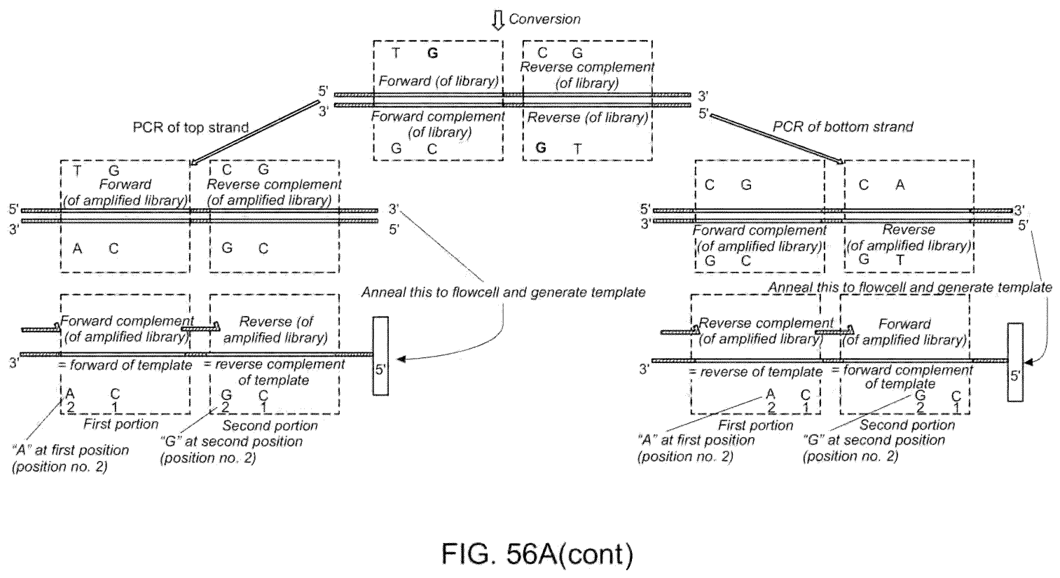

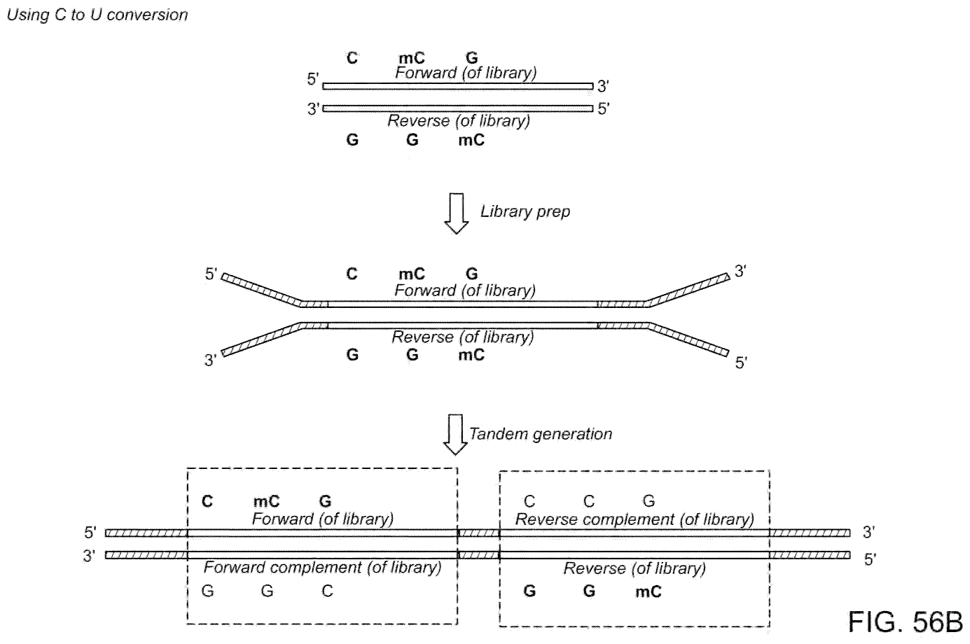

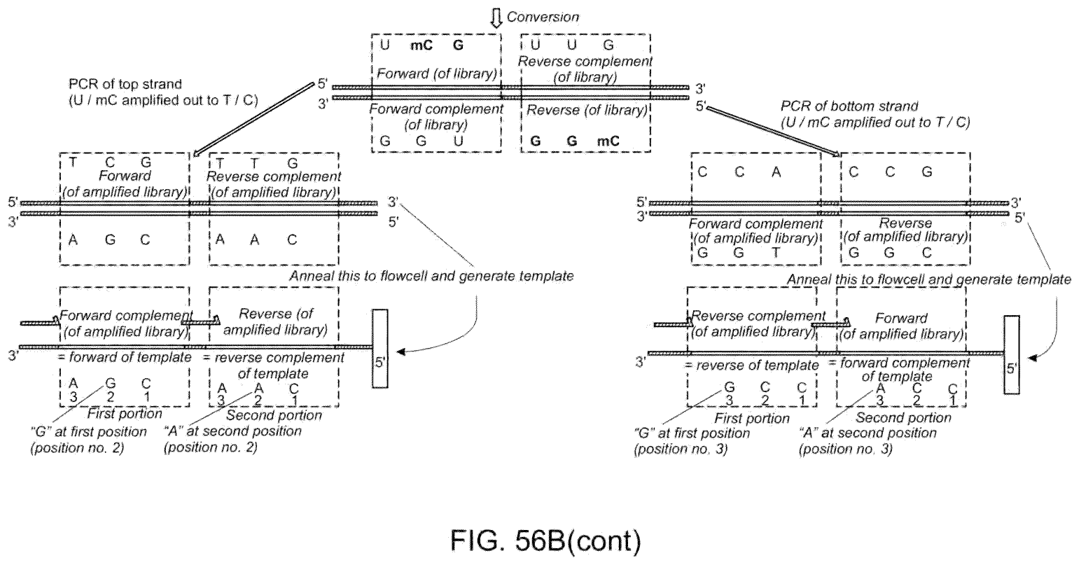

In one example, the combined signals may be mapped to the distributions by using a Gaussian Mixture Model (GMM). For example, as shown in FIG. 54, raw intensities for each cluster may first be separately normalized. A GMM with four sources may then be fitted and used to predict the brighter or “major” read (bottom left-hand panel). The intensities with the same major read may then be used to train a GMM with four sources for the dimmer or “minor” read (top right-hand panel), and the trained GMM may be used to predict the minor read for each major read, resulting in 16 cluster centers (bottom right-hand panel). A GMM with 16 sources may then be initialized with these 16 cluster centers, and trained with all cluster intensities for each cycle. The two reads may then be predicted using GMM cluster assignment and correlation with the expected cloud constellation.

### Simplified Sequencing Workflow

FIG. 14 is a flow diagram showing a method 1700 of base calling according to the present disclosure. The described method allows for simultaneous sequencing of two or more sequence portions in a single sequencing run from a single combined signal obtained from the two or more portions, thus requiring less sequencing reagent consumption and faster generation of data from both portions. Further, the simplified method may reduce the number of workflow steps while producing the same yield as compared to existing next-generation sequencing methods. Thus, the simplified method may result in reduced sequencing runtime. As shown in FIG. 14, the disclosed method 1700 may start from block 1701. The method may then move to block 1710.

At block 1710, intensity data is obtained. The intensity data includes first intensity data and second intensity data. The first intensity data comprises a combined intensity of a first signal obtained based upon a respective first nucleobase of at least one first polynucleotide sequence portion and a second signal obtained based upon a respective second nucleobase of at least one second polynucleotide sequence portion. Similarly, the second intensity data comprises a combined intensity of a third signal obtained based upon the respective first nucleobase of the at least one first polynucleotide sequence portion and a fourth signal obtained based upon the respective second nucleobase of the at least one second polynucleotide sequence portion.

As described above, polynucleotide molecules comprising the at least one first polynucleotide sequence portion and the at least one second polynucleotide sequence portion may be arranged on the flow cell such that light emissions from the first and second portions are detected by a single sensing portion and/or may comprise a single cluster such that light emissions from each of the respective two polynucleotide sequence portions cannot be spatially resolved.

In one example, the signals may be generated according to the method shown in FIG. 55.

In one example, obtaining the intensity data comprises selecting intensity data that corresponds to two or more different sequence portions. In one example, intensity data is selected based upon a chastity score. A chastity score may be calculated as the ratio of the brightest base intensity divided by the sum of the brightest and second brightest base intensities. The desired chastity score may be different depending upon the expected intensity ratio of the light emissions associated with the different portions. As described above, it may be desired to produce clusters comprising two different sequence portions of interest, which give rise to signals in a ratio of 2:1. In one example, high-quality data corresponding to two sequence portions with an intensity ratio of 2:1 may have a chastity score of around 0.8 to 0.9. In one example, clusters may be identified as containing one or more than one polynucleotide sequence portion of interest (e.g. by chastity score) and processed accordingly. For example, clusters containing more than one sequence portion of interest may be base called according to the present methods, whereas clusters containing a single sequence portion of interest may be base called according to known methods. After the intensity data has been obtained, the method may proceed to block 1720. In this step, one of a plurality of classifications is selected based on the intensity data. Each classification represents a possible combination of respective first and second nucleobases. In one example, the plurality of classifications comprises sixteen classifications as shown in FIG. 13, each representing a unique combination of first and second nucleobases. Where there are two polynucleotide sequence regions of interest, there are sixteen possible combinations of first and second nucleobases. Selecting the classification based on the first and second intensity data comprises selecting the classification based on the combined intensity of the first and second signals and the combined intensity of the third and fourth signals, for example using GMMs as described above.

The method may then proceed to block 1730, where the respective first and second nucleobases are base called based on the classification selected in block 1720. The light emissions generated during a cycle of a sequencing-by-synthesis method are indicative of the identity of the nucleobase(s) added to the sequencing primers undergoing extension. It will be appreciated that there is a direct correspondence between the identity of the nucleobases incorporated into the sequencing primers and the identity of the complementary base at the corresponding position of the sequence portion bound to the flow cell. Therefore, any references herein to the base calling of respective nucleobases of polynucleotide sequence portions encompasses the base calling of nucleobases hybridized to the polynucleotide sequence portions and, alternatively or additionally, the identification of the corresponding nucleobases of the portions. The method may then end at block 1740.

FIG. 55 is a flow diagram showing a method 800 by which the signals discussed in relation to block 1710 of FIG. 14 may be generated. The method may start from block 801.

The method may then move to block 810, default oligo grafting, which may include the attachment of oligonucleotide anchors/graft sequences to a planar, optically transparent surface of the flow cell. The method may then move to block 820, generating DNA libraries from a sample, where template polynucleotides in a sample may be end-repaired to generate 5′-phosphorylated blunt ends, and the polymerase activity of Klenow fragment may be used to add a single A base to the 3′ end of the blunt phosphorylated nucleic acid fragments. This addition prepares the nucleic acid fragments for ligation to oligonucleotide adapters, which have an overhang of a single T base at their 3′ end to increase ligation efficiency. The adapter oligonucleotides are complementary to the flow cell anchor oligos.

After DNA library generation, the method may then move to block 830, denaturing the double stranded DNA libraries to generate single stranded template polynucleotides for seeding on the flow cell. The method may then move to block 840, clustering from the single stranded template polynucleotides. Under limiting-dilution conditions, adapter-modified, single-stranded template polynucleotides are added to the flow cell and immobilized by hybridization to the anchor oligos. Attached nucleic acid fragments are extended and bridge amplified to create an ultra-high density sequencing flow cell with hundreds of millions of clusters, each containing about 1,000 copies of the same template. Details regarding enrichment of nucleic acids using cluster amplification may be found in Kozarewa et al., Nature Methods 6:291-295 (2009), which is incorporated herein by reference.

After cluster generation, the method may directly move to block 850, hybridizing/annealing first and second primers 402a, 402b simultaneously to both the first and second polynucleotide sequence portions 401a, 401b on the flow cell 410. Next, the method may move to block 860 of signal generation. Signal generation proceeds by simultaneously extending the hybridized primers 402a, 402b. With each cycle, fluorescently tagged nucleotides compete for addition to the growing chains of extended primers. Only one is incorporated at a primer location based on the sequence of the template strand. After the addition of nucleotides, the cluster is excited by a light source, and characteristic fluorescent signals are emitted. The emission spectra and the signal intensities uniquely determine the base call. Hundreds of millions of nucleic acid clusters, or thousands to tens of thousands of millions of clusters, may be sequenced in a massively parallel manner. After sequencing the polynucleotide sequence portions 401a, 401b on the flow cell 410, the method may end at block 870.

Methods of library preparation, cluster generation and amplification, sequencing, and selective processing which are suitable for use with the present base calling methods will now be described in further detail.

### Data Analysis Using 16 QaM

FIG. 13 is a scatter plot showing an example of sixteen distributions of signals generated by polynucleotide sequences disclosed herein.

The scatter plot of FIG. 13 shows sixteen distributions (or bins) of intensity values from the combination of a brighter signal (i.e. a first signal as described herein) and a dimmer signal (i.e. a second signal as described herein); the two signals may be co-localized and may not be optically resolved as described above. The intensity values shown in FIG. 13 may be up to a scale or normalisation factor; the units of the intensity values may be arbitrary or relative (i.e., representing the ratio of the actual intensity to a reference intensity). The sum of the brighter signal generated by the first portions and the dimmer signal generated by the second portions results in a combined signal. The combined signal may be captured by a first optical channel and a second optical channel. Since the brighter signal may be A, T, C or G, and the dimmer signal may be A, T, C or G, there are sixteen possibilities for the combined signal, corresponding to sixteen distinguishable patterns when optically captured. That is, each of the sixteen possibilities corresponds to a bin shown in FIG. 13. The computer system can map the combined signal generated into one of the sixteen bins, and thus determine the added nucleobase at the first portion and the added nucleobase at the second portion, respectively.

For example, when the combined signal is mapped to bin 1612 for a base calling cycle, the computer processor base calls both the added nucleobase at the first portion and the added nucleobase at the second portion as C. When the combined signal is mapped to bin 1614 for the base calling cycle, the processor base calls the added nucleobase at the first portion as C and the added nucleobase at the second portion as T. When the combined signal is mapped to bin 1616 for the base calling cycle, the processor base calls the added nucleobase at the first portion as C and the added nucleobase at the second portion as G. When the combined signal is mapped to bin 1618 for the base calling cycle, the processor base calls the added nucleobase at the first portion as C and the added nucleobase at the second portion as A.

When the combined signal is mapped to bin 1622 for the base calling cycle, the processor base calls the added nucleobase at the first portion as T and the added nucleobase at the second portion as C. When the combined signal is mapped to bin 1624 for the base calling cycle, the processor base calls both the added nucleobase at the first portion and the added nucleobase at the second portion as T. When the combined signal is mapped to bin 1626 for the base calling cycle, the processor base calls the added nucleobase at the first portion as T and the added nucleobase at the second portion as G. When the combined signal is mapped to bin 1628 for the base calling cycle, the processor base calls the added nucleobase at the first portion as T and the added nucleobase at the second portion as A.

When the combined signal is mapped to bin 1632 for the base calling cycle, the processor base calls the added nucleobase at the first portion as G and the added nucleobase at the second portion as C. When the combined signal is mapped to bin 1634 for the base calling cycle, the processor base calls the added nucleobase at the first portion as G and the added nucleobase at the second portion as T. When the combined signal is mapped to bin 1636 for the base calling cycle, the processor base calls both the added nucleobase at the first portion and the added nucleobase at the second portion as G. When the combined signal is mapped to bin 1638 for the base calling cycle, the processor base calls the added nucleobase at the first portion as G and the added nucleobase at the second portion as A.

When the combined signal is mapped to bin 1642 for the base calling cycle, the processor base calls the added nucleobase at the first portion as A and the added nucleobase at the second portion as C. When the combined signal is mapped to bin 1644 for the base calling cycle, the processor base calls the added nucleobase at the first portion as A and the added nucleobase at the second portion as T. When the combined signal is mapped to bin 1646 for the base calling cycle, the processor base calls the added nucleobase at the first portion as A and the added nucleobase at the second portion as G. When the combined signal is mapped to bin 1648 for the base calling cycle, the processor base calls both the added nucleobase at the first portion and the added nucleobase at the second portion as A.

In this particular example, T is configured to emit a signal in both the IMAGE 1 channel and the IMAGE 2 channel, A is configured to emit a signal in the IMAGE 1 channel only, C is configured to emit a signal in the IMAGE 2 channel only, and G does not emit a signal in either channel. However, different permutations of nucleobases can be used to achieve the same effect by performing dye swaps. For example, A may be configured to emit a signal in both the IMAGE 1 channel and the IMAGE 2 channel, T may be configured to emit a signal in the IMAGE 1 channel only, C may be configured to emit a signal in the IMAGE 2 channel only, and G may be configured to not emit a signal in either channel.

Further details regarding performing base-calling based on a scatter plot having sixteen bins may be found in U.S. Patent Application Publication No. 2019/0212294, the disclosure of which is incorporated herein by reference.

FIG. 14 is a flow diagram showing a method 1700 of base calling according to the present disclosure. The described method allows for simultaneous sequencing of two (or more) portions (e.g. the first portion and the second portion) in a single sequencing run from a single combined signal obtained from the first portion and the second portion, thus requiring less sequencing reagent consumption and faster generation of data from both the first portion and the second portion. Further, the simplified method may reduce the number of workflow steps while producing the same yield as compared to existing next-generation sequencing methods. Thus, the simplified method may result in reduced sequencing runtime.

As shown in FIG. 14, the disclosed method 1700 may start from block 1701. The method may then move to block 1710.

At block 1710, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data that corresponds to two (or more) different portions (e.g. the first portion and the second portion). In one example, intensity data is selected based upon a chastity score. A chastity score may be calculated as the ratio of the brightest base intensity divided by the sum of the brightest and second brightest base intensities. The desired chastity score may be different depending upon the expected intensity ratio of the light emissions associated with the different portions. As described above, it may be desired to produce clusters comprising the first portion and the second portion, which give rise to signals in a ratio of 2:1. In one example, high-quality data corresponding to two portions with an intensity ratio of 2:1 may have a chastity score of around 0.8 to 0.9.

After the intensity data has been obtained, the method may proceed to block 1720. In this step, one of a plurality of classifications is selected based on the intensity data. Each classification represents a possible combination of respective first and second nucleobases. In one example, the plurality of classifications comprises sixteen classifications as shown in FIG. 13, each representing a unique combination of first and second nucleobases. Where there are two portions, there are sixteen possible combinations of first and second nucleobases. Selecting the classification based on the first and second intensity data comprises selecting the classification based on the combined intensity of the first and second signal components and the combined intensity of the third and fourth signal components.

The method may then proceed to block 1730, where the respective first and second nucleobases are base called based on the classification selected in block 1720. The signals generated during a cycle of a sequencing are indicative of the identity of the nucleobase(s) added during sequencing (e.g. using sequencing-by-synthesis). It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1740.

More generally, when there are n portions, there are 4ⁿ possible combinations of n nucleobases. Each combination can be attributed to a particular classification as each of the n portions generates a different intensity signal.

The method may then proceed to block 1730, where the respective first and second nucleobases are base called based on the classification selected in block 1720. The signals generated during a cycle of a sequencing are indicative of the identity of the nucleobase(s) added during sequencing (e.g. using sequencing-by-synthesis). It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1740.

Generalisation to n-mer Polynucleotides

The disclosure has described a specific case of (concatenated) polynucleotide sequences comprising two portions (i.e. a first portion and a second portion). However, embodiments of the present invention are not limited to two portions. In particular, methods described herein may also be applied to (concatenated) polynucleotide sequences, comprising not just two portions to be identified, but rather n portions to be identified.

As such, each of the concepts above relating to at least one polynucleotide sequence comprising a first portion and a second portion may instead refer to at least one polynucleotide sequence comprising n portions.

Such polynucleotide sequences can also be prepared by methods described herein, for example using PCR stitching.

Accordingly, we describe a method of preparing at least one polynucleotide sequence for identification, comprising:
selectively processing at least one polynucleotide sequence comprising n portions, such that a proportion of each of the n portions are each capable of generating a respective n^{th} signal,
wherein n is 2 or more, and
wherein the selective processing causes an intensity of an i^{th} signal to be different compared to an intensity of a j^{th} signal, for all i between 1 to n, and for all j between 1 to n, and where i is not equal to j.

In other words, the selective processing causes an intensity of each n^{th} signal to be different compared to an intensity of each other n^{th} signal.

Advantageously, it is this selective processing that primes the n portions to be ready for concurrent sequencing. This therefore allows each of the n portions to be identified simultaneously, which leads to an increase in sequencing efficiency and throughput. This means that massively parallel sequencing is enabled in a third dimension (z-axis), and not just over two dimensions.

For the purposes of labelling, the n portions in the at least one polynucleotide sequence may be ordered sequentially. In other words, from one end of the at least one polynucleotide sequence to the other end of the at least one polynucleotide sequence, the at least one polynucleotide sequence comprises a first portion, a second portion, etc., up to the n^{th} portion. This may be from 5′-end to 3′-end of the at least one polynucleotide sequence; alternatively, this may be from 3′-end to 5′-end of the at least one polynucleotide sequence.

The order of intensities for each n^{th} signal may not necessarily follow the sequential order of the n portions within the at least one polynucleotide sequence. Different permutations of signal intensities are possible, and all of these permutations represent ways of achieving various embodiments of the present invention. As an illustrative example, if the at least one polynucleotide sequence comprises a first portion, a second portion, a third portion and a fourth portion, it may be the third portion that gives rise to the most intense signal, followed by the first portion giving rise to the second most intense signal, followed by the fourth portion giving rise to the third most intense signal, followed by the second portion giving rise to the fourth most intense signal; alternatively again for the purposes of illustration, it may be the second portion that gives rise to the most intense signal, followed by the fourth portion that gives rise to the second most intense signal, followed by the third portion that gives rise to the third most intense signal, followed by the first portion that gives rise to the fourth most intense signal.

The at least one polynucleotide sequence may be a plurality of polynucleotide sequences each comprising their respective n portions.

Accordingly, the method may comprise:
selectively processing a plurality of polynucleotide sequences each comprising n portions, such that a proportion of each of the n portions are each capable of generating a respective n^{th} signal,
wherein n is 2 or more, and
wherein the selective processing causes an intensity of an i^{th} signal to be different compared to an intensity of a j^{th} signal, for all i between 1 to n, and for all j between 1 to n, and where i is not equal to j.

As mentioned above, selective processing refers to performing an action that changes relative properties of each n portions within the at least one polynucleotide sequence. This property may be, for example, a concentration of each of the n portions.

In some embodiments, a concentration of each of the i^{th} portions capable of generating the i^{th} signal may be different compared to a concentration of each of the j^{th} portions capable of generating the j^{th} signal. In other words, a concentration of each of the n portions capable of generating the n^{th} signal may be different compared to a concentration of each of the other n portions capable of generating the n^{th} signal.

In one embodiment, a ratio between a concentration of one of the n portions capable of generating the (m−1)^{th} most intense signal and a concentration of another of the n portions capable of generating the m^{th} most intense signal may be between 1.25:1 to 5:1, or between 1.5:1 to 3:1, or about 2:1, wherein m is between 2 to n. In other words, when comparing an n^{th} signal of a particular intensity with an n^{th} signal of the next highest intensity (i.e. having an intensity less than the n^{th} signal of the particular intensity), the ratio between the concentration of one of the n portions capable of generating the n^{th} signal of the particular intensity and the concentration of one of the n portions capable of generating the n^{th} signal of the next highest intensity may be between 1.25:1 to 5:1, or between 1.5:1 to 3:1, or about 2:1.

In one aspect, a ratio between each concentration of one of the n portions capable of generating the (m−1)^{th} most intense signal and each concentration of another of the n portions capable of generating the m^{th} most intense signal may be between 1.25:1 to 5:1, or between 1.5:1 to 3:1, or about 2:1, for all m between 2 to n. In other words, when comparing an n^{th} signal of a particular intensity with an n^{th} signal of the next highest intensity (i.e. having an intensity less than the n^{th} signal of the particular intensity), the ratio between the concentration of each of the n portions capable of generating the n^{th} signal of the particular intensity and the concentration of each of the n portions capable of generating the n^{th} signal of the next highest intensity may be between 1.25:1 to 5:1, or between 1.5:1 to 3:1, or about 2:1.

In some embodiments, each of the n^{th} signals may be spatially unresolved.

In some embodiments, selectively processing may comprise conducting selective sequencing. Alternatively, selective processing may refer to preparing for selective sequencing.

In some embodiments, selectively processing may comprise:
contacting n^{th} sequencing primer binding sites located after a 3′-end of each of the respective n portions with respective n^{th} primers, wherein at least one of the n^{th} primers comprises a mixture of blocked n^{th} primers and unblocked n^{th} primers, and
of the n^{th} primers that do comprise a mixture of blocked n^{th} primers and unblocked n^{th} primers, a ratio of blocked n^{th} primers to unblocked n^{th} primers is different compared to a ratio of blocked primers and unblocked primers of all other primers comprising a mixture of respective blocked and unblocked primers.

Each of the n^{th} sequencing primer binding sites are of a different sequence to each other and bind different sequencing primers.

In some embodiments, all but one of the n^{th} primers may comprise a mixture of blocked n^{th} primers and unblocked n^{th} primers. In other words, one of the n^{th} primers may comprise only unblocked n^{th} primers, and no blocked n^{th} primers. For all of the other n^{th} primers, each of these may comprise a mixture of blocked n^{th} primers and unblocked n^{th} primers, and for each of these types of n^{th} primers, a ratio of blocked n^{th} primers to unblocked n^{th} primers is different compared to a ratio of blocked primers and unblocked primers of all other primers comprising a mixture of respective blocked and unblocked primers.

Again, by “blocked” is meant that the n^{th} sequencing primer comprises a blocking group at a 3′ end of the sequencing primer. In particular, each blocked n^{th} primer may comprise a blocking group at a 3′ end of the blocked n^{th} primer. Suitable blocking groups include a hairpin loop (e.g. a polynucleotide attached to 3′-end, comprising in a 5′ to 3′ direction, a cleavable site such as a nucleotide comprising uracil, a loop portion, and a complement portion, wherein the complement portion is substantially complementary to all or a portion of the sequencing primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group)), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase.

In one embodiment, one of the blocked n^{th} primers may comprise a sequence as defined in SEQ ID NO. 31 to 36 or a variant or fragment thereof and/or the corresponding unblocked n^{th} primer may comprise a sequence as defined in SEQ ID NO. 31 to 34 or a variant or fragment thereof.

The number “n” may be chosen by balancing the accuracy of reads and the overall throughput. As n decreases, the signal-to-noise ratio may increase and as such the accuracy of reads may also increase. As n increases, the overall throughput may increase. In some embodiments, n may be between 2 to 6, or between 2 to 4. In an alternative embodiment, n may be 3 or more, or between 3 to 6, or 3 or 4. Such values of n can achieve a balance between accuracy of reads and overall throughput.

In general, embodiments can be applied to the sequencing of multiple different sequences on the same strand simultaneously. Accordingly, one of the n portions may have a different polynucleotide sequence compared to another of the n portions, wherein the respective sequences may be genetically unrelated and/or obtained from different sources. As mentioned above, genetically unrelated sequences may be different fragment sequences which are derived from the same source, but are different fragments from that source (e.g. from the same fragmented library preparation process). Genetically unrelated sequences may also include sequences that can be overlapping in sequence (but not identical in sequence). In one embodiment, each of the n portions has a different polynucleotide sequence compared to each of the other n portions, wherein the respective sequences may be genetically unrelated and/or obtained from different sources.

In one embodiment, each of the n portions comprises or consists of a sequence derived from a nucleic acid sample (e.g. an insert).

In one embodiment, each of the n portions is at least 25 base pairs or at least 50 base pairs.

As mentioned above, methods of the present invention may be conducted on a solid support. Accordingly, in some embodiments, the at least one polynucleotide sequence comprising the n portions is/are attached (e.g. via a 5′-end of the polynucleotide sequence comprising the n portions) to a solid support, wherein the solid support may be a flow cell. In one embodiment, the polynucleotide comprising the n portions is attached to the solid support in a single well of the solid support.

In one embodiment, the at least one polynucleotide sequence comprising the n portions forms a cluster on the solid support.

In one embodiment, the cluster may be formed by bridge amplification.

In one embodiment, the at least one polynucleotide sequence comprising the n portions may form a monoclonal cluster.

In one embodiment, the solid support comprises at least one first immobilised primer and at least one second immobilised primer. In on aspect, the first immobilised primer comprises a sequence as defined in SEQ ID NO. 1 or 5, or a variant or fragment thereof; and the second immobilised primer comprises a sequence as defined in SEQ ID NO. 2, or a variant or fragment thereof.

In one embodiment, each polynucleotide sequence comprising the n portions may be attached (via 5′-end of the polynucleotide sequence comprising the n portions) to a first immobilised primer. Each polynucleotide sequence comprising the n portions may comprise a second adaptor sequence, wherein the second adaptor comprises a portion which is substantially complementary to the second immobilised primer (or is substantially complementary to the second immobilised primer). The second adaptor sequence may be at a 3′-end of the polynucleotide sequence comprising the n portions.

It may be advantageous to conduct amplification techniques that increase signal strength for (concatenated) n-mer polynucleotides. This can be done, for example, by increasing the number of (concatenated) n-mer polynucleotides that are present within a given cluster.

As mentioned above, a typical amplification process to form a monoclonal cluster involves amplifying both the template strand and the template complement strand, and then selectively cleaving either the template complement strands, or the template strands. During amplification, the presence of both the template strands and the template complement strands cause saturation of the well (e.g. due to steric hindrance), and thus some first immobilised primers and second immobilised primers on the solid support may not actually be used. When both the template strands and the template complement strands are present, close to 100% strand density (or saturation) is obtained. Nevertheless, after cleavage of either the template complement strands, or the template strands, further space for amplification is possible because the well has only 50% strand density (with only either the remaining of the template strands or template complement strands).

As such, in one embodiment, the method comprises:
providing a solid support comprising a plurality of first immobilised primers and a plurality of second immobilised primers, wherein an initial proportion of the first immobilised primers have each been extended to form the polynucleotide sequence comprising n portions and substantially all of the second immobilised primers have not been extended, wherein each polynucleotide sequence comprising n portions comprises a second adaptor sequence which is substantially complementary to the second immobilised primer,
selectively blocking a proportion of second immobilised primers that have not been extended using a primer blocking agent, wherein the primer blocking agent is configured to limit or prevent synthesis of a strand extending from the second immobilised primer, and
conducting at least two amplification cycles in order provide a new proportion of first immobilised primers that have been extended to form the polynucleotide sequence comprising n portions and a proportion of second immobilised primers that have been extended to form polynucleotide complement sequences comprising n complement portions, wherein the new proportion of first immobilised primers is greater than the initial proportion of first immobilised primers.

Such a method step advantageously allows more polynucleotide sequences comprising n portions to be produced. This allows greater than 50% strand density of solely the polynucleotide sequences comprising n portions to be achieved, thus increasing signal strength for the polynucleotide sequences comprising n portions.

In one aspect, for the step conducting at least two amplification cycles, the number of amplification cycles is chosen such that a saturation point is reached (e.g. between 5 to 20 cycles, between 7 to 15 cycles, or between 8 to 10 cycles). In other words, amplification may be conducted until there is no further change in the number of polynucleotide sequences comprising n portions (or polynucleotide complement sequences comprising n complement portions), for example where close to total 100% strand density is obtained. This advantageously leads to even higher strand densities to be obtained of solely the polynucleotide sequences comprising n portions, which can approach strand densities of around 90% (or higher).

It may be desirable to regenerate the monoclonal cluster for the purposes of conducting sequencing. Accordingly, the method may further comprise a step of cleaving substantially all of the polynucleotide complement sequences comprising n complement portions.

In one embodiment, between 60% to 95% of second immobilised primers that have not been extended (relative to a total number of second immobilised primers that have not been extended) are blocked using the primer blocking agent; between 75% to 90%, between 80% to 90%, or between 85% to 90%.

One way of selectively blocking a proportion of second immobilised primers is to use extended primer sequences, wherein such sequences can bind (e.g. hybridise) free immobilised primers (e.g. P5 or P7), and wherein the extended primer sequences further comprise at least one 5′ additional nucleotide. By using the extended primer sequence as a template, it is possible to add a primer blocking agent, where the primer blocking agent is complementary to 5′ additional nucleotide.

As such, the method may comprise contacting some of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to 5′ additional nucleotide.

In one embodiment, the extended primer sequences are substantially complementary to the first or second immobilised primers (e.g. P5 or P7), or substantially complementary to a portion of the first or second immobilised primer.

The 5′ additional nucleotide may be selected from A, T, C or G, but may be T (or U) or C. In one embodiment, 5′ additional nucleotide is not a complement of 3′ nucleotide of the second immobilised primer (where the extended primer sequence binds the first immobilised primer) or is not a complement of 3′ nucleotide of the first immobilised primer (where the extended primer sequence binds the second immobilised primer). For example, where the first immobilised primer is P5 (for example as defined in SEQ ID NO. 1 or 5) and the second immobilised primer is P7 for example as defined in SEQ ID NO. 2), and where the extended primer sequence binds the first immobilised primer, 5′ additional nucleotide is not A. Similarly, where the extended primer sequence binds the second immobilised primer, the 5′ additional nucleotide is not G.

In one embodiment, the primer-blocking agent is a blocked nucleotide. As such, the blocked nucleotide may comprise a blocking group. Suitable blocking groups include a hairpin loop, a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase. In one embodiment, the blocked nucleotide may be A, C, T or G, but may be selected from A or G. Accordingly, where 5′ additional nucleotide is T or U, the primer-blocking agent is A, and where 5′ additional nucleotide is C, the primer-blocking agent is G.

In one embodiment, the extended primer sequence is selected from SEQ ID NO. 13 to 24 or a variant or fragment thereof.

There are different ways available for achieving the blocking of the proportion of second immobilised primers using a primer blocking agent by use of the extended primer sequence.

In one embodiment, the extended primer sequence may comprise a first extended primer sequence which is substantially complementary to the second immobilised primer and comprises a first 5′ additional nucleotide, and a second extended primer sequence which is substantially complementary to the second immobilised primer and comprises a second 5′ additional nucleotide, wherein the first 5′ additional nucleotide and the second 5′ additional nucleotide are configured to base pair with different nucleotides, and the primer blocking agent is complementary to the first 5′ additional nucleotide. Flowing a primer blocking agent that is complementary to the first 5′ additional nucleotide (and not complementary to the second 5′ additional nucleotide) allows first immobilised primers that are annealed to the first extended primer sequence to be selectively blocked.

In one embodiment, the first extended primer sequence may form between 60% to 95% of the total population of extended primer sequences (wherein the total population may refer to a combined population of first extended primer sequences and second extended primer sequences); between 75% to 90%, between 80% to 90%, or between 85% to 90%. The second extended primer sequence may form between 5% to 40% of the total population of extended primer sequences; between 10% to 25%, between 10% to 20%, or between 10% to 15% (for example, the first extended primer sequence may form between 60% to 95% of the total population of extended primer sequences and the second extended primer sequence may form between 5% to 40% of the total population of extended primer sequences; in one embodiment, the first extended primer sequence may form between 75% to 90% of the total population of extended primer sequences and the second extended primer sequence may form between 10% to 25% of the total population of extended primer sequences; in another embodiment, the first extended primer sequence may form between 80% to 90% of the total population of extended primer sequences and the second extended primer sequence may form between 10% to 20% of the total population of extended primer sequences; in another embodiment, the first extended primer sequence may form between 85% to 90% of the total population of extended primer sequences and the second extended primer sequence may form between 10% to 15% of the total population of extended primer sequences).

Alternatively (or in addition to using the first extended primer sequence and the second extended primer sequence) the primer blocking agent may be provided as a mixture of blocked nucleotides (e.g. as described above) and unblocked nucleotides, wherein the blocked nucleotide and the unblocked nucleotide comprise the same base. In one embodiment, both the blocked nucleotide and unblocked nucleotide are selected from A, C, T or G, but may be selected from A or G. Here, it is not strictly necessary to use the different first extended primer sequences and second extended primer sequences, and instead all of the extended primer sequences may be the same.

In one embodiment, the blocked nucleotide may form between 60% to 95% of the total population of the mixture (wherein the total population may refer to a combined population of blocked nucleotides and unblocked nucleotides); between 75% to 90%, between 80% to 90%, or between 85% to 90%. The unblocked nucleotide may form between 5% to 40% of the total population of the mixture; between 10% to 25%, between 10% to 20%, or between 10% to 15% (for example, the blocked nucleotide may form between 60% to 95% of the total population of the mixture and the unblocked nucleotide may form between 5% to 40% of the total population of the mixture; in one embodiment, the blocked nucleotide may form between 75% to 90% of the total population of the mixture and the unblocked nucleotide may form between 10% to 25% of the total population of the mixture; in another embodiment, the blocked nucleotide may form between 80% to 90% of the total population of the mixture and the unblocked nucleotide may form between 10% to 20% of the total population of the mixture; in another embodiment, the blocked nucleotide may form between 85% to 90% of the total population of the mixture and the unblocked nucleotide may form between 10% to 15% of the total population of the mixture).

In one embodiment, the step of providing the solid support comprising the plurality of first immobilised primers and a plurality of second immobilised primers (where a proportion of first immobilised primers have each been extended to form the polynucleotide sequence comprising n portions, and substantially all of the second immobilised primers have not been extended) involves:
providing a solid support comprising a plurality of first immobilised primers and a plurality of second immobilised primers, wherein substantially all of the first immobilised primers have not been extended and substantially all of the second immobilised primers have not been extended,
annealing a target polynucleotide comprising n complement portions, a first adaptor sequence at one end of the target polynucleotide and a second adaptor complement sequence at another end of the target polynucleotide, wherein the first adaptor sequence is substantially complementary to the first immobilised primer, and wherein the second adaptor complement sequence is substantially identical to the second immobilised primer,
synthesising the polynucleotide sequence comprising n portions and the second adaptor sequence by extending the first immobilised primer,
forming a plurality of first immobilised primers that have each been extended to form a polynucleotide sequence comprising n portions and a plurality of second immobilised primers that have each been extended to form a polynucleotide complement sequence comprising n complement portions, and
selectively cleaving substantially all of the polynucleotide complement sequences comprising n complement portions from the second immobilised primers.

Such a method is also applicable more generally to advantageously increasing signal strength for any monoclonal cluster.

Accordingly, in another aspect of the invention, there is provided a method of synthesising template polynucleotides, comprising:
providing a solid support comprising a plurality of first immobilised primers and a plurality of second immobilised primers, wherein an initial proportion of the first immobilised primers have each been extended to form a template polynucleotide and substantially all of the second immobilised primers have not been extended, wherein each template polynucleotide comprises a second adaptor sequence which is substantially complementary to the second immobilised primer,
selectively blocking a proportion of second immobilised primers that have not been extended using a primer blocking agent, wherein the primer blocking agent is configured to limit or prevent synthesis of a strand extending from the second immobilised primer, and
conducting at least two amplification cycles in order provide a new proportion of first immobilised primers that have been extended to form template polynucleotides and a proportion of second immobilised primers that have been extended to form template complement polynucleotides, wherein the new proportion of first immobilised primers is greater than the initial proportion of first immobilised primers.

The template polynucleotides are typically attached via a 5′-end of the template polynucleotide to the first immobilised primer. The second adaptor sequence is typically attached to a 3′-end of the template polynucleotide.

In one embodiment, for the step conducting at least two amplification cycles, the number of amplification cycles is chosen such that a saturation point is reached (e.g. between 5 to 20 cycles, between 7 to 15 cycles, or between 8 to 10 cycles). In other words, amplification may be conducted until there is no further change in the number of template polynucleotides (or template complement polynucleotides).

In one embodiment, the method may further comprise a step of cleaving substantially all of the template complement polynucleotides.

In one embodiment, between 60% to 95% of second immobilised primers that have not been extended (relative to a total number of second immobilised primers that have not been extended) are blocked using the primer blocking agent; between 75% to 90%, between 80% to 90%, or between 85% to 90%.

In one embodiment, the method may comprise contacting some of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to 5′ additional nucleotide.

In one embodiment, the extended primer sequences, primer blocking agents and the 5′ additional nucleotides are as described herein.

In one embodiment, the step of providing the solid support comprising the plurality of first immobilised primers and a plurality of second immobilised primers (where a proportion of first immobilised primers have each been extended to form the template polynucleotide, and substantially all of the second immobilised primers have not been extended) involves:
providing a solid support comprising a plurality of first immobilised primers and a plurality of second immobilised primers, wherein substantially all of the first immobilised primers have not been extended and substantially all of the second immobilised primers have not been extended,
annealing a target polynucleotide comprising a first adaptor sequence at one end of the target polynucleotide and a second adaptor complement sequence at another end of the target polynucleotide, wherein the first adaptor sequence is substantially complementary to the first immobilised primer, and wherein the second adaptor complement sequence is substantially identical to the second immobilised primer,
synthesising the template polynucleotide comprising the second adaptor sequence by extending the first immobilised primer,
forming a plurality of first immobilised primers that have each been extended to form a template polynucleotide and a plurality of second immobilised primers that have each been extended to form a template complement polynucleotide, and
selectively cleaving substantially all of the template complement polynucleotides from the second immobilised primers.

### Data Analysis Using 9 QaM

For two portions of polynucleotide sequences (e.g. a first portion and a second portion as described herein), there are sixteen possible combinations of nucleobases at any given position (i.e., an A in the first portion and an A in the second portion, an A in the first portion and a T in the second portion, and so on). When the same nucleobase is present at a given position in both portions, the light emissions associated with each target sequence during the relevant base calling cycle will be characteristic of the same nucleobase. In effect, the two portions behave as a single portion, and the identity of the bases at that position are uniquely callable.

However, when a nucleobase of the first portion is different from a nucleobase at a corresponding position of the second portion, the signals associated with each portion in the relevant base calling cycle will be characteristic of different nucleobases. In one embodiment, the first signal coming from the first portion have substantially the same intensity as the second signal coming from the second portion. The two signals may also be co-localised, and may not be spatially and/or optically resolved. Therefore, when different nucleobases are present at corresponding positions of the two portions, the identity of the nucleobases cannot be uniquely called from the combined signal alone. However, useful sequencing information can still be determined from these signals.

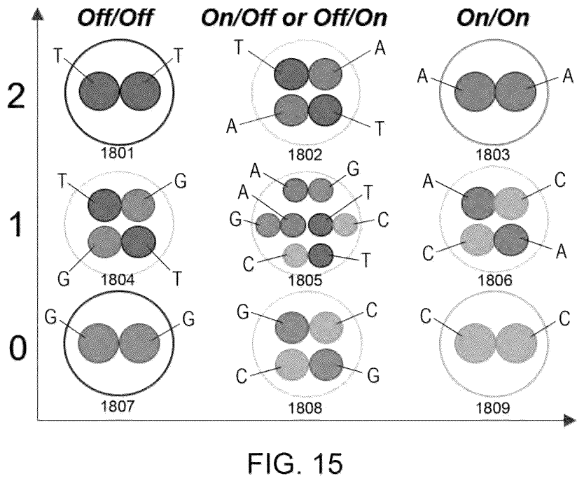

The scatter plot of FIG. 15 shows nine distributions (or bins) of intensity values from the combination of two co-localised signals of substantially equal intensity.

The intensity values shown in FIG. 15 may be up to a scale or normalisation factor; the units of the intensity values may be arbitrary or relative (i.e., representing the ratio of the actual intensity to a reference intensity). The sum of the first signal generated from the first portion and the second signal generated from the second portion results in a combined signal. The combined signal may be captured by a first optical channel and a second optical channel. The computer system can map the combined signal generated into one of the nine bins, and thus determine sequence information relating to the added nucleobase at the first portion and the added nucleobase at the second portion.

Bins are selected based upon the combined intensity of the signals originating from each target sequence during the base calling cycle. For example, bin 1803 may be selected following the detection of a high-intensity (or “on/on”) signal in the first channel and a high-intensity signal in the second channel. Bin 1806 may be selected following the detection of a high-intensity signal in the first channel and an intermediate-intensity (“on/off” or “off/on”) signal in the second channel. Bin 1809 may be selected following the detection of a high-intensity signal in the first channel and a low-intensity or zero-intensity (“off/off”) signal in the second channel. Bin 1802 may be selected following the detection of an intermediate-intensity signal in the first channel and a high-intensity signal in the second channel. Bin 1805 may be selected following the detection of an intermediate-intensity signal in the first channel and an intermediate-intensity signal in the second channel. Bin 1808 may be selected following the detection of an intermediate-intensity signal in the first channel and a low-intensity or zero-intensity signal in the second channel. Bin 1801 may be selected following the detection of a low-intensity signal in the first channel and a high-intensity signal in the second channel. Bin 1804 may be selected following the detection of a low-intensity or zero-intensity signal in the first channel and an intermediate-intensity signal in the second channel. Bin 1807 may be selected following the detection of a low-intensity or zero-intensity signal in the first channel and a low-intensity signal in the second channel.

Four of the nine bins represent matches between respective nucleobases of the two portions sensed during the cycle (bins 1801, 1803, 1807, and 1809). In response to mapping the combined signal to a bin representing a match, the computer processor may detect a match between the first portion and the second portion at the sensed position. In response to mapping the combined signal to a bin representing a match, the computer processor may base call the respective nucleobases. For example, when the combined signal is mapped to bin 1801 for a base calling cycle, the computer processor base calls both the added nucleobase at the first portion and the added nucleobase at the second portion as T. When the combined signal is mapped to bin 1803 for the base calling cycle, the processor base calls both the added nucleobase at the first portion and the added nucleobase at the second portion as A. When the combined signal is mapped to bin 1807 for the base calling cycle, the processor base calls both the added nucleobase at the first portion and the added nucleobase at the second portion as G. When the combined signal is mapped to bin 1809 for the base calling cycle, the processor base calls both the added nucleobase at the first portion and the added nucleobase at the second portion as C.

The remaining five bins are “ambiguous”. That is to say that these bins each represent more than one possible combination of first and second nucleobases. Bins 1802, 1804, 1806, and 1808 each represent two possible combinations of first and second nucleobases. Bin 1805, meanwhile, represents four possible combinations. Nevertheless, mapping the combined signal to an ambiguous bin may still allow for sequencing information to be determined. For example, bins 1802, 1804, 1805, 1806, and 1808 represent mismatches between respective nucleobases of the two portions sensed during the cycle. Therefore, in response to mapping the combined signal to a bin representing a mismatch, the computer processor may detect a mismatch between the first portion and the second portion at the sensed position.

In this particular example, A is configured to emit a signal in both the first channel and the second channel, C is configured to emit a signal in the first channel only, T is configured to emit a signal in the second channel only, and G does not emit a signal in either channel. However, different permutations of nucleobases can be used to achieve the same effect by performing dye swaps. For example, A may be configured to emit a signal in both the first channel and the second channel, T may be configured to emit a signal in the first channel only, C may be configured to emit a signal in the second channel only, and G may be configured to not emit a signal in either channel.

The number of classifications, which may be selected based upon the combined signal intensities may be predetermined, for example based on the number of portions expected to be present in the nucleic acid cluster. Whilst FIG. 15 shows a set of nine possible classifications, the number of classifications may be greater or smaller.

In addition to identifying matches and mismatches, the mapping of the combined signal to each of the different bins (e.g. in combination with additional knowledge, such as the library preparation methods used) can provide additional information about the first portion and the second portion, or about sequences from which the first portion and the second portion were derived. For example, given the nucleic acid material input and the processing methods used to generate the nucleic acid clusters, the first portion and the second portion may be expected to be identical at a given position. In this case, the mapping of the combined signal to a bin representing a mismatch may be indicative of an error introduced during library preparation. In addition, the first portion and the second portion may be expected to be different, for example due to deliberate sequence modifications introduced during library preparation to detect modified cytosines.

Errors arise during NGS library preparation, for example due to PCR artefacts or DNA damage. The error rate is determined by the library preparation method used, for example the number of cycles of PCR amplification carried out, and a typical error rate may be of the order of 0.1%. This limits the sensitivity of diagnostic assays based on the sequencing method, and may obscure true variants. The present methods allow for the identification of library preparation errors from fewer sequencing reads.

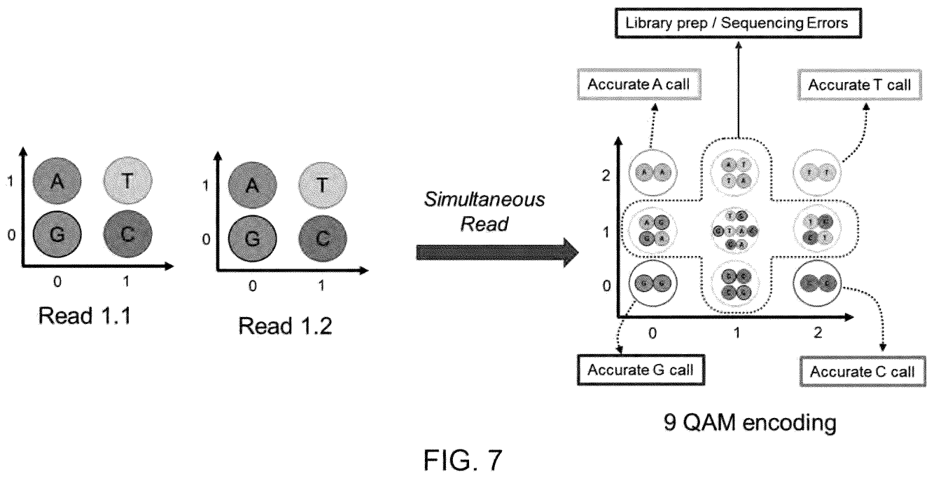

In the absence of any library preparation/sequencing errors, the signals produced by sequencing the two portions (e.g. using sequencing-by-synthesis) will match. The combined signal may therefore be mapped to one of the four “corner” clouds shown in FIGS. 7 and 8, and FIG. 15, and the identity of the nucleobase at the corresponding position of the original library polynucleotide can be determined. Should the identity of the nucleobase at that position suggest a rare, or even unknown, variant, it can be determined with a high level of confidence that the base call represents a true variant, as opposed to a library preparation error. If, on the other hand, the combined signal is mapped to any of the other clouds, this indicates that the sequences of the first portion and the second portion do not match, and that an error has occurred in library preparation. Therefore, in response to mapping the combined signal to a classification representing a mismatch between the two nucleobases, a library preparation error may be identified.

FIG. 21 is a flow diagram showing a method 1900 of determining sequence information according to the present disclosure. The described method allows for the determination of sequence information from two (or more) portions (e.g. the first portion and the second portion) in a single sequencing run from a single combined signal obtained from the first portion and the second portion.

As shown in FIG. 21, the disclosed method 1900 may start from block 1901. The method may then move to block 1910.

At block 1910, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data, for example based upon a chastity score. A chastity score may be calculated as the ratio of the brightest base intensity divided by the sum of the brightest and second brightest base intensities. In one example, high-quality data corresponding to two portions with a substantially equal intensity ratio may have a chastity score of around 0.8 to 0.9, for example 0.89-0.9.

After the intensity data has been obtained, the method may proceed to block 1920. In this step, one of a plurality of classifications is selected based on the intensity data. Each classification represents one or more possible combinations of respective first and second nucleobases, and at least one classification of the plurality of classifications represents more than one possible combination of respective first and second nucleobases. In one example, the plurality of classifications comprises nine classifications as shown in FIG. 15. Selecting the classification based on the first and second intensity data comprises selecting the classification based on the combined intensity of the first and second signal components and the combined intensity of the third and fourth signal components.

The method may then proceed to block 1930, where sequence information of the respective first and second nucleobases is determined based on the classification selected in block 1920. The signals generated during a cycle of a sequencing are indicative of the identity of the nucleobase(s) added during sequencing (e.g. using sequencing-by-synthesis). For example, it may be determined that there is a match or a mismatch between the respective first and second nucleobases. Where it is determined that there is a match between the first and second respective nucleobases, the nucleobases may be base called. Whether there is a match or a mismatch, additional or alternative information may be obtained, as described above. It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1940.

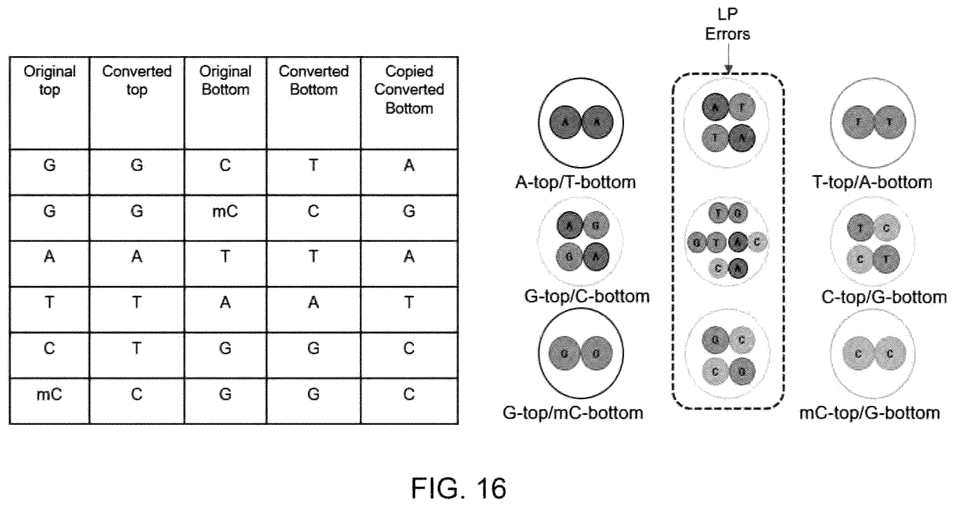

As mentioned herein, the library preparation may involve treatment with a conversion agent. In cases where the conversion reagent is configured to convert an unmodified cytosine to uracil or a nucleobase which is read as thymine/uracil, the correspondence between bases in the original polynucleotide and in the converted strands is shown in FIG. 16, alongside a scatter plot showing potential resulting distributions for the combined signal intensities resulting from the simultaneous sequencing of the target sequences. An A-T or T-A base pair in the original molecule will result in a match (A/A or T/T) at the corresponding position of the forward and reverse complement strands of the library. An mC-G or G-mC base pair in the library will also result in a match (G/G or C/C) at the corresponding position of the forward and reverse complement strands of the library. For a C-G base pair, however, the conversion of unmodified cytosine to uracil (or a nucleobase which is read as thymine/uracil) in the forward strand of the library (“top” strand) will result in a T at the corresponding position of the forward strand of the library. Meanwhile, the corresponding position on the reverse complement strand of the library (“bottom” strand) will be occupied by C. Alternatively, for a G-C base pair, the conversion of unmodified cytosine to uracil (or a nucleobase which is read as thymine/uracil) in the reverse strand of the library (“bottom” strand) will result in an A at the corresponding position of the reverse complement strand of the library. Meanwhile, the corresponding position of the forward strand of the library (“top” strand) will be occupied by G. Therefore, in response to mapping the combined signal to the distribution representing G/G or C/C, the presence of a modified cytosine can be determined at the corresponding position in the original polynucleotide.

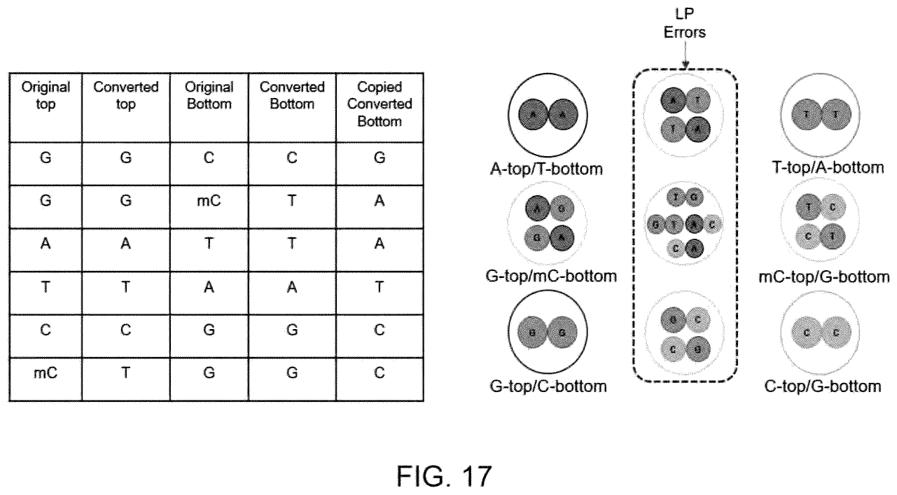

In other cases where the conversion reagent is configured to convert a modified cytosine to thymine or a nucleobase which is read as thymine/uracil, FIG. 17 shows the correspondence between bases in the original polynucleotide and in the converted strands, alongside a scatter plot showing potential resulting distributions for the combined signal intensities resulting from the simultaneous sequencing of the target sequences. An A-T or T-A base pair in the library will result in a match (A/A or T/T) at the corresponding position of the forward and reverse complement strands of the library. A C-G or G-C base pair in the library will also result in a match (G/G or C/C) at the corresponding position of the forward and reverse complement strands of the library. For a mC-G base pair, however, the conversion of 5-methylcytosine to thymine in the forward strand of the library (“top” strand) will result in a T at the corresponding position of the forward strand of the library. Meanwhile, the corresponding position on the reverse complement strand of the library (“bottom” strand) will be occupied by C. Alternatively, the conversion of 5-methylcytosine to thymine in the reverse strand of the library (“bottom” strand) will result in an A at the corresponding position of the reverse complement strand of the library. Meanwhile, the corresponding position of the forward strand of the library (“top” strand) will be occupied by G. Therefore, in response to mapping the combined signal to the distribution representing an A/G, G/A, T/C, or C/T mismatch, the presence of a modified cytosine can be determined at the corresponding position in the original polynucleotide.

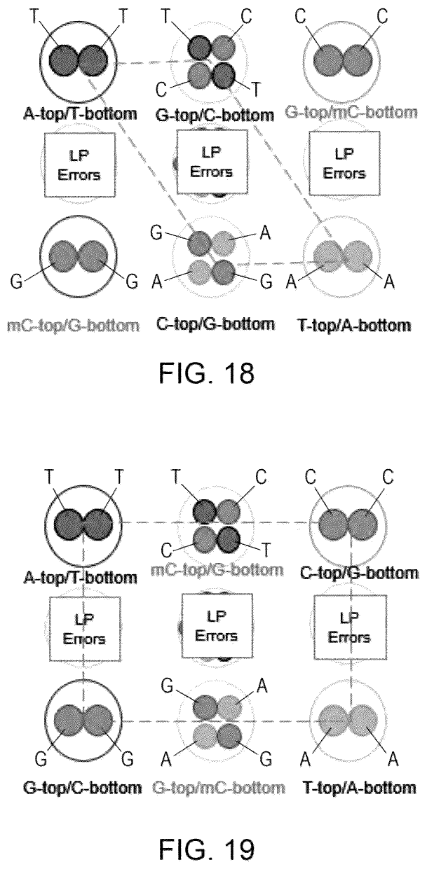

FIG. 18 represents the distributions resulting from the use of an alternative dye-encoding scheme following use of a conversion reagent configured to convert an unmodified cytosine to uracil or a nucleobase which is read as thymine/uracil, and FIG. 19 represents the distributions resulting from the use of an alternative dye-encoding scheme following use of a conversion reagent configured to convert a modified cytosine to thymine or a nucleobase which is read as thymine/uracil.

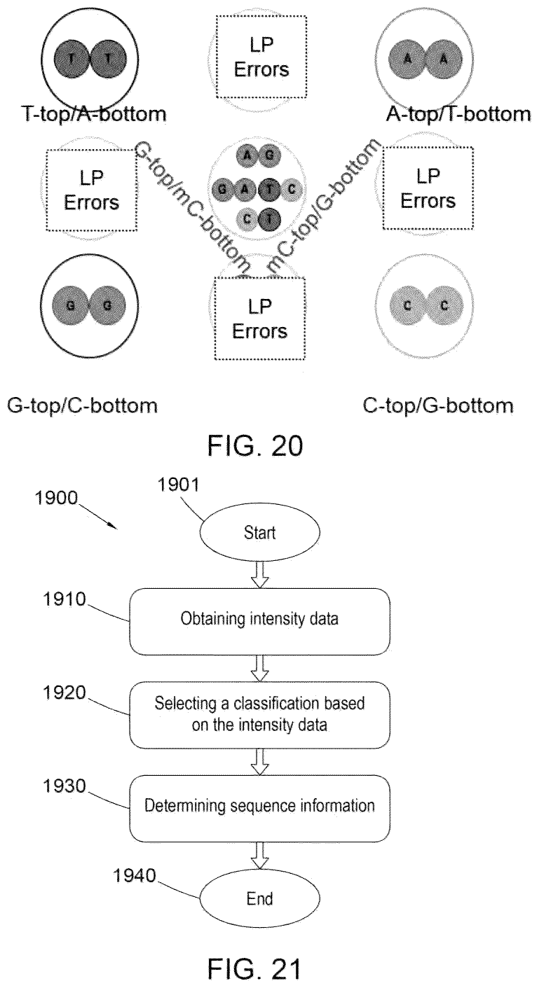

FIG. 20 represents yet another distribution resulting from the use of an alternative dye-encoding scheme following use of a conversion reagent configured to convert a modified cytosine to thymine or a nucleobase, which is read as thymine/uracil. In this case, modified cytosines fall within a central bin.

In the present example, for each base pair in the original double-stranded DNA molecule, it may be assumed that there are six possibilities: A-T, T-A, C-G, G-C, mC-G and G-mC. As shown in FIGS. 16 to 19, each of these possibilities is uniquely represented by one of the plurality of classifications. According to the present methods, it is therefore possible to determine both the sequence and “methylation” status (i.e. presence of modified cytosines) of a double-stranded polynucleotide in a single sequencing run.

In addition to determining “methylation” status, it may also be possible to identify library preparation/sequencing errors. Using the dye-encoding scheme shown in FIGS. 16 and 17, the central column of distributions is indicative of such errors. Using the dye encoding scheme shown in FIGS. 18 and 19, the central row of distributions is indicative of such errors.

The dye-encoding scheme may be optimised to allow for different combinations of first and second nucleobases to be resolved. This may be particularly useful where sequence modifications of a known type have been introduced into the first portions and the second portions. For example, where sequence modifications have been introduced that result in the conversion of unmodified cytosines to uracil or nucleobases which is read as thymine/uracil, or the conversion of modified cytosines to thymine or nucleobases which are read as thymine/uracil, the dye-encoding scheme may be selected such that the resulting combination of first and second nucleobases do not fall within the central bin (which represents four different nucleobase combinations).

In the case of conversion of modified cytosines to thymine (or nucleobases which are read as thymine/uracil), a T/C or G/A mismatch between the forward and reverse complement strands is indicative of the presence of a mC-G or G-mC base pair at the corresponding position of the library. The dye-encoding scheme may therefore be designed such that these mismatches may be resolved from other possible combinations of nucleobases. This may be achieved by detecting light emissions from A and T bases in a first illumination cycle, and from C and T bases in a second illumination cycle. In another example, light emissions may be detected from C and G bases in a first illumination cycle, and from C and T bases in a second illumination cycle. In another example, light emissions may be detected from C and A bases in a first illumination cycle, and from C and G bases in a second illumination cycle.

In the case of unmodified cytosines to uracil (or nucleobases which is read as thymine/uracil), a C/C or G/G match between the forward and reverse complement strands is indicative of the presence of a mC-G or G-mC base pair at the corresponding position of the library. In this case, a mC-G or G-mC base pair will always be resolvable. However, the dye-encoding scheme can still be designed to optimise the resolution between unmodified bases.

FIG. 21 is a flow diagram showing a method 1900 of determining sequence information according to the present disclosure. The described method allows for the determination of sequence information from two (or more) portions (e.g. the first portion and the second portion) in a single sequencing run from a single combined signal obtained from the first portion and the second portion.

In one embodiment, the first portion comprises or consists of a sequence derived from a nucleic acid sample (e.g. an insert) and the second portion comprises or consists of a sequence derived from a nucleic acid sample (e.g. an insert).

In one embodiment, the first portion is at least 25 or at least 50 base pairs and the second portion is at least 25 base pairs or at least 50 base pairs.

As shown in FIG. 21, the disclosed method 1900 may start from block 1901. The method may then move to block 1910.

At block 1910, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data, for example based upon a chastity score. A chastity score may be calculated as the ratio of the brightest base intensity divided by the sum of the brightest and second brightest base intensities. In one example, high-quality data corresponding to two portions with a substantially equal intensity ratio may have a chastity score of around 0.8 to 0.9, for example 0.89-0.9.

After the intensity data has been obtained, the method may proceed to block 1920. In this step, one of a plurality of classifications is selected based on the intensity data. Each classification represents one or more possible combinations of respective first and second nucleobases, and at least one classification of the plurality of classifications represents more than one possible combination of respective first and second nucleobases. In one example, the plurality of classifications comprises nine classifications as shown in FIG. 15. Selecting the classification based on the first and second intensity data comprises selecting the classification based on the combined intensity of the first and second signal components and the combined intensity of the third and fourth signal components.

The method may then proceed to block 1930, where sequence information of the respective first and second nucleobases is determined based on the classification selected in block 1920. The signals generated during a cycle of a sequencing are indicative of the identity of the nucleobase(s) added during sequencing (e.g. using sequencing-by-synthesis). For example, it may be determined that there is a match or a mismatch between the respective first and second nucleobases. Where it is determined that there is a match between the first and second respective nucleobases, the nucleobases may be base called. Whether there is a match or a mismatch, additional or alternative information may be obtained, as described above. It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1940.

### Methods of Preparing and Sequencing a Tandem Library

In one aspect of the invention, there is provided a method of preparing at least one polynucleotide library strand, wherein the method comprises:
attaching a first adaptor to a first end of a double-stranded polynucleotide sequence, wherein the first end comprises 3′ end of the forward strand and 5′ end of the reverse strand of the double-stranded polynucleotide sequence; and
attaching a second adaptor to a second end of a double-stranded polynucleotide sequence, wherein the second end comprises 5′ end of the forward strand and 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease.

In another aspect of the invention, there is provided a method of preparing at least one polynucleotide library strand, wherein the method comprises:
attaching a first adaptor to a first end of a double-stranded polynucleotide sequence, wherein the first end comprises 3′ end of the forward strand and 5′ end of the reverse strand of the double-stranded polynucleotide sequence; and
attaching a second adaptor to a second end of a double-stranded polynucleotide sequence, wherein the second end comprises 5′ end of the forward strand and 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the second adaptor comprises a cleavable site and/or a complement of a cleavable site.

In another aspect of the invention, there is provided a method of preparing at least one polynucleotide library strand, wherein the method comprises:
attaching a first adaptor to a first end of a double-stranded polynucleotide sequence, wherein the first end comprises 3′ end of the forward strand and 5′ end of the reverse strand of the double-stranded polynucleotide sequence; and
attaching a second adaptor to a second end of a double-stranded polynucleotide sequence, wherein the second end comprises 5′ end of the forward strand and 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease and wherein the second adaptor comprises a cleavable site and/or a complement of a cleavable site.

In another aspect of the invention, there is provided a polynucleotide library strand for sequencing comprising a first adaptor, a double-stranded polynucleotide sequence to be identified and a second adaptor, wherein the first adaptor is attached to a first end of the double-stranded polynucleotide sequence, wherein the first end comprises 3′ end of the forward strand and 5′ end of the reverse strand of the double-stranded polynucleotide sequence; and the second adaptor is attached to a second end of the double-stranded polynucleotide sequence, wherein the second end comprises 5′ end of the forward strand and 3′ end of the reverse strand of the double-stranded polynucleotide sequence; wherein the first adaptor comprises a loop that connects 3′ end of the forward strand and 5′ end of the reverse strand, and wherein the second adaptor comprises a base-paired stem, a primer-binding complement sequence and a primer-binding sequence, and wherein the first adaptor comprises at least one restriction site for an endonuclease.

The first and second adaptors may be attached to the polynucleotide using processes as described in more detail in e.g. WO 07/052006, or “tagmentation” methods as described above.

In a further embodiment, the second adaptor may also comprise at least one cleavable site. In other words, the first adaptor comprises at least one restriction site and the second adaptor comprises at least one cleavable site. The cleavable site may also be a restriction site.

By “restriction site” is meant a sequence of nucleotides recognised by an endonuclease, such as a single-stranded endonuclease. A restriction site may also be referred to as a “recognition site” or “recognition sequence”, and such terms may be used interchangeably.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands.

Examples of suitable nicking enzymes that may be used include, but are not limited to, Nb.BbvCI, Nb.Bsml, Nb.BsrDI, Nb.Btsl, Nt.Alwl, Nt.BsmAl, Nt.BspQI, Nt.BstNBI, BssSI, Nb.Bpu101 and Nt.CviPll. These nickases can be used either alone or in various combinations. Other suitable nicking endonucleases are available from commercial sources, including New England Biolabs and Fisher Scientific.

The restriction sites vary depending on the nickase used, and are well known in the art. In one example, the restriction site is selected from the following:

In one embodiment, the nickase is Nb.BssSI, and the restriction site is CACGAG, wherein Nb.BssSI catalyzes a single strand break within the recognition sequence.

In one embodiment, the nickase is Nt.BspQI, and the restriction site is GCTCTTC (1/−7), wherein Nt.BspQI catalyzes a single strand break one base beyond 3′ side of the restriction site.

In one embodiment, the nickase is Nt.CviPll and the restriction site is (0/−1) CCD, wherein Nt. CviPII catalyzes a single strand break at 5′ side of the restriction site.

In one embodiment, the nickase is Nt.BstNBI and the restriction site is GAGTC (4/−5), wherein Nt.BstNBI catalyzes a single strand break four bases beyond 3′ side of the restriction site.

In one embodiment, the nickase is Nb.BsrDI and the restriction site is GCAATG, wherein Nb.BsrDI catalyzes a single strand break within the restriction site.

In one embodiment, the nickase is Nb.Btsl and the restriction site is GCAGTG, wherein Nb.Btsl catalyzes a single strand break within the restriction site.

In one embodiment, the nickase is Nt.Alwl and the restriction site is GGATC (4/−5), wherein Nt.Alwl catalyzes a single strand break four bases beyond 3′ side of the restriction site.

In one embodiment, the nickase is Nb.BbvCI and the restriction site is CCTCAGC, wherein Nb.BbvCI catalyzes a single strand break within the restriction site.

In one embodiment, the nickase is Nb.Bsml and the restriction site is GAATGC, wherein Nb.Bsml catalyzes a single strand break within the restriction site.

In one embodiment, the nickase is Nt.BsmAl and the restriction site is GTCTC (1/−5), wherein Nt.BsmAl catalyzes a single strand break one base beyond 3′ side of the restriction site.

In one embodiment, the nickase is Nb.Bpu101 and the restriction site is CCTNAGC, wherein Nb.Bpu101 catalyzes a single strand break within the restriction site.

Where the restriction site is described in the following format (x/−y), x is the number of nucleotides beyond (i.e. 3′ of) 3′ end of the restriction site where cleavage occurs; and y is the number of nucleotides in the restriction site

In an alternative embodiment, the endonuclease is a Cas9 nickase.

Examples of a Cas9 nickase include Cas9 D10A and Cas9 H840A. For example, in one embodiment, the Cas9 protein may comprise the D10A or H840A amino acid substitutions. These nickases cleave only the DNA strand that is complementary to and recognized by a gRNA.

In one embodiment, the restriction site may be or may comprise a PAM (protospacer adjacent motif) sequence. Examples of suitable PAM sequences include NGG, NGAG, NGCG, NGN, NG, GAA, GAT, NNG, NGN, NRN, YG, NNGRRT, NNNRRT, NNAGAA, NNNNGATT and NNNNCRAA and complements thereof.

In a further embodiment, the Cas9 protein may alternatively or additionally comprise the N863A or N854A amino acid substitutions.

In a further embodiment, the Cas9 protein has been modified to improve activity. For example, in one embodiment, the Cas9 protein may additionally comprise a D1135E substitution. Alternatively, the Cas9 protein may also be the VQR variant.

In one embodiment, where the first and second adaptors both comprise a restriction site, the restriction sites are different sequences. Accordingly, in one embodiment, the first adaptor comprises a first restriction site and the second adaptor comprises a second restriction site.

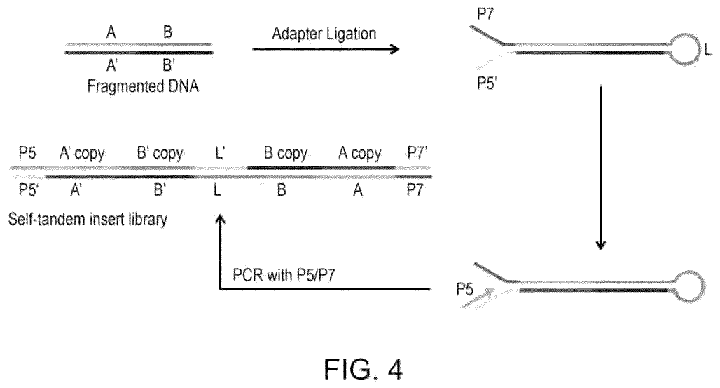

In one embodiment, the target polynucleotide to be sequenced is a double stranded polynucleotide molecule (also referred to herein as a duplex), for example, as shown in FIG. 4. Accordingly, the target polynucleotide may be considered to have a first portion to be identified and a second portion to be identified, wherein the first portion is the forward strand and wherein the second portion is the reverse strand. As shown in FIG. 4, A represents the 5′ “half” of the forward strand and B represents 3′ “half” of the forward strand. Similarly, A′ represents the complement of 5′ “half” of the forward strand (i.e. it is 3′ “half” of the reverse strand) and B′ represents the complement of 3′ “half” of the forward strand (i.e. it is 5′ “half” of the reverse strand.

The first adaptor may be attached to the 5′ end of the first portion and 3′ end of the second portion. Similarly, the second adaptor may be attached to the 3′ end of the first portion and 5′ end of the second portion.

In one embodiment, the first adaptor is added to the 3′ end of the polynucleotide duplex (that is, 3′ end of the forward strand and 5′ end of the reverse strand). The first adaptor may be an oligonucleotide of any structure or any sequence that allows the forward and reverse strands to be connected. For example, the adaptor may be capable of forming a loop. In one example, as shown in FIG. 4, the first adaptor comprises a base-paired stem and a hairpin loop (e.g. a loop structure with unpaired or non-Watson-Crick paired nucleotides) and connects 3′ end of the forward strand with 5′ end of the reverse strand.

In one embodiment, the (first) restriction site is in the base-paired stem, at either the 5′ or 3′ end of the base-paired stem. In one aspect, the restriction site is at the 5′ end. Where the first adaptor comprises a first restriction site, the location of the restriction sequence will depend on whether the cleavage site for the target endonuclease is immediately 3′ of the restriction site or whether, as described above, the endonuclease cleaves (nicks) a number of nucleotides 3′ of the restriction site. It is of course desirable that the endonuclease does not cleave in the target polynucleotide to be sequenced or in its complement on the template (i.e. in the first or second portions, which are the portions that allow the target polynucleotide to be sequenced).

In one embodiment, the second adaptor comprises at least one primer-binding sequence. In another embodiment, the second adaptor comprises at least one primer-binding complement sequence. In an alternative embodiment, the second adaptor comprises both a primer-binding sequence and a primer-binding complement sequence. The primer-binding sequence may be capable of binding to a lawn or immobilised primer that is immobilised on the surface of a solid support. For example, the primer-binding sequence may be either P5′ (for example, SEQ ID NO: 3 or a variant or fragment thereof) or P7′ (for example, SEQ ID NO: 4 or a variant or fragment thereof). Similarly, the primer-binding complement sequence may be either P5 (for example, SEQ ID NO: 1 or 5 or a variant or fragment thereof) or P7 (for example, SEQ ID NO: 2 or a variant or fragment thereof). If the primer-binding sequence is P5′, the primer-binding complement sequence is P7. If the primer-binding sequence is P7′, the primer-binding complement sequence is P5.

As shown in FIG. 4, the second adaptor comprises a base-paired stem, a primer-binding sequence and a primer-binding complement sequence. Specifically, the second adaptor may comprise a first and second strand, wherein the first and second strands are base-paired for a portion of their sequence (forming the base-paired stem) and are non-complementary for the remainder of their sequence, for example, P5′ and P7 or P7′ and P5, which subsequently forms a fork structure, wherein a first arm of the fork structure comprises a primer-binding sequence and the second arm of the fork structure comprises a primer-binding complement sequence.

In one embodiment the second adaptor comprises a (first) cleavable site. In one embodiment, the cleavable site is in the base-paired stem. As described above, the base-paired stem comprises two strands. In one example, the first strand comprises a cleavable site and the second strand comprises a complement of the cleavable site. In one embodiment, it is the strand that is attached to the primer-binding complement sequence that comprises the cleavable site, and the strand that is attached to the primer-binding sequence that comprises a complement of the cleavable site. The cleavable site and the complement of the cleavable site may be cleavable by the same cleaving agent (i.e. they are complementary sequences), although it is possible for the sequences to be cleavable by different agents (i.e. they are not complementary sequences of each other).

Alternatively, the second adaptor does not comprise a cleavable site in the base-paired stem.

In another embodiment, the second adaptor comprises a base-paired stem and a first arm of a fork and a second arm of a fork, where the first arm comprises a primer-binding sequence and a complement of a cleavable site, and the second arm comprises a primer-binding complement sequence and a cleavable site. Again, the cleavable site and complement thereof may be cleavable by the same cleaving agent or different cleaving agents, as described above.

Alternatively, the second adaptor may comprise a base-paired stem and a hairpin loop, where the loop comprises a primer-binding sequence, a second cleavable site and primer-binding complement sequence, where the cleavable site is in-between the primer-binding sequence and the primer-binding complement sequence. In one embodiment, the first adaptor comprises a first cleavable site in the base-paired stem as described above, and a second cleavable site in the loop and in-between the primer-binding sequence and the primer-binding complement sequence. Alternatively, the second adaptor does not comprise the first cleavable site.

As used herein, by “cleavable site” is meant any moiety, such as a modified nucleotide, that allows selective cleavage of the adaptor sequence. By way of non-limiting example, the cleavable site may comprise uracil bases, phosphorothioate groups, ribonucleotides, diol linkages, disulphide linkages, peptides etc.

In one example, the cleavable site is a uracil. Uracil can be cleaved using a uracil glycosylase or USER enzyme mix (which is a cocktail of uracil glycosylase and endonuclease VIII).

In another example, the cleavable site is 8-oxoguanine. 8-oxoguanine can be cleaved using a FPG glycosylase.

Alternatively, the cleavable site is a restriction site. In one embodiment, the first cleavable site is a restriction site. As referred to herein the first cleavable site may therefore be referred to as the second restriction site, and the second cleavable site may be referred to herein as the third restriction site. In some embodiments, the first, second and third restriction sites are all different (i.e. different restriction site sequences).

In one embodiment, the method may comprise cleaving the loop of the second adaptor at the cleavable site to open the loop. This will generate a fork structure, as described above. Specifically, following cleavage the second adaptor will form a base-paired stem and then a fork.

Although not shown in FIG. 4, the first and second adaptors also comprise one or more sequencing primer-binding sites and/or sequencing primer-binding sites. Both are referred to generally as primer-binding sites.

In the first adaptor the sequencing primer-binding sites may be in the loop sequence or in the base-paired stem. In one embodiment, the base-paired stem comprises at least one sequencing primer-binding site. In one embodiment, the sequencing primer-binding site is in the base-paired stem, and in the part of the stem that connects to the reverse strand of the double-stranded polynucleotide. In another embodiment, the loop may comprise two sequencing primer sites. In one example, the loop comprises two sequencing primer sites and a restriction site, wherein the sequencing primer sites are either side of the restriction site.

In the second adaptor the sequencing primer-binding site(s) may also be in the base-paired stem. Alternatively, each fork of the second adaptor may additionally comprise a sequencing primer-binding site.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

The sequence of the sequencing primers and the sequence primer binding sites are not material to the methods of the invention, as long as the sequencing primers are able to bind to the sequence primer binding site to enable amplification and sequencing of the regions to be identified.

In a further embodiment, as also not shown in FIG. 4, the first and/or second adaptors may further comprise one or more index sequences (or one or more index sequence complements).

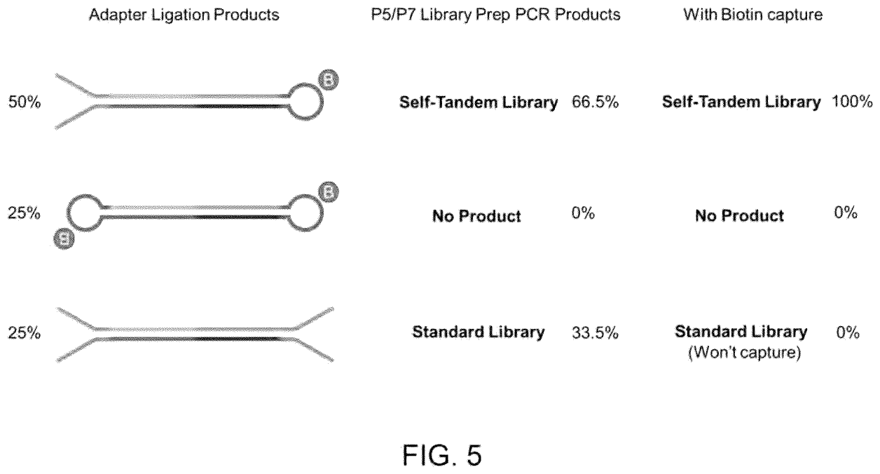

As shown in FIG. 5, after ligation of the adapters three configurations will result, one of which represents the desired loop/fork configuration. The loop/loop configuration does not contain any primer binding sites and will therefore be automatically eliminated during PCR and/or clustering steps. The fork/fork configuration, however, poses an inefficiency risk to the process.

Accordingly, in one embodiment, the first adaptor comprises at least one affinity tag. As such, where required, unwanted fork/fork molecules could easily be eliminated from the workflow via a single affinity-based purification system. As such, the affinity tag may be any tag that can be used in this system. Examples include, but are not limited to, biotin, avidins (e.g. streptavidin), antibodies, haptens, cucubiturils, adamantanes (e.g. 1-adamantylamine), ammonium ions (e.g. amino acids), ferrocenes, cyclodextrins, calixarenes, crown ethers (e.g. 18-crown-6, 15-crown-5, 12-crown-4), cryptands (e.g. [2.2.2]cryptand), His tags (e.g. His₆ tag), or the like.

In one embodiment, the affinity tag is biotin. This would enable the elimination of fork/fork molecules using streptavidin beads (e.g. magnetic streptavidin beads) before/after PCR (FIG. 5). Accordingly, in a further embodiment of the method, the method comprises eliminating polynucleotide library strands with a second adaptor attached to a first end and a second adaptor attached to a second end.

In one embodiment, the method may comprise preparing a polynucleotide library strand as described above, and applying an epigenetic conversion strategy. Such conversion strategies involve treating the polynucleotide library strand with a conversion reagent, wherein the conversion reagent is configured to convert a modified cytosine to thymine or a nucleobase which is read as thymine/uracil, and/or wherein the conversion reagent is configured to convert an unmodified cytosine to uracil or a nucleobase which is read as thymine/uracil. Suitable strategies are well appreciated by the skilled person. Non-limiting examples of such conversion strategies include bisulfite sequencing (BS-seq), oxidative bisulfite sequencing (oxBS-seq), reduced bisulfite sequencing (redBS-seq), TET-assisted bisulfite sequencing (TAB-seq), APOBEC-coupled epigenetic sequencing (ACE-seq), Enzymatic Methyl sequencing (EM-seq), TET-assisted pyridine borane sequencing (TAPS), TET-assisted pyridine borand sequencing with with β-glucosyltransferase blocking (TAPSβ), chemical-assisted pyridine borane sequencing (CAPS), pyridine borane sequencing (PS), and pyridine borane sequencing for 5-caC (PS-c). Non-limiting examples of conversion reagents include sulfites (e.g. bisulfite), cytidine deaminases (e.g. wild-type or mutant enzymes of the APOBEC family), and boron-based reducing agents (e.g. amine-borane compounds or azine-borane compounds, such as t-butylamine borane, ammonia borane, ethylenediamine borane, dimethylamine borane, pyridine borane and 2-picoline borane),

As used herein, the term “modified cytosine” may refer to any one or more of 5-methylcytosine (5-mC), 5-hydroxymethylcytosine (5-hmC), 5-formylcytosine (5-fC) and 5-carboxylcytosine (5-caC):

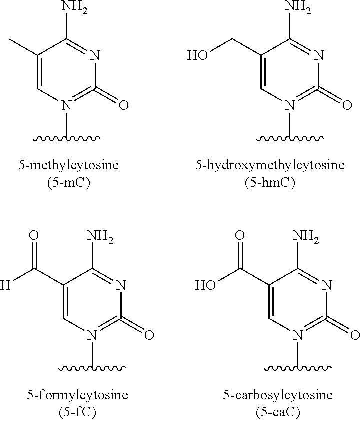

wherein the wavy line indicates an attachment point of the modified cytosine to the polynucleotide.

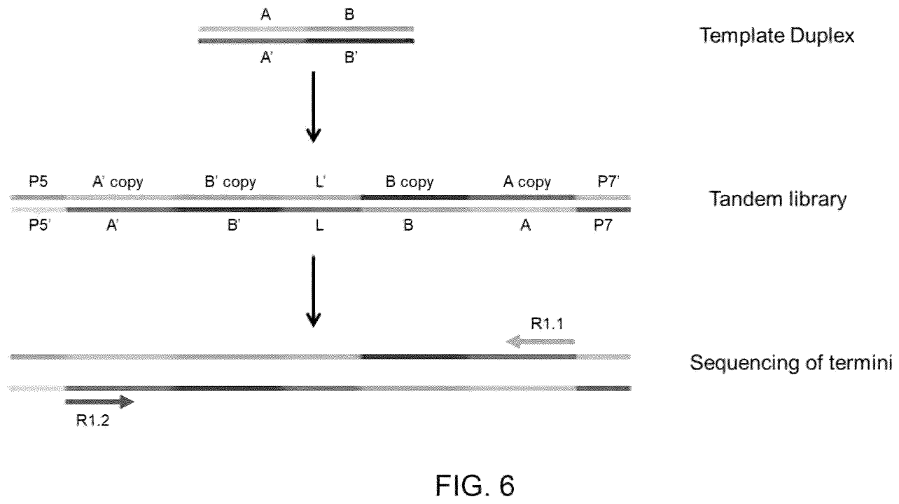

The resulting libraries may either be further amplified via PCR or be directly used for clustering in PCR-free workflows. If amplified, the resulting amplified (double-stranded) library strand is shown in FIG. 6.

As shown in FIG. 6, following binding of a primer (e.g. an immobilised lawn primer, for example P7 (but this could be P5 depending on the arrangement of the forked adaptors)) to a primer-binding sequence (for example, P7′ (but this could be P5′ depending on the arrangement of the forked adaptors)) the library strand can be amplified. Following the first round of amplification, the resulting double-stranded polynucleotide library strands generated from the original library fragment will comprise a forward strand, which corresponds to a complement of the original library fragment (including a complement of the restriction sites) and a reverse strand, which corresponds to the original library fragment.

Accordingly, the forward strand of the resulting amplified library strand will comprise (in 5′ to 3′ direction):
a complement of a first strand of the first adaptor (comprising a primer-binding complement sequence (e.g. P5, for example, SEQ ID NO: 1 or 5 or a variant or fragment thereof) and a complement of the first strand of the base-paired stem);
a copy of 3′ end of the reverse strand (of the original library fragment) (A′copy);
a copy of 5′ end of the reverse strand (of the original library fragment) (B′copy);
a complement of the first adaptor (comprising a complement of the original loop sequence (L′) flanked by complements of the base-paired stem of the first adaptor); a copy of 3′ end of the forward strand (of the original library fragment) (B copy);
a copy of 5′ end of the forward strand (of the original library fragment) (A copy); and
a complement of a second strand of the first adaptor (comprising a complement of the second strand of the base-paired stem of the first adaptor and a complement of the primer-binding complement sequence (e.g. a first primer-binding sequence —e.g. P7′ for example, SEQ ID NO: 4 or a variant or fragment thereof)).

The reverse strand of the resulting amplified library strand will comprise (in 3′ to 5′ direction);
a first strand of the second adaptor (comprising a second primer-binding sequence (e.g. P5′, for example, SEQ ID NO: 3 or 6 or a variant or fragment thereof) and a first strand of the base-paired stem);
the complement of 5′ “half” of the original forward strand (i.e. 3′ “half” of the reverse strand) (A′);
the complement of 3′ “half” of the forward strand (i.e. 5′ “half” of the reverse strand (B′);
the first adaptor, comprising a loop sequence (L) flanked by the base-paired stem of the first adaptor;
the 3′ “half” of the forward strand (B);
the 5′ “half” of the forward strand (A); and
a second strand of the first adaptor (comprising the second strand of the base-paired stem of the first adaptor and second primer-binding complement sequence (e.g. P7, for example, SEQ ID NO: 2 or a variant or fragment thereof)).

As shown in FIG. 4, although the amplified library strands are described to comprise a loop sequence (or loop complement sequence), this refers to the structure of the sequence when present in the first adaptor. The loop sequence in the amplified library strand may be a linear sequence. As such, this sequence may also be referred to as a linear first adaptor sequence (or just first adaptor sequence) or a loop sequence, and such terms may be used interchangeably herein, although when a “loop sequence” is used, for ease of reference, in the context of the amplified library strand it is not intended to limit its structure to a loop (i.e. a linear sequence is encompassed).

As also shown in FIG. 4, the orientation of the polynucleotide sequence (i.e. the insert) to be identified is reversed either side of the loop—i.e. the sequence is A-B-loop-B′-A′ (rather than A-B-loop-A′-B′, for example). This results in an inverted repeat tandem insert polynucleotide library strand. Such a polynucleotide may be referred to herein as an inverted-repeat tandem-insert polynucleotide library strand. As explained above, the expectation is that the complementary sequence of a double-stranded DNA molecule should contain the same (i.e. exactly complementary) information. This may not be the reality in practice for a number of reasons (for example DNA damage, e.g. oxidative damage to one or more bases of one strand). Sequencing an inverted-repeat tandem-insert polynucleotide library strand can be used to determine mismatches (e.g. asymmetry) between complementary strands.

Accordingly, in a further aspect of the invention, there is provided, as described further above, an inverted-repeat tandem-insert polynucleotide library strand, wherein the library strand comprises a primer-binding complement sequence, a first portion to be identified, a loop sequence, a second portion to be identified and a primer-binding sequence, wherein the first and second portions are complementary sequences and wherein the sequence of the second portion is inverted with respect to the first portion, and wherein the loop sequence comprises at least one restriction site for a nicking endonuclease. In a further embodiment, the primer-binding sequence and primer-binding complement sequence comprise at least one cleavable site and/or complement of a cleavable site. In one embodiment, the cleavable site is a restriction site. The inverted-repeat tandem-insert polynucleotide library strand may be single or double-stranded.

In one embodiment, the first portion comprises or consists of a sequence derived from a nucleic acid sample (e.g. an insert) and the second portion comprises or consists of a sequence derived from a nucleic acid sample (e.g. an insert).

In one embodiment, the first portion is at least 25 or at least 50 base pairs and the second portion is at least 25 base pairs or at least 50 base pairs.

Sequencing of the termini of such inverted-repeat tandem-insert library strands results in equivalent sequences in the same direction (e.g. A-B-loop-B′-A′), whereby each end represents the sequence of a different strand of the original duplex (FIG. 4).

Where the library strand has not undergone modification, for example, an epigenetic conversion strategy has not been applied as described above the inverted-repeat tandem-insert library strand is susceptible to re-hybridisation during SBS. A solution to this problem is described below.

In one aspect of the invention, there is provided a method of identifying at least a first region of a polynucleotide sequence, wherein the method comprises
a. preparing at least one polynucleotide library strand as described above;
b. amplifying the polynucleotide library strand to generate a first and second library strand, wherein each library strand comprises a first and second region;
c. hybridising the first or second library strands to first and second immobilised primers respectively on a solid support and carrying out a first extension reaction to generate a first or second immobilised template strand;
d. hybridising the first or second immobilised template strands to a second or first immobilised primer respectively and carrying out a second extension reaction to generate a second and first immobilised template strand;
e. hybridising the first and second immobilised template strands;
f. applying a first endonuclease; and
g. sequencing the first and second immobilised template strands, wherein sequencing the first and second immobilised template strands identifies the first region.

In a further embodiment, the method comprises displacing or de-hybridising the (non-immobilised) library strands from the first or second immobilised strands and hybridising the first immobilised template strand to 5′ end of the second immobilised strand (which comprises a 5′ primer sequence) or hybridising the second immobilised template strand to the 5′ end of the first immobilised strand (which also comprises a 5′ primer sequence). This allows extension of the second or first immobilised strands using the bridged first extension strand as a template. This step is referred to as clustering. In one embodiment, the cluster is generated by bridge amplification.

By “identification” or “identifying” is meant here obtaining genetic information from the polynucleotide strand or polynucleotide strands. This may include identification of the genetic sequence of the polynucleotide strand or polynucleotide strands (i.e. sequencing). Furthermore, this may instead, or additionally, include identification of mismatched base pairs. In addition, this may instead, or additionally, include identification of any epigenetic modifications, for example methylation. Accordingly, “identification” may mean identification of the genetic sequence of the polynucleotide strand or polynucleotide strands, mismatched base pairs, and/or identification of any epigenetic modifications.

In one embodiment, amplifying the polynucleotide library strand generates a first region to be identified and a second region (that may be also identified), such as on a single polynucleotide strand. As described above, the first and second regions may be complementary sequences, and are orientated as inverted-repeat tandem inserts—that is, both regions are on the same polynucleotide strand, and are inverted in sequence with respect to each other (as shown in FIG. 4). Accordingly, in one embodiment, the method comprises generating a plurality of inverted-repeat tandem-insert library strands, wherein each library strand comprises a first and second region. In one embodiment, the method further comprises de-hybridising the library strand to produce single-stranded inverted-repeat tandem-insert library strands.

In one embodiment, each first and second library strands comprises a primer-binding complement sequence, a first portion to be identified, a loop sequence, a second portion to be identified and a primer-binding sequence, wherein the first and second portions are complementary sequences and wherein the sequence of the second portion is inverted with respect to the first portion, and wherein the loop sequence comprises at least one restriction site (a first restriction site) for an endonuclease. In a further embodiment, the primer-binding sequence and primer-binding complement sequence comprise at least one cleavable site and/or at least one complement of a cleavable site. In one embodiment, the cleavable site/complement of cleavable site is a restriction site/complement of a restriction site.

The inverted-repeat tandem-insert polynucleotide library strand may be single or double-stranded.

In a further embodiment, the method comprises converting any epigenetic modifications (e.g. modified cytosines) using a conversion reagent, as described above.

In a further embodiment, the method comprises applying the plurality of inverted-repeat tandem-insert library strands in solution to a solid support (such as a flow cell), wherein, as described above, each inverted-repeat tandem-insert library strand comprises a first or second 3′ primer-binding sequence (e.g. P5′ or P7′), and wherein the solid support has immobilised thereon a plurality of lawn primer sequences complementary to the first and second 3′ primer-binding sequences.

In a further embodiment, the method comprises hybridising 3′ primer binding sequence of the first library strand (a single stranded inverted-repeat tandem-insert library strand) to a first lawn primer or hybridising 3′ primer binding sequence of the second library strand (a single stranded inverted-repeat tandem-insert library strand) to a second lawn primer; and carrying out an extension reaction to extend the lawn primers to generate a first or second immobilised (also referred to herein as extended) template strand complementary to the library strands, wherein the immobilised strands comprise a 3′ (second or first respectively) primer binding sequence. Accordingly, in one embodiment, the first and second library strands comprise a first and second 3′ primer-binding sequence, the solid support comprises a first and second immobilised primer, and the first and second library strands hybridise by their 3′ primer-binding sequences to the first and second immobilised primers.

In a further embodiment, the method comprises displacing or de-hybridising the (non-immobilised) library strands from the first or second immobilised strands and hybridising the first immobilised template strand to 5′ end of the second immobilised strand (which comprises a 5′ primer sequence) or hybridising the second immobilised template strand to the 5′ end of the first immobilised strand (which also comprises a 5′ primer sequence). This allows extension of the second or first immobilised strands using the bridged first extension strand as a template. This step is referred to as clustering. In one embodiment, the cluster is generated by bridge amplification.

In a further embodiment, the method comprises hybridising the first immobilised template strand to 5′ end of the second immobilised strand (which comprises a 5′ primer sequence) and hybridising the second immobilised template strand to 5′ end of the first immobilised strand (which also comprises a 5′ primer sequence). This structure may be referred to herein as a sequence bridge. The sequence bridge is hybridised at a least three places: (1) 5′ primer of the first extended strand is hybridised to 3′ primer-binding region of the second extended strand (e.g. P5′); (2) the loop sequences of both the first and second extended strands and (3) 5′ primer of the second extended strand (e.g. P7) is hybridised to 3′ primer-binding region of the first extended strand (e.g. P7′). Accordingly, this structure may be referred to herein as a loop-hybridised sequence bridge.

In a further embodiment, the method comprises applying (i.e. adding/flowing over the surface of the solid support), a first nicking enzyme. In one example, the nicking enzyme cleaves the first or second restriction sites within the template strand.

In one embodiment, the first nicking enzyme cleaves the first restriction sites. These are the restriction sites within the first adaptor (or present originally in the adaptor). In one embodiment, the first restriction site is in the loop sequence. In an alternative embodiment, the second restriction site is in the base-paired stem (that flank the loop sequence).

In another embodiment, the first nicking enzyme cleaves the second restriction sites. These are the restriction sites within the second adaptor. In one embodiment, the second restriction site is in base-paired stem (at 3′ end of the second adaptor sequences in the single stranded template).

In one embodiment, following cleavage the sequences located 3′ of the cleaved sequence are de-hybridised and washed off.

In a further embodiment, the method comprises carrying out a first sequencing read to determine the sequence of the first and second immobilised strands simultaneously, such as by a sequencing-by-synthesis technique or by a sequencing-by ligation technique.

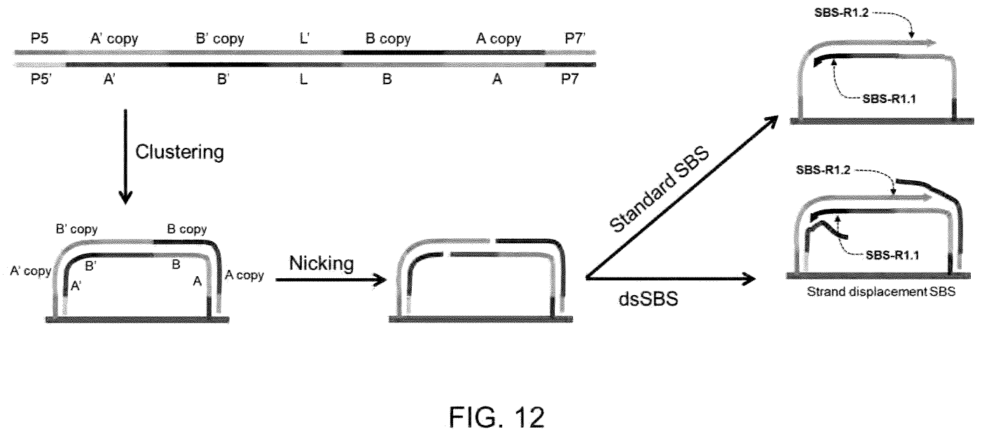

An example of a method of sequencing an inverted-repeat tandem-insert library strand is shown in FIG. 12. Each inverted-repeat tandem-insert duplex is de-hybridized, and the single strands flowed across a solid support (e.g. a flow cell) to attach to the solid support via Watson-Crick binding to a complementary lawn primer (P5 or P7) and become immobilised. The lawn primers (P5 and P7) are then extended (using the hybridised strand as a “template”) to generate a first or second immobilised template strand. For example, the first extended immobilised strand may comprise a first primer sequence at its' 5′ end (e.g. P5), and a first primer-binding sequence at its 3′ end (e.g. P7′). Similarly, the second extended immobilised strand may comprise a second primer sequence at its' 5′ end (e.g. P7), and a second primer-binding sequence at its 3′ end (e.g. P5′).

Following extension of the lawn primers to generate the first and second extended strands, 3′ ends of each extended strand bend over to bind to the other, non-bound lawn adaptor (P7 or P5) to form a sequence bridge. As described above, this sequence bridge differs from conventional sequence bridges, as the sequence bridge is hybridised at at least three places-(1) 5′ primer (e.g. P5) of the first extended strand is hybridised to 3′ primer-binding region of the second extended strand (e.g. P5′); (2) the loop sequences of both the first and second extended strands and (3) 5′ primer of the second extended strand (e.g. P7) is hybridised to 3′ primer-binding region of the first extended strand (e.g. P7′). As described above, this structure may be referred to herein as a loop-hybridised sequence bridge. The sequence bridge may be further hybridised within the regions to be identified.

In the next step, nicking enzymes are added. The nicking enzymes may be flowed across the solid support following clustering and formation of the loop-hybridised sequence bridge as described above.

As shown in FIG. 12, where the loop sequences (or loop complement sequences) comprise a 3′ restriction site (that is, the restriction site is at 3′ end of the loop sequence) nicking enzymes may be applied to nick the sequence bridges at a pair of recognition sequences in the loop stem (e.g. the base-paired stem). This leaves the first extended strand and the second extended strand hybridised at the loop structure, each of which provide a sequencing start site for a different strand of the original duplex template. These strands can be simultaneously sequenced by standard SBS or double-stranded SBS (e.g. strand displacement SBS), as shown in FIG. 12. However, in all configurations of this workflow, the sequencing start sites are formed simultaneously by nicking enzymes, which therefore, allows both strands of the duplex to be sequenced simultaneously.

In standard SBS sequencing, the non-immobilised sequences—that is, the sequences 3′ of the nicked site—are washed off before addition of a read 1.1 (SBS-R1.2) and read 1.2 (SBS-R1.2) sequencing primer, which anneal to the nicked sites in the loop sequence of the first and second extended strands respectively, and a polymerase. As shown in FIG. 12, read 1.1 will sequence B′ and A′ (i.e. the reverse strand of the original duplex in 3′ to 5′ direction) and read 1.2 will sequence B copy and A copy (the copy of the forward strand of the original duplex in 3′ to 5′ direction). This allows for any errors in the reverse strand to be identified.

In double-stranded SBS (e.g. strand displacement SBS), the non-immobilised sequences 3′ of the nicked site are not washed off.

Single-strand displacement SBS is an effective method for the sequencing of the prepared duplex. This method requires a nick in the duplex sequence and primers for DNA polymerase to utilise, to incorporate reversibly-terminated labelled dNTPs into a complementary strand of one strand of the template.

Single-strand displacement SBS combines the principles of single strand replication and sequencing-by-synthesis technologies to sequence duplexes. In single-strand displacement SBS, a DNA polymerase capable of strand-displacement but lacking exonuclease activity, such as phi29 DNA polymerase, is utilised. DNA polymerases lacking exonuclease activity in both 5′-3′ and 3′-5′ direction are required, to allow for both Reads 1 and 2. The nick site within the duplex target and annealed primer provides a binding site for such a DNA polymerase to bind. After docking, the DNA polymerase extends the primer adjacent to the nick site to generate a sequencing strand. The sequencing strand is formed by incorporating labelled deoxynucleoside triphosphates (dNTP), complementary to the relevant template strand. The labelled dNTPs act as a terminator for polymerization, so after each dNTP incorporation, the fluorescent dye is imaged to identify the base and then enzymatically cleaved to allow incorporation of the next nucleotide. Since all four reversible terminator-bound dNTPs (A, C, T, G) are present as single, separate molecules, natural competition minimizes incorporation bias. Simultaneous to polymerising a complementary strand, the DNA polymerase uses its strand displacement activity to displace the other “non-template”strand for access. In this invention, this workflow occurs simultaneously for each read (R1.1 and R1.2/R2.1 and R2.2).

FIG. 6 describes an alternative method of sequencing an inverted-repeat tandem-insert template. A sequence bridge is formed as described in FIG. 3. In this example, 3′ end of the lawn primer sequences (e.g. both P5 and P7) comprise a restriction site (the second restriction site) as described above. This restriction site is the complement of the restriction site present in the base-paired stem of the second adaptor. Simultaneous nicking of these restriction sites provides two sequencing start sites that allow simultaneous sequencing from the opposite end of both inserts, i.e. 5′ to 3′ direction—and at opposite ends of the insert to FIG. 12. As described in FIG. 6, these strands can be simultaneously sequenced by double-stranded SBS, such as strand displacement SBS. As shown in FIG. 6, read 1.1 (SBS R1.1) will sequence A′ copy and B′ copy (the copy of the reverse strand of the original duplex in 5′ to 3′ direction) and read 1.2 (SBS R1.2) will sequence A and B (the forward strand of the original duplex in 5′ to 3′ direction). This allows for any errors in the forward strand to be identified.

As shown in FIG. 7, a 9QAM encoding scheme can be used to accurately differentiate between two simultaneously received base calls. By plotting relative intensities of light signals obtained from Read 1.1 and Read 1.2 a constellation of 9 clouds is obtained. Each of these clouds allows sequence information to be identified from the two reads; in this particular encoding scheme, the top left corner of four clouds corresponds with base calls corresponding to A, the top right corner of four clouds corresponds with base calls corresponding to T, the bottom left corner of four clouds corresponds with base calls corresponding to G, and the bottom right corner of four clouds corresponds with base calls corresponding to C; however, other encoding schemes are possible and each of C, G, A and T may be mapped to different cloud permutations. By plotting the light intensities in this manner it is possible to determine an accurate base call from a library prep or sequencing error (and by library prep or sequencing error is meant here that there is a mismatch between read 1.1 and read 1.2, which may be indicative of asymmetry between the forward and reverse strands, for example, because of DNA damage to one strand).

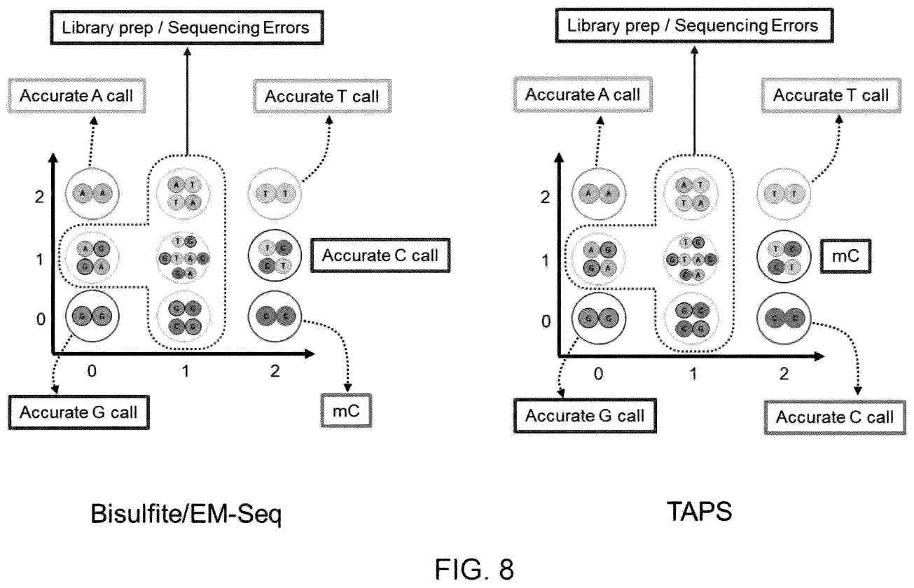

The method described herein can also be used to simultaneously sequence genomic and epigenetic data. Following preparation of the polynucleotide library strand, an epigenetic conversion is applied. The modified library strand can then be sequenced as described above and the sequences of the duplex strands read simultaneously. A 9QaM system is used to decode the simultaneously-received read signals. Depending on which technology for epigenetic conversion is used, the C/C cloud may either represent a mC (Bisulfite/EM-Seq) or accurate C call (TAPS) and vice versa, the C/T cloud will represent the mC or accurate C calls respectively (FIG. 8).

Following sequencing of one strand of the duplex (i.e. read 1) as described above, sequencing of the other, second strand of the duplex can be carried out using either single stranded or double stranded SBS.

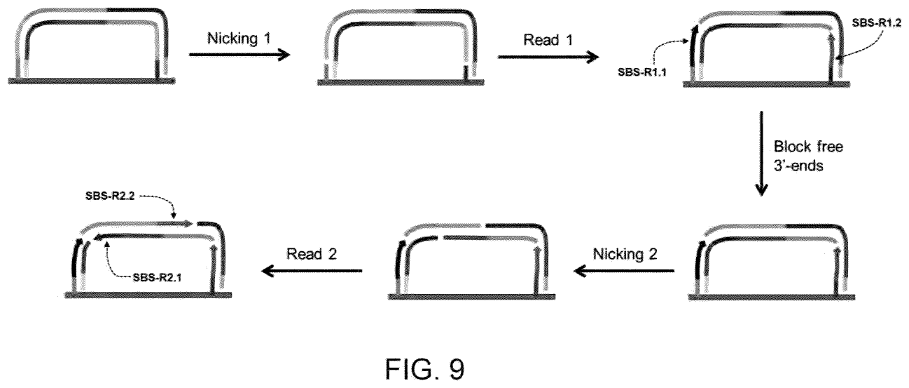

In one example, as shown in FIG. 9, following nicking of the lawn primers (as shown in FIG. 6 or 12) and sequencing of the first strand (read 1), the free ends of the sequenced strands are blocked. By “free ends” is meant the free 3′ hydroxyl group of 3′ end or 3′ nucleotide of an extended polynucleotide strand.

Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the lawn primer), a hydrogen atom instead of a 3′-OH group, a phosphate group, a propyl spacer (e.g. —O—(CH₂)₃—OH instead of a 3′—OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the free end by a polymerase. Alternatively, instead of blocking the free ends, these strands are extended to regenerate the polynucleotide strand (i.e. to resynthesized to generate 3′ primer-binding sequences).

In the next step, nicking enzymes may be applied to nick the sequence bridges at the restriction sites within the loop sequence (or loop complement sequence), using an alternative recognition site to the first nicking event. That is, nicking occurs at the restriction sites at the 3′ end of the loop sequence. As shown in FIG. 9, this generates two start sites for sequencing allowing simultaneous sequencing of the other strand of the original polynucleotide duplex. For example, as shown in FIG. 9, read 2.1 (SBS-R2.1) will sequence B′ and A′ (i.e. the reverse strand of the original duplex in 3′ to 5′ direction) and read 2.2 (SBS-R2.2) will sequence B copy and A copy (the copy of the forward strand of the original duplex in 3′ to 5′ direction). This allows for any errors in the reverse strand to be identified. In this example, read 2 may be sequenced by either single or double-stranded SBS, as described above.

The two reads, each with simultaneous sequencing of two strands—as described for example in FIGS. 6 and 9—allows the entire inverted-repeat tandem-insert duplex to be sequenced.

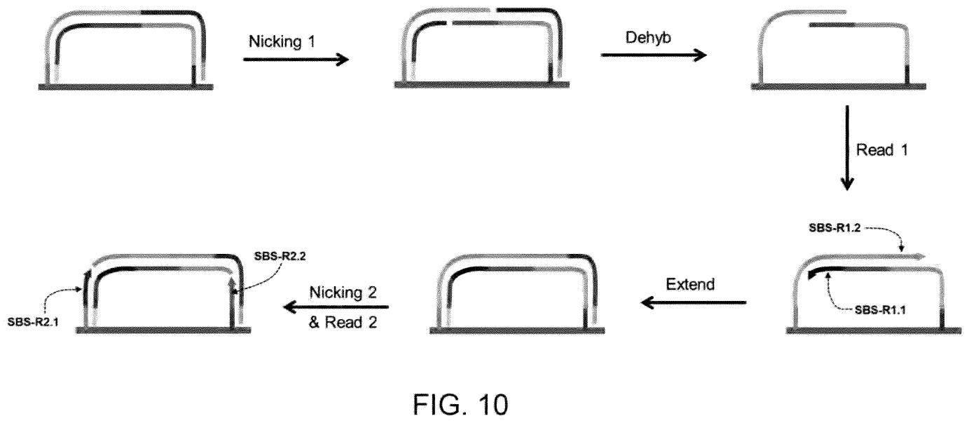

The order of nicking reactions can also be reversed. For example, the first nicking step may be nicking of the loop sequence and the second nicking step may be nicking of 3′ end of the primer sequence. This is shown, for example in FIG. 10.

As shown in FIG. 10, read 1 is generated following the method described in FIG. 12. This allows for any errors in the forward strand to be identified. Sequencing may be single-stranded or double-stranded SBS.

The sequenced strands are then extended (i.e. resynthesized) to regenerate 3′ primer-binding sequences. In the next step, nicking enzymes may be applied to nick the sequence bridges at the 3′ end of the primer sequences (as described, for example, in FIG. 10). Simultaneous nicking of these restriction sites provides two sequencing start sites that allow simultaneous sequencing from the opposite end of both inserts, i.e. 5′ to 3′ direction—and at opposite ends of the insert to FIG. 12. As described in FIG. 10, these strands can be simultaneously sequenced by double-stranded SBS, such as strand displacement SBS. As shown in FIG. 10, read 2.1 (SBS R2.1) will sequence A′ copy and B′ copy (the copy of the reverse strand of the original duplex in 5′ to 3′ direction) and read 2.2 (SBS R2.2) will sequence A and B (the forward strand of the original duplex in 5′ to 3′ direction). This allows for any errors in the forward strand to be identified.

Accordingly, in a further embodiment, following read 1, the method comprises blocking all or substantially all free 3′ ends of the immobilised strands. Alternatively, following read 1, each immobilised strand is extended to regenerate the loop-hybridised sequence bridge described (as shown in FIG. 10). Therefore, in one embodiment, the method comprises carrying out an extension reaction to extend each immobilised strand.

In a further embodiment, the method further comprises applying (i.e. adding/flowing over the surface of the solid support), a second nicking enzyme. In one embodiment, the second nicking enzyme cleaves the first or second restriction sites within the template strand. In another embodiment, the second nicking enzyme cleaves a different restriction site from the first nicking enzyme. Accordingly, where the first nicking enzyme cleaves the first restriction site, the second nicking enzyme cleaves the second restriction site (as shown in FIG. 10). Similarly, where the first nicking enzyme cleaves the second restriction site, the second nicking enzyme cleaves the first restriction site (as shown in FIG. 9).

In one embodiment, following read 1, and where the first nicking enzyme has cleaved the second restriction site, the method comprises blocking all or substantially all free 3′ ends of the immobilised strands, and applying a second nicking enzyme where the second nicking enzyme cleaves the first restriction site (as shown in FIG. 9).

In an alternative embodiment, following read 1, and where the first nicking enzyme has cleaved the first restriction site, the method comprises carrying out an extension reaction to extend the immobilised strands, and applying a second nicking enzyme where the second nicking enzyme cleaves the second restriction site as shown in FIG. 10).

In a further embodiment, the method comprises carrying out a second sequencing read to determine the sequence of the first and second immobilised strands simultaneously, such as by a sequencing-by-synthesis technique or by a sequencing-by ligation technique. This sequence read is read 2.

In an alternative embodiment, the method comprises generating a sequence bridge, as described above, and simultaneously cleaving both strands of the bridge. This is possible if the first restriction site is in the middle of the loop or substantially the middle of the loop.

In one embodiment, the endonuclease is a double strand restriction endonuclease or restriction enzyme. By either of these terms is meant an enzyme that can hydrolyze both strands of the double-stranded polynucleotide (duplex), to produce DNA molecules that are cleaved on both strands. In one embodiment, the restriction enzyme is a type II restriction enzyme. In one example, the type II restriction enzyme is EcoRI and the restriction enzyme is G/AATTC wherein EcoRI catalyzes a double stranded break within the recognition site. In another example, the type II restriction enzyme is Bg1ll and the restriction site is A/GATCT, wherein Bg1ll catalyzes a double stranded break within the recognition site. In a further example, the type II restriction enzyme is Notl and the restriction site is GC/GGCCGC, wherein Notl catalyses a double stranded break within the recognition site.

Furthermore, in this embodiment, the loop sequence in the first adaptor will comprise the following structure: first sequencing primer-binding sequence-restriction site-complement of a second sequencing primer-binding sequence. As a result, the first immobilised template (within the loop sequence) will comprise a first sequencing primer-binding sequence, a restriction site and a complement of a second sequencing primer-binding sequence, and the second immobilised template will comprise a complement of a first sequencing primer-binding sequence, a restriction site and a complement of a second sequencing primer-binding sequence. The first and second sequencing primer-binding sequences bind a sequencing primer, which may be the same sequence. That is, they bind the same sequencing primer. Alternatively, the first and second sequencing primer-binding sequences are different. That is, they bind different sequencing primers. The sequencing primer-binding sequences may be in the base-paired stem of the loop sequence.

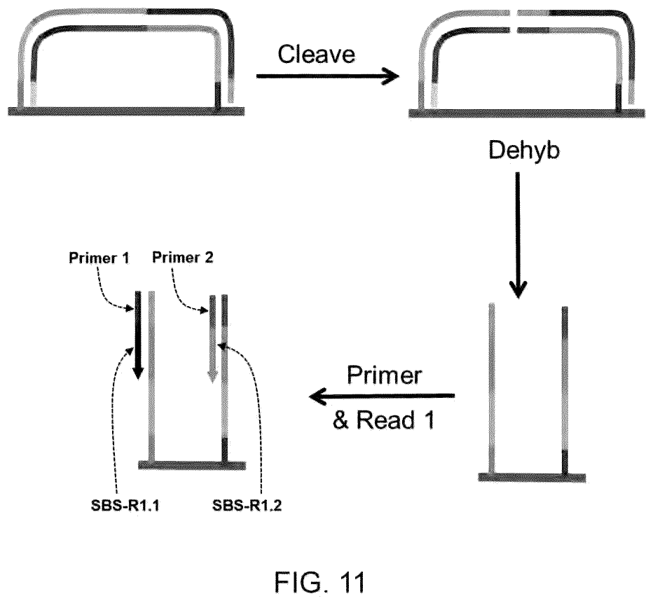

Following nicking of the loop sequence two immobilised extended strands are generated—a first immobilised extended strand and a second immobilised extended strand, as shown in FIG. 11. In effect, this step halves the tandem insert. Each immobilised extended strand has a 3′ sequencing primer-binding sequence (either a first sequencing primer-binding sequence or a second sequencing primer-binding sequence). Non-immobilised strands may be washed off.

Binding of a first sequencing primer to the first sequencing primer-binding sequence will allow sequencing of read 1.1. As shown in FIG. 11.

Binding of a second sequencing primer to the second sequencing primer-binding sequence will allow sequencing of read 1.2. As shown in FIG. 11.

In one embodiment, binding of first sequencing primers to the first sequencing primer-binding sequence generates a first signal and binding of second sequencing primers to the second sequencing primer-binding sequence generates a second signal, where the intensity of the first signal is greater than the intensity of the second signal. This allows read 1.1 and 1.2 to be read simultaneously. This is achieved using a mixed population of blocked and unblocked second sequencing primers that bind the second sequencing primer-binding site. Any ratio of blocked: unblocked second primers can be used that generates a second signal that is of a lower intensity than the first signal, for example, the ratio of blocked: unblocked primers may be: 20:80 to 80:20, or 1:2 to 2:1. In one embodiment, a ratio of 50:50 of blocked: unblocked second primers is used, which in turn generates a second signal that is around 50% of the intensity of the first signal.

The first and second sequencing primers may be added to the flow cell at the same time, or separately but sequentially.

By “blocked” is meant that the sequencing primer comprises a blocking group at a 3′ end of the sequencing primer. Suitable blocking groups include a hairpin loop (e.g. a polynucleotide attached to 3′-end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase.

The sequence of the sequencing primers and the sequence primer binding sites are not material to the methods of the invention, as long as the sequencing primers are able to bind to the sequence primer-binding site to enable amplification and sequencing of the regions to be identified.

In summary, the above-described example would allow spatially separated clusters to be read in a temporally simultaneous manner through the generation of an optically unresolved signal that can be analytically separated using 16QaM.

In a further embodiment, the method may additionally comprise generating a complement of the read 1 sequences (i.e. a complement of the halves the tandem insert shown in FIG. 10), and sequencing the complements as described above (i.e. following the same method of FIG. 10 with sequencing primers that bind complements of the first and second primer-binding sequences). This allows sequencing of read 2. Again, binding of first sequencing primers to the complement of the first sequencing primer-binding sequence generates a first signal and binding of second sequencing primers to the complement of the second sequencing primer-binding sequence generates a second signal, where the intensity of the first signal is greater than the intensity of the second signal allows read 2.1 and 2.2 to be read simultaneously. In one embodiment, the complements of the read 1 sequences may be obtained by modifying the solid support such that the solid support additionally comprises lawn primers (third and fourth lawn primers) that are complementary to the or at least a portion of the first and second primer-binding sequences. Binding of 3′ end of the immobilised read 1 sequences (e.g. last diagram of FIG. 11) to third and fourth primers (not shown) leads to formation of a bridge. The third and fourth lawn primers can be extended using bridge amplification and sequenced using the methods described above.

Accordingly, in an alternative embodiment, the method of identifying a polynucleotide, comprises applying (i.e. adding/flowing over the surface of the solid support), a first restriction enzyme, wherein the restriction enzyme cleaves the first restriction site, wherein the first restriction site is in the loop sequence of the first adaptor. In one embodiment, following cleavage the sequences 3′ of the cleaved sequence are de-hybridised and washed off.

In a further embodiment, the method comprises carrying out a first sequencing read to determine the sequence of the first and second immobilised strands simultaneously, such as by a sequencing-by-synthesis technique or by a sequencing-by ligation technique.

### Detection of Mismatched Base Pairs

Embodiments are directed to a method of polynucleotide sequences for detection of mismatched base pairs, comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template.

Advantageously, by synthesising at least one first polynucleotide sequence comprising a first portion and the at least one second polynucleotide sequence comprising a second portion, wherein the first portion comprises a forward strand of the template (or reverse strand of the template), and the second portion comprises a reverse complement strand of the template (or forward complement strand of the template), mismatched base pairs can be detected quickly and reliably, which in turn allows errors in the sequencing output to be corrected. The odds of an error appearing from a typical library preparation method are usually in the order of 1 in 103. However, the odds that two identical library preparation errors occur in both the forward strand of the template and the reverse complement strand of the template (or the reverse strand of the template and the forward complement strand of the template) is in the order of 1 in 107. Thus, sequencing output and accuracy can be increased drastically.

In some embodiments, selective processing methods may be used when preparing the templates. This leads to further advantages, as it also becomes possible to attribute specific nucleobases of the mismatched base pair to particular strands of the original library, thus leading to more precise error detection, whilst maintaining reductions in time taken to detect mismatched base pairs.

The first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). Alternatively, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand). In some embodiments, the first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced. In these particular embodiments, concurrent sequencing of both the forward and reverse complement strands (or the reverse and forward complement strands) allows mismatched base pairs and/or epigenetic modification to be detected.

Where mismatched base pairs are detected, the forward strand of the template may not be identical to the reverse complement strand of the template. Alternatively, the reverse strand of the template may not be identical to the forward complement strand of the template.

The method may further comprise a step of preparing the first portion and the second portion for concurrent sequencing.

For example, the method may comprise simultaneously contacting first sequencing primer binding sites located after a 3′-end of the first portions with first primers and second sequencing primer binding sites located after a 3′-end of the second portions with second primers. Thus, the first portions and second portions are primed for concurrent sequencing.

The method may alternatively or additionally comprise nicking the at least one first polynucleotide sequence and nicking the at least one second polynucleotide sequence. In some embodiments, the nick on the at least one first polynucleotide sequence may be located after a 3′-end of the first portion, and the nick on the at least one second polynucleotide sequence may be located after a 3′-end of the second portion. In some embodiments, the nick on the at least one first polynucleotide sequence may be located before a 5′-end of the first portion, and the nick on the at least one second polynucleotide sequence may be located before a 5′-end of the second portion. Thus, the first portions and second portions are primed for concurrent sequencing as sequencing may begin from the nick (e.g. by using strand displacement SBS, or after washing off non-immobilised strands).

In some embodiments, a proportion of first portions may be capable of generating a first signal and a proportion of second portions may be capable of generating a second signal, wherein an intensity of the first signal is substantially the same as an intensity of the second signal.

In other embodiments (e.g. where selective processing methods are used as described herein), a proportion of first portions may be capable of generating a first signal and a proportion of second portions may be capable of generating a second signal, wherein an intensity of the first signal is substantially the same as an intensity of the second signal.

The first signal and the second signal may be spatially unresolved (e.g. generated from the same region or substantially overlapping regions).

Further aspects relating to selective processing methods (e.g. conducting selective sequencing or preparing for selective sequencing) have already been described herein and apply to the methods of preparing polynucleotide sequences for detection of mismatched base pairs as described herein.

The first portion may be referred to herein as read 1.1 (R1.1). The second portion may be referred to herein as read 1.2 (R1.2).

In one embodiment, the first portion is at least 25 or at least 50 base pairs and the second portion is at least 25 base pairs or at least 50 base pairs.

The first and second strand may be separately attached to a solid support. This solid support may be a flow cell. In one embodiment, each of the first and second strands are attached to the solid support (e.g. flow cell) in a single well of the solid support.

The polynucleotide strands may form or be part of a cluster on the solid support.

As used herein, the term “cluster” may refer to a clonal group of template polynucleotides (e.g. DNA or RNA) bound within a single well of a solid support (e.g. flow cell). As such, a cluster may refer to the population of polynucleotide molecules within a well that are then sequenced. A “cluster” may contain a sufficient number of copies of template polynucleotides such that the cluster is able to output a signal (e.g. a light signal) that allows sequencing reads to be performed on the cluster. A “cluster” may comprise, for example, about 500 to about 2000 copies, about 600 to about 1800 copies, about 700 to about 1600 copies, about 800 to 1400 copies, about 900 to 1200 copies, or about 1000 copies of template polynucleotides.

A cluster may be formed by bridge amplification, as described above.

Where the method of the invention involves a first polynucleotide strand and a second polynucleotide strand, the cluster formed may be a duoclonal cluster.

By “duoclonal” cluster is meant that the population of polynucleotide sequences that are then sequenced (as the next step) are substantially of two types—e.g. a first sequence and a second sequence. As such, a “duoclonal” cluster may refer to the population of single first sequences and single second sequences within a well that are then sequenced. A “duoclonal” cluster may contain a sufficient number of copies of a single first sequence and copies of a single second sequence such that the cluster is able to output a signal (e.g. a light signal) that allows sequencing reads to be performed on the “monoclonal” cluster. A “duoclonal” cluster may comprise, for example, about 500 to about 2000 combined copies, about 600 to about 1800 combined copies, about 700 to about 1600 combined copies, about 800 to 1400 combined copies, about 900 to 1200 combined copies, about 1000 combined copies of single first sequences and single second sequences. The copies of single first sequences and single second sequences together may comprise at least about 50%, at least about 60%, at least about 70%, at least about 80%, at least about 90%, or about 95%, 98%, 99% or 100% of all polynucleotides within a single well of the flow cell, and thus providing a substantially duoclonal “cluster”.

The at least one first polynucleotide sequence comprising a first portion and at least one second polynucleotide sequence may be prepared using a loop fork method as described herein (see FIG. 27).

Accordingly, in one embodiment, the step of synthesising at least one first polynucleotide sequence comprising a first portion and at least one second polynucleotide sequence comprising a second portion may comprise:
synthesising a loop-ligated precursor polynucleotide by connecting a 3′-end of the forward strand of the target polynucleotide and a 5′-end of the reverse strand of the target polynucleotide with a loop, or connecting a 5′-end of the forward strand of the target polynucleotide and a 3′-end of the reverse strand of the target polynucleotide with a loop,
synthesising the at least one first polynucleotide sequence comprising the first portion by forming a complement of the loop-ligated precursor polynucleotide, and
synthesising the at least one second polynucleotide sequence comprising the at least one second polynucleotide sequence by forming a complement of the at least one first polynucleotide sequence.

Typically, the loop may be generated by attaching a first flanking adaptor to the target (double-stranded) polynucleotide.

The first flanking adaptor may be an oligonucleotide of any structure or any sequence that allows the forward and reverse strands to be connected via a loop. In one embodiment, the first flanking adaptor comprises a base-paired stem and a hairpin loop (e.g. a loop structure with unpaired or non-Watson-Crick paired nucleotides) and connects 3′ end of the forward strand with 5′ end of the reverse strand, or 5′ end of the forward strand with 3′ end of the reverse strand.

The step of synthesising the loop-ligated precursor polynucleotide may further comprise connecting a 5′-end of the forward strand of the target polynucleotide and a 3′-end of the reverse strand of the target polynucleotide (when 3′-end of the forward strand of the target polynucleotide and 5′-end of the reverse strand of the target polynucleotide are connected with a loop), or a 3′-end of the forward strand of the target polynucleotide and a 5′-end of the reverse strand of the target polynucleotide (when 5′-end of the forward strand of the target polynucleotide and 3′-end of the reverse strand of the target polynucleotide are connected with a loop), with a second flanking adaptor.

In one embodiment, the second flanking adaptor comprises a base-paired stem, a primer-binding sequence and a primer-binding complement sequence. Specifically, the second flanking adaptor may comprise a first and second strand, wherein the first and second strands are base-paired for a portion of their sequence (forming the base-paired stem) and are non-complementary for the remainder of their sequence, for example, P5′ and P7 or P7′ and P5, which subsequently forms a fork structure, wherein a first arm of the fork structure comprises a primer-binding sequence and the second arm of the fork structure comprises a primer-binding complement sequence. In an alternative embodiment, the second flanking adaptor may comprise a base-paired stem and a hairpin loop, where the loop comprises a primer-binding sequence, a cleavable site and primer-binding complement sequence, where the cleavable site is in-between the primer-binding sequence and the primer-binding complement sequence. In this alternative embodiment, the method may comprise cleaving the loop of the second flanking adaptor at the cleavable site to open the loop. This will generate a fork structure, as described above. Specifically, following cleavage the second flanking adaptor will form a base-paired stem and then a fork.

As used herein for the second flanking adaptor, by “cleavable site” is meant any moiety, such as a modified nucleotide, that allows selective cleavage of the second flanking adaptor sequence. By way of non-limiting example, the cleavable site may comprise uracil bases, phosphorothioate groups, ribonucleotides, diol linkages, disulphide linkages, peptides etc.

In one example, the cleavable site is a uracil. Uracil can be cleaved using a uracil glycosylase or USER enzyme mix (which is a cocktail of uracil glycosylase and endonuclease VIII). In another example, the cleavable site is 8-oxoguanine. 8-oxoguanine can be cleaved using a FPG glycosylase. Alternatively, the cleavable site is a restriction site.

By “restriction site” is meant a sequence of nucleotides recognised by an endonuclease, such as a single-stranded endonuclease. A restriction site may also be referred to as a “recognition site” or “recognition sequence”, and such terms may be used interchangeably.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands. Examples of suitable nicking enzymes that may be used include, but are not limited to, Nb.BbvCI, Nb.Bsml, Nb.BsrDI, Nb.Btsl, Nt.Alwl, Nt.BsmAl, Nt.BspQI, Nt.BstNBI, BssSI, Nb.Bpu101 and Nt.CviPII, These nickases can be used either alone or in various combinations. Other suitable nicking endonucleases are available from commercial sources, including New England Biolabs and Fisher Scientific.

In one embodiment, the second flanking adaptor comprises at least one primer-binding sequence. In one example, the second flanking adaptor comprises at least one primer-binding complement sequence. In another embodiment, the second flanking adaptor comprises both a primer-binding sequence and a primer-binding complement sequence. The primer-binding sequence may be capable of binding to a lawn or immobilised primer that is immobilised on the surface of a solid support. For example, the primer-binding sequence may be either P5′ (for example, SEQ ID NO. 3 or 6 or a variant or fragment thereof) or P7′ (for example, SEQ ID NO. 4 or a variant or fragment thereof). Similarly, the primer-binding complement sequence may be either P5 (for example, SEQ ID NO. 1 or 5 or a variant or fragment thereof) or P7 (for example, SEQ ID NO. 2 or a variant or fragment thereof). If the primer-binding sequence is P5′, the primer-binding complement sequence is P7. If the primer-binding sequence is P7′, the primer-binding complement sequence is P5.

At least one of the first flanking adaptor and the second flanking adaptor comprises a restriction site for an endonuclease, such as a single-stranded endonuclease. If the second flanking adaptor comprises a base-paired stem and a hairpin loop structure, then the restriction site for an endonuclease is additional to the cleavable site. Where the restriction site is present in the first flanking adaptor, this allows a nick to be generated in the template and/or template complement strands in the loop (and/or loop complement) formed from the first flanking adaptor. Where the restriction site is present in the second flanking adaptor, this allows a nick to be generated close to the first immobilised primer and/or the second immobilised primer. Where nicking is used, such a nick prepares the strands for sequencing, since sequencing can be initiated from the nick (e.g. using strand displacement SBS), or allows non-immobilised polynucleotide sequences to be washed away to enable binding of sequencing primers.

In one embodiment, the endonuclease is a single strand restriction endonuclease, a nicking endonuclease or nicking enzyme or nickase (again, such terms may be used interchangeably). By any of these terms is meant an enzyme that can hydrolyze only one strand of the double-stranded polynucleotide (duplex), to produce DNA molecules that are “nicked”, rather than fully cleaved on both strands. Examples of suitable nicking enzymes that may be used include, but are not limited to, Nb.BbvCI, Nb.Bsml, Nb.BsrDI, Nb.Btsl, Nt.Alwl, Nt.BsmAl, Nt.BspQI, Nt.BstNBI, BssSI, Nb.Bpu101 and Nt.CviPII, These nickases can be used either alone or in various combinations. Other suitable nicking endonucleases are available from commercial sources, including New England Biolabs and Fisher Scientific.

The first and second flanking adaptors also may comprise one or more sequencing primer-binding sites (or sequencing primer-binding site complements). The sequencing primer-binding sites and the sequencing primer-binding site complements may allow binding of a sequencing primer.

In the first flanking adaptor the sequencing primer-binding sites may be in the loop sequence or in the base-paired stem. In one embodiment, the base-paired stem comprises at least one sequencing primer-binding site. In one embodiment, the sequencing primer-binding site is in the base-paired stem, and in the part of the stem that connects to the reverse strand of the double-stranded polynucleotide. In another embodiment, the loop may comprise two sequencing primer-binding sites. In another embodiment, the loop comprises two sequencing primer-binding sites and a restriction site, wherein the sequencing primer-binding sites are either side of the restriction site.

In the second flanking adaptor the sequencing primer-binding site(s) may also be in the base-paired stem. Alternatively, each fork of the second flanking adaptor may additionally comprise a sequencing primer-binding site.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

The sequence of the sequencing primers and the sequence primer binding sites are not material to the methods of the invention, as long as the sequencing primers are able to bind to the sequence primer binding site (or sequencing binding site complement) to enable amplification and sequencing of the regions to be identified.

In some embodiments, the restriction site in the first flanking adaptor is in the middle of the loop or substantially the middle of the loop. In particular, the restriction site may be cleavable by a double strand restriction endonuclease or restriction enzyme. By either of these terms is meant an enzyme that can hydrolyze both strands of the double-stranded polynucleotide (duplex), to produce polynucleotide molecules that are cleaved on both strands. In one embodiment, the restriction enzyme is a type II restriction enzyme.

FIGS. 11, 12 and 34 illustrate various ways in which first portions and second portions can be prepared for concurrent sequencing.

FIG. 12 shows how concurrent sequencing is enabled by nicking after a 3′-end of the first portion, and nicking after a 3′-end of the second portion. Here, the nicks are made at a 3′-end of both the loop and loop complement. In one case, non-immobilised strands may be washed away and standard SBS can be conducted, resulting in concurrent sequencing of the first and second portions. In an alternative case, the non-immobilised strands are not washed away and SBS can be conducted using a strand displacement polymerase, again resulting in concurrent sequencing of the first and second portions.

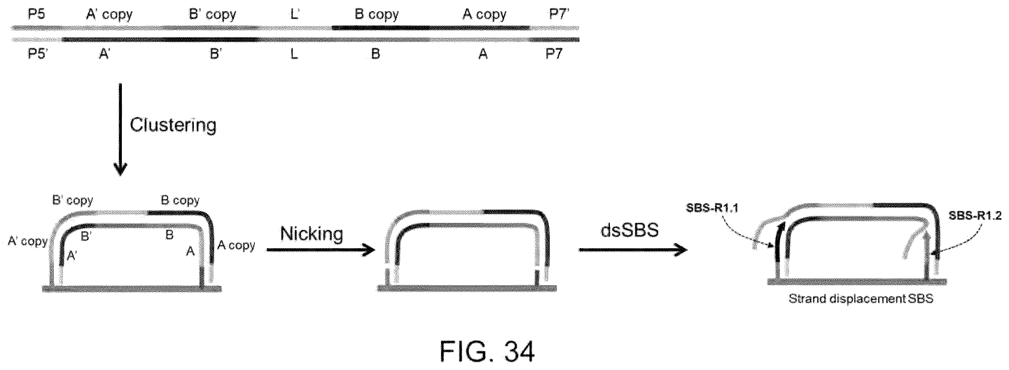

FIG. 34 shows how concurrent sequencing is enabled by nicking before a 5′-end of the first portion, and nicking before a 5′-end of the second portion. Here, the nicks are made after a 3′-end of the first immobilised primer and after a 3′-end of the second immobilised primer. SBS can then be conducted using a strand displacement polymerase, resulting in concurrent sequencing of the first and second portions.

FIG. 11 shows how concurrent sequencing is enabled by contacting first sequencing primer binding sites located after a 3′-end of the first portions with first primers and second sequencing primer binding sites located after a 3′-end of the second portions with second primers. Here, a middle portion of the loop and loop complement may be cleaved (e.g. with a double strand restriction endonuclease or restriction enzyme). The non-immobilised strands may be washed away, and any remaining sections of the loop and loop complement can act as sequencing primer binding sites, allowing standard SBS to be conducted resulting in concurrent sequencing of the first and second portions.

It is also possible to conduct paired end reads using these methods. FIGS. 9 and 10 illustrate various ways in which paired end reads can be achieved.

FIG. 10 shows paired end reads being conducted after a first round of concurrent sequencing as shown in FIG. 12. Further nicks can be made after a 3′-end of the first immobilised primer and after a 3′-end of the second immobilised primer. SBS can then be conducted using a strand displacement polymerase, resulting in concurrent sequencing of complements of the first and second portions.

FIG. 9 shows paired end reads being conducted after a first round of concurrent sequencing as shown in FIG. 34. Any free 3′-ends can be blocked. Further nicks can be made after a 3′-end of the first portion, and after a 3′-end of the second portion, then SBS can then be conducted using a strand displacement polymerase, resulting in concurrent sequencing of complements of the first and second portions.

Although not shown in FIG. 11 paired end reads can also be conducted after Read 1.1 and Read 1.2. This can be achieved by having further immobilised primers on the solid support that are substantially complementary to the remaining sections of the loop and loop complement acting as sequencing primer binding sites. This allows resynthesis of the strands, and subsequent binding of further sequencing primers for concurrent sequencing of complements of the first and second portions.

In some embodiments, the method may further comprise a step of concurrently sequencing nucleobases in the first portion and the second portion.

In another embodiment, the disclosure is directed to a method of preparing at least one polynucleotide sequence for detection of mismatched base pairs, comprising:
synthesising at least one polynucleotide sequence comprising a first portion and a second portion,
wherein the at least one polynucleotide sequence comprises portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template.

Advantageously, by synthesising at least one polynucleotide sequence comprising the first portion and the second portion, wherein the first portion comprises a forward strand of the template (or reverse strand of the template), and the second portion comprises a reverse complement strand of the template (or forward complement strand of the template), mismatched base pairs can be detected quickly and reliably, which in turn allows errors in the sequencing output to be corrected. The odds of an error appearing from a typical library preparation method are usually in the order of 1 in 10³. However, the odds that two identical library preparation errors occur in both the forward strand of the template and the reverse complement strand of the template (or the reverse strand of the template and the forward complement strand of the template) is in the order of 1 in 10⁷. Thus, sequencing output and accuracy can be increased drastically.

In some embodiments, selective processing methods may be used when preparing the templates. This leads to further advantages, as it also becomes possible to attribute specific nucleobases of the mismatched base pair to particular strands of the original library, thus leading to more precise error detection, whilst maintaining reductions in time taken to detect mismatched base pairs.

The first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). Alternatively, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand). In some embodiments, the first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced. In these particular embodiments, concurrent sequencing of both the forward and reverse complement strands (or the reverse and forward complement strands) allows mismatched base pairs and/or epigenetic modification to be detected.

Where mismatched base pairs are detected, the forward strand of the template may not be identical to the reverse complement strand of the template. Alternatively, the reverse strand of the template may not be identical to the forward complement strand of the template.

The method may further comprise a step of preparing the first portion and the second portion for concurrent sequencing.

For example, the method may comprise simultaneously contacting first sequencing primer binding sites located after a 3′-end of the first portions with first primers and second sequencing primer binding sites located after a 3′-end of the second portions with second primers. Thus, the first portions and second portions are primed for concurrent sequencing.

In some embodiments, a proportion of first portions may be capable of generating a first signal and a proportion of second portions may be capable of generating a second signal, wherein an intensity of the first signal is substantially the same as an intensity of the second signal.

In other embodiments (e.g. where selective processing methods are used as described herein), a proportion of first portions may be capable of generating a first signal and a proportion of second portions may be capable of generating a second signal, wherein an intensity of the first signal is substantially the same as an intensity of the second signal.

The first signal and the second signal may be spatially unresolved (e.g. generated from the same region or substantially overlapping regions).

Further aspects relating to selective processing methods (e.g. conducting selective sequencing or preparing for selective sequencing) have already been described herein and apply to the methods of preparing at least one polynucleotide sequence for detection of mismatched base pairs as described herein.

The first portion may be referred to herein as read 1 (R1). The second portion may be referred to herein as read 2 (R2). In one embodiment, the first portion is at least 25 or at least 50 base pairs and the second portion is at least 25 base pairs or at least 50 base pairs.

The single (concatenated) polynucleotide strand may be attached to a solid support. In one embodiment, this solid support is a flow cell. In one embodiment, the polynucleotide strand is attached to the solid support in a single well of the solid support. The polynucleotide strand or strands may form or be part of a cluster on the solid support.

As used herein, the term “cluster” may refer to a clonal group of template polynucleotides (e.g. DNA or RNA) bound within a single well of a solid support (e.g. flow cell). As such, a cluster may refer to the population of polynucleotide molecules within a well that are then sequenced. A “cluster” may contain a sufficient number of copies of template polynucleotides such that the cluster is able to output a signal (e.g. a light signal) that allows sequencing reads to be performed on the cluster. A “cluster” may comprise, for example, about 500 to about 2000 copies, about 600 to about 1800 copies, about 700 to about 1600 copies, about 800 to 1400 copies, about 900 to 1200 copies, or about 1000 copies of template polynucleotides.

A cluster may be formed by bridge amplification, as described above.

Where the method of the invention involves a single polynucleotide strand with a first and second portion, before sequencing one group of strands (either the group of template polynucleotides, or the group of template complement polynucleotides thereof) may be removed from the solid support, leaving either the templates or the template complements, as explained above. Such a cluster may be considered to be a “monoclonal” cluster.

By “monoclonal” cluster is meant that the population of polynucleotide sequences that are then sequenced (as the next step) are substantially the same—i.e. copies of the same sequence. As such, a “monoclonal” cluster may refer to the population of single polynucleotide molecules within a well that are then sequenced. A “monoclonal” cluster may contain a sufficient number of copies of a single template polynucleotide (or copies of a single template complement polynucleotide) such that the cluster is able to output a signal (e.g. a light signal) that allows sequencing reads to be performed on the “monoclonal” cluster. A “monoclonal” cluster may comprise, for example, about 500 to about 2000 copies, about 600 to about 1800 copies, about 700 to about 1600 copies, about 800 to 1400 copies, about 900 to 1200 copies, or about 1000 copies of a single template polynucleotide (or copies of a single template complement polynucleotide). The copies of the single template polynucleotide (and/or single template complement polynucleotides) may comprise at least about 50%, at least about 60%, at least about 70%, at least about 80%, at least about 90%, or about 95%, 98%, 99% or 100% of all polynucleotides within a single well of the flow cell, and thus providing a substantially monoclonal “cluster”.

The at least one polynucleotide sequence comprising a first portion and a second portion may be prepared using a tandem insert method as described herein. Accordingly, in one embodiment, the step of synthesising the at least one polynucleotide sequence comprising a first portion and a second portion may comprise:
synthesising a first precursor polynucleotide fragment comprising a complement of the first portion and a hybridisation complement sequence,
synthesising a second precursor polynucleotide fragment comprising a second portion and a hybridisation sequence,
annealing the hybridisation complement sequence of the first precursor polynucleotide fragment with the hybridisation sequence on the second precursor polynucleotide fragment to form a hybridised adduct,
synthesising a first precursor polynucleotide sequence by extending the first precursor polynucleotide fragment to form a complement of the second portion, and
synthesising the at least one polynucleotide sequence by forming a complement of the first precursor polynucleotide sequence.

In one embodiment, the first precursor polynucleotide fragment may comprise a first sequencing primer binding site complement.

In one embodiment, the first sequencing primer binding site complement may be located before a 5′-end of the complement of the first portion, such as immediately before the 5′-end of the complement of the first portion.

In one aspect, the first precursor polynucleotide fragment may comprise a second adaptor complement sequence.

In one example, the second adaptor complement sequence may be located before a 5′-end of the complement of the first portion.

In another embodiment, the first precursor polynucleotide fragment may comprise a first sequencing primer binding site complement and a second adaptor complement sequence.

In one embodiment, the first sequencing primer binding site complement may be located before a 5′-end of the complement of the first portion, and wherein the second adaptor complement sequence may be located before a 5′-end of the first sequencing primer binding site complement.

In one aspect, the first precursor polynucleotide fragment may comprise a second sequencing primer binding site complement.

In one embodiment, the hybridisation sequence complement may comprise the second sequencing primer binding site complement.

In one embodiment, the second precursor polynucleotide fragment may comprise a first adaptor complement sequence.

In some embodiments, the method may further comprise a step of concurrently sequencing nucleobases in the first portion and the second portion.

### Kits

In another aspect of the invention, there is provided a library preparation kit, wherein the kit comprises a plurality of first adaptors, a plurality of second adaptors. In one embodiment, the kits further comprises instructions for use. In a further embodiment, the kit may further comprise at least one single-stranded endonuclease or restriction endonuclease. In one aspect, the endonuclease is selected from Nt. BspQI, Cas9 D10A and Cas9 H840A. In another embodiment, the kit may additionally comprise an agent for epigenetic conversion. For example, the agent for epigenetic conversion may be a conversion agent as described herein Non-limiting examples of conversion reagents include sulfites (e.g. bisulfite), cytidine deaminases (e.g. wild-type or mutant enzymes of the APOBEC family), and boron-based reducing agents (e.g. amine-borane compounds or azine-borane compounds, such as t-butylamine borane, ammonia borane, ethylenediamine borane, dimethylamine borane, pyridine borane and 2-picoline borane).

In another embodiment the kit may additionally comprise a uracil glycosylase or USER enzyme mix (which is a cocktail of uracil glycosylase and endonuclease VIII).

In another aspect of the invention there is provided a solid support comprising a plurality of a third and/or fourth primer immobilised thereon, as described above.

Methods as described herein may be performed by a user physically. In other words, a user may themselves conduct the methods of preparing polynucleotide sequences for detection of mismatched base pairs as described herein, and as such the methods as described herein may not need to be computer-implemented.

In another aspect of the invention, there is provided a kit comprising instructions for preparing polynucleotide sequences for detection of mismatched base pairs as described herein, and/or for sequencing polynucleotide sequences to detect mismatched base pairs as described herein.

In one embodiment, the kit may further comprise a sequencing primer comprising or consisting of a sequence selected from SEQ ID NO. 27-36 or a variant or fragment thereof.

In one embodiment, the kit may comprise a sequencing composition comprising a sequencing primer selected from SEQ ID NO. 27-30 or a variant or fragment thereof, and a sequencing primer selected from SEQ ID NO. 31-36 or a variant or fragment thereof.

In another aspect of the invention, there is provided a kit comprising instructions for preparing at least one polynucleotide sequence or region of a polynucleotide sequence for identification and/or sequencing at least one polynucleotide sequence or region of a polynucleotide sequence according to the methods described herein.

In one embodiment, the kit may further comprise a sequencing primer comprising or consisting of a sequence selected from SEQ ID NO: 31 to 36 or a variant or fragment thereof.

A sequencing composition comprising a sequencing primer selected from SEQ ID NO: 35 or 36 or a variant or fragment thereof, and a sequencing primer selected from SEQ ID NO: 33 or 34 or a variant or fragment thereof.

In another embodiment, the kit may further comprise an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

In another embodiment, the kit may comprise a primer-blocking agent(s), wherein the primer-blocking agent is preferably a blocked nucleotide, more preferably a blocked A or G. The kit may additionally further comprise at least one extended primer sequence(s), wherein the extended primer sequence is selected from SEQ ID NO: 13 to 23, and wherein the extended primer sequence further comprises a 5′ additional nucleotide, wherein 5′ additional nucleotide is complementary to the primer-blocking agent. In another embodiment, the kit may further comprise an amplification mixture comprising a recombinase, a DNA polymerase, a single-stranded DNA binding protein (SSB) and primer-blocking agent, wherein the primer-blocking agent is preferably a blocked nucleotide, more preferably a blocked A or G. In a further embodiment, the kit may additionally comprise at least one extended primer sequence(s), wherein the extended primer sequence is selected from SEQ ID NO: 13 to 23, and wherein the extended primer sequence further comprises a 5′ additional nucleotide, wherein 5′ additional nucleotide is complementary to the primer-blocking agent.

Methods of Sequencing n-mers

Also described herein is a method of sequencing at least one polynucleotide sequence, comprising:
preparing at least one polynucleotide sequence for identification using a method as described herein; and
concurrently sequencing nucleobases in each of the n portions based on the intensity of each of the n^{th} signals.

In one embodiment, sequencing is performed by sequencing-by-synthesis or sequencing-by-ligation.

In one embodiment, the method may further comprise a step of conducting paired-end reads.

In some embodiments, the step of concurrently sequencing nucleobases may comprise:
(a) obtaining first intensity data comprising a combined intensity of respective first signal components generated by each of the n portions obtained based upon respective n^{th} nucleobases in each of the n portions, wherein each of the respective first signal components are obtained simultaneously;
(b) obtaining second intensity data comprising a combined intensity of respective second signal components generated by each of the n portions obtained based upon respective n^{th} nucleobases in each of the n portions, wherein each of the respective second signal components are obtained simultaneously;
(c) selecting one of a plurality of classifications based on the first and the second intensity data, wherein each classification represents a possible combination of respective n^{th} nucleobases; and
(d) based on the selected classification, base calling the respective n^{th} nucleobases for all n portions.

In one embodiment, selecting the classification based on the first and second intensity data may comprise selecting the classification based on the combined intensity of respective first signal components and second signal components.

In one embodiment, the plurality of classifications may comprise 4n classifications, each classification representing one of 4″ unique combinations of n^{th} nucleobases.

In one embodiment, the first signal components and the second signal components may be generated based on light emissions associated with the respective nucleobase.

In one embodiment, the light emissions may be detected by a sensor, wherein the sensor is configured to provide a single output based upon the n signals.

In one example, the sensor may comprise a single sensing element.

In one embodiment, the method may further comprise repeating steps (a) to (d) for each of a plurality of base calling cycles.

### Samples

In some embodiments, the sample comprises or consists of a purified or isolated polynucleotide derived from a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting embodiment, the sample is a maternal sample that is obtained from a pregnant female, for example a pregnant woman. The maternal sample can be a tissue sample, a biological fluid sample, or a cell sample. In another illustrative, but non-limiting embodiment, the maternal sample is a mixture of two or more biological samples, e.g., the biological sample can comprise two or more of a biological fluid sample, a tissue sample, and a cell culture sample.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

In some embodiments, the use of the disclosed sequencing technology does not involve the preparation of sequencing libraries. In other embodiments, the sequencing technology contemplated herein involve the preparation of sequencing libraries. In one illustrative approach, sequencing library preparation involves the production of a random collection of adapter-modified DNA fragments (e.g., polynucleotides) that are ready to be sequenced.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originated in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Methods of isolating nucleic acids from biological sources may differ depending upon the nature of the source. One of skill in the art can readily isolate nucleic acids from a source as needed for the method described herein. In some instances, it can be advantageous to fragment large nucleic acid molecules (e.g. cellular genomic DNA) in the nucleic acid sample to obtain polynucleotides in the desired size range. Fragmentation can be random, or it can be specific, as achieved, for example, using restriction endonuclease digestion. Methods for random fragmentation may include, for example, limited DNase digestion, alkali treatment and physical shearing. Fragmentation can also be achieved by any of a number of methods known to those of skill in the art. For example, fragmentation can be achieved by mechanical means including, but not limited to nebulization, sonication and hydroshear.

In some embodiments, sample nucleic acids are obtained from as cfDNA, which is not subjected to fragmentation. For example, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl. Standard protocols, e.g., protocols for sequencing using, for example, the Illumina platform, instruct users to end-repair sample DNA, to purify the end-repaired products prior to dA-tailing, and to purify the dA-tailing products prior to the adaptor-ligating steps of the library preparation.

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

### Methods of Base Calling Nucleobases

In another aspect, the disclosed technology provides systems and methods that can dramatically shorten the total sequencing time and reduce the number of reagents used in next generation sequencing workflows. In addition, sequencing yield per flow cell area may be increased. In some embodiments, the disclosed method enables simultaneous sequencing of two or more polynucleotide sequence portions without the need for the signals generated from the different portions to be separately detectable given the configuration of the portions and the sequencing equipment. It is therefore possible to simultaneously sequence multiple polynucleotide sequence portions that are not possible to spatially resolve, for example sequencing two polynucleotide sequence portions from a signal detected at a single sensing region (for example a single pixel of an imaging sensor) and/or from a signal obtained from a single cluster (i.e. a single contiguous cluster containing both of the two or more sequence portions), thus increasing the efficiency of the sequencing workflow. Typically, sequencing data from clusters comprising more than one polynucleotide sequence portion of interest (“polyclonal clusters”) are filtered out and are excluded from the sequencing output. Therefore, the present methods may also allow for an increase in the number of usable clusters in a given area of a substrate.

In some embodiments, the primer for sequencing a first sequence portion and the primer for sequencing a second sequence portion are annealed/hybridized to the molecules in the same reaction step to reduce chemical reaction steps, thus saving time and increasing the efficiency of sequencing-by-synthesis (SBS) workflows. Then, both sequence portions may be read-out through SBS chemistry cycles in the same reaction run.

In some embodiments, in order to separate the signals received from the dye-labeled nucleobases hybridized to each sequence portion, the signal from one of the portions is diminished, e.g., by 50%, in comparison to the signal generated by the other portion. In one example, the difference in signal intensity may be achieved by blocking the addition of labeled nucleobases to some of the primers. For example, half of the primers which bind to a first portion may be blocked so that no fluorescent nucleotides can be added during the sequencing reactions. Thus, the overall intensity of the nucleobases added to the first portion will be 50% lower than the intensity of the nucleobases added to a second portion in this example. By reviewing not only the wavelength of light emitted from the dyes from each nucleic acid cluster on the flow cell, but also the intensity of that light, the labeled nucleobase hybridized to the first portion can be distinguished from the labeled nucleobase hybridized to the second portion. This will be discussed more completely in the sections below. In another example, the difference in signal intensity may be achieved by selectively controlling the number of copies of a first sequence portion relative to the number of copies of a second sequence portion. For example, the number of copies of the second sequence portion may be lower, e.g. by 50%, in comparison to the number of copies of the first sequence portion.

In some embodiments, the disclosed technology comprises obtaining sequence information using Illumina's sequencing-by-synthesis and reversible terminator-based sequencing chemistry with removable fluorescent dyes (e.g., as described in Bentley et al., Nature 6:53-59 [2009]). Short sequence reads of about tens to a few hundred base pairs may be aligned against a reference genome and unique mapping of the short sequence reads to the reference genome may be identified. Further details regarding the sequencing-by-synthesis and dye labeling methods which can be used by the disclosed technology are described in U.S. Patent Application Publication Numbers 2007/0166705, 2006/0188901, 2006/0240439, 2006/0281109, 2005/0100900, 2013/0079232, U.S. Pat. No. 7,057,026, PCT Application Publication Numbers WO 2005/065814, WO 2006/064199, WO 2007/010251, and WO 2018/165099, U.S. patent application Ser. No. 17/338,590, U.S. Pat. Nos. 7,601,499, 9,267,173, and U.S. Patent Publication No. 2012/0053063, the disclosures of which are incorporated herein by reference in their entireties.

### Example Sequencer

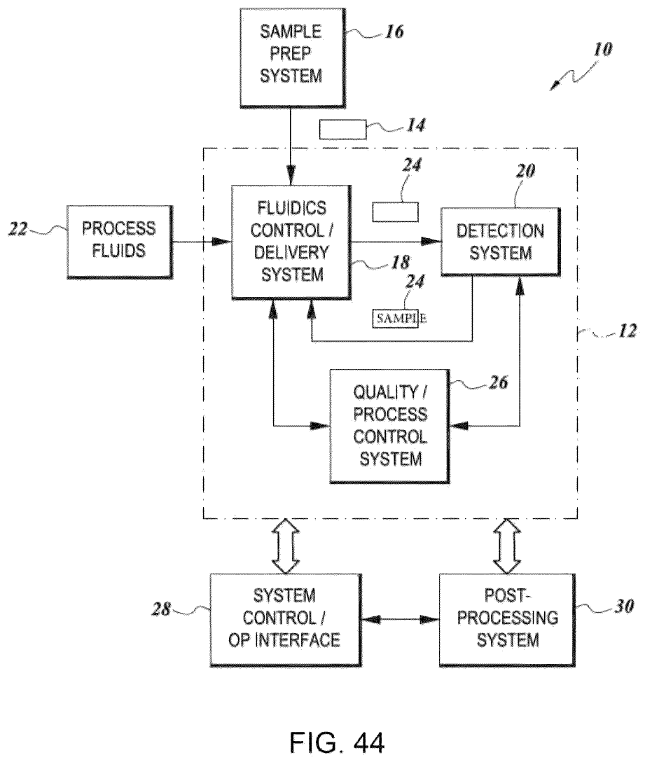

Referring to FIG. 44, a diagrammatical representation of an example sequencing system 10 is illustrated as including a sequencer 12 designed to determine sequences of genetic material of a sample 14. The sequencer may function in a variety of manners, and based upon a variety of techniques, including sequencing by primer extension using labeled nucleotides, as in a presently contemplated embodiment, as well as other sequencing techniques such as sequencing by ligation or pyrosequencing. In some embodiments, the sequencer 12 progressively moves samples through reaction cycles and imaging cycles to progressively build oligonucleotides by binding nucleotides to templates at individual sites on the sample. In some embodiments, the sample may be prepared by a sample preparation system 16. This process may include amplification of fragments of DNA or RNA on a support to create a multitude of sites of DNA or RNA fragments the sequence of which are determined by the sequencing process. Exemplary methods for producing sites of amplified nucleic acids suitable for sequencing include, but are not limited to, rolling circle amplification (RCA) (Lizardi et al., Nat. Genet. 19:225-232 (1998)), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28: E87 (2000); Pemov et al., Nucl. Acids Res. 33: e11 (2005); or U.S. Pat. No. 5,641,658), polony generation (Mitra et al., Proc. Natl. Acad. Sci. USA 100:5926-5931 (2003); Mitra et al., Anal. Biochem. 320:55-65 (2003)), or clonal amplification on beads using emulsions (Dressman et al., Proc. Natl. Acad. Sci. USA 100:8817-8822 (2003)) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002)), each of the aforementioned publications is incorporated herein by reference. The sample preparation system 16 may dispose the sample, which may be in the form of an array of sites, in a sample container for processing and imaging.

In some embodiments, the sequencer 12 includes a fluidics control/delivery system 18 and a detection system 20. The fluidics control/delivery system 18 may receive a plurality of process fluids as indicated by reference numeral 22, for circulation through the sample containers of the samples in process, designated by reference numeral 24. As will be appreciated by those skilled in the art, the process fluids may vary depending upon the particular stage of sequencing. For example, in sequencing-by-synthesis (SBS) using labeled nucleotides, the process fluids introduced to the sample may include a polymerase and tagged nucleotides of the four common DNA types, each nucleotide having a unique fluorescent tag and a blocking agent linked to it. The fluorescent tag allows the detection system 20 to detect which nucleotides were last added to primers hybridized to template nucleic acids at individual sites in the array, and the blocking agent prevents addition of more than one nucleotide per cycle at each site.

At other phases of the sequencing cycles, the process fluids 22 may include other fluids and reagents, such as reagents for removing extension blocks from nucleotides or cleaving nucleotide linkers to release a newly extendable primer terminus. For example, once reactions have taken place at individual sites in the array of the samples, the initial process fluid containing the tagged nucleotides may be washed from the sample in one or more flushing operations. The sample may then undergo detection, such as by the optical imaging at the detection system 20. Subsequently, reagents may be added by the fluidics control/delivery system 18 to de-block the last added nucleotide and remove the fluorescent tag from each. The fluidics control/delivery system 18 may then again wash the sample, which is then prepared for a subsequent cycle of sequencing. Exemplary fluidic and detection configurations that can be used in the methods and devices set forth herein are described in WO 07/123744, which is incorporated herein by reference. In some embodiments, such sequencing may continue until the quality of data derived from sequencing degrades due to cumulative loss of yield or until a predetermined number of cycles have been completed.

In some embodiments, the quality of samples 24 in process as well as the quality of the data derived by the system, and the various parameters used for processing the samples is controlled by a quality/process control system 26. The quality/process control system 26 may include one or more programmed processors, or general purpose or application-specific computers which communicate with sensors and other processing systems within the fluidics control/delivery system 18 and the detection system 20. A number of process parameters may be used for sophisticated quality and process control, for example, as part of a feedback loop that can change instrument operation parameters during the course of a sequencing run.

In some embodiments, the sequencer 12 also communicates with a system control/operator interface 28 and ultimately with a post-processing system 30. The system control/operator interface 28 may include a general purpose or application-specific computer designed to monitor process parameters, acquired data, system settings, and so forth. The operator interface may be generated by a program executed locally or by programs executed within the sequencer 12. In some embodiments, these may provide visual indications of the health of the systems or subsystems of the sequencer, the quality of the data acquired, and so forth. The system control/operator interface 28 may also permit human operators to interface with the system to regulate operation, initiate and interrupt sequencing, and any other interactions that may be desired with the system hardware or software. For instance, the system control/operator interface 28 may automatically undertake and/or modify steps to be performed in a sequencing procedure, without input from a human operator. Alternatively or additionally, the system control/operator interface 28 may generate recommendations regarding steps to be performed in a sequencing procedure and display these recommendations to the human operator. This mode may allow for input from the human operator before undertaking and/or modifying steps in the sequencing procedure. In addition, the system control/operator interface 28 may provide an option to the human operator allowing the human operator to select certain steps in a sequencing procedure to be automatically performed by the sequencer 12 while requiring input from the human operator before undertaking and/or modifying other steps. In any event, allowing both automated and operator interactive modes may provide increased flexibility in performing the sequencing procedure. In addition, the combination of automation and human-controlled interaction may further allow for a system capable of creating and modifying new sequencing procedures and algorithms through adaptive machine learning based on the inputs gathered from human operators.

The post-processing system 30 may further include one or more programmed computers that receive detected information, which may be in the form of pixilated image data and derive sequence data from the image data. The post-processing system 30 may include image recognition algorithms which distinguish between colors of dyes (e.g., fluorescent emission spectra of dyes) attached to nucleotides that bind at individual sites as sequencing progresses (e.g., by analysis of the image data encoding specific colors and/or intensities), and logs the sequence of the nucleotides at the individual site locations. Progressively, then, the post-processing system 30 may build sequence lists for the individual sites of the sample array which can be further processed to establish genetic information for extended lengths of material by various bioinformatics algorithms.

The sequencing system 10 may be configured to handle individual samples or may be designed for higher throughput in a manner in which multiple stations are provided for the delivery of reagents and other fluids, and for detection of progressively building sequences of nucleotides. Further details can be found in U.S. Pat. No. 9,797,012, which is incorporated herein by reference.

Samples may be removed from processing, reprocessed, and scheduling of such processing may be altered in real time, particularly where the fluidics control system 18 or the quality/process control system 26 detect that one or more operations were not performed in an optimal or desired manner. In embodiments wherein a sample is removed from the process or experiences a pause in processing that is of a substantial duration, the sample can be placed in a storage state. Placing the sample in a storage state can include altering the environment of the sample or the composition of the sample to stabilize biomolecule reagents, biopolymers or other components of the sample. Exemplary methods for altering the sample environment include, but are not limited to, reducing temperature to stabilize sample constituents, addition of an inert gas to reduce oxidation of sample constituents, and removing from a light source to reduce photobleaching or photodegradation of sample constituents.

Exemplary methods of altering sample composition include, without limitation, adding stabilizing solvents such as antioxidants, glycerol and the like, altering pH to a level that stabilizes enzymes, or removing constituents that degrade or alter other constituents. In addition, certain steps in the sequencing procedure may be performed before removing the sample from processing. For instance, if it is determined that the sample should be removed from processing, the sample may be directed to the fluidics control/delivery system 18 so that the sample may be washed before storage. These steps may be taken to ensure that no information from the sample is lost.

Moreover, sequencing operations may be interrupted by the sequencer 12 at any time upon the occurrence of certain predetermined events. These events may include, without limitation, unacceptable environmental factors such as undesirable temperature, humidity, vibrations or stray light; inadequate reagent delivery or hybridization; unacceptable changes in sample temperature; unacceptable sample site number/quality/distribution; decayed signal-to-noise ratio; insufficient image data; and so forth. It should be noted that the occurrence of such events need not require interruption of sequencing operations. Rather, such events may be factors weighed by the quality/process control system 26 in determining whether sequencing operations should continue. For example, if an image of a particular cycle is analyzed in real time and shows a low signal for that optical channel, the image can be re-exposed using a longer exposure time, or have a particular chemical treatment repeated. If the image shows a bubble in a flow cell, the instrument can automatically flush more reagent to remove the bubble, then re-record the image. If the image shows low signal for a particular optical channel in one cycle due to a fluidics problem, the instrument can automatically halt scanning and reagent delivery for that particular optical channel, thus saving on analysis time and reagent consumption.

Although the system has been exemplified above with regard to a system in which a sample interfaces with different stations by physical movement of the sample, it will be understood that the principles set forth herein are also applicable to a system in which the steps occurring at each station are achieved by other means not requiring movement of the sample. For example, reagents present at the stations can be delivered to a sample by means of a fluidic system connected to reservoirs containing the various reagents. Similarly, an optics system can be configured to detect a sample that is in fluid communication with one or more reagent stations. Thus, detection steps can be carried out before, during or after delivery of any particular reagent described herein. Accordingly, samples can be effectively removed from processing by discontinuing one or more processing steps, be it fluid delivery or optical detection, without necessarily physically removing the sample from its location in the device.

Disclosed systems can be used to continuously sequence nucleic acids in a plurality of different samples. Disclosed systems can be configured to include an arrangement of samples and an arrangement of stations for carrying out sequencing steps. The samples in the arrangement of samples can be placed in a fixed order and at fixed intervals relative to each other. For example, an arrangement of nucleic acid arrays can be placed along the outer edge of a circular table. Similarly, the stations can be placed in a fixed order and at fixed intervals relative to each other. For example, the stations can be placed in a circular arrangement having a perimeter that corresponds to the layout for the arrangement of sample arrays. Each of the stations can be configured to carry out a different manipulation in a sequencing protocol. The arrangements of sample arrays and stations can be moved relative to each other such that the stations carry out desired steps of a reaction scheme at each reaction site. The relative locations of the stations and the schedule for the relative movement can correlate with the order and duration of reaction steps in the sequencing reaction scheme such that once a sample array has completed a cycle of interacting with the full set of stations, then a single sequencing reaction cycle is complete. For example, primers that are hybridized to nucleic acid targets on an array can each be extended by addition of a single nucleotide, detected and de-blocked if the order of the stations, spacing between the stations, and rate of passage for the array corresponds to the order of reagent delivery and reaction time for a complete sequencing reaction cycle.

In accordance with the configuration set forth above, each lap (or full revolution in embodiments where a circular table is used) completed by an individual sample array can correspond to determination of a single nucleotide for each of the target nucleic acids on the array (e.g., including the steps of incorporation, imaging, cleavage and de-blocking carried out in each cycle of a sequencing run). Furthermore, several sample arrays present in the system (for example, on the circular table) concurrently move along similar, repeated laps through the system, thereby resulting in continuous sequencing by the system. Using the disclosed systems or methods, reagents can be actively delivered or removed from a first sample array in accordance with a first reaction step of a sequencing cycle while incubation, or some other reaction step in the cycle, occurs for a second sample array. Thus, a set of stations can be configured in a spatial and temporal relationship with an arrangement of sample arrays such that reactions occur at multiple sample arrays concurrently even as the sample arrays are subjected to different steps of the sequencing cycle at any given time, thereby allowing continuous and simultaneous sequencing to be performed. Such a circular system may be used when the chemistry and imaging times are disproportionate. For small flow cells that only take a short time to scan, the system may have a number of flow cells running in parallel in order to optimize the time the instrument spends acquiring data. When the imaging time and chemistry time are equal, a system that is sequencing a sample on a single flow cell spends half the time performing a chemistry cycle rather than an imaging cycle, and therefore a system that can process two flow cells could have one on the chemistry cycle and one on the imaging cycle. When the imaging time is ten-fold less than the chemistry time, the system can have ten flow cells at various stages of the chemistry process whilst continually acquiring data.

In some embodiments, the disclosed system is configured to allow replacement of a first sample array with a second sample array while the system continuously sequences nucleic acids of a third sample array. Thus, a first sample array can be individually added or removed from the system without interrupting sequencing reactions occurring at another sample array, thereby allowing continuous sequencing for the set of sample arrays. Moreover, sequencing runs of different lengths can be performed continuously and simultaneously in the system because individual sample arrays can complete a different number of laps through the system and the sample arrays can be removed or added to the system in an independent fashion such that reactions occurring at other sites are not perturbed.

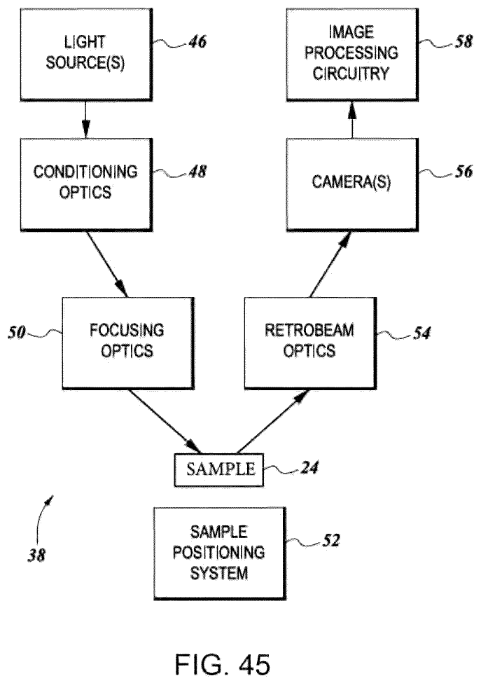

FIG. 45 illustrates an exemplary detection station 38 which can detect nucleotides added at sites of an array and can be used in conjunction with the example sequencing system of FIG. 44. As set forth above, a sample can be moved to two or more stations of the device that are located in physically different locations or alternatively one or more steps can be carried out on a sample that is in communication with the one or more stations without necessarily being moved to different locations. Accordingly, the description herein with regard to particular stations is understood to relate to stations in a variety of configurations whether or not the sample moves between stations, the stations move to the sample, or the stations and sample are static with respect to each other. In the embodiment illustrated in FIG. 45, one or more light sources 46 provide light beams that are directed to conditioning optics 48. The light sources 46 may include one or more lasers, with multiple lasers being used for detecting dyes that fluoresce at different corresponding wavelengths. The light sources may direct beams to the conditioning optics 48 for filtering and shaping of the beams in the conditioning optics. For example, in a presently contemplated embodiment, the conditioning optics 48 combine beams from multiple lasers and generate a substantially linear beam of radiation that is conveyed to focusing optics 50. The laser modules can additionally include a measuring component that records the power of each laser. The measurement of power may be used as a feedback mechanism to control the length of time an image is recorded in order to obtain a uniform exposure energy, and therefore signal, for each image. If the measuring component detects a failure of the laser module, then the instrument can flush the sample with a “holding buffer” to preserve the sample until the error in the laser can be corrected.

The sample 24 is positioned on a sample positioning system 52 that may appropriately position the sample in three dimensions, and may displace the sample for progressive imaging of sites on the sample array. In a presently contemplated embodiment, the focusing optics 50 confocally direct radiation to one or more surfaces of the array at which individual sites are located that are to be sequenced. Depending upon the wavelengths of light in the focused beam, a retrobeam of radiation is returned from the sample due to fluorescence of dyes bound to the nucleotides at each site.

The retrobeam is then returned through retrobeam optics 54 which may filter the beam, such as to separate different wavelengths in the beam, and direct these separated beams to one or more cameras 56. The cameras 56 may be based upon any suitable technology, such as including charge coupled devices that generate pixilated image data based upon photons impacting locations in the devices. In some embodiments, the cameras 56 may include CMOS sensors. In some embodiments, the cameras 56 may include one or more point-and-shoot cameras. In some embodiments, the cameras 56 may include one or more time delay and integration (TDI) cameras. The cameras generate image data that is then forwarded to image processing circuitry 58. In some embodiments, the processing circuitry 58 may perform various operations, such as analog-to-digital conversion, scaling, filtering, and association of the data in multiple frames to appropriately and accurately image multiple sites at specific locations on the sample. The image processing circuitry 58 may store the image data, and may ultimately forward the image data to the post-processing system 30 where sequence data can be derived from the image data. Example detection devices that can be used at a detection station include, for example, those described in US 2007/0114362 (U.S. patent application Ser. No. 11/286,309) and WO 07/123744, each of which is incorporated herein by reference.

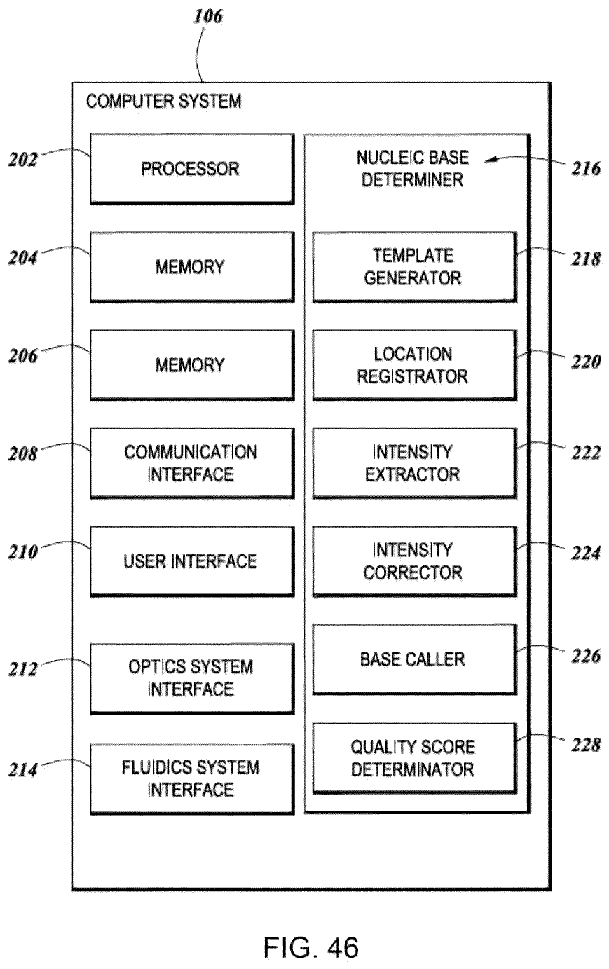

A computer system 106 as illustrated in FIG. 46 may be used to implement the system control/operator interface 28 and the post-processing system 30 of the example sequencing system 10 in FIG. 44. As shown in FIG. 46, the computer system 106 can include functionalities for controlling optics/fluidics systems and determining nucleobase sequences of polynucleotides.

In one embodiment, the computer system 106 includes a processor 202 that is in electrical communication with a memory 204, a storage 206, and a communication interface 208. The processor 202 can be configured to execute instructions that cause the fluidics system 104 to supply reagents to the flow cell 114 during sequencing reactions. The processor 202 can execute instructions that control the light source 120 of the optics system 102 to generate light at around a predetermined wavelength. The processor 202 can execute instructions that control the detector 126 of the optics system 102 and receive data from the detector 126. The processor 202 can execute instructions to process data, for example fluorescent images, received from the detector 126 and to determine the nucleotide sequences of polynucleotides based on the data received form the detector 126. The memory 204 can be configured to store instructions for configuring the processor 202 to perform the functions of the computer system 106 when the sequencing system 100 is powered on. When the sequencing system 100 is powered off, the storage 206 can store the instructions for configuring the processor 202 to perform the functions of the computer system 106. The communication interface 208 can be configured to facilitate the communications between the computer system 106, the optics system 102, and the fluidics system 104.

The computer system 106 can include a user interface 210 configured to communicate with a display device (not shown) for displaying the sequencing results of the sequencing system 100. The user interface 210 can be configured to receive inputs from users of the sequencing system 100. An optics system interface 212 and a fluidics system interface 214 of the computer system 106 can be configured to control the optics system 102 and the fluidics system 104 through communication links (not shown). For example, the optics system interface 212 can communicate with the computer interface 110 of the optics system 102 through a communication link 108a.

The computer system 106 can include a nucleic base determiner 216 configured to determine the nucleotide sequence of polynucleotides using the data received from the detector 126. The nucleic base determiner 216 can include one or more of: a template generator 218, a location registrator 220, an intensity extractor 222, an intensity corrector 224, a base caller 226, and a quality score determiner 228. The template generator 218 can be configured to generate a template of the locations of polynucleotide clusters in the flow cell 114 using the fluorescent images captured by the detector 126. The location registrator 220 can be configured to register the locations of polynucleotide clusters in the flow cell 114 in the fluorescent images captured by the detector 126 based on the location template generated by the template generator 218. The intensity extractor 222 can be configured to extract intensities of the fluorescent emissions from the fluorescent images to generate extracted intensities. For example, the peak intensity value found in a diffraction-limited spot of a DNA cluster may be extracted from the image and used to represent the signal of the DNA cluster. For another example, the total intensity included within a diffraction-limited spot of a DNA cluster may be extracted from the image and used to represent the signal of the DNA cluster. Alternatively, the intensity estimate can be made through the use of equalization and channel estimation.

The intensity corrector 224 can be configured to reduce or eliminate noise or aberration inherent in the sequencing reaction or optical system. For example, intensity may be influenced by laser intensity fluctuation, DNA cluster shape/size variation, uneven illumination, optical distortions or aberrations, and/or phasing/pre-phasing that occur in the DNA clusters. In some embodiments, the intensity corrector 224 can phase correct or pre-phase correct extracted intensities. In some embodiments, the intensity corrector 224 can normalize extracted fluorescence intensities to reduce or eliminate the effect of DNA cluster size variation. For example, each DNA template may contain the same calibration oligonucleotide. Thus, the extracted fluorescence intensity of a cluster obtained from sequencing a known nucleotide in the calibration oligonucleotide can be used as a normalization factor for that cluster. The intensity corrector 224 can divide the extracted fluorescence intensities of that cluster obtained from sequencing nucleotides in other regions of the DNA template by the normalization factor to obtain the normalized extracted fluorescence intensities. The base caller 226 can be configured to determine the nucleobases of a polynucleotide from the corrected intensities. The bases of a polynucleotide determined by the base caller 226 can be associated with quality scores determined by the quality score determiner 228. Quality scoring refers to the process of assigning a quality score to each base call. To evaluate the quality of a base call from a sequencing read, example processes can include calculating a set of predictor values for the base call and using the predictor values to look up a quality score in a quality table. The quality score can be presented in any suitable format that allows a user to determine the probability of error of any given base call. In some embodiments, the quality score is presented as a numerical value. For example, the quality score can be quoted as QXX where the XX is the score and it means that that particular call has a probability of error of 10^{-XX/10}. Thus, as an example, Q30 equates to an error rate of 1 in 1000, or 0.1% and Q40 equates to an error rate of 1 in 10,000 or 0.01%. The error rate can be calculated using a control nucleic acid. Additionally, some metrics displays can include the error rate on a per-cycle basis. In some embodiments, the quality table is generated using on a calibration data set, the calibration set being representative of run and sequence variability. Further details of the computations that can be performed by the nucleic base determiner, calculation of error rate and quality score may be found in U.S. Pat. No. 8,392,126, U.S. Patent Application Publication Numbers 2020/0080142 and 2012/0020537, each of which is incorporated by reference herein in its entirety. While nucleic base determiner 216 is shown as part of computer system 106 in FIG. 46, it will be appreciated that nucleic base determiner 216 may be a separate computing device from the other components shown in FIG. 46 such that nucleic base determiner 216 may receive and process image data in a computing device that is different to a computing device that provides optics and fluidics control.

### Clusters

FIGS. 47A-4B each illustrate a respective plurality of polynucleotide molecules 400 comprising multiple copies of two polynucleotide sequence portions of interest 401a, 401b for base calling simultaneously based upon a single combined signal obtained from the two portions according to the present methods. For example, the plurality of polynucleotide molecules 400 illustrated in FIGS. 47A and 47B may be configured on a substrate 410 such that light emissions from the plurality of polynucleotide molecules are detected by a single sensing portion (for example a single pixel of an imaging sensor 420). Additionally or alternatively, the plurality of polynucleotide molecules 400 may comprise a single cluster (i.e. a single contiguous cluster containing both of the two or more sequence portions 401a, 401b) such that light emissions from each of the two respective portions cannot be spatially resolved. The substrate 410 may be a flow cell, which may be patterned or unpatterned. In one example, the substrate 410 may be a patterned flow cell comprising a number of discrete nanowells 411, with each well containing polynucleotide molecules comprising two or more polynucleotide sequence portions for sequencing and each well having a single respective sensor associated with the well. Because each a single sensor is associated with the well, signals from the two or more portions of interest cannot be resolved, irrespective of whether the different portions (or respective clusters) are spatially resolved within the well. Two or more polynucleotide sequence portions of interest contained within a single well in this way is sometimes referred to herein as a “cluster” irrespective of whether the different portions are spatially resolved in the well given that light emissions from such a well form a single combined signal.

In one example, as shown in FIG. 47A, the first and second sequence portions 401a, 401b are present in different polynucleotide molecules 400. In the example shown in FIG. 47B, the first and second sequence portions 401a, 401b are present as respective portions of the same molecules 400. As described in detail below, by diminishing the signal from one of the portions 401b relative to the other of the portions 401a it is possible to separate the signals received from the dye-labeled nucleobases hybridized to each portion. FIGS. 47A and 47B each illustrate a respective way in which the signal intensity from the first portion 401a may be modified relative to the signal intensity from the second portion 401b (i.e. in which the signal intensity of one portion may be diminished relative to the signal intensity of the other portion). In the example illustrated in FIG. 47A, the number of first portions 401a relative to the number of second portions 401b is uneven, with one second portion for each two first portions). In the example illustrated in FIG. 47B, the number of first portions 401a and second portions 401b is the same, however some of the primers 402b used to sequence the second portions are blocked such that during a sequencing run, an uneven number of first portions relative to second portions emit light. While blocking is illustrated with respect to FIG. 4B, in which first and second sequence portions 401a, 401b are present as respective portions of the same polynucleotide molecules 400, it will be appreciated that blocking can also be used to diminish the signal from one sequence portion relative to another sequence portion where first and second portions are present in different polynucleotide molecules. Various ways of providing a plurality of polynucleotide molecules 400 comprising multiple copies of two polynucleotide sequence portions of interest 401a, 401b in which a signal intensity of one polynucleotide sequence portion is greater relative to the other polynucleotide sequence portion are outlined below.

### Differential Signal Intensity

Both the first and second sequence portions 401a, 401b in the cluster can be sequenced simultaneously using first primers 402a specific to the first portion 401a, or to a region 403a adjacent to the first portion, and second primers 402b specific to the second portion 401b, or to a region 403b adjacent to the second portion, in the same reaction run. For example, the first and second sequence portions 401a, 401b may be flanked at one or both ends by respective primer binding sites 403a, 403b having a known sequence. Sequencing primers 402a, 402b specific to the different primer binding sites 403a, 403b can therefore be designed and used for simultaneous sequencing of the two sequence portions 401a, 401b.

As described above, a single combined signal may be obtained from the two polynucleotide sequence portions of interest 401a, 401b according to the present methods. For example, the plurality of polynucleotide molecules 400 may be configured on the flow cell 410 such that light emissions from the plurality of polynucleotide molecules are detected by a single sensing portion 420. Alternatively or additionally, the plurality of template polynucleotides may comprise a single cluster such that light emissions from each of the respective polynucleotide sequence portions cannot be spatially resolved.

Since the fluorescent signal associated with the extended first portion sequencing primers 402a and the fluorescent signal associated with the extended second portion sequencing primers 402b is combined, the signals may not be optically resolved. Therefore, methods for determining whether a fluorescent signal is associated with the extended first portion sequencing primers 402a or the extended second portion sequencing primers 402b are needed, at least when the dye-labeled nucleotide analogs at the extended first portion sequencing primers are not the same as the dye-labeled nucleotide analogs at the extended second portion sequencing primers (e.g., when “A”s are added at the first sequence portion 401a and “C”s are added at the second sequence portion 401b), in order to correctly determine the nucleic acid sequences of both the first and second portions.

In some embodiments, whether a fluorescent signal is associated with the first sequence portion 401a or the second sequence portion 401b can be determined by using distinguishable levels of signal intensity. In particular, the polynucleotide molecules 400 may be selectively processed such that an intensity of the light emissions associated with respective nucleobases in each of the different sequence portions of interest is different.

It will be appreciated that for dye labeling schemes which include an unlabeled or “dark” base (e.g., G), the signal intensity will be zero for both portions. Similarly, the signal associated with a nucleobase may be zero for an image captured in one particular channel of a base calling cycle. Accordingly, it will be appreciated that the polynucleotide molecules may be selectively processed such that, for signals of non-zero intensity, an intensity of the signals obtained based upon respective nucleobases of the different sequence portions is different.

### Computer Programs and Products

In other embodiments, methods as described herein may be performed by a computer. In other words, a computer may contain instructions to conduct the methods of preparing polynucleotide sequences for detection of mismatched base pairs as described herein, and as such the methods as described herein may be computer-implemented.

Accordingly, in another aspect of the invention, there is provided a data processing device comprising means for carrying out the methods as described herein.

The data processing device may be a polynucleotide sequencer.

The data processing device may comprise reagents used for synthesis methods as described herein.

The data processing device may comprise a solid support, such as a flow cell, bead or well.

In another aspect of the invention, there is provided a computer program product comprising instructions which, when the program is executed by a processor, cause the processor to carry out the methods as described herein.

In another aspect of the invention, there is provided a computer-readable storage medium comprising instructions which, when executed by a processor, cause the processor to carry out the methods as described herein.

In another aspect of the invention, there is provided a computer-readable data carrier having stored thereon the computer program product as described herein.

In another aspect of the invention, there is provided a data carrier signal carrying the computer program product as described herein.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Computer-executable instructions may be stored in a (transitory or non-transitory) computer readable storage medium (e.g., memory, storage system, etc.) storing code, or computer readable instructions. In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

As used herein, a “flow cell” can include a device having a lid extending over a reaction structure to form a flow channel therebetween that is in communication with a plurality of reaction sites of the reaction structure, and can include a detection device that is configured to detect designated reactions that occur at or proximate to the reaction sites. A flow cell may include a solid-state light detection or “imaging” device, such as a Charge-Coupled Device (CCD) or Complementary Metal-Oxide Semiconductor (CMOS) (light) detection device. As one specific example, a flow cell may be configured to fluidically and electrically couple to a cartridge (having an integrated pump), which may be configured to fluidically and/or electrically couple to a bioassay system. A cartridge and/or bioassay system may deliver a reaction solution to reaction sites of a flow cell according to a predetermined protocol (e.g., sequencing-by-synthesis), and perform a plurality of imaging events. For example, a cartridge and/or bioassay system may direct one or more reaction solutions through the flow channel of the flow cell, and thereby along the reaction sites. At least one of the reaction solutions may include four types of nucleotides having the same or different fluorescent labels. The nucleotides may bind to the reaction sites of the flow cell, such as to corresponding oligonucleotides at the reaction sites. The cartridge and/or bioassay system may then illuminate the reaction sites using an excitation light source (e.g., solid-state light sources, such as light-emitting diodes (LEDs)). The excitation light may have a predetermined wavelength or wavelengths, including a range of wavelengths. The fluorescent labels excited by the incident excitation light may provide emission signals (e.g., light of a wavelength or wavelengths that differ from the excitation light and, potentially, each other) that may be detected by the light sensors of the flow cell.

Flow cells described herein may be configured to perform various biological or chemical processes. More specifically, the flow cells described herein may be used in various processes and systems where it is desired to detect an event, property, quality, or characteristic that is indicative of a designated reaction. For example, flow cells described herein may include or be integrated with light detection devices, biosensors, and their components, as well as bioassay systems that operate with biosensors. The flow cells may be configured to facilitate a plurality of designated reactions that may be detected individually or collectively. The flow cells may be configured to perform numerous cycles in which the plurality of designated reactions occurs in parallel. For example, the flow cells may be used to sequence a dense array of DNA features through iterative cycles of enzymatic manipulation and light or image detection/acquisition. As such, the flow cells may be in fluidic communication with one or more microfluidic channels that deliver reagents or other reaction components in a reaction solution to a reaction site of the flow cells. The reaction sites may be provided or spaced apart in a predetermined manner, such as in a uniform or repeating pattern. Alternatively, the reaction sites may be randomly distributed. Each of the reaction sites may be associated with one or more light guides and one or more light sensors that detect light from the associated reaction site. In one example, light guides include one or more filters for filtering certain wavelengths of light. The light guides may be, for example, an absorption filter (e.g., an organic absorption filter) such that the filter material absorbs a certain wavelength (or range of wavelengths) and allows at least one predetermined wavelength (or range of wavelengths) to pass therethrough. In some flow cells, the reaction sites may be located in reaction recesses or chambers, which may at least partially compartmentalize the designated reactions therein.

As used herein, the term “spot radius” or “cluster radius” refers to a defined radius which encompasses a diffraction-limited spot or a cluster of signals. Accordingly, by defining a cluster radius as larger or smaller, a greater number of signals can fall within the radius for subsequent ordering and selection. A cluster radius can be defined by any distance measure, such as pixels, meters, millimeters, or any other useful measure of distance.

As used herein, a “signal” refers to a detectable event such as an emission, such as light emission, for example, in an image. Thus, in some embodiments, a signal can represent any detectable light emission that is captured in an image (i.e., a “spot”). Thus, as used herein, “signal” can refer to an actual emission from a feature of the specimen, or can refer to a spurious emission that does not correlate to an actual feature. Thus, a signal could arise from noise and could be later discarded as not representative of an actual feature of a specimen.

As used herein, an “intensity” of an emitted light refers to the intensity of the light transferred per unit area, where the area is measured on the plane perpendicular to the direction of propagation of the light ray, and where the intensity is the amount of energy transferred per unit time. In some embodiments, signal “strength”, “amplitude”, “magnitude” or “level” may be used synonymously with signal intensity. In some embodiments, an image taken by a detector is approximately or proportional to an intensity map integrated over some amount of time. In some embodiments, the signal of a diffraction-limited spot of a DNA cluster is extracted from the image as the total intensity included in the spot, up to a factor of the integration time. For example, the signal of a DNA cluster may be defined as the intensity included within the spot radius of the DNA cluster, up to a factor of the integration time. In other embodiments, the peak intensity value found within the spot radius may be used to represent the signal of the DNA cluster, up to a factor of the integration time.

As used herein, the process of aligning the template of signal positions onto a given image is referred to as “registration”, and the process for determining an intensity value or an amplitude value for each signal in the template for a given image is referred to as “intensity extraction”. For registration, the methods and systems provided herein may take advantage of the random nature of signal clump positions by using image correlation to align the template to the image.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

The polymerase used is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentRR DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, Tfl DNA Polymerase, Tth DNA Polymerase, RepliPHI Phi29 Polymerase, Tli DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and U.S. Pat. No. 6,395,524 (incorporated by reference). These polymerases include wild-type, mutant isoforms, and genetically engineered variants. “Encode” or “parse” are verbs referring to transferring from one format to another, and refers to transferring the genetic information of target template base sequence into an arrangement of reporters.

Nucleosides and nucleotides may be labeled at sites on the sugar or nucleobase. A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. A linker group may be used to covalently attach a dye to the nucleoside or nucleotide. As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

Various different types of linkers having different lengths and chemical properties can be used. The term “linker” encompasses any moiety that is useful to connect one or more molecules or compounds to each other, to other components of a reaction mixture, and/or to a reaction site. For example, a linker can attach a reporter molecule or “label” (e.g., a fluorescent dye) to a reaction component. In certain embodiments, the linker is a member selected from substituted or unsubstituted alkyl (e.g., a 2-5 carbon chain), substituted or unsubstituted heteroalkyl, substituted or unsubstituted aryl, substituted or unsubstituted heteroaryl, substituted or unsubstituted cycloalkyl, and substituted or unsubstituted heterocycloalkyl. In one example, the linker moiety is selected from straight—and branched carbon-chains, optionally including at least one heteroatom (e.g., at least one functional group, such as ether, thioether, amide, sulfonamide, carbonate, carbamate, urea and thiourea), and optionally including at least one aromatic, heteroaromatic or non-aromatic ring structure (e.g., cycloalkyl, phenyl). In certain embodiments, molecules that have trifunctional linkage capability are used, including, but are not limited to, cynuric chloride, mealamine, diaminopropanoic acid, aspartic acid, cysteine, glutamic acid, pyroglutamic acid, S-acetylmercaptosuccinic anhydride, carbobenzoxylysine, histine, lysine, serine, homoserine, tyrosine, piperidinyl-1,1-amino carboxylic acid, diaminobenzoic acid, etc. In certain specific embodiments, a hydrophilic PEG (polyethylene glycol) linker is used.

In certain embodiments, linkers are derived from molecules which comprise at least two reactive functional groups (e.g., one on each terminus), and these reactive functional groups can react with complementary reactive functional groups on the various reaction components or used to immobilize one or more reaction components at the reaction site. “Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like.

Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps. In some embodiments, one or more dye or label molecules may attach to the nucleotide base by non-covalent interactions, or by a combination of covalent and non-covalent interactions via a plurality of intermediating molecules. In one example, a nucleotide or a nucleotide analog, being newly incorporated by the polymerase synthesizing from a target polynucleotide, is initially unlabeled. Then, one or more fluorescent labels may be introduced to the nucleotide or nucleotide analog by binding to labeled affinity reagents containing one or more fluorescent dyes. Uses of unlabeled nucleotides and affinity reagents in sequencing by synthesis have been disclosed in U.S. Publication No. 2013/0079232, which is incorporated herein by reference. For example, one, two, three or each of the four different types of nucleotides (e.g., dATP, dCTP, dGTP and dTTP or dUTP) in the reaction mix may be initially unlabeled. Each of the four types of nucleotides (e.g., dNTPs) may have a 3′ hydroxy blocking group to ensure that only a single base can be added by a polymerase to the 3′ end of a copy polynucleotide being synthesized from the target polynucleotide. After incorporation of an unlabeled nucleotide, an affinity reagent may be then introduced that specifically binds to the incorporated dNTP to provide a labeled extension product comprising the incorporated dNTP.

The affinity reagent may be designed to specifically bind to the incorporated dNTP via antibody-antigen interaction or ligand-receptor interaction, for example. The dNTP may be modified to include a specific antigen, which will pair with a specific antibody included in the corresponding affinity reagent. Thus, one, two, three or each of the four different types of nucleotides may be specifically labeled via their corresponding affinity reagents. In some embodiments, the affinity reagents may include small molecules or protein tags that may bind to a hapten moiety of the nucleotide (such as streptavidin-biotin, anti-DIG and DIG, anti-DNP and DNP), antibody (including but not limited to binding fragments of antibodies, single chain antibodies, bispecific antibodies, and the like), aptamers, knottins, affimers, or any other known agent that binds an incorporated nucleotide with a suitable specificity and affinity. In some embodiments, the hapten moiety of the unlabeled nucleotide may be attached to the nucleobase through a cleavable linker, which may be cleaved under the same reaction condition as that for removing 3′ blocking group. In some embodiments, one affinity reagent may be labeled with multiple copies of the same fluorescent dye, for example, 1, 2, 3, 4, 5, 6, 8, 10, 12, 15 copies of the same dye. In some embodiments, each affinity reagent may be labeled with a different number of copies of the same fluorescent dye. In some embodiments, a first affinity reagent may be labeled with a first number of a first fluorescent dye, a second affinity reagent may be labeled with a second number of a second fluorescent dye, a third affinity reagent may be labeled with a third number of a third fluorescent dye, and a fourth affinity reagent may be labeled with a fourth number of a fourth fluorescent dye. In some embodiments, each affinity reagent may be labeled with a distinct combination of one of more types of dye, where each type of dye has a certain copy number. In some embodiments, different affinity reagents may be labeled with different dyes that can be excited by the same light source, but each dye will have a distinguishable fluorescent intensity or a distinguishable emission spectrum. In some embodiments, different affinity reagents may be labeled with the same dye in different molar ratios to create measurable differences in their fluorescent intensities.

A nucleotide analog may be attached to or associated with one or more photo-detectable labels to provide a detectable signal. In some embodiments, a photo-detectable label may be a fluorescent compound, such as a small molecule fluorescent label. Fluorescent molecules (fluorophores) suitable as a fluorescent label include, but are not limited to: 1,5 IAEDANS; 1,8-ANS; 4-methylumbelliferone; 5-carboxy-2,7-dichlorofluorescein; 5-carboxyfluorescein (5-FAM); fluorescein amidite (FAM); 5-carboxynapthofluorescein; tetrachloro-6-carboxyfluorescein (TET); hexachloro-6-carboxyfluorescein (HEX); 2,7-dimethoxy-4,5-dichloro-6-carboxyfluorescein (JOE); VIC®; NED™; tetramethylrhodamine (TMR); 5-carboxytetramethylrhodamine (5-TAMRA); 5-HAT (Hydroxy Tryptamine); 5-hydroxy tryptamine (HAT); 5-ROX (carboxy-X-rhodamine); 6-carboxyrhodamine 6G; 6-JOE; Light Cycler® red 610; Light Cycler® red 640; Light Cycler® red 670; Light Cycler® red 705; 7-amino-4-methylcoumarin; 7-aminoactinomycin D (7-AAD); 7-hydroxy-4-methylcoumarin; 9-amino-6-chloro-2-methoxyacridine; 6-methoxy-N-(4-aminoalkyl) quinolinium bromide hydrochloride (ABQ); Acid Fuchsin; ACMA (9-amino-6-chloro-2-methoxyacridine); Acridine Orange; Acridine Red; Acridine Yellow; Acriflavin; Acriflavin Feulgen SITSA; AFPs-AutoFluorescent Protein-(Quantum Biotechnologies); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamine-IsoThioCyanate); True Blue; TruRed; Ultralite; Uranine B; Uvitex SFC; WW 781; X-Rhodamine; X-Rhodamine-5-(and-6)-Isothiocyanate (5 (6)-XRITC); Xylene Orange; Y66F; Y66H; Y66 W; YO-PRO-1; YO-PRO-3; YOYO-1; interchelating dyes such as YOYO-3, Sybr Green, Thiazole orange; members of the Alexa Fluor® dye series (from Molecular Probes/Invitrogen) which cover a broad spectrum and match the principal output wavelengths of common excitation sources such as Alexa Fluor 350, Alexa Fluor 405, 430, 488, 500, 514, 532, 546, 555, 568, 594, 610, 633, 635, 647, 660, 680, 700, and 750; members of the Cy Dye fluorophore series (GE Healthcare), also covering a wide spectrum such as Cy3, Cy3B, Cy3.5, Cy5, Cy5.5, Cy7; members of the Oyster® dye fluorophores (Denovo Biolabels) such as Oyster-500, -550, -556, 645, 650, 656; members of the DY-Labels series (Dyomics), for example, with maxima of absorption that range from 418 nm (DY-415) to 844 nm (DY-831) such as DY-415, -495, -505, -547, -548, -549, -550, -554, -555, -556, -560, -590, -610, -615, -630, -631, -632, -633, -634, -635, -636, -647, -648, -649, -650, -651, -652, -675, -676, -677, -680, -681, -682, -700, -701, -730, -731, -732, -734, -750, -751, -752, -776, -780, -781, -782, -831, -480XL, -481XL, -485XL, -510XL, -520XL, -521XL; members of the ATTO series of fluorescent labels (ATTO-TEC GmbH) such as ATTO 390, 425, 465, 488, 495, 520, 532, 550, 565, 590, 594, 610, 611X, 620, 633, 635, 637, 647, 647N, 655, 680, 700, 725, 740; members of the CAL Fluor® series or Quasar® series of dyes (Biosearch Technologies) such as CAL Fluor® Gold 540, CAL Fluor® Orange 560, Quasar® 570, CAL Fluor® Red 590, CAL Fluor® Red 610, CAL Fluor® Red 635, Quasar® 570, and Quasar® 670. In some embodiments, a first photo-detectable label interacts with a second photo-detectable moiety to modify the detectable signal, e.g., via fluorescence resonance energy transfer (“FRET”; also known as Förster resonance energy transfer).

The fluorescent labels utilized by the systems and methods disclosed herein can have different peak absorption wavelengths, for example, ranging from 400 nm to 800 nm. In some embodiments, the peak absorption wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak absorption wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different peak emission wavelength, for example, ranging from 400 nm to 800 nm. In some embodiments, the peak emission wavelengths of the fluorescent labels can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments the peak emission wavelengths of the fluorescent labels can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm.

The fluorescent labels can have different Stokes shift, for example, ranging from 10 nm to 200 nm. In some embodiments, the stoke shift can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the stoke shift can be at least, or at most, 10, 20, 30, 40, 50, 60, 70, 80, 90, 100, 110, 120, 130, 140, 150, 160, 170, 180, 190, or 200 nm.

In some embodiments, the distance between the peak emission wavelengths of any two fluorescent labels can vary, for example, ranging from 10 nm to 200 nm. In some embodiments, the distance between the peak emission wavelengths of any two fluorescent labels can be, or be about, 10, 20, 30, 40, 50, 60, 70, 80, 90, 100, 110, 120, 130, 140, 150, 160, 170, 180, 190, 200 nm, or a number or a range between any two of these values. In some embodiments, the distance between the peak emission wavelengths of any two fluorescent labels can be at least, or at most, 10, 20, 30, 40, 50, 60, 70, 80, 90, 100, 110, 120, 130, 140, 150, 160, 170, 180, 190, or 200 nm.

A “light source” may be any device capable of emitting energy along the electromagnetic spectrum. A light source may be a source of visible light (VIS), ultraviolet light (UV) and/or infrared light (IR). “Visible light” (VIS) generally refers to the band of electro-magnetic radiation with a wavelength from about 400 nm to about 750 nm. “Ultraviolet (UV) light” generally refers to electromagnetic radiation with a wavelength shorter than that of visible light, or from about 10 nm to about 400 nm range. “Infrared light” or infrared radiation (IR) generally refers to electromagnetic radiation with a wavelength greater than the VIS range, or from about 750 nm to about 50,000 nm. A light source may also provide full spectrum light. Light sources may output light from a selected wavelength or a range of wavelengths. In some embodiments of the invention, the light source may be configured to provide light above or below a predetermined wavelength, or may provide light within a predetermined range. A light source may be used in combination with a filter, to selectively transmit or block light of a selected wavelength from the light source. A light source may be connected to a power source by one or more electrical connectors; an array of light sources may be connected to a power source in series or in parallel. A power source may be a battery, or a vehicle electrical system or a building electrical system. The light source may be connected to a power source via control electronics (control circuit); control electronics may comprise one or more switches. The one or more switches may be automated, or controlled by a sensor, timer or other input, or may be controlled by a user, or a combination thereof. For example, a user may operate a switch to turn on a UV light source; the light source may be applied on a constant basis until it is turned off, or it may be pulsed (repeated on/off cycles) until it is turned off. In some embodiments, the light source may be switched from a continuously-on state to a pulsed state, or vice versa. In some embodiments, the light source may be configured to be brightening or darkening over time.

For operation, the light source may be connected to a power source capable of providing sufficient intensity to illuminate the sample. Control electronics may be used to switch the intensity on or off based on input from a user or some other input, and can also be used to modulate the intensity to a suitable level (e.g. to control brightness of the output light). Control electronics may be configured to turn the light source on and off as desired. Control electronics may include a switch for manual, automatic, or semi-automatic operation of the light sources. The one or more switches may be, for example, a transistor, a relay or an electromechanical switch. In some embodiments, the control circuit may further comprise an AC-DC and/or a DC-DC converter for converting the voltage from the voltage source to an appropriate voltage for the light source. The control circuit may comprise a DC-DC regulator for regulation of the voltage. The control circuit may further comprise a timer and/or other circuitry elements for applying electric voltage to the optical filter for a fixed period of time following the receipt of input. A switch may be activated manually or automatically in response to predetermined conditions, or with a timer. For example, control electronics may process information such as user input, stored instructions, or the like.

One or more of a plurality of light sources may be provided. In some embodiments, each of the plurality of light sources may be the same. Alternatively, one or more of the light sources may vary. The light characteristics of the light emitted by the light sources may be the same or may vary. A plurality of light sources may or may not be independently controllable. One or more characteristic of the light source may or may not be controlled, including but not limited to whether the light source is on or off, brightness of light source, wavelength of light, intensity of light, angle of illumination, position of light source, or any combination thereof.

In some embodiments, light output from a light source may be from about 350 to about 750 nm, or any amount or range therebetween, for example from about 350 nm to about 360, 370, 380, 390, 400, 410, 420, 430 or about 450 nm, or any amount or range therebetween. In other embodiments, light from a light source may be from about 550 to about 700 nm, or any amount or range therebetween, for example from about 550 to about 560, 570, 580, 590, 600, 610, 620, 630, 640, 650, 660, 670, 680, 690 or about 700 nm, or any amount or range therebetween. In some embodiments, the wavelength of the light generated by the light source can vary, for example, ranging from 400 nm to 800 nm. In some embodiments, the wavelength of the light generated by the light source can be, or be about, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 nm, or a number or a range between any two of these values. In some embodiments, the wavelength of the light generated by the light source can be at least, or at most, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm. The light source may be capable of emitting electromagnetic waves in any spectrum. In some embodiments, the light source may have a wavelength falling between 10 nm and 100 μm. In some embodiments, the wavelength of light may fall between 100 nm to 5000 nm, 300 nm to 1000 nm, or 400 nm to 800 nm. In some embodiments, the wavelength of light may be less than, and/or equal to 10 nm, 100 nm, 200 nm, 300 nm, 400 nm, 500 nm, 600 nm, 700 nm, 800 nm, 900 nm, 1000 nm, 1100 nm, 1200 nm, 1300 nm, 1500 nm, 1750 nm, 2000 nm, 2500 nm, 3000 nm, 4000 nm, or 5000 nm.

In one example, a light source may be a light-emitting diode (LED) (e.g., gallium arsenide (GaAs) LED, aluminum gallium arsenide (AlGaAs) LED, gallium arsenide phosphide (GaAsP) LED, aluminum gallium indium phosphide (AlGalnP) LED, gallium (III) phosphide (GaP) LED, indium gallium nitride (InGaN)/gallium (III) nitride (GaN) LED, or aluminum gallium phosphide (AlGaP) LED). In another example, a light source can be a laser, for example a vertical cavity surface emitting laser (VCSEL) or other suitable light emitter such as an Indium-Gallium-Aluminum-Phosphide (InGaAIP) laser, a Gallium-Arsenic Phosphide/Gallium Phosphide (GaAsP/GaP) laser, or a Gallium-Aluminum-Arsenide/Gallium-Aluminum-Arsenide (GaAlAs/GaAs) laser. Other examples of light sources may include but are not limited to electron stimulated light sources (e.g., Cathodoluminescence, Electron Stimulated Luminescence (ESL light bulbs), Cathode ray tube (CRT monitor), Nixie tube), incandescent light sources (e.g., Carbon button lamp, Conventional incandescent light bulbs, Halogen lamps, Globar, Nernst lamp), electroluminescent (EL) light sources (e.g., Light-emitting diodes-Organic light-emitting diodes, Polymer light-emitting diodes, Solid-state lighting, LED lamp, Electroluminescent sheets Electroluminescent wires), gas discharge light sources (e.g.,

Fluorescent lamps, Inductive lighting, Hollow cathode lamp, Neon and argon lamps, Plasma lamps, Xenon flash lamps), or high-intensity discharge light sources (e.g., Carbon arc lamps, Ceramic discharge metal halide lamps, Hydrargyrum medium-arc iodide lamps, Mercury-vapor lamps, Metal halide lamps, Sodium vapor lamps, Xenon arc lamps). Alternatively, a light source may be a bioluminescent, chemiluminescent, phosphorescent, or fluorescent light source.

As used herein, an “optical channel” is a predefined profile of optical frequencies (or equivalently, wavelengths). For example, a first optical channel may have wavelengths of 500 nm-600 nm. To take an image in the first optical channel, one may use a detector which is only responsive to 500 nm-600 nm light, or use a bandpass filter having a transmission window of 500 nm-600 nm to filter the incoming light onto a detector responsive to 300 nm-800 nm light. A second optical channel may have wavelengths of 300 nm-450 nm and 850 nm-900 nm. To take an image in the second optical channel, one may use a detector responsive to 300 nm-450 nm light and another detector responsive to 850 nm-900 nm light and then combine the detected signals of the two detectors. Alternatively, to take an image in the second optical channel, one may use a bandstop filter which rejects 451 nm-849 nm light in front of a detector responsive to 300 nm-900 nm light.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

It is noted that other embodiments of this application are described in the following applications, to which this application claims priority, and the applications as a whole are incorporated by reference in their entiries herein, including PCT Application No. PCT/EP23/56641 filed on Mar. 15, 2023, which claims priority to U.S. Provisional Application No. 63/439,519 filed on Jan. 17, 2023; PCT Application No/PCT/EP23/56634 filed on Mar. 15, 2023 which claims priority to U.S. Provisional Application No. 63/439,466 filed on Jan. 17, 2023; PCT Application No. PCT/EP23/56656 filed on Mar. 15, 2023 which claims priority to U.S. Provisional Application No. 63/429,501 filed on Jan. 17, 2023; PCT Application No. PCT/EP23/56669 filed on Mar. 15, 2023 which claims priority to U.S. Provisional Application No. 63/439,415 filed on Jan. 17, 2023; PCT Application No. PCT/EP23/056626 filed on Mar. 15, 2023 which claims priority to U.S. Provisional Application No. 63/439,438 filed on Jan. 17, 2023; PCT Application No. PCT/EP23/056653 filed on Mar. 15, 2023 which claims priority to U.S. Provisional Application No. 63/439,443 filed on Jan. 17, 2023; and PCT Application No. PCT/EP23/056672 filed on Mar. 15, 2023 which claims priority to U.S. Provisional Application No. 63/439,417 filed on Jan. 17, 2023.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The term “comprising” may be considered to encompass “consisting”.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value. The term “partially” is used to indicate that an effect is only in part or to a limited extent.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

Certain embodiments will now be described by way of the following non-limiting examples.

### EXAMPLES

### Example 1—Mismatched Base Pair Analysis on NA12878 Sample Using 9 QaM

### Oligo Sequences:

Asterisk (*) indicates a phosphorothioate linkage.

Bold indicates nicking restriction site (or its complement) of Nt.BspQI, which recognises the following sequence (nicking site is indicated by arrow):

5′ . . . G C T C T T C N^{▾} . . . 3′
3′ . . . C G A G A A G N . . . 5′

[Biotin-T] indicates the following structure:

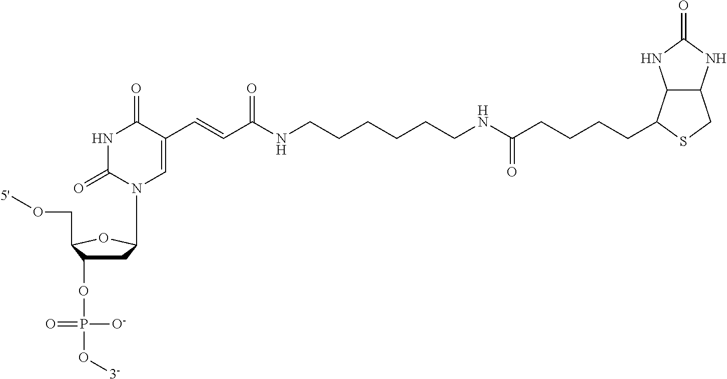

P5_BbvCI_P7 (SEQ ID NO: 7):
GCTGAGGATCTCGTATGCCGTCTTCTGCTTGUAATGATACGGCGACCACC
GAGATCTACACTCCTCAGC*T
BspQI_iSce_Loop (SEQ ID NO: 8):
GAAGAGCACACGTCTGAACTCCAGTCACTAGGGA[Biotin-T]AACAGG
GTAATCTTTCCCTACACGACGCTCTTC*T

### Adaptor Annealing:

1. A mixture of 4 μl of 100 μM P5_BbvCl_P7 oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed P5_BbvCI_P7 adaptor.

2. Separately, a mixture of 4 μl of 100 μM BspQI_iSce_Loop oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed BspQI_iSce_Loop adaptor.

3. Equal volumes of the 20 μM stock of annealed P5_BbvCI_P7 adaptor from step 1 and 20 μM stock of annealed BspQI_iSce_Loop adaptor from step 2 are mixed together, giving a stock solution with 10 μM each of annealed P5_BbvCl_P7 adaptor and annealed BspQI_iSce_Loop adaptor.

### Preparation of Library:

1. NEB Ultra II FS reagents were thawed at room temperature and kept on ice until use.

2. The Ultra II FS Enzyme mix was vortexed for 5-8 seconds prior to use and placed on ice.

3. In a 0.2 ml PCR tube on ice, 26 μl DNA (100 ng of input DNA (NA12878 sample) diluted to 26 μl with Milli-Q grade water), 7 μl of NEBNext Ultra II FS Reaction Buffer and 2 μl of NEBNext Ultra II FS Enzyme Mix were added, briefly vortexed and spun in a microcentrifuge to mix.

4. In a Thermocycler with the heated lid set to 75 C, the tubes were incubated for 5 mins at 37 C, then 30 mins at 65 C then held at 4 C.

5. The following were added to the FS reaction mixture from step 4: 30 μl of NEBNext Ultra II Ligation Master Mix, 1 μl of NEBNext Ligation Enhancer and 2.5 μl of the loop adaptors P5_BbvCI_P7 and BspQI_iSce_Loop (10 μM each) prepared from step 3 of “Adaptor annealing”.

6. The entire volume was pipetted up and down 10× to mix, followed by a brief spin in a microcentrifuge.

7. The mixture was incubated at 20 C for 15 mins in a thermocycler with the heated lid off.

8. 3 μl of USER Enzyme (NEB) was added to the ligation mix.

9. The mixture was mixed well and incubated at 37 C for 15 mins with the heated lid set to >47 C.

10. Adaptor ligated DNA was then size selected via a 0.8× SPRI (iTune beads) selection: 40 μl iTune beads (ILMN) were added to 68.5 μl of ligation reaction, mixed and incubated at RT for 5 mins.

11. The mixture was placed on a magnet for 5 mins, and the supernatant was discarded.

12. The beads were washed twice with 200 μl of 80% ethanol-200 μl 80% ethanol was added with beads on the magnet, followed by a 30 s wait, and ethanol was removed, then the wash was repeated once more.

13. The last remnants of ethanol were removed with a P10 pipette and tip.

14. Beads were then air dried for 5 mins.

15. DNA was eluted from beads with 40 μl of 0.1× TE buffer.

16. A second size selection was conducted via another 0.8× SPRI (iTune beads) selection: 20 μl iTune beads (ILMN) were added to 68.5 μl of ligation reaction, mixed and incubated at RT for 5 mins.

17. The mixture was placed on a magnet for 5 mins, and the supernatant was discarded.

18. The beads were washed twice with 200 μl of 80% ethanol-200 μl 80% ethanol was added with beads on the magnet, followed by a 30 s wait, and ethanol was removed, then the wash was repeated once more.

19. The last remnants of ethanol were removed with a P10 pipette and tip.

20. Beads were then air dried for 5 mins.

21. DNA was eluted from beads with 15 μl of 0.1× TE buffer, of which 7.5 μl was taken forward to the next step.

22. 175 μl of HT1 buffer (ILMN Hybridisation buffer) and 10 μl of HT1 washed MyOne Streptavidin T1 beads (Thermofisher) were added. The tubes were incubated on a rocker at RT for 30 mins. (This step selects for material which has the biotinylated loop adaptor, and removes the material which has the P5/P7 adaptors on both ends).

23. The tubes were placed on a magnet until the beads pelleted.

24. The beads were washed twice with 200 μl of Tagmentation Wash Buffer (TWB, Illumina).

25. The beads were then washed once with 200 μl of Resuspension Buffer (RSB, Illumina).

26. The beads were resuspended in 20 μl of Milli-Q grade water and transferred to 0.2 ml tubes for the final PCR.

27. 20 μl of beads+DNA were combined with 25 μl of Illumina Enhanced PCR Mix (EPM) and 5 μl of PPC (PCR Primer Cocktail, Illumina).

28. The mixture was amplified by PCR: cycling procedure—98 C for 3 min followed by 12 cycles of (98 C 45 s, 60 C 2 min, 68 C 2 min), then 68 C for 5 mins and then hold at 4 C.

29. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq.
1. 400 μl BspQI mix was made up-360 μl Milli-Q grade water, 40 μl of rNEB3.1 buffer (NEB) and 8 μl of Nt.BspQI (NEB were combined). The mixture was vortexed to mix and briefly spun down. The mixture was pipetted into the “EXT” position of the MiniSeq cartridge (position to the left of the Custom Primer positions).

2. The library was denatured (0.1N NaOH) and diluted to 0.5 pM final concentration in HT1 buffer according to Illumina protocol. 500 μl was loaded into the “Library” position of the MiniSeq cartridge.

3. Setup was run using MiniSeq Control Software, using a standard MiniSeq run.

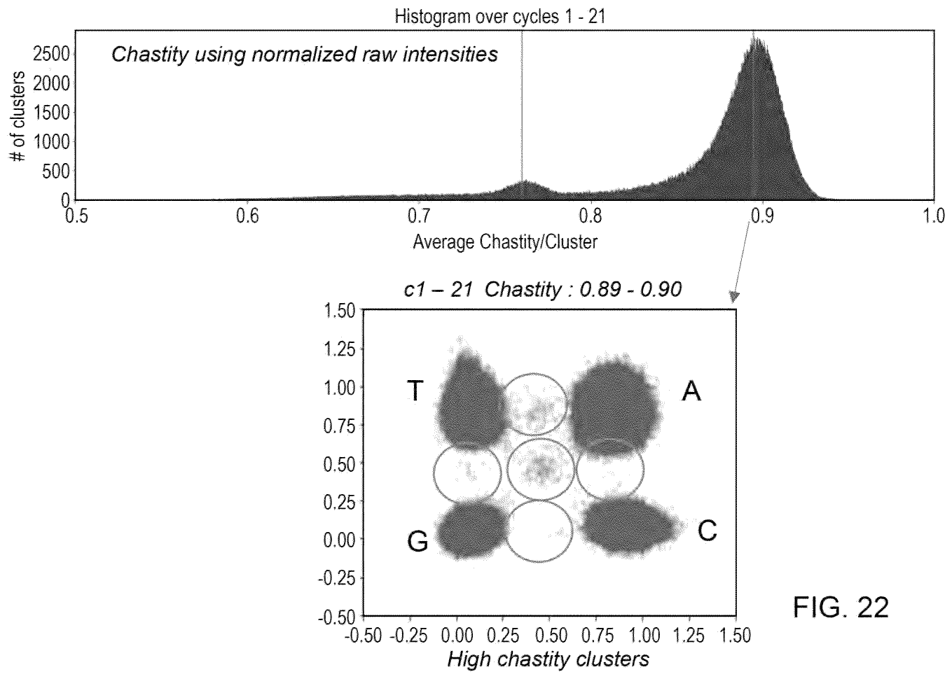

The 9 QaM results are shown in FIG. 22, where mismatched base pairs can be identified by analysing base calls that appear in the side or central clouds, rather than the four corner clouds. The centre middle cloud is one of the more populated clouds corresponding to mismatched base pairs, and this can primarily be attributed to (oxo-G)-A mismatched base pairs.

Overall, these results show that analysis can be conducted on polynucleotide sequences to identify mismatched base pairs. In particular, by enabling concurrent sequencing of the forward and reverse complement strands of the template (or reverse and forward complement strands of the template), mismatched base pairs can be identified quickly and accurately. Such a process is made viable by using the methods of preparing polynucleotide libraries as described herein.

### Example 2—Methylation Analysis on Methylated pUC 19 Sample Using 9 QaM

### Oligo Sequences:

Asterisk (*) indicates a phosphorothioate linkage.

Underline indicates 5-methylcytosine instead of cytosine (in “P5_BbvCI_P7-methylated” and “BspQI_iSce_Loop-methylated”, all cytosines are replaced with 5-methylcytosines to prevent unwanted conversion of cytosine to uracil in the adaptor sequence during bisulfite conversion).

Bold indicates nicking restriction site (or its complement) of Nt.BspQI, which recognises the following sequence (nicking site is indicated by arrow):

5′ . . . G C T C T T C N^{▾} . . . 3′
3′ . . . C G A G A A G N . . . 5′

[Biotin-T] indicates the following structure:

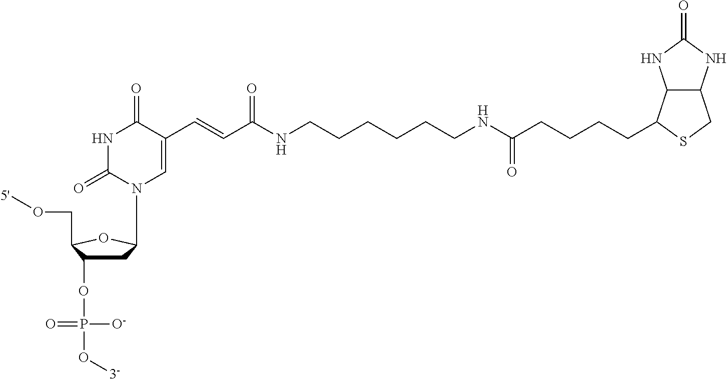

P5_BbvCI_P7 (SEQ ID NO: 7):
GCTGAGGATCTCGTATGCCGTCTTCTGCTTGUAATGATACGGCGACCACC
GAGATCTACACTCCTCAGC*T
BspQI_iSce_Loop (SEQ ID NO: 8):
GAAGAGCACACGTCTGAACTCCAGTCACTAGGGA[Biotin-T]AACAGG
GTAATCTTTCCCTACACGACGCTCTTC*T
P5_BbvCI_P7-methylated (SEQ ID NO: 9):
GCTGAGGATCTCGTATGCCGTCTTCTGCTTGUAATGATACGGCGACCACC
GAGATCTACACTCCTCAGC*T
BspQI_iSce_Loop-methylated (SEQ ID NO: 10):
GAAGAGCACACGTCTGAACTCCAGTCACTAGGGA[Biotin-T]AACAGG
GTAATCTTTCCCTACACGACGCTCTTC*T

### Adaptor Annealing:

1. A mixture of 4 μl of 100 μM P5_BbvCI_P7-methylated oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed P5_BbvCI_P7-methylated adaptor.

2. Separately, a mixture of 4 μl of 100 μM BspQI_iSce_Loop-methylated oligo, 11 μl water, 2 μl 10× TEN buffer (Illumina) and 3 μl IDTE buffer was heated to 98 C for 30 s, then a slow cool to room temperature (eg. 0.1 C/s ramp down to RT). This gives a 20 μM stock of annealed BspQI_iSce_Loop-methylated adaptor.

3. Equal volumes of the 20 μM stock of annealed P5_BbvCI_P7-methylated adaptor from step 1 and 20 μM stock of annealed BspQI_iSce_Loop-methylated adaptor from step 2 are mixed together, giving a stock solution with 10 μM each of annealed P5_BbvCI_P7-methylated adaptor and annealed BspQI_iSce_Loop-methylated adaptor.

### Preparation of Library:

1. NEB Ultra II FS reagents were thawed at room temperature and kept on ice until use.

2. The Ultra II FS Enzyme mix was vortexed for 5-8 seconds prior to use and placed on ice.

3. In a 0.2 ml PCR tube on ice, 26 μl DNA (100 ng of input DNA (methylated pUC19 sample) diluted to 26 μl with Milli-Q grade water), 7 μl of NEBNext Ultra II FS Reaction Buffer and 2 μl of NEBNext Ultra II FS Enzyme Mix were added, briefly vortexed and spun in a microcentrifuge to mix.

4. In a Thermocycler with the heated lid set to 75 C, the tubes were incubated for 5 mins at 37 C, then 30 mins at 65 C then held at 4 C.

5. The following were added to the FS reaction mixture from step 4: 30 μl of NEBNext Ultra II Ligation Master Mix, 1 μl of NEBNext Ligation Enhancer and 2.5 μl of the loop adaptors P5_BbvCI_P7-methylated and BspQI_iSce_Loop-methylated (10 μM each) prepared from step 3 of “Adaptor annealing”.

6. The entire volume was pipetted up and down 10× to mix, followed by a brief spin in a microcentrifuge.

7. The mixture was incubated at 20 C for 15 mins in a thermocycler with the heated lid off.

8. 3 μl of USER Enzyme (NEB) was added to the ligation mix.

9. The mixture was mixed well and incubated at 37 C for 15 mins with the heated lid set to >47 C.

10. Adaptor ligated DNA was then size selected via a 0.8× SPRI (iTune beads) selection: 57 μl iTune beads (ILMN) were added to 68.5 μl of ligation reaction, mixed and incubated at RT for 5 mins.

11. The mixture was placed on a magnet for 5 mins, and the supernatant was discarded.

12. The beads were washed twice with 200 μl of 80% ethanol-200 μl 80% ethanol was added with beads on the magnet, followed by a 30 s wait, and ethanol was removed, then the wash was repeated once more.

13. The last remnants of ethanol were removed with a P10 pipette and tip.

14. Beads were then air dried for 5 mins.

15. DNA was eluted from beads with 40 μl of 0.1× TE buffer. At this stage, 20 μl was saved as a “non-converted” control, the remaining 20 μl was treated to bisulfite conversion, following the Zymo Research EZ-96 DNA Methylation Gold MagPrep kit (steps 16-25 are taken from the instructions for this kit).

16. In a 0.2 ml PCR tube, 20 μl of 0.8× SPRI selected ligation and 130 μl of CT Conversion Reagent (comprises sodium metabisulfite) were added.

17. The mixture was incubated on a thermocycler at 98 C for 10 mins, then 64 C for 2.5 hours, followed by holding at 4 C for up to 20 hours.

18. The sample was transferred to 1.7 ml tubes for subsequent steps. 600 μl of M-Binding Buffer and 10 μl of MagBinding Beads were added. The mixture was vortexed for 30 s.

19. Incubate at RT for 5 mins, then place on a magnet for 5 mins.

20. The supernatant was removed and discarded. 400 μl of M-Wash buffer was added to the beads, and then vortexed for 30 s. The mixture was placed back on magnet until the beads pelleted.

21. The supernatant was removed and discarded.

22. 200 μl of M-Desulphonation Buffer was added to the beads, and then vortexed for 30 s. The mixture was incubated at RT for 15-20 mins. The mixture was then placed back on magnet until beads pelleted.

23. The supernatant was removed and discarded. 400 μl of M-Wash buffer was added to the beads, then vortexed for 30 s. The mixture was placed back on magnet until beads pelleted. This wash step was repeated once.

24. The supernatant after 2nd wash was removed, and the tubes were transferred to a hot block at 55 C to air dry the beads for 20-30 mins and remove residual M-Wash buffer.

25. 25 μl of M-Elution Buffer was added to the dried beads and vortexed for 30 s. The elution mixture was heated at 55 C for 4 mins then the tubes were placed back on the magnet for 1 min (or until the beads pelleted). The eluate was removed and transferred to a new 1.7 ml tube.

26. 175 μl of HT1 buffer (ILMN Hybridisation buffer) and 10 μl of HT1 washed MyOne Streptavidin T1 beads (Thermofisher) were added. The tubes were incubated on a rocker at RT for 30 mins. (This step selects for material which has the biotinylated loop adaptor, and removes the material which has the P5/P7 adaptors on both ends).

27 The tubes were placed on a magnet until the beads pelleted.

28. The beads were washed twice with 200 μl of Tagmentation Wash Buffer (TWB, Illumina).

29. The beads were then washed once with 200 μl of Resuspension Buffer (RSB, Illumina).

30. The beads were resuspended in 20 μl of Milli-Q grade water and transferred to 0.2 ml tubes for the final PCR.

31. 20 μl of beads+DNA were combined with 25 μl of Q5U Mastermix (NEB) and 5 μl of PPC (PCR Primer Cocktail, Illumina).

32. The mixture was amplified by PCR: cycling procedure—98 C for 3 min followed by 12 cycles of (98 C 45 s, 60 C 2 min, 68 C 2 min), then 68 C for 5 mins and then hold at 4 C.

33. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq.

1. 400 μl BspQI mix was made up-360 μl Milli-Q grade water, 40 μl of rNEB3.1 buffer (NEB) and 8 μl of Nt.BspQI (NEB were combined). The mixture was vortexed to mix and briefly spun down. The mixture was pipetted into the “EXT” position of the MiniSeq cartridge (position to the left of the Custom Primer positions).

2. The library was denatured (0.1N NaOH) and diluted to 0.5 pM final concentration in HT1 buffer according to Illumina protocol. 500 μl was loaded into the “Library” position of the MiniSeq cartridge.

3. Setup was run using MiniSeq Control Software, using a standard MiniSeq run.

4. For a CA dye swap, standard IMX was removed from the IMX position of the MiniSeq cartridge, then the position was washed 5 times with Milli-Q grade water, and replaced with 20 mls of custom IMX, where the standard two-dye system for A (A represented by red and green) and one-dye system for C (C represented by red) is replaced with a two-dye system for C (C represented by red and green) and one-dye system for A (A represented by red).

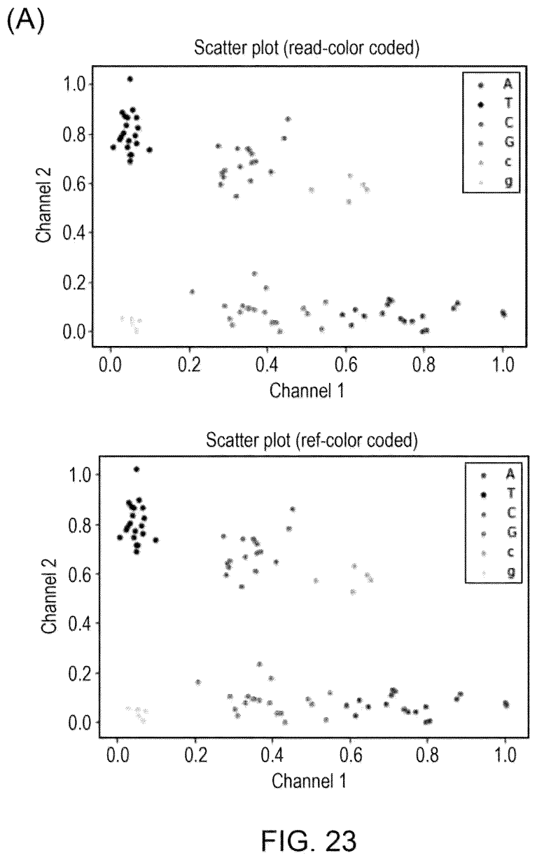

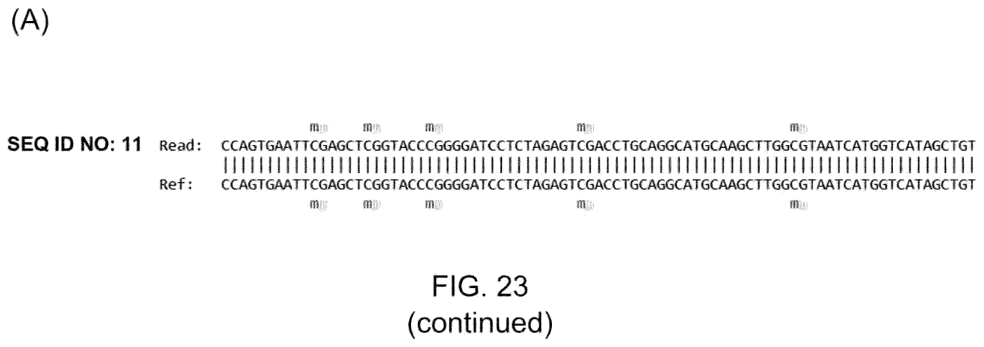

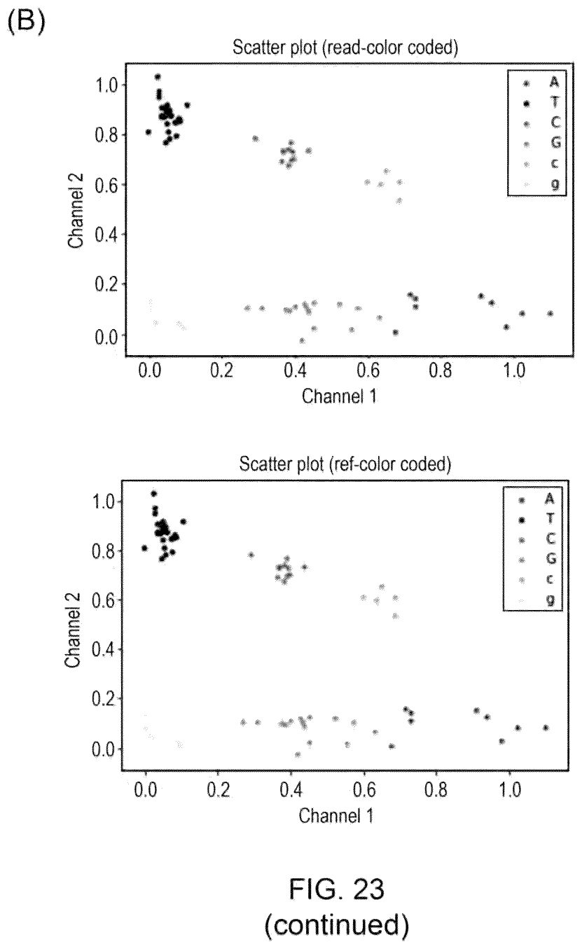

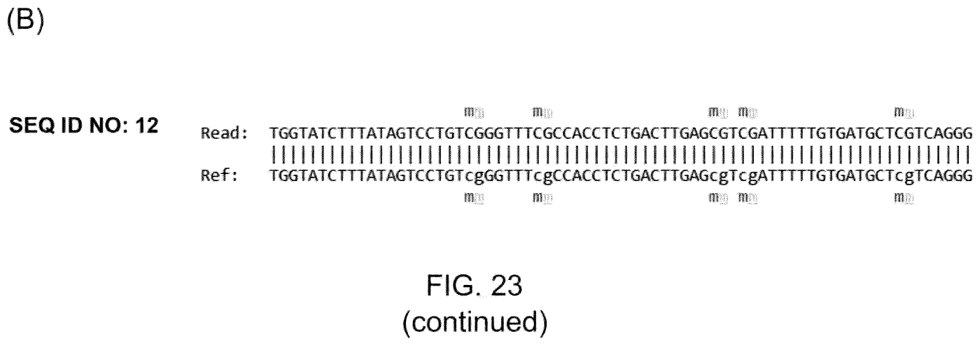

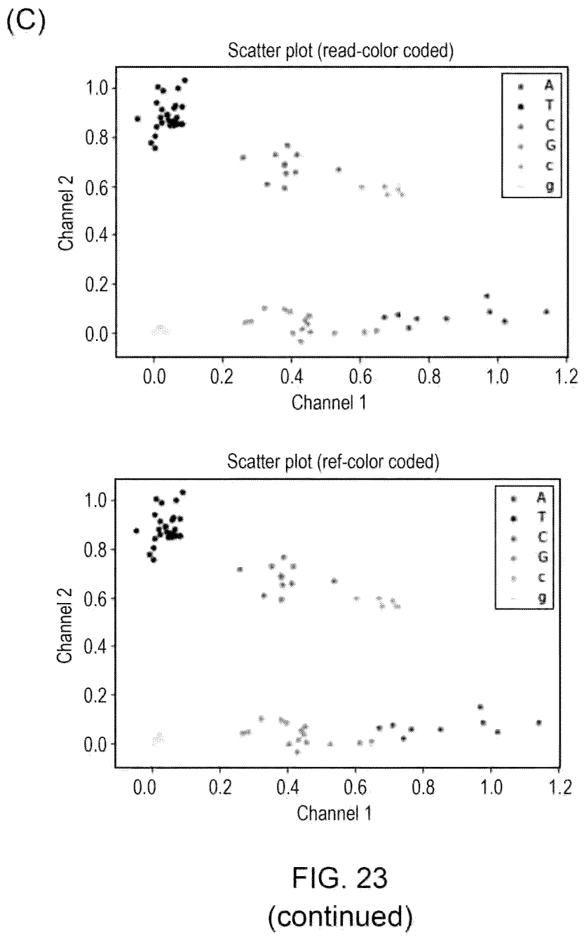

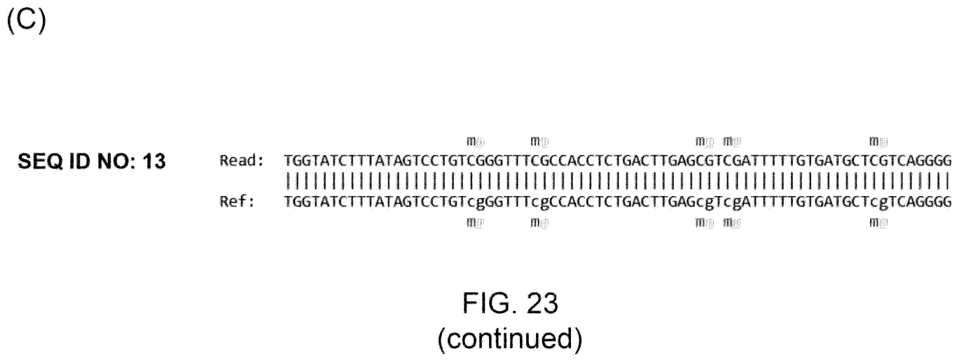

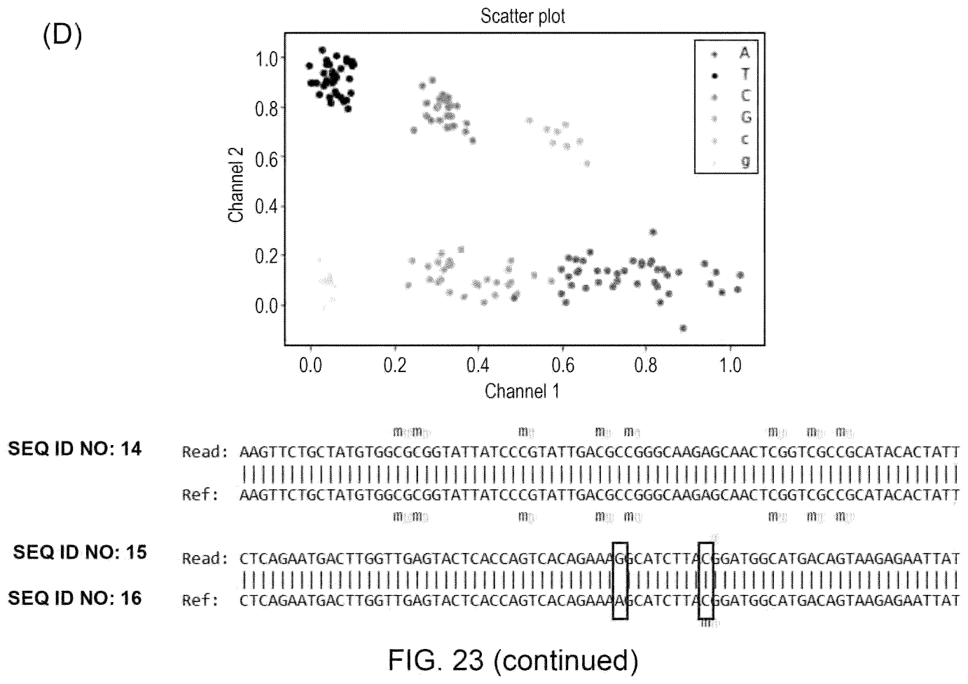

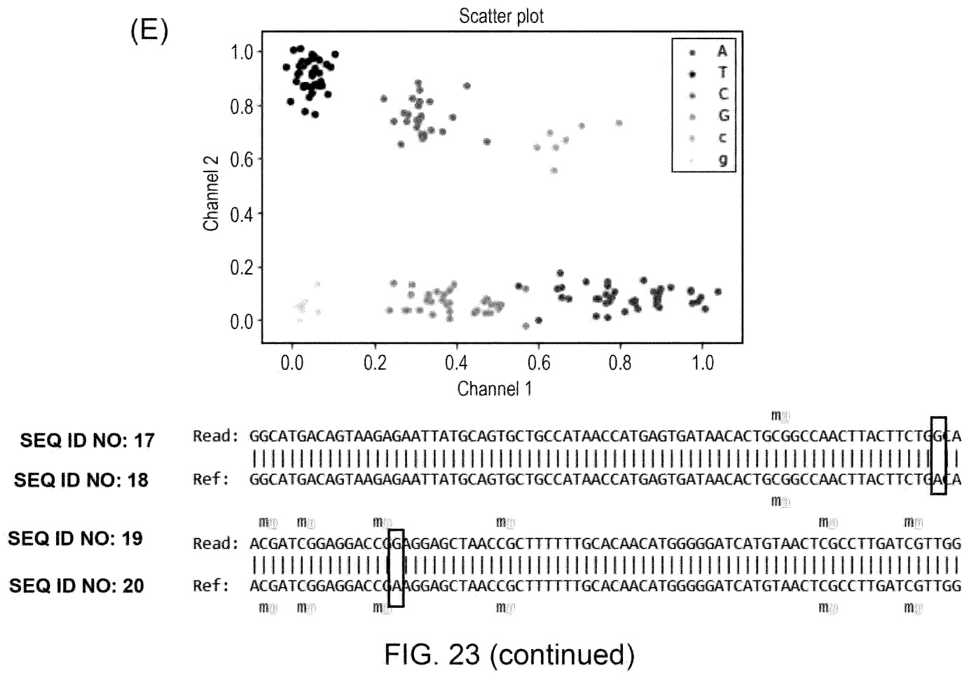

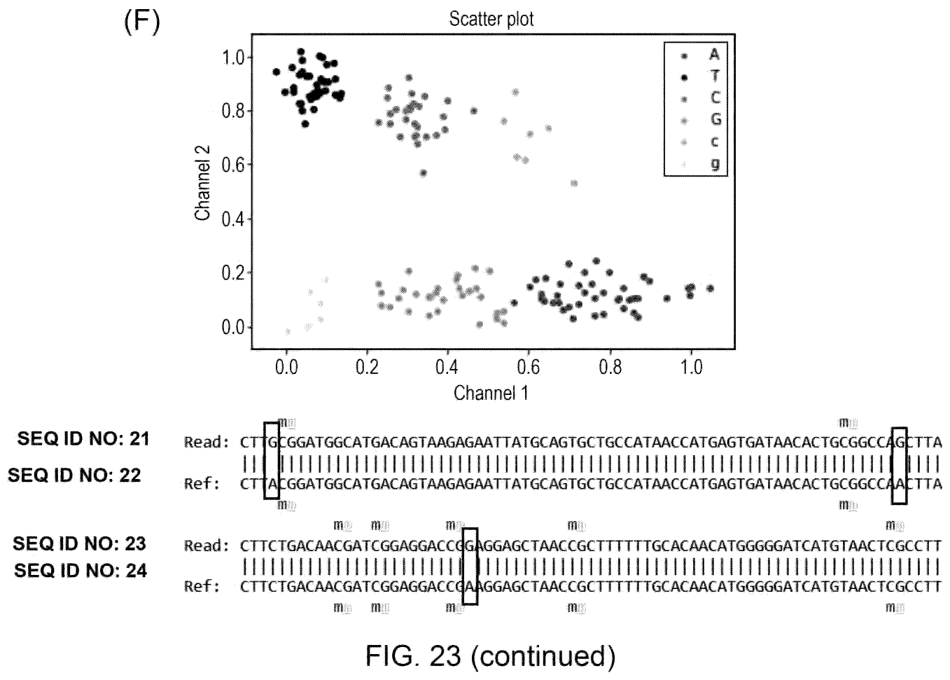

The 9 QaM results are shown in FIGS. 23A to 23F for six different library fragments, where modified cytosines can be identified by characteristic clouds in the top right corner and the bottom left corner in the plot. If the original strands in the library contained a (5mC)-G base pair (the first base corresponding to the forward strand of the library polynucleotide, and the second base corresponding to the reverse strand of the library polynucleotide), this corresponds to a C-G base pair after bisulfite conversion. As such, the forward strand of the template provides a C read (as the forward strand of the template has a G at the corresponding position), and the reverse complement strand of the template provides a C read too (as the reverse complement strand of the template has a G at the corresponding position too), which therefore appears in the top right corner of the plots in FIGS. 23A to 23F (a (C,C) read).

In addition, if the original strands in the library contained a G-(5mC) base pair (the first base corresponding to the forward strand of the library polynucleotide, and the second base corresponding to the reverse strand of the library polynucleotide), this corresponds to a G-C base pair after bisulfite conversion. As such, the forward strand of the template provides a G read (as the forward strand of the template has a C at the corresponding position), and the reverse complement strand of the template provides a G read too (as the reverse complement strand of the template has a C at the corresponding position too), which therefore appears in the bottom left corner of the plots in FIGS. 23A to 23F (a (G,G) read).

By contrast, if the original strands in the library contained a C-G base pair (the first base corresponding to the forward strand of the library polynucleotide, and the second base corresponding to the reverse strand of the library polynucleotide), this corresponds to a T-G mismatched base pair after bisulfite conversion (where C is converted to U, and U is read as T). As such, the forward strand of the template provides a T read (as the forward strand of the template has an A at the corresponding position), and the reverse complement strand of the template provides a C read (as the reverse complement strand of the template has a G at the corresponding position), which therefore appears in the top middle portion of the plots in FIGS. 23A to 23F (a (T,C) read).

If the original strands in the library contained a G-C base pair (the first base corresponding to the forward strand of the library polynucleotide, and the second base corresponding to the reverse strand of the library polynucleotide), this corresponds to a G-T mismatched base pair after bisulfite conversion (where C is converted to U, and U is read as T). As such, the forward strand of the template provides a G read (as the forward strand of the template has a C at the corresponding position), and the reverse complement strand of the template provides an A read (as the reverse complement strand of the template has a T at the corresponding position), which therefore appears in the bottom middle portion of the plots in FIGS. 23A to 23F (a (G,A) read).

If the original strands in the library contained a T-A base pair (the first base corresponding to the forward strand of the library polynucleotide, and the second base corresponding to the reverse strand of the library polynucleotide), this remains as a T-A base pair after bisulfite conversion. As such, the forward strand of the template provides a T read (as the forward strand of the template has an A at the corresponding position), and the reverse complement strand of the template provides a T read too (as the reverse complement strand of the template has an A at the corresponding position too), which therefore appears in the top left corner of the plots in FIGS. 23A to 23F (a (T,T) read).

Finally, if the original strands in the library contained an A-T base pair (the first base corresponding to the forward strand of the library polynucleotide, and the second base corresponding to the reverse strand of the library polynucleotide), this remains as an A-T base pair after bisulfite conversion. As such, the forward strand of the template provides an A read (as the forward strand of the template has a T at the corresponding position), and the reverse complement strand of the template provides an A read too (as the reverse complement strand of the template has a T at the corresponding position too), which therefore appears in the bottom right corner of the plots in FIGS. 23A to 23F (an (A,A) read).

| Library | Accuracy | Sensitivity | Specificity |
|---|---|---|---|
| Library | 85/85 | 10/10 | 75/75 |
| fragment 1 | (100%) | (100%) | (100%) |
| (FIG. 23A) | | | |
| Library | 72/72 | 10/10 | 62/62 |
| fragment 2 | (100%) | (100%) | (100%) |
| (FIG. 23B) | | | |
| Library | 73/73 | 10/10 | 63/63 |
| fragment 3 | (100%) | (100%) | (100%) |
| (FIG. 23C) | | | |
| Library | 148/150 | 17/18 | 133/133 |
| fragment 4 | (98.67%) | (94.44%) | (100%) |
| (FIG. 23D) | | | |
| Library | 148/150 | 14/14 | 136/136 |
| fragment 5 | (98.67%) | (100%) | (100%) |
| (FIG. 23E) | | | |
| Library | 147/150 | 14/14 | 136/136 |
| fragment 6 | (98%) | (100%) | (100%) |
| (FIG. 23F) | | | |

(Accuracy = number of correct base calls (GCAT, irrespective of methylation status)/total number of bases; Sensitivity = number of true positive methylated base calls/total number of methylated bases; Specificity = number of true negative methylated base calls/(number of true negative methylated base calls + number of false positive methylated base calls))

Overall, these results show that methylation analysis can be conducted on polynucleotide sequences to identify modified cytosines. In particular, by enabling concurrent sequencing of the forward and reverse complement strands of the template (or reverse and forward complement strands of the template), modified cytosines can be identified quickly and accurately. Again, such a process is made viable by using the methods of preparing polynucleotide libraries as described herein.

### Example 3—Mismatched Base Pair Analysis and Methylation Analysis on Methylated pUC19 Sample Using 9 QaM

Oligo sequences:

For transposon annealing (underline indicates ME′ or ME):

ME′-HYB2
(SEQ ID NO. 41)
/5Phos/CTGTCTCTTATACACATCTGAGTAAGTGGAAGAGATAGGAAGG
ME′-HYB2′
(SEQ ID NO. 42)
/5Phos/CTGTCTCTTATACACATCTCCTTCCTATCTCTTCCACTTACTC
Biotin-A14-ME
(SEQ ID NO. 29)
Biotin-TCGTCGGCAGCGTCAGATGTGTATAAGAGACAG
Biotin-B15-ME
(SEQ ID NO. 30)
Biotin-GTCTCGTGGGCTCGGAGATGTGTATAAGAGACAG

Sequencing oligos (underline indicates ME):

HYB2-ME
(SEQ ID NO. 32)
GAGTAAGTGGAAGAGATAGGAAGGAGATGTGTATAAGAGACAG
HYB2′-ME
(SEQ ID NO. 34)
CCTTCCTATCTCTTCCACTTACTCAGATGTGTATAAGAGACAG

### Preparation of Forked Adaptors:

30. 5 μl of 200 μM stock of biotin-A14-ME oligo was combined with 10 μl of 100 μM stock of ME′-HYB2 oligo. 2 μl of 10× TEN Annealing buffer (Illumina) and 3 μl of IDTE buffer (Illumina) was added (“A14” transposome mixture).

31. Separately, 5 μl of 200 μM stock of biotin-B15-ME oligo was combined with 10 μl of 100 μM stock of ME′-HYB2′ oligo. 2 μl of 10× TEN Annealing buffer (Illumina) and 3 μl of IDTE buffer (Illumina) was added (“B15” transposome mixture) with 10× TEN and IDTE buffers.

32. Each mixture was heated to 95 C for 30 s followed by a slow cool (0.1 C/s ramp rate) to 10 C.

33. 2 μl of each annealed mixture was combined with 46 μl of Standard Storage Buffer (contains 50% glycerol, Illumina) and 2 μl of Tn5 transposase (˜90 μM stock).

34. Each mixture was mixed and incubated overnight at 37 C. Following the incubation step, the two separately prepared transposome complexes were combined together by adding 50 μl of each to another 100 μl of Standard Storage Buffer to give 200 μl of 1 μM transposome mix.

Loading of Forked Adaptors onto Beads:

1. 200 μl of MyOne T1 Streptavidin beads (Thermofisher) were washed twice with 200 μl Tagmentation Wash buffer (TWB, Illumina).

2. Beads were resuspended in 960 μl of TWB and 40 μl of 1 μM transposome mix from step 5 of “Preparation of forked adaptors” was added.

3. Beads were mixed on a rotator for 30 mins to 1 hr at room temperature.

4. Beads were put on a magnet and beads were washed twice with TWB.

5. Beads were resuspended in original volume (200 μl) of BLT Storage Buffer (Illumina). The BLTs were stored at 4 C until needed.

### Tagmentation:

1. 10 μl BLT (bead linked transposomes) from step 5 of “Loading of forked adaptors onto beads” were combined with 100 ng DNA in 30 μl (pUC19 methylated control DNA) and 10 μl of TB1 (5× Tag buffer, Illumina).

2. The combination was mixed and incubated at 55 C for 5 min, followed by a hold step at 10 C.

3. 10 μl ST2 Stop buffer was added and mixed.

4. The mixture was incubated at room temp for 5 mins.

5. The tubes were transferred to a magnet.

6. The beads were washed twice with 100 μl Tagmentation Wash buffer (TWB, Illumina).

7. The beads were resuspended in 50 μl of ELM (Extension Ligation Mix, Illumina).

8. The mixture was incubated at 37 C for 5 mins, then 50 C for 5 mins, followed by a hold step at 10 C.

### Hybridisation and Extension on Beads:

1. The tubes from step 8 of “Tagmentation” were placed on a magnet until the BLT beads pelleted.

2. The beads were washed once with 200 μl of Tagmentation Wash Buffer (TWB, Illumina).

3. The beads were washed once with 200 μl of 0.1N NaOH—the beads were left to sit in 0.1N NaOH for 30 s during this wash step.

4. Beads were washed once with 200 μl of TWB.

5. Bears were resuspended in 100 μl of HT1 (Hybridisation Buffer, Illumina).

6. Beads were heated in HT1 to 70 C for 30 s followed by a slow cool (0.1 C/s) down to 10 C.

7. Beads were washed twice with 200 μl of TWB.

8. Beads were resuspended in 100 μl of PAM (Patterned Amplification Mix, Illumina) supplemented with 50 mM KCl.

9. Beads were heated in PAM to 50 C for 5 mins, then 60 C for 5 mins.

10. Beads were washed twice with 200 μl of TWB.

11. Beads were resuspended in 50 μl of RSB (Resuspension Buffer, Illumina).

### Methylation Analysis Conversion Method:

(N.B. For the purposes of detecting mismatched base pairs in the library, the methylation analysis conversion method is not strictly necessary. As such, this step may be skipped if the end goal is to identify only mismatched base pairs, rather than both mismatched base pairs and methylation status.)

1. The following TET master mix (TET MM) was prepared and kept on ice:

| | 1× (μl) | 4.5× (μl) |
|---|---|---|
| Water | 9.00 | 40.50 |
| Reconstituted TET2 Reaction Buffer | 10 | 45 |
| (NEB EM-seq kit) | | |
| Oxidation Supplement (NEB EM-seq kit) | 1 | 4.5 |
| DTT (NEB EM-seq kit) | 1 | 4.5 |
| TET2 (NEB EM-seq kit) | 4 | 18 |
| Total | 25 | 112.5 |

2. On ice, 25 μl of TET MM was added to 20 μl of adaptor-ligated DNA in the form of BLTs in RSB (from step 11 of “Hybridisation and extension on beads”).

3. The mixture was vortexed and centrifuged briefly.

4. 500 mM of Fe (II) solution (NEB EM-seq kit) was freshly prepared and diluted by adding 1 μl to 1249 μl of water.

5. 5 μl of the diluted Fe (II) solution was added to the 45 μl of adaptor-ligated DNA with TET MM prepared in step 2.

6. The mixture was vortexed (or pipette mixed 10×), centrifuged briefly, incubated for 1 hr at 37 C, then put on ice.

7. 1 μl of Stop reagent was added, vortexed (or pipette mixed 10×), and incubated at 37 C for 30 mins.

8. The beads were washed once with 100 μl Wash buffer, and then resuspended in 35 μl water.

9. In a PCR tube, the 35 μl of TET-oxidised DNA from step 8 was combined with 10 μl of sodium acetate/acetic acid buffer (pH 4.3) and 5 μl of 1 M pyridine borane. The mixture was incubated overnight at 40 C.

10. The beads were washed twice with 100 μl Wash buffer, then resuspended in 20 μl of RSB.

11. The 20 μl of beads+DNA in RSB from step 10 was combined with 25 μl of Q5U Mastermix (NEB) and 5 μl of UDI primers (Unique Dual Index primers, Illumina).

12. The mixture was amplified by PCR: cycling procedure—98 C for 30 s followed by 3 cycles of (98 C 10 s, 62 C 30 s, 65 C 3 min), then 6 cycles of (98 C 10 s, 62 C 30 s, 65 C 30 s), 65 C for 5 mins and then hold at 4 C.

13. PCR products were analysed by TapeStation D1000 (Agilent), and then subjected to a further SPRI clean-up before quantification using a Qubit Broad Range dsDNA assay kit (Thermofisher).

### Sequencing:

Sequencing was conducted on the MiniSeq. Standard clustering on the MiniSeq and a standard first hyb was conducted for the 1^{st} 36 cycles of sequencing.

A custom second hyb was used from the “Cust3” position of the reagent cartridge. This primer hyb maintains a higher temperature (60 C) than normal during the post-hyb wash (which usually drops to 40 C). This higher temperature was to ensure that the right sequencing primers hyb to the right places on the cluster strands.

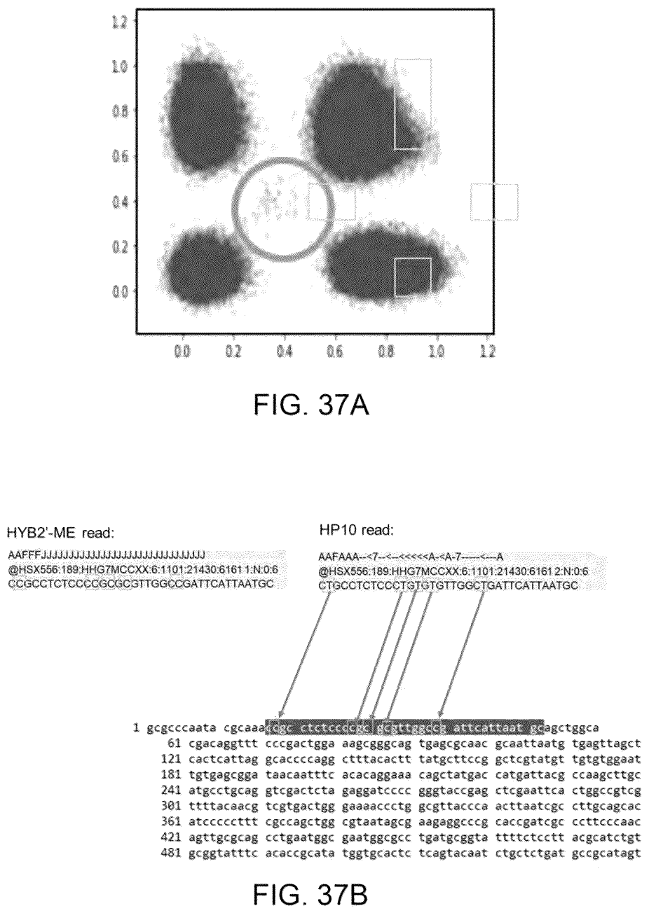

The primer mix for this custom hyb was HP10 R1 primer mix (Illumina) spiked with 0.5 μM each of HYB2′-ME and HYB2-ME primers. These primers are all unblocked and allow concurrent sequencing of both the first portion and the second portion, and so generate the 9 QaM signal during sequencing. The converted library was loaded onto the MiniSeq cartridge at 1 pM final concentration. The MiniSeq was set up to save 3 tiles of images per cycle, for later off-line analysis. The 9 QaM results are shown in FIG. 37A, where modified cytosines can be identified by a characteristic central cloud in the plot (indicated by circled region). Of course, the (5-mC)-G base pair (or a G-(5-mC) base pair), which is subsequently converted to a mismatched T-G base pair (or a G-T base pair) by TAPS, represents a type of mismatched base pair. Other mismatched base pairs can be identified by side clouds (top middle, bottom middle, centre left, centre right-indicated by boxed regions) The actual genetic sequences are shown in FIG. 37B, where modified cytosines can be assigned to cases where a C-T mismatch is observed between the HYB2′-ME read and the HP10 read.

Overall, these results (in particular the custom second hyb results) show that analysis can be conducted on polynucleotide sequences to find mismatched base pairs. In addition, methylation analysis can be conducted on polynucleotide sequences to identify modified cytosines-however, this is not strictly necessary for the purposes of various embodiments of the present invention if the methylation analysis conversion method is skipped. In particular, by enabling concurrent sequencing of the forward and reverse complement strands of the template (or reverse and forward complement strands of the template), mismatched base pairs can be identified quickly and accurately.

### Example 4—Mismatched Base Pair Analysis on Human DNA Sample Using 9 QaM

A similar experiment to Example 3 was conducted except that the DNA during the “Tagmentation” section was replaced with a Promega human blend DNA spiked with 5% Phix (as control). In addition, the steps from “Methylation analysis conversion method” were not conducted-thus, any errors would be indicative of mismatched base pairs, for example, as a result of errors resulting from library preparation.

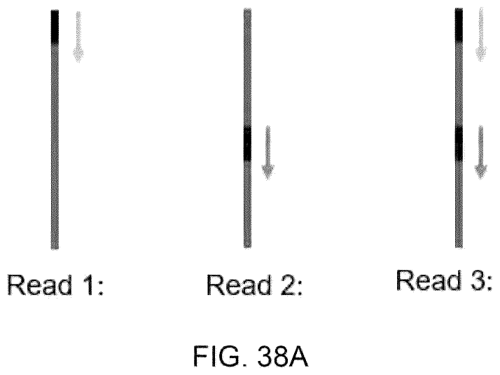

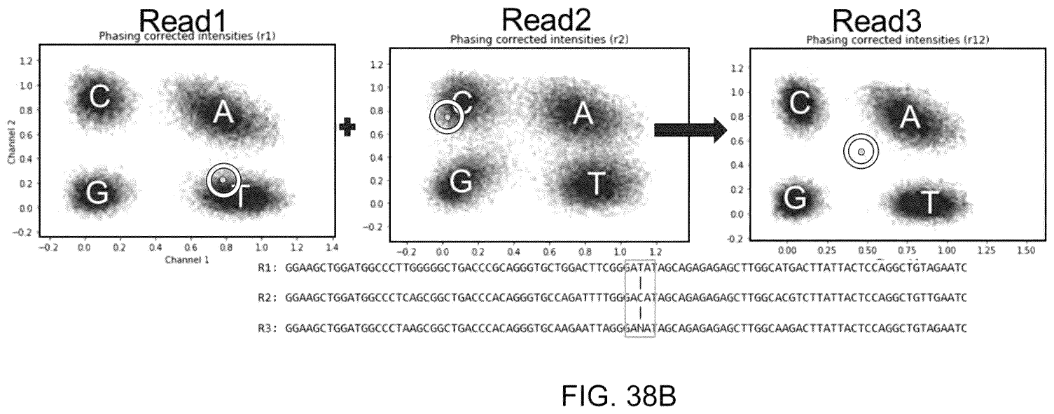

Sequencing was conducted on the NextSeq 2000. A custom hyb was conducted where the usual primer mix was replaced with HP10 primer mix (Illumina) spiked with HYB2′-ME primer (0.3 μM each). These primers are all unblocked and allow concurrent sequencing of both the first portion and the second portion, and so generate the 9 QaM signal during sequencing. The library was loaded onto the NextSeq 2000 at 650 pM final concentration. These results are presented in FIG. 38B (Read 3-combined Read 1 and Read 2), where mismatched base pairs can be identified by characteristic off-corner clouds in the plot (indicated by point in circled region). In this case, a C-T mismatch (a middle cloud) was detected, leading to an “N” readout in the Read 3 sequence.

Control experiments were also conducted where individual reads were done separately using only one sequencing primer type (Read 1 and Read 2 separately). One of the reads on the tandem insert corresponds to a readout for the forward strand, whilst the other read on the tandem insert corresponds to a readout for the reverse complement strand. In the Read 1 case, using a HP21 primer mix (Illumina), one of the bases is detected as T (indicated by point in circled region); in the Read 2 case, using a HYB2′-ME primer, one of the bases is detected as C (indicated by point in circled region). The control experiment confirms that the detection of the C-T mismatch in the Read 3 case was correct, using only one read run.

Overall, these results show that analysis can be conducted on polynucleotide sequences to find mismatched base pairs. Again, by enabling concurrent sequencing of the forward and reverse complement strands of the template (or reverse and forward complement strands of the template), mismatched base pairs can be identified quickly and accurately.

### Example 5: Concurrent Sequencing of a Concatenated Strand (Different Inserts, Human and PhiX)

### 1.1 Oligo Sequences for Stitch PCR Method:

HYB2-ME-SEQ ID NO: 32; HYB2′-ME-SEQ ID NO: 34 ME sequences are underlined. These were to be used with P5-UDI-A14 and P7-UDI-B15 oligos to PCR up different genomic DNA libraries, making the libraries P5-insert-HYB2′ or P7-insert-HYB2. These libraries were then combined using SOE (splicing by overhang extension) PCR to combine them together. In this experiment the following two oligos were used as partners as examples:

Dual-Biotin 6T-P5-nonlin
(SEQ ID NO: 47)
5′Dual-biotin-TTTTTTAATGATACGGCGACCACCGAGATCTACAC
Dual-Biotin 6T-P7-nonlin
(SEQ ID NO: 48)
5′Dual-biotin-TTTTTTCAAGCAGAAGACGGCATACGAGAT

The 5′ dual biotin is nonetheless, irrelevant for this experiment.

### 1.2 Method

1. Illumina DNA Flex libraries containing human or PhiX (bacteriophage) inserts were prepared following the standard Illumina protocol:
https://emea.illumina.com/products/by-type/sequencing-kits/library-prep-kits/nextera-dna-flex.html

2. Two initial PCRs were set up containing:
25 μl 2× Phusion Mastermix (New England Biolabs)
0.25 μl 100 μM dual-biotin 6T-P5-nonlin
0.25 μl 100 μM HYB2-ME
1 μl Human Flex library (˜10 ng)
23.5 μl H2O

The other PCR used the dual-biotin 6T-P7-nonlin and HYB2′-ME primer pair on the PhiX Flex library.

3. PCRs were cycled:
98 C for 30 s, followed by 10 cycles of 98 C for 10 s, 50 C for 30 s and 72 C for 30 s, then a 5 min extension step at 72 C and then held at 4 C

4. After checking that material had been made in the initial PCRs via gel electrophoresis, “Splice Overlap Extension” (SOE) PCRs were assembled by combining 20 μl of each of the initial PCRs.

5. SOE PCRs were cycled:
98 C for 30 s, followed by 8 cycles of 98 C for 10 s, 50 C for 60 s and 72 C for 60 s, then a 5 min extension step at 72 C and then held at 4 C.

6. SOE PCRs were cleaned up via a 1× SPRI bead clean-up and quantified using the Qubit Broad Range dsDNA assay (Thermofisher), prior to use in sequencing experiments.

1.3 iSeq100 Sequencing Details:

An iSeq100 cartridge was cracked open, and premixed HCX (90 μl ECX1+45 μl of EXC2+90 μl HCXE3-ExAmp mix for iSeq100) added to the HCX Mixing well. The standard HP10 read 1 primer mix was removed from its well, washed with 200 μl water 5× and then replaced with 150 μl of the 16QAM sequencing primer mix.

16QAM sequencing primer mix-addition of equal concentrations of HYB2′-ME and HYB2′-ME-block in the standard sequencing primer mix from Illumina. The standard sequencing primers are at 0.3 μM each within HP10, and we mix the HYB2′-ME (SEQ ID NO: 34) and HYB2′-ME-block (SEQ ID NO: 36) primers into this to give 0.5 μM of each of these primers. The 50:50 ratio of blocked/unblocked primers for HYB2′-ME gives us the “50%” signal required at this primer site during 16QAM sequencing.

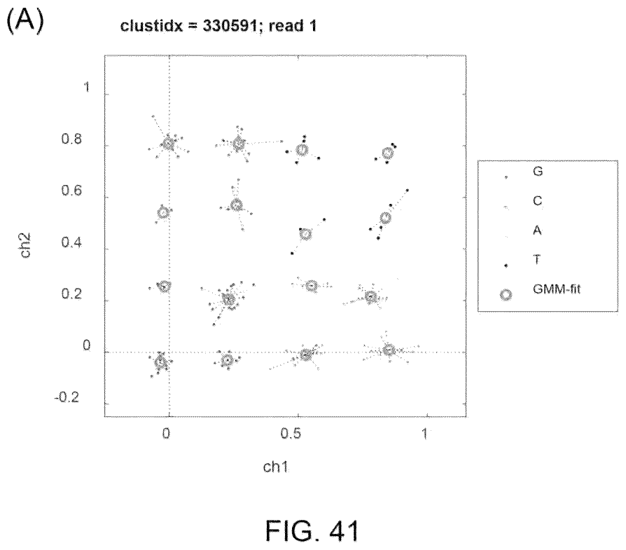

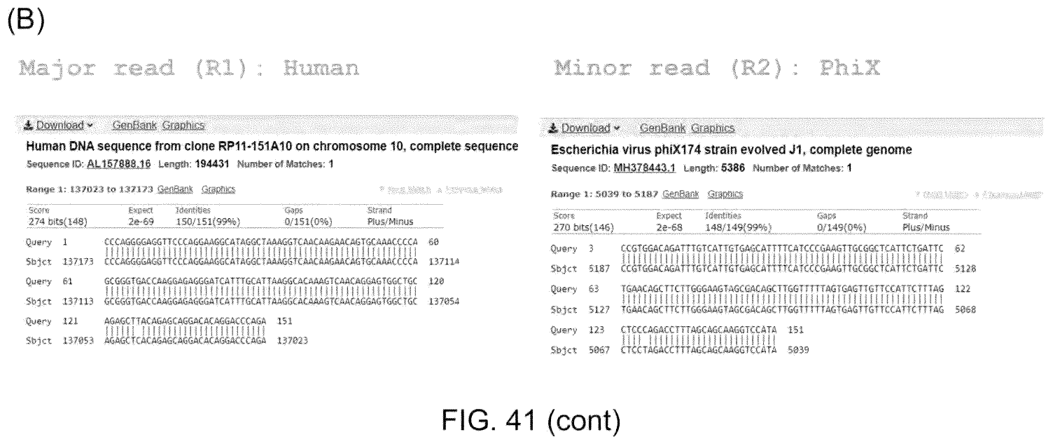

As shown in FIG. 41A, by plotting relative intensities of light signals obtained from a first channel (ch1) and a second channel (ch2), a constellation of 16 clouds is obtained. Each of these clouds allows sequence information to be identified on both the human insert and the PhiX insert, where the top left corner of four clouds corresponds with base calls corresponding to C, the top right corner of four clouds corresponds with base calls corresponding to T, the bottom left corner of four clouds corresponds with base calls corresponding to G, and the bottom right corner of four clouds corresponds with base calls corresponding to A. The basecall read out (R1 and R2) of both the human insert and the PhiX insert is also shown. As shown in FIG. 41B, alignment of R1 and R2 (minor and major reads respectively) with the known human and Phix sequence confirmed that the method accurately sequenced the inserts. In particular the sequence identity of R1 and R2 with the known sequences was 99% (150 out of 151 correct base calls for R1 and 148 out of 149 correct base calls for R2).

### Example 6: Concurrent Sequencing of Separate Strands (Forward and Forward Complement, Human)

P5f-Adaptor
(SEQ ID NO: 49)
AATGATACGGCGACCACCGAGATCTACAC*T
P7f-Adaptor
(SEQ ID NO: 50)
CAAGCAGAAGACGGCATACGAGA*T
(* indicates a phosphorothioate linkage)

### 2.1 Method

1. 1 ug mix human genomic DNA from Promega in 50 μl was fragmented to 400-500 bp fragment using the TruSeq-450 program on the Covaris.

2. End prep was performed using NEBNext Ultra II kit.

3. For adapters: 15 ul of F/R oligos from each P5 and P7 were mixed and 1.5 ul 10× NEBuffer 2 was added. The mix was annealed using AK_ANNEAL program (96 C for 2 mins, then to 25 C at −0.1/sec). 30 ul of each of the annealed oligos was then added to 140 ul of water to make 200 ul of 15 uM adapter solution (7.5 uM each side).

4. This mix was used for standard ligation using NEBNext Ultra II kit. The resulting 93 ul was mixed with 3 ul of water and 22.5 ul of iTune (SPRI-like) beads for the 1st size selection cut.

5. Supernatant was then mixed with 12.5 ul of beads for the second size selection. DNA was eluted in 20 ul.

6. 15 ul was used for 6 cycles of Q5 PCR using 10 ul of P5f/P7f primer mix (5 uM each).

7. The PCR product was purified using 0.9× iTune bead selection. It was measured to be at 23 ng/ul, or almost 68 nM.

2.2 16QAM sequencing of library

The goal is to first block 50% of P7 ends with ddNTP spiked IMX, and then nick P5 end and perform dsSBS sequencing from both ends at the same time (16QAM).

1. ShAdp Human library (with 10% PhiX as a control) was used. After initial denaturation and neutralization of library to give a 20 pM stock, 15 ul of this was added in 485 HT1 to give a 0.6 pM loading concentration for the MiniSeq run.

2. 2.25 ul of 1 mM ddNTPs was added to 500 ul of MiniSeq IMX in the Cust2 position of the MiniSeq cartridge.

3. 250 ul of BMX (Blocking Mix, Illumina) was added to the “EXT” position (cartridge well to the left of the Cust positions).

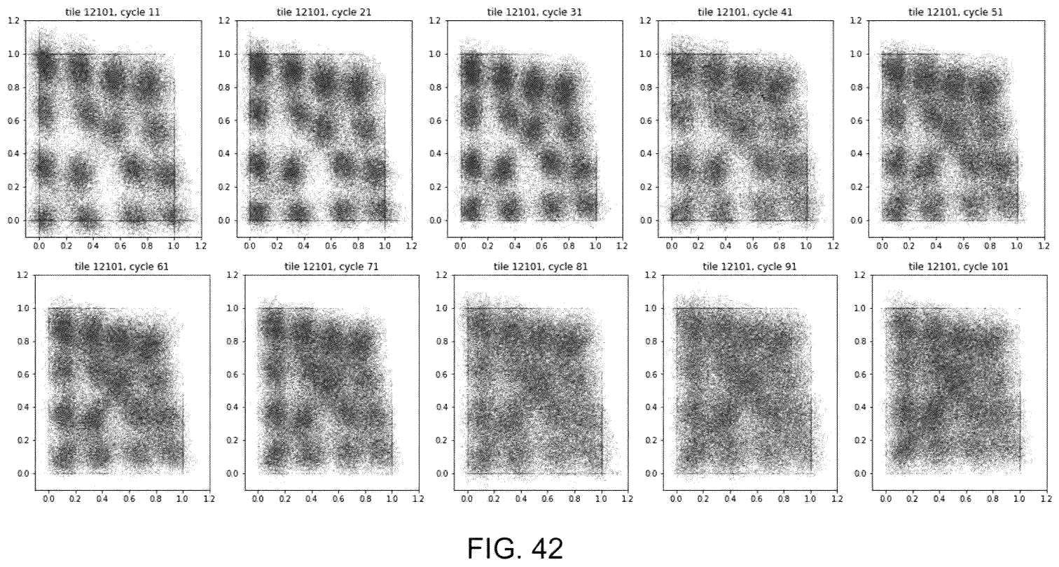

As shown in FIG. 42, by plotting relative intensities of light signals obtained from a first channel (x-axis) and a second channel (y-axis), a constellation of 16 clouds is obtained over multiple cycles. Again, each of these clouds allows sequence information to be identified on both the human insert and the PhiX insert.

### Example 7: Concurrent Sequencing of Separate Strands (Different Strands, Separate Parts of Phix)

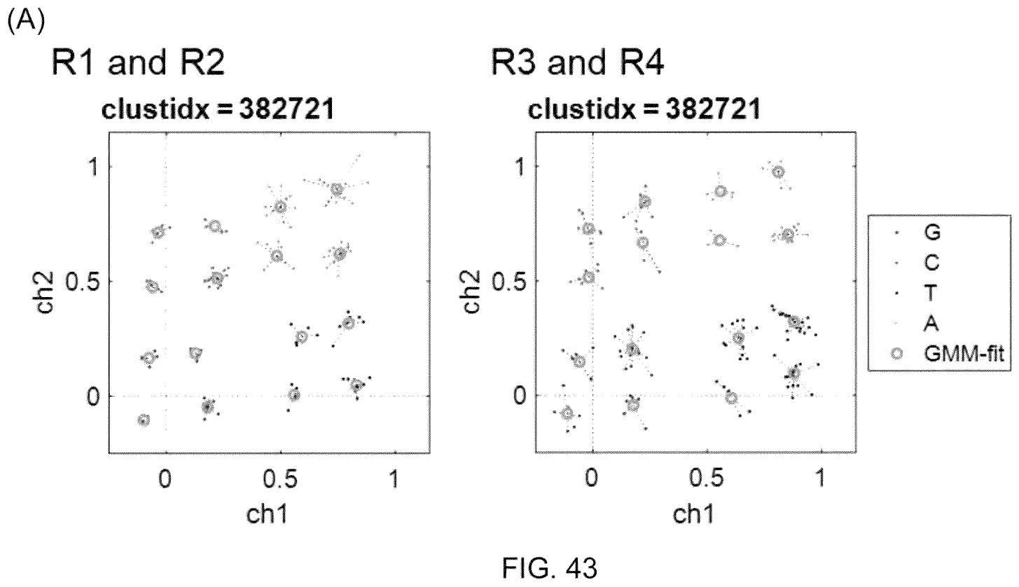

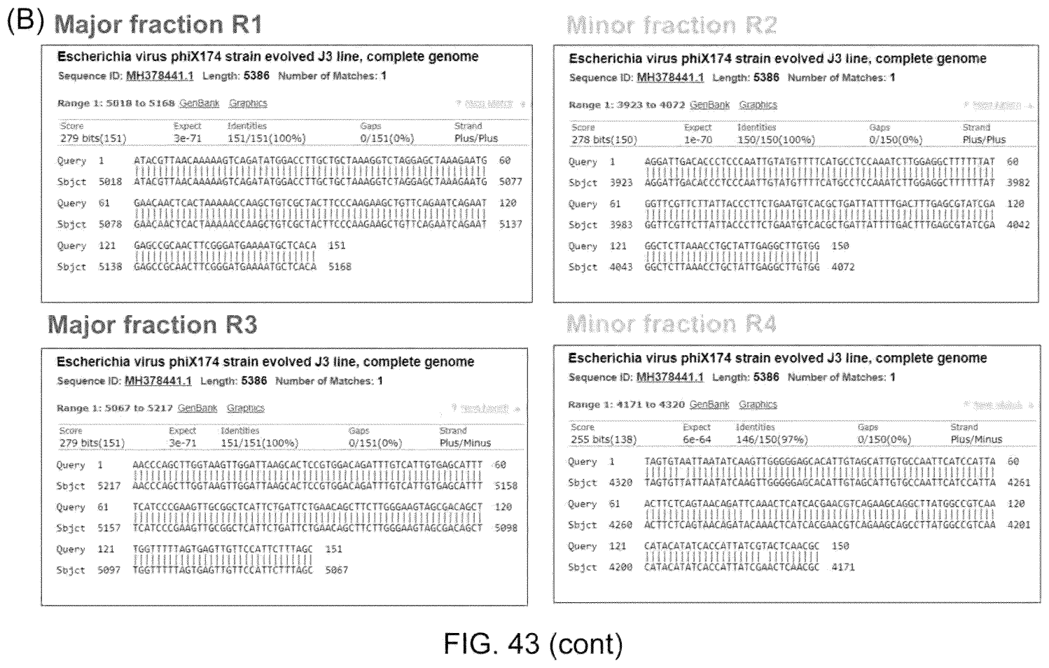

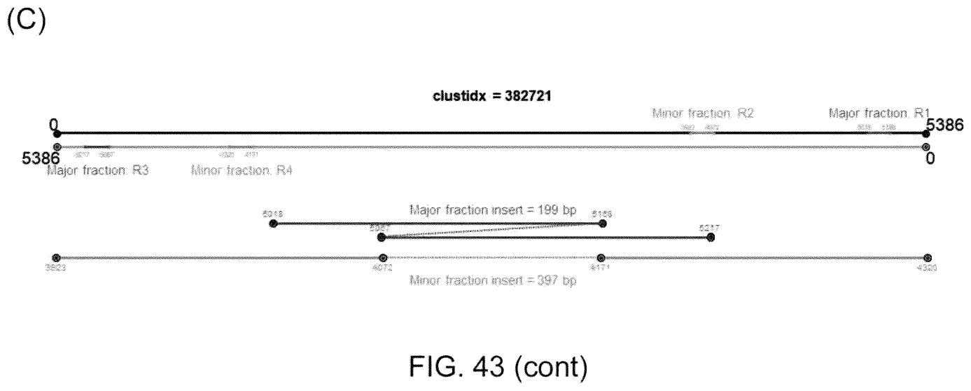

As shown in FIG. 43A, by plotting relative intensities of light signals obtained from a first channel (ch1) and a second channel (ch2), a constellation of 16 clouds is obtained. Each of these clouds allows sequence information to be identified on both the different inserts from the PhiX genome, where the top left corner of four clouds corresponds with base calls corresponding to C, the top right corner of four clouds corresponds with base calls corresponding to A, the bottom left corner of four clouds corresponds with base calls corresponding to G, and the bottom right corner of four clouds corresponds with base calls corresponding to T. The basecall read out (R1 and R2) of both the different inserts from the PhiX genome is also shown.

A subsequent resynthesis step allows “paired end” read to be conducted. This allows a further basecall read out to be obtained (R3 and R4).

As shown in FIG. 43B, alignment of R1, R2, R3 and R4 with the known sequence confirmed that the method accurately sequenced the inserts (in particular the sequence identity of R1, R2 and R3 with the known sequence was 100%).

SEQUENCE LISTING

SEQ ID NO: 1: P5 sequence
AATGATACGGCGACCACCGAGATCTACAC
SEQ ID NO: 2: P7 sequence
CAAGCAGAAGACGGCATACGAGAT
SEQ ID NO: 3: P5′ sequence (complementary to P5)
GTGTAGATCTCGGTGGTCGCCGTATCATT
SEQ ID NO: 4: P7′ sequence (complementary to P7)
ATCTCGTATGCCGTCTTCTGCTT
SEQ ID NO: 5: Alternative P5 sequence
AATGATACGGCGACCGA
SEQ ID NO: 6: Alternative P5′ sequence (complementary to alternative P5 sequence)
TCGGTCGCCGTATCATT
SEQ ID NO:7- P5_BbvCI_P7:
GCTGAGGATCTCGTATGCCGTCTTCTGCTTGUAATGATACGGCGACCACCGAGATCTA
CACTCCTCAGC*T
SEQ ID NO: 8 BspQI_iSce_Loop
GAAGAGCACACGTCTGAACTCCAGTCACTAGGGA[Biotin-T]AACAGGGTAATCTTTCCCT
ACACGACGCTCTTC*T
SEQ ID NO:9 P5_BbvCI_P7-methylated:
GCTGAGGATCTCGTATGCCGTCTTCTGCTTGUAATGATACGGCGACCACCGAGATCTA
CACTCCTCAGC*T
SEQ ID NO:10 BspQI_iSce_Loop-methylated:
GAAGAGCACACGTCTGAACTCCAGTCACTAGGGA[Biotin-T]AACAGGGTAATCTT
TCCCTACACGACGCTCTTC*T
SEQ ID NO. 11: Removable P5 sequence
TTTTTTTTTTAATGATACGGCGACCACCGAUCTACAC (where U = 2-deoxyuridine)
SEQ ID NO. 12: Removable P7 sequence
TTTTTTTTTTCAAGCAGAAGACGGCATACGA[G^{oxo}]AT (where [G^{oxo}] = 8-
oxoguanine)
SEQ ID NO. 13: Extended primer sequence with A as 5′ additional nucleotide and P5′
sequence (complementary to P5)
AGTGTAGATCTCGGTGGTCGCCGTATCATT
SEQ ID NO. 14: Extended primer sequence with T as 5′ additional nucleotide and P5′
sequence (complementary to P5)
TGTGTAGATCTCGGTGGTCGCCGTATCATT
SEQ ID NO. 15: Extended primer sequence with C as 5′ additional nucleotide and
P5′ sequence (complementary to P5)
CGTGTAGATCTCGGTGGTCGCCGTATCATT
SEQ ID NO. 16: Extended primer sequence with G as 5′ additional nucleotide and P5′
sequence (complementary to P5)
GGTGTAGATCTCGGTGGTCGCCGTATCATT
SEQ ID NO. 17: Extended primer sequence with A as 5′ additional nucleotide and P7′
sequence (complementary to P7)
AATCTCGTATGCCGTCTTCTGCTTG
SEQ ID NO. 18: Extended primer sequence with T as 5′ additional nucleotide and P7′
sequence (complementary to P7)
TATCTCGTATGCCGTCTTCTGCTTG
SEQ ID NO. 19: Extended primer sequence with C as 5′ additional nucleotide and P7′
sequence (complementary to P7)
CATCTCGTATGCCGTCTTCTGCTTG
SEQ ID NO. 20: Extended primer sequence with G as 5′ additional nucleotide and P7′
sequence (complementary to P7)
GATCTCGTATGCCGTCTTCTGCTTG
SEQ ID NO. 21: Extended primer sequence with A as 5′ additional nucleotide and
alternative P5′ sequence (complementary to alternative P5)
ATCGGTCGCCGTATCATT
SEQ ID NO. 22: Extended primer sequence with T as 5′ additional nucleotide and
alternative P5′ sequence (complementary to alternative P5)
TTCGGTCGCCGTATCATT
SEQ ID NO. 23: Extended primer sequence with C as 5′ additional nucleotide and
alternative P5′ sequence (complementary to alternative P5)
CTCGGTCGCCGTATCATT
SEQ ID NO. 24: Extended primer sequence with G as 5′ additional nucleotide and
alternative P5′ sequence (complementary to alternative P5)
GTCGGTCGCCGTATCATT
SEQ ID NO. 25: P5_BbvCI_P7
GCTGAGGATCTCGTATGCCGTCTTCTGCTTGUAATGATACGGCGACCACCGAG
ATCTACACTCCTCAGC*T (where asterisk (*) indicates phosphorothioate linkage)
SEQ ID NO. 26: BspQI_iSce_Loop
GAAGAGCACACGTCTGAACTCCAGTCACTAGGGA[Biotin-T]AACAGGGTAATCTT
TCCCTACACGACGCTCTTC*T (where asterisk (*) indicates phosphorothioate linkage,
[Biotin-T] is a modified thymine residue comprising biotin)
(Underlined sequences are ME or ME′ sequences)
SEQ ID NO. 27: A14- TCGTCGGCAGCGTC
SEQ ID NO. 28: B15-GTCTCGTGGGCTCGG
SEQ ID NO. 29: A14-ME- TCGTCGGCAGCGTCAGATGTGTATAAGAGACAG
SEQ ID NO. 30: B15-ME- GTCTCGTGGGCTCGGAGATGTGTATAAGAGACAG
SEQ ID NO. 31: HYB2- GAGTAAGTGGAAGAGATAGGAAGG
SEQ ID NO. 32: HYB2-ME
GAGTAAGTGGAAGAGATAGGAAGGAGATGTGTATAAGAGACAG
SEQ ID NO. 33: HYB2′- CCTTCCTATCTCTTCCACTTACTC
SEQ ID NO. 34: HYB2′-ME -
CCTTCCTATCTCTTCCACTTACTCAGATGTGTATAAGAGACAG
SEQ ID NO. 35: HYB2′-block
CCTTCCTATCTCTTCCACTTACT-3′propanol
SEQ ID NO. 36: HYB2′-ME-block
CCTTCCTATCTCTTCCACTTACTCAGATGTGTATAAGAGACAG-3′propanol
SEQ ID NO. 37: ME′-A14′
CTGTCTCTTATACACATCTGACGCTGCCGACGA
SEQ ID NO. 38: A14′- GACGCTGCCGACGA
SEQ ID NO. 39: ME′-B15′- CTGTCTCTTATACACATCTCCGAGCCCACGAGAC
SEQ ID NO. 40: B15′- CCGAGCCCACGAGAC
SEQ ID NO. 41: ME′-HYB2
CTGTCTCTTATACACATCTGAGTAAGTGGAAGAGATAGGAAGG
SEQ ID NO. 42: ME′-HYB2′
CTGTCTCTTATACACATCTCCTTCCTATCTCTTCCACTTACTC
SEQ ID NO: 43 SBS3- ACACTCTTTCCCTACACGACGCTCTTCCGATCT
SEQ ID NO: 44 SBS3′- AGATCGGAAGAGCGTCGTGTAGGGAAAGAGTGT
SEQ ID NO: 45 SBS12- GTGACTGGAGTTCAGACGTGTGCTCTTCCGATCT
SEQ ID NO: 46 SBS12′- AGATCGGAAGAGCACACGTCTGAACTCCAGTCAC
SEQ ID NO: 47- 5′Dual-biotin-TTTTTTAATGATACGGCGACCACCGAGATCTACAC
SEQ ID NO: 48- Dual-Biotin 6T-P7-nonlin 5′Dual-biotin-
TTTTTTCAAGCAGAAGACGGCATACGAGAT
SEQ ID NO: 49- P5f-Adaptor -AATGATACGGCGACCACCGAGATCTACAC*T
SEQ ID NO: 50 P7f-Adaptor CAAGCAGAAGACGGCATACGAGA*T
SEQ ID NO: 51 Human DNA sequence from clone RP11-151A10 on chromosome 10, complete sequence.
GenBank Accession No: AL157888.16, Region, 137023 to 137173. 3′ to 5′ shown in FIG.
41. tctgggtcct gtgtcctgct ctgtgagctc tgcagccact cctgttgact ttgtgcctta atgcaaatga tccctctcct
tggtcacccg ctggggtttg cactgttctt gttgaccttt agcctatgcc ttcctgggaa cctcccctgg g
SEQ ID NO: 52. Escherichia phiX174 virus strain evolved J1, complete genome.
GenBank Accession No: MH378443.1. Region 5039 to 5187. 3′ to 5′ shown in FIG. 41.
Tatggaccttgctgctaaaggtctaggagctaaagaatggaacaactcactaaaaaccaagctgtcgctacttccca
agaagctgttcagaatcagaatgagccgcaacttcgggatgaaaatgctcacaatgacaaatctgtccacgg
SEQ ID NO: 53. Escherichia phiX174 virus strain evolved J1, complete genome.
GenBank Accession No: MH378443.1. Region 3923 to 4072. 3′ to 5′ shown in FIG. 43.
aggattgaca ccctcccaat tgtatgtttt catgcctcca aatcttggag gcttttttat ggttcgtcct tattaccctt
ctgaatgtca cgctgattat tttgactttg agcgtatcga ggctcttaaa cctgctattg aggcttgtgg
SEQ ID NO: 54. Escherichia phiX174 virus strain evolved J1, complete genome.
GenBank Accession No: MH378443.1. Region 5067 to 5217. 3′ to 5′ shown in FIG. 43.
gctaaagaat ggaacaactc actaaaaacc aagctgtcgc tacttcccaa gaagctgttc agaatcagaa tgagccgcaa
cttcgggatg aaaatgctca caatgacaaa tctgtccacg gagtgcttaa tccaacttac caagctgggt t
SEQ ID NO: 55. Escherichia phiX174 virus strain evolved J1, complete genome.
GenBank Accession No: MH378443.1. Region 4171 to 4320. 3′ to 5′ shown in FIG. 43.
gcgttgagtt cgataatggt gatatgtatg ttgacggcca taaggctgct tctgacgttc gtgatgagtt tgtatctgtt
actgagaagt taatggatga attggcacaa tgctacaatg tgctccccca acttgatatt aataacacta
SEQ ID NO: 56. Escherichia phiX174 virus strain evolved J1, complete genome. GenBank
Accession No: MH378443.1. Region 5016-5168. 3′ to 5′ shown in FIG. 43.
atacgttaac aaaaagtcag atatggacct tgctgctaaa ggtctaggag ctaaagaatg gaacaactca
ctaaaaacca agctgtcgct acttcccaag aagctgttca gaatcagaat gagccgcaac ttcgggatga aaatgctcac
a

1. A method of preparing at least one polynucleotide library strand template, wherein the method comprises:
attaching a first adaptor to a first end of a double-stranded polynucleotide sequence, wherein the first end comprises 3′ end of the forward strand and 5′ end of the reverse strand of the double-stranded polynucleotide sequence; and
attaching a second adaptor to a second end of a double-stranded polynucleotide sequence, wherein the second end comprises 5′ end of the forward strand and 3′ end of the reverse strand of the double-stranded polynucleotide sequence;
wherein the first adaptor comprises a polynucleotide loop and the second adaptor comprises at least one primer-binding sequence and at least one primer-binding complement sequence;
wherein the first adaptor comprises a first restriction site for an endonuclease and/or wherein the second adaptor further comprises at least one cleavable site and/or a complement of a cleavable site.

2. The method according to claim 1, wherein the first adaptor comprises a base-paired stem and a loop, wherein the first restriction site is in the base-paired stem or is in the loop, and wherein the first restriction site is a restriction site for a nicking endonuclease or a restriction endonuclease.

3.-4. (canceled)

5. The method according to claim 1, wherein the first adaptor comprises a first restriction site for an endonuclease and the second adaptor comprises at least one cleavable site and/or a complement of a cleavable site.

6. The method according to claim 1, wherein the second adaptor comprises a base-paired stem and a fork, wherein the fork comprises a primer-binding complement sequence and a primer-binding sequence, optionally wherein the second adaptor's at least one cleavable site and/or a complement of a cleavable site is in the base-paired stem.

7. (canceled)

8. The method according to claim 1, wherein the second adaptor comprises a base-paired stem and a loop, wherein the loop comprises a second cleavable site, and optionally wherein the second cleavable site is a restriction site for a nicking endonuclease.

9.-10. (canceled)

11. A polynucleotide library strand for sequencing comprising a first adaptor, a double-stranded polynucleotide sequence to be identified and a second adaptor;
wherein the first adaptor is attached to a first end of the double-stranded polynucleotide sequence, wherein the first end comprises 3′ end of the forward strand and 5′ end of the reverse strand of the double-stranded polynucleotide sequence; and
wherein the second adaptor is attached to a second end of the double-stranded polynucleotide sequence, wherein the second end comprises 5′ end of the forward strand and 3′ end of the reverse strand of the double-stranded polynucleotide sequence;
wherein the first adaptor comprises a base-paired stem and a loop; and
wherein the second adaptor comprises a base-paired stem, a primer-binding complement sequence and a primer-binding sequence; and
wherein the first adaptor comprises at least one restriction site for an endonuclease; optionally
wherein the second adaptor comprises at least one cleavable site and/or a complement of a cleavable site, wherein the cleavable site and/or a complement of a cleavable site is preferably a restriction site for a nicking endonuclease

12. (canceled)

13. A method of identifying at least a first region of a polynucleotide sequence, wherein the method comprises:
a. preparing at least one polynucleotide library strand according to the method of claim 1;
b. amplifying the polynucleotide library strand to generate a first and second library strand, wherein each library strand comprises a first and second region;
c. hybridising the first or second library strands to first and second immobilised primers respectively on a solid support and carrying out a first extension reaction to generate a first or second immobilised template strand;
d. hybridising the first or second immobilised template strands to a second or first immobilised primer respectively and carrying out a second extension reaction to generate a second and first immobilised template strand;
e. hybridising the first and second immobilised template strands;
f. applying a first endonuclease; and
g. sequencing the first and second immobilised template strands, wherein sequencing the first and second immobilised template strands identifies the first region.

14. (canceled)

15. The method of claim 13, wherein each first and second library strands comprise a primer-binding complement sequence, a first portion, a first adaptor sequence, a second portion and a primer-binding sequence, and wherein the first adaptor comprises a first restriction site for an endonuclease, optionally wherein the primer-binding sequence and primer-binding complement sequence comprise at least one cleavable and/or a complement of a cleavable site.

16.-22. (canceled)

23. The method of claim 13, wherein the method further comprises blocking all or substantially all 3′ ends of the sequenced immobilised strands.

24. The method of claim 13, wherein the method further comprises applying a second nicking endonuclease and sequencing the first and second immobilised template strands identifies the second region, wherein the second nicking endonuclease cleaves a different restriction site from the first nicking endonuclease.

25.-26. (canceled)

27. A library preparation kit comprising of a plurality of first adaptors and a plurality of second adaptors, wherein the first adaptors comprise a base-paired stem and a loop, and wherein the first adaptors comprise at least one restriction site, and wherein the second adaptors comprise a base-paired stem, a primer-binding sequence and a primer-binding complement sequence, wherein optionally the second adaptors comprise at least one restriction site.

28. A method of preparing polynucleotide sequences for detection of mismatched base pairs, comprising:
synthesising at least one first polynucleotide sequence comprising a first portion and at least one second polynucleotide sequence comprising a second portion,
wherein the at least one first polynucleotide sequence comprising a first portion and the at least one second polynucleotide sequence comprising a second portion each comprise portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template.

29.-72. (canceled)

73. A method of preparing at least one polynucleotide sequence for detection of mismatched base pairs, comprising:
synthesising at least one polynucleotide sequence comprising a first portion and a second portion,
wherein the at least one polynucleotide sequence comprises portions of a double-stranded nucleic acid template, and the first portion comprises a forward strand of the template, and the second portion comprises a reverse complement strand of the template; or wherein the first portion comprises a reverse strand of the template, and the second portion comprises a forward complement strand of the template.

74.-117. (canceled)

118. A method of preparing at least one polynucleotide sequence for identification, comprising:
selectively processing at least one polynucleotide sequence comprising a first portion and a second portion, or at least one first polynucleotide sequence comprising a first portion and at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal; optionally wherein the first signal and the second signal are spatially unresolved.

119. The method according to claim 118, wherein a concentration of the first portions capable of generating the first signal is greater than a concentration of the second portions capable of generating the second signal, and a ratio between the concentration of the first portions capable of generating the first signal and the concentration of the second portions capable of generating the second signal is between 1.25:1 to 5:1, preferably between 1.5:1 to 3:1, more preferably about 2:1.

120. (canceled)

121. The method according to claim 118, comprising selectively processing at least one polynucleotide sequence comprising a first portion and a second portion, or selectively processing at least one first polynucleotide sequence comprising a first portion and at least one second polynucleotide sequence comprising a second portion.

122.-143. (canceled)

144. The method according to claim 118, wherein the selective processing comprises selectively removing some or substantially all of second immobilised primers that are not yet extended, and conducting a further amplification cycle in order to selectively amplify the first polynucleotide sequence(s) relative to the second polynucleotide sequence(s), or alternatively comprises selectively blocking some or substantially all of second immobilised primers that are not yet extended using a primer blocking agent, wherein the primer blocking agent is configured to limit or prevent synthesis of a strand extending from the second immobilised primer, and conducting a further amplification cycle in order to selectively amplify the first polynucleotide sequence(s) relative to the second polynucleotide sequence(s).

145.-172. (canceled)

173. A method of base calling nucleobases of two or more polynucleotide sequence portions, the method comprising:
(a) obtaining first intensity data comprising a combined intensity of a first signal obtained based upon a respective first nucleobase of at least one first polynucleotide sequence portion and a second signal obtained based upon a respective second nucleobase of at least one second polynucleotide sequence portion;
(b) obtaining second intensity data comprising a combined intensity of a third signal obtained based upon the respective first nucleobase of the at least one first polynucleotide sequence portion and a fourth signal obtained based upon the respective second nucleobase of the at least one second polynucleotide sequence portion;
(c) selecting one of a plurality of classifications based on the first and the second intensity data, wherein each classification represents a possible combination of respective first and second nucleobases; and
(d) based on the selected classification, base calling the respective first and second nucleobases,
wherein said polynucleotide sequence portions have been selectively processed such that an intensity of the signals obtained based upon the respective first nucleobase is greater than an intensity of the signals obtained based upon the respective second nucleobase; and optionally wherein the first and second signals and/or the third and fourth signals are obtained substantially simultaneously.

174. (canceled)

175. The method of claim 173, wherein selecting the classification based on the first and second intensity data comprises selecting the classification based on the combined intensity of the first and second signals and the combined intensity of the third and fourth signals.

176.-207. (canceled)

208. A method of preparing at least one polynucleotide sequence for identification, comprising:
selectively processing at least one polynucleotide sequence comprising n portions, such that a proportion of each of the n portions are each capable of generating a respective n^{th} signal,
wherein n is 2 or more, and
wherein the selective processing causes an intensity of an i^{th} signal to be different compared to an intensity of a j^{th} signal, for all i between 1 to n, and for all j between 1 to n, and where i is not equal to j.

209. The method according to claim 208, wherein a concentration of each of the i^{th} portions capable of generating the i^{th} signal is different compared to a concentration of each of the j^{th} portions capable of generating the j^{th} signal, and wherein a ratio between a concentration of one of the n portions capable of generating the (m−1)^{th} most intense signal and a concentration of another of the n portions capable of generating the m^{th} most intense signal is between 1.25:1 to 5:1, optionally between 1.5:1 to 3:1, or about 2:1, wherein m is between 2 to n.

210.-264. (canceled)

265. The library preparation kit of claim 27, wherein the second adaptors comprise at least one restriction site, optionally wherein the at least one restriction site of the first adaptor is a restriction site for a nicking endonuclease or a restriction endonuclease and wherein the at least one restriction site of the second adaptors is a restriction site for a nicking endonuclease.


SEQUENCE LISTING (56 sequences)

SEQ ID NO: 1 - DNA, 29 bp, synthetic construct
  source 1..29: mol_type = other DNA
aatgatacggcgaccaccgagatctacac

SEQ ID NO: 2 - DNA, 24 bp, synthetic construct
  source 1..24: mol_type = other DNA
caagcagaagacggcatacgagat

SEQ ID NO: 3 - DNA, 29 bp, synthetic construct
  source 1..29: mol_type = other DNA
gtgtagatctcggtggtcgccgtatcatt

SEQ ID NO: 4 - DNA, 23 bp, synthetic construct
  source 1..23: mol_type = other DNA
atctcgtatgccgtcttctgctt

SEQ ID NO: 5 - DNA, 17 bp, synthetic construct
  source 1..17: mol_type = other DNA
aatgatacggcgaccga

SEQ ID NO: 6 - DNA, 17 bp, synthetic construct
  source 1..17: mol_type = other DNA
tcggtcgccgtatcatt

SEQ ID NO: 7 - DNA, 70 bp, synthetic construct
  source 1..70: mol_type = other DNA
  modified_base 32: mod_base = OTHER; note = uracil
  misc_feature 69^70: note = phosphorothioate linkage
gctgaggatctcgtatgccgtcttctgcttgtaatgatacggcgaccaccgagatctacactcctcagct

SEQ ID NO: 8 - DNA, 69 bp, synthetic construct
  source 1..69: mol_type = other DNA
  misc_feature 1..7: note = nicking restriction site (or its complement) of Nt.BspQI
  misc_feature 62..69: note = nicking restriction site (or its complement) of Nt.BspQI
  misc_feature 68^69: note = phosphorothioate linkage
  modified_base 35: mod_base = OTHER; note = Biotin-T
gaagagcacacgtctgaactccagtcactagggataacagggtaatctttccctacacgacgctcttct

SEQ ID NO: 9 - DNA, 70 bp, synthetic construct; identical to SEQ ID NO: 7
  source 1..70: mol_type = other DNA
  modified_base 32: mod_base = OTHER; note = uracil
  misc_feature 69^70: note = phosphorothioate linkage
  modified_base 2: mod_base = OTHER; note = 5-methylcytosine
  modified_base 10: mod_base = OTHER; note = 5-methylcytosine
  modified_base 12: mod_base = OTHER; note = 5-methylcytosine
  modified_base 18..19: mod_base = OTHER; note = 5-methylcytosine
  modified_base 22: mod_base = OTHER; note = 5-methylcytosine
  modified_base 25: mod_base = OTHER; note = 5-methylcytosine
  modified_base 28: mod_base = OTHER; note = 5-methylcytosine
  modified_base 40: mod_base = OTHER; note = 5-methylcytosine
  modified_base 43: mod_base = OTHER; note = 5-methylcytosine
  modified_base 46..47: mod_base = OTHER; note = 5-methylcytosine
  modified_base 49..50: mod_base = OTHER; note = 5-methylcytosine
  modified_base 56: mod_base = OTHER; note = 5-methylcytosine
  modified_base 59: mod_base = OTHER; note = 5-methylcytosine
  modified_base 61..62: mod_base = OTHER; note = 5-methylcytosine
  modified_base 64: mod_base = OTHER; note = 5-methylcytosine
  modified_base 67: mod_base = OTHER; note = 5-methylcytosine

SEQ ID NO: 10 - DNA, 69 bp, synthetic construct; identical to SEQ ID NO: 8
  source 1..69: mol_type = other DNA
  misc_feature 1..7: note = nicking restriction site (or its complement) of Nt.BspQI
  misc_feature 62..69: note = nicking restriction site (or its complement) of Nt.BspQI
  modified_base 35: mod_base = OTHER; note = Biotin-T
  misc_feature 68^69: note = phosphorothioate linkage
  modified_base 7: mod_base = OTHER; note = 5-methylcytosine
  modified_base 9: mod_base = OTHER; note = 5-methylcytosine
  modified_base 11: mod_base = OTHER; note = 5-methylcytosine
  modified_base 14: mod_base = OTHER; note = 5-methylcytosine
  modified_base 19: mod_base = OTHER; note = 5-methylcytosine
  modified_base 21..22: mod_base = OTHER; note = 5-methylcytosine
  modified_base 28: mod_base = OTHER; note = 5-methylcytosine
  modified_base 38: mod_base = OTHER; note = 5-methylcytosine
  modified_base 47: mod_base = OTHER; note = 5-methylcytosine
  modified_base 51..53: mod_base = OTHER; note = 5-methylcytosine
  modified_base 56: mod_base = OTHER; note = 5-methylcytosine
  modified_base 58: mod_base = OTHER; note = 5-methylcytosine
  modified_base 61: mod_base = OTHER; note = 5-methylcytosine
  modified_base 63: mod_base = OTHER; note = 5-methylcytosine
  modified_base 65: mod_base = OTHER; note = 5-methylcytosine
  modified_base 68: mod_base = OTHER; note = 5-methylcytosine

SEQ ID NO: 11 - DNA, 37 bp, synthetic construct
  source 1..37: mol_type = other DNA
  modified_base 31: mod_base = OTHER; note = 2-deoxyuridine
ttttttttttaatgatacggcgaccaccgatctacac

SEQ ID NO: 12 - DNA, 34 bp, synthetic construct
  source 1..34: mol_type = other DNA
  modified_base 31: mod_base = OTHER; note = 8-oxoguanine
ttttttttttcaagcagaagacggcatacgagat

SEQ ID NO: 13 - DNA, 30 bp, synthetic construct
  source 1..30: mol_type = other DNA
agtgtagatctcggtggtcgccgtatcatt

SEQ ID NO: 14 - DNA, 30 bp, synthetic construct
  source 1..30: mol_type = other DNA
tgtgtagatctcggtggtcgccgtatcatt

SEQ ID NO: 15 - DNA, 30 bp, synthetic construct
  source 1..30: mol_type = other DNA
cgtgtagatctcggtggtcgccgtatcatt

SEQ ID NO: 16 - DNA, 30 bp, synthetic construct
  source 1..30: mol_type = other DNA
ggtgtagatctcggtggtcgccgtatcatt

SEQ ID NO: 17 - DNA, 25 bp, synthetic construct
  source 1..25: mol_type = other DNA
aatctcgtatgccgtcttctgcttg

SEQ ID NO: 18 - DNA, 25 bp, synthetic construct
  source 1..25: mol_type = other DNA
tatctcgtatgccgtcttctgcttg

SEQ ID NO: 19 - DNA, 25 bp, synthetic construct
  source 1..25: mol_type = other DNA
catctcgtatgccgtcttctgcttg

SEQ ID NO: 20 - DNA, 25 bp, synthetic construct
  source 1..25: mol_type = other DNA
gatctcgtatgccgtcttctgcttg

SEQ ID NO: 21 - DNA, 18 bp, synthetic construct
  source 1..18: mol_type = other DNA
atcggtcgccgtatcatt

SEQ ID NO: 22 - DNA, 18 bp, synthetic construct
  source 1..18: mol_type = other DNA
ttcggtcgccgtatcatt

SEQ ID NO: 23 - DNA, 18 bp, synthetic construct
  source 1..18: mol_type = other DNA
ctcggtcgccgtatcatt

SEQ ID NO: 24 - DNA, 18 bp, synthetic construct
  source 1..18: mol_type = other DNA
gtcggtcgccgtatcatt

SEQ ID NO: 25 - DNA, 70 bp, synthetic construct; identical to SEQ ID NO: 7
  source 1..70: mol_type = other DNA
  modified_base 32: mod_base = OTHER; note = uracil
  misc_feature 69^70: note = phosphorothioate linkage

SEQ ID NO: 26 - DNA, 69 bp, synthetic construct; identical to SEQ ID NO: 8
  source 1..69: mol_type = other DNA
  modified_base 35: mod_base = OTHER; note = Biotin-T
  misc_feature 68^69: note = phosphorothioate linkage

SEQ ID NO: 27 - DNA, 14 bp, synthetic construct
  source 1..14: mol_type = other DNA
tcgtcggcagcgtc

SEQ ID NO: 28 - DNA, 15 bp, synthetic construct
  source 1..15: mol_type = other DNA
gtctcgtgggctcgg

SEQ ID NO: 29 - DNA, 33 bp, synthetic construct
  source 1..33: mol_type = other DNA
  misc_feature 15..33: note = ME sequence
tcgtcggcagcgtcagatgtgtataagagacag

SEQ ID NO: 30 - DNA, 34 bp, synthetic construct
  source 1..34: mol_type = other DNA
  misc_feature 16..34: note = ME sequence
gtctcgtgggctcggagatgtgtataagagacag

SEQ ID NO: 31 - DNA, 24 bp, synthetic construct
  source 1..24: mol_type = other DNA
gagtaagtggaagagataggaagg

SEQ ID NO: 32 - DNA, 43 bp, synthetic construct
  source 1..43: mol_type = other DNA
  misc_feature 25..43: note = ME sequence
gagtaagtggaagagataggaaggagatgtgtataagagacag

SEQ ID NO: 33 - DNA, 23 bp, synthetic construct
  source 1..23: mol_type = other DNA
cttcctatctcttccacttactc

SEQ ID NO: 34 - DNA, 43 bp, synthetic construct
  source 1..43: mol_type = other DNA
  misc_feature 25..43: note = ME sequence
ccttcctatctcttccacttactcagatgtgtataagagacag

SEQ ID NO: 35 - DNA, 23 bp, synthetic construct
  source 1..23: mol_type = other DNA
  misc_feature 23: note = 3' propanol
ccttcctatctcttccacttact

SEQ ID NO: 36 - DNA, 43 bp, synthetic construct; identical to SEQ ID NO: 34
  source 1..43: mol_type = other DNA
  misc_feature 25..43: note = ME sequence
  misc_feature 43: note = 3'Propanol

SEQ ID NO: 37 - DNA, 33 bp, synthetic construct
  source 1..33: mol_type = other DNA
  misc_feature 1..19: note = ME sequence
ctgtctcttatacacatctgacgctgccgacga

SEQ ID NO: 38 - DNA, 14 bp, synthetic construct
  source 1..14: mol_type = other DNA
gacgctgccgacga

SEQ ID NO: 39 - DNA, 34 bp, synthetic construct
  source 1..34: mol_type = other DNA
  misc_feature 1..19: note = ME sequence
ctgtctcttatacacatctccgagcccacgagac

SEQ ID NO: 40 - DNA, 15 bp, synthetic construct
  source 1..15: mol_type = other DNA
ccgagcccacgagac

SEQ ID NO: 41 - DNA, 43 bp, synthetic construct
  source 1..43: mol_type = other DNA
  misc_feature 1..19: note = ME sequence
ctgtctcttatacacatctgagtaagtggaagagataggaagg

SEQ ID NO: 42 - DNA, 43 bp, synthetic construct
  source 1..43: mol_type = other DNA
  misc_feature 1..19: note = ME sequence
ctgtctcttatacacatctccttcctatctcttccacttactc

SEQ ID NO: 43 - DNA, 33 bp, synthetic construct
  source 1..33: mol_type = other DNA
acactctttccctacacgacgctcttccgatct

SEQ ID NO: 44 - DNA, 33 bp, synthetic construct
  source 1..33: mol_type = other DNA
agatcggaagagcgtcgtgtagggaaagagtgt

SEQ ID NO: 45 - DNA, 34 bp, synthetic construct
  source 1..34: mol_type = other DNA
gtgactggagttcagacgtgtgctcttccgatct

SEQ ID NO: 46 - DNA, 34 bp, synthetic construct
  source 1..34: mol_type = other DNA
agatcggaagagcacacgtctgaactccagtcac

SEQ ID NO: 47 - DNA, 35 bp, synthetic construct
  source 1..35: mol_type = other DNA
  misc_feature 1: note = 5' dual biotin
ttttttaatgatacggcgaccaccgagatctacac

SEQ ID NO: 48 - DNA, 30 bp, synthetic construct
  source 1..30: mol_type = other DNA
  misc_feature 1: note = 5' dual biotin
ttttttcaagcagaagacggcatacgagat

SEQ ID NO: 49 - DNA, 24 bp, synthetic construct
  source 1..24: mol_type = other DNA
  misc_feature 23^24: note = phosphorothioate linkage
caagcagaagacggcatacgagat

SEQ ID NO: 50 - DNA, 24 bp, synthetic construct
  source 1..24: mol_type = other DNA
  misc_feature 23^24: note = phosphorothioate linkage
caagcagaagacggcatacgagat

SEQ ID NO: 51 - DNA, 151 bp, synthetic construct
  source 1..151: mol_type = other DNA
tctgggtcctgtgtcctgctctgtgagctctgcagccactcctgttgactttgtgccttaatgcaaatgatccctctccttggtcacccgctggggtttgcactgttcttgttgacctttagcctatgccttcctgggaacctcccctggg

SEQ ID NO: 52 - DNA, 149 bp, synthetic construct
  source 1..149: mol_type = other DNA
tatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacgg

SEQ ID NO: 53 - DNA, 150 bp, synthetic construct
  source 1..150: mol_type = other DNA
aggattgacaccctcccaattgtatgttttcatgcctccaaatcttggaggcttttttatggttcgtccttattacccttctgaatgtcacgctgattattttgactttgagcgtatcgaggctcttaaacctgctattgaggcttgtgg

SEQ ID NO: 54 - DNA, 151 bp, synthetic construct
  source 1..151: mol_type = other DNA
gctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggtt

SEQ ID NO: 55 - DNA, 150 bp, synthetic construct
  source 1..150: mol_type = other DNA
gcgttgagttcgataatggtgatatgtatgttgacggccataaggctgcttctgacgttcgtgatgagtttgtatctgttactgagaagttaatggatgaattggcacaatgctacaatgtgctcccccaacttgatattaataacacta

SEQ ID NO: 56 - DNA, 151 bp, synthetic construct
  source 1..151: mol_type = other DNA
atacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcaca